(19) United States
(12) Patent Application Publication
(10) Pub. No.: US 2024/0390484 A1
(43) Pub. Date: Nov. 28, 2024

(54) RNA FORMULATIONS AND LIPIDS

(71) Applicant: Immorna (Hangzhou) Biotechnology Co., Ltd., Hangzhou (CN)

(72) Inventors: Zihao Wang, Hangzhou (CN); Yuanqing Liu, Hangzhou (CN); Yanni Chen, Hangzhou (CN); Qizhi Hu, Hangzhou (CN); Zhijun Guo, Hangzhou (CN); Hongyue Wu, Hangzhou (CN)

(21) Appl. No.: 18/667,934

(22) Filed: May 17, 2024

Related U.S. Application Data
(63) Continuation of application No. PCT/CN2022/137326, filed on Dec. 7, 2022.

(51) Int. Cl. A61K 39/25; A61K 9/127; A61K 9/51; A61K 39/00; A61P 31/22; C12N 7/00

(52) U.S. Cl. CPC A61K 39/25; A61K 9/1271; A61K 9/5123; A61P 31/22; C12N 7/00; A61K 2039/53; A61K 2039/55555; C12N 2710/16734; C12N 2770/20034

(57) ABSTRACT

The disclosure relates to the method of lyophilizing RNA and mixing with a liquid LNP solution, e.g., to make an RNA vaccine or therapeutic. Included are methods for preparing and administering the vaccine or therapeutic.

### CROSS-REFERENCE TO RELATED APPLICATIONS

This application is a continuation of International Application Number PCT/CN2022/137326 filed on Dec. 7, 2022, which claims the benefit of and priority to PCT/CN2021/136110, filed on Dec. 7, 2021, and PCT/CN2022/118152, filed Sep. 9, 2022, each of which are hereby incorporated herein by reference in their entireties for all purposes.

### REFERENCE TO A SEQUENCE LISTING

The present application contains a Sequence Listing which has been submitted electronically in XML format. The Sequence Listing XML is hereby incorporated by reference in its entirety. Said XML file, created on Dec. 7, 2022, is named IMO-001W03-SL-as-filed.xml and is 36,864 bytes in size.

### BACKGROUND

Messenger RNA (mRNA) is a single-stranded, anionic RNA molecule that can affect expression of a desired (disease-specific antigen) protein, once reaching the cytoplasm of the target cells. To date, RNA has demonstrated great potential for vaccine and therapeutic applications, although efficient intracellular delivery of RNA remains a major challenge. Lipid nanoparticles (LNPs) have emerged as the most promising nonviral delivery vehicles, which encapsulate and protect the delicate RNA from degradation as well as promote its intracellular uptake and expression. Currently, the only FDA-approved mRNA products are mRNA vaccines against COVID-19 (mRNA-1273 and BNT162b2), in which the anionic mRNA is encapsulated inside LNPs through electrostatic interactions with cationic/ionizable lipid components, generating a LNP-encapsulated mRNA liquid drug product. However, because of the intrinsic instability of mRNA, cold chain management and storage at ultra-low temperature are required for LNP-encapsulated mRNA liquid drug products, limiting their global use.

Varicella is an acute infectious disease caused by varicella zoster virus (VZV). Varicella zoster virus is one of eight herpesviruses known to infect humans and vertebrates. VZV is also known as chickenpox virus, varicella virus, zoster virus, and human herpesvirus type 3 (HHV-3). VZV only affects humans, and commonly causes chickenpox in children, teens and young adults and herpes zoster (shingles) in adults (rarely in children). The primary VZV infection, which results in chickenpox (varicella), may result in complications, including viral or secondary bacterial pneumonia. Even when the clinical symptoms of chickenpox have resolved, VZV remains dormant in the nervous system of the infected person (virus latency) in the trigeminal and dorsal root ganglia. In about 10-20% of cases, with the risk rising to 50% after the age of 80 (Johnson R W et al., Ther Adv Vaccines, 3:109-120, 2015), VZV reactivates later in life, travelling from the sensory ganglia back to the skin where it produces a disease (rash) known as shingles or herpes zoster. VZV can also cause a number of neurologic conditions ranging from aseptic meningitis to encephalitis. Other serious complications of VZV infection include postherpetic neuralgia, Mollaret's meningitis, zoster multiplex, thrombocytopenia, myocarditis, arthritis, and inflammation of arteries in the brain leading to stroke, myelitis, herpes ophthalmicus, or zoster sine herpete. In rare instances, VZV affects the geniculate ganglion, giving lesions that follow specific branches of the facial nerve. Symptoms may include painful blisters on the tongue and ear along with one sided facial weakness and hearing loss.

Currently, there are two approved vaccines against Shingles: Zostavax of Merck and Shingrix of GSK. Zostavax is a live attenuated virus vaccine. Zostavax is a concentrated version of vaccine Oka, which was approved by US FDA in 2006. The effectiveness of Zostavax decreases with age of the vaccines, and thus is not recommended for people over the age of 60. It has now been shown to provide 50% protection in about five years, followed by a progressive decrease in efficacy during 5-8 years after vaccination, and its protection efficacy is no longer statistically significant after 8 years after vaccination (Morrison V A, et al., Clin Infect Dis, 60:900-909, 2015). Shingrix of GSK adopts gene recombination technology to express varicella zoster virus glycoprotein E (gE) and it was approved by the FDA in 2017 to be used in people aged 50 or more. Shingrix is a recombinant subunit protein-based vaccine. Shingrix, with a 90% protection rate for shingles, reduces the risk of postherpetic neuralgia and is a preferred substitute of Zostavax. However, the adjuvant used in Shingrix is AS01 from GSK, which has relatively severe side effects. The use of this adjuvant can also face limited supply issues as one of the components is naturally sourced (Quillaja saponaria Molina tree bark).

Described herein is a new modality of VZV vaccine that uses self-replicating RNA encoding a VZV viral protein(s) and, when delivered in vivo, triggers strong anti-viral antibodies and T cell responses.

### SUMMARY

It has now been discovered that storage and transportation challenges for RNA vaccine and therapeutics may be overcome by manufacturing LNP-formulated RNA products in a different manner, whereby the RNA is lyophilized and the lyophilized RNA is electrostatically adsorbed to the exterior of LNP instead of being encapsulated within when initially mixed with LNP. Following this novel approach, in practice, the clinical presentation involves two vials: one is LNP in liquid form stored at about 2-8° C.; the other is lyophilized RNA stored at about 2-8° C. or even at room temperature. The LNP and the RNA contents are mixed just prior to the clinical use at bedside, making the product “ready-to-use.” This novel approach eliminates the ultra-low temperature cold chain storage required for the currently commercially available LNP-encapsulated RNA liquid drug products. Despite the change in product construct, RNA products developed based on this new concept are expected to be as safe and effective as the widely evaluated, LNP-encapsulated counterpart.

Described herein is a method of lyophilizing RNA and mixing with a liquid LNP solution, and uses thereof.

For example, described herein is a composition or set of compositions separately comprising each of (1) a lyophilized polynucleotide composition and (2) a liquid lipid nanoparticle (LNP) solution.

In some embodiments, the polynucleotide encodes a protein, a polypeptide, or an antigen.

In some embodiments, the polynucleotide comprises at least one of a ribosomal RNA (rRNA), transfer RNA (tRNA), viral RNA, retroviral RNA, self-replicating RNA (replicon RNA), small interfering RNA (siRNA), microRNA, small nuclear RNA (snRNA), small-hairpin (sh) RNA, a riboswitch, a ribozyme, an oligonucleotide, or an aptamer.

In some embodiments, the polynucleotide comprises an RNA, optionally a messenger RNA (mRNA) or a self-replicating RNA.

In some embodiments, the RNA is a not a self-replicating RNA.

In some embodiments, the RNA is a self-replicating RNA.

In some embodiments, the water content of the lyophilized RNA composition is less than 10%.

In some embodiments, the water content of the lyophilized RNA composition is less than 5%.

In some embodiments, the water content of the lyophilized RNA composition is less than 3%.

In some embodiments, the lyophilized RNA composition is at about 2-8° C.

In some embodiments, the lyophilized RNA composition comprises at least one suitable protective agent.

In some embodiments, the suitable protective agent comprises a polyol, a polymer, a sugar or any combination thereof.

In some embodiments, the suitable protective agent comprises sucrose.

In some embodiments, the concentration of the protective agent ranges from 0.5% to 30% by weight of the lyophilized RNA composition.

In some embodiments, the LNP comprises an ionizable lipid of Formula I

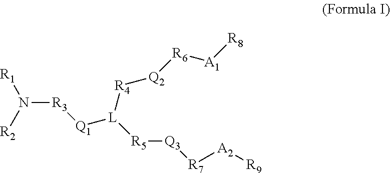

wherein:
R₁ and R₂ are each, independently C₁-C₆ alkyl;
R₃ is C₁-C₅ alkyl;
Q₁, Q₂ and Q₃ are each independently —O—, —S—, —C(O)O—, —OC(O)—, —S—S—, —C(O)S—, —SC(O)—, —OC(S)—, or —C(S)O—;
L is C₁-C₃ alkyl
R₄ and R₅ are each, independently C₁-C₁₀ alkyl;
R₆ and R₇ are each, independently C₁-C₁₀ alkyl, C₁-C₁₀ alkenyl
A₁ and A₂ are each independently a bond, —O—, —S—, —C(O)O—, —OC(O)—, —S—S—, —C(O)S—, —SC(O)—, —OC(S)—, or —C(S)O—;
R₈ and R₉ are each, independently C₁-C₁₀ alkyl.

In some embodiments, L is

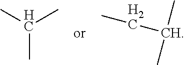

In some embodiments, the LNP comprises an ionizable lipid of Formula II

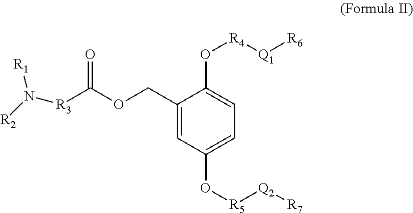

wherein:
R₁ and R₂ are each independently C₁ to C₆ alkyl;
R₃ is C₁ to C₅ alkyl;
R₄ and R₅ are each independently C₁ to C₁₈ alkyl group
Q₁ and Q₂ are each independently —O—C(O)—, —C(O)—O—, —O—C(S)—, —C(S)—O—; —S—S—, and
R₆ and R₇ are each independently C₁ to C₃₂ alkyl.

In some embodiments, R₄ and R₅ are each independently C₅-C₁₀ alkyl group.

In some embodiments, R₆ and R₇ are each independently C₁-C₂₂ alkyl. In some embodiments, R₆ and R₇ are each independently C₇-C₁₇ alkyl.

In some embodiments, R₁ and R₂ are methyl. In some embodiments, Q₁ and Q₂ are each, independently, —C(O)—O— or —O—C(O)—. In some embodiments, R₃ is C₃ alkyl.

In some embodiments, the LNP comprises a lipid selected from the group consisting of:

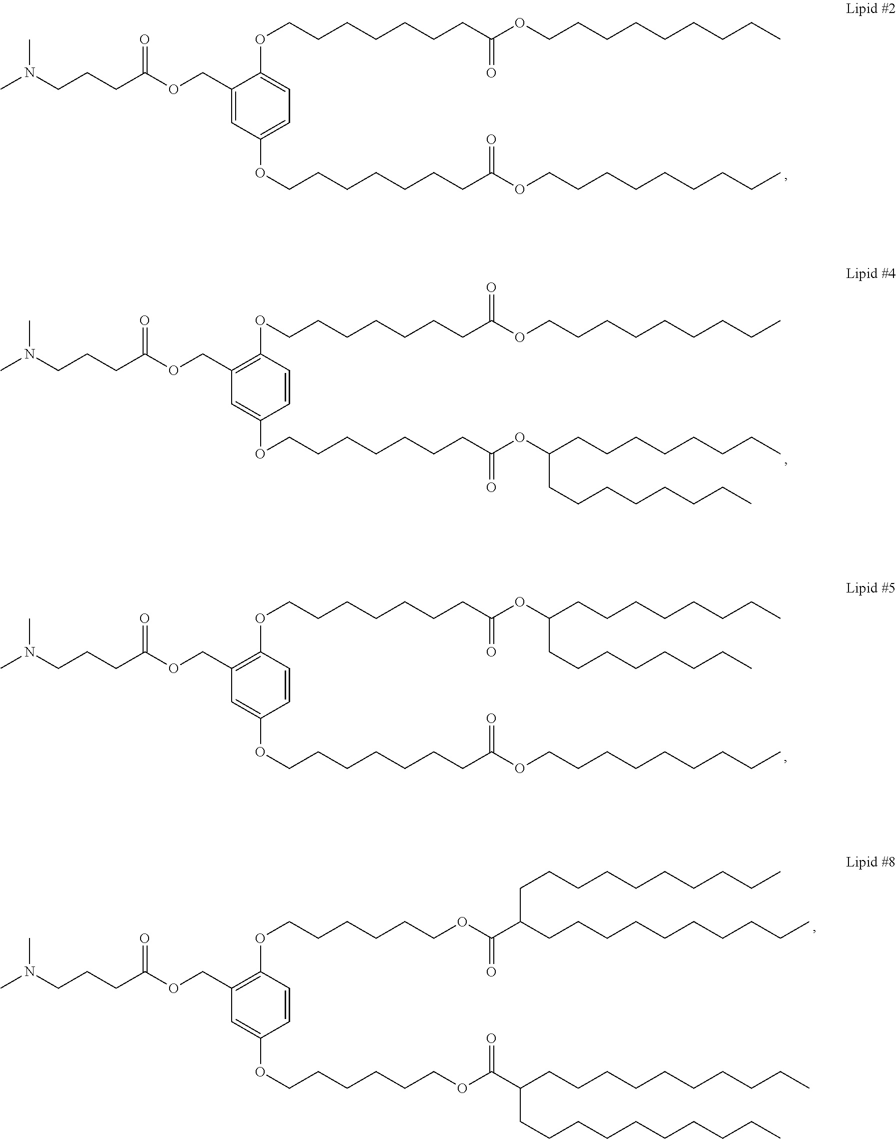

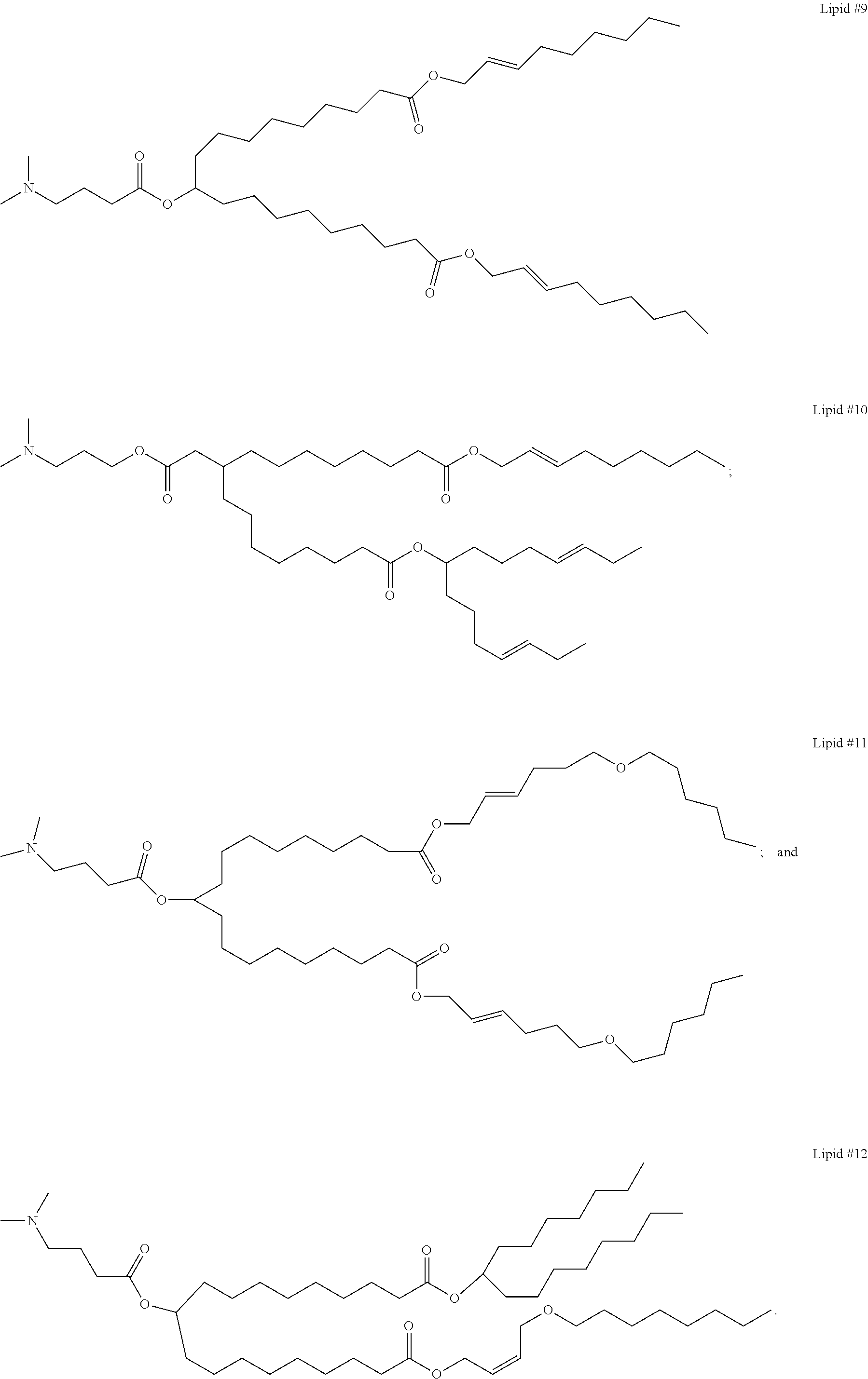

In some embodiments, the LNP comprises an ionizable lipid, a phospholipid, a structural lipid, and a pegylated lipid.

In some embodiments, the LNP comprises an ionizable lipid, 1,2-Dioleoyl-sn-glycero-3-phosphoethanolamine (DOPE), cholesterol, and a pegylated lipid, optionally 1,2-dimyristoyl-rac-glycero-3-methoxypolyethylene glycol-2000 (DMG-PEG 2000). In some embodiments, the LNP comprises an ionizable lipid, 1,2-diastearoyl-sn-glycero-3-phosphocholine (DSPC), cholesterol, and a pegylated lipid, optionally 1,2-dimyristoyl-rac-glycero-3-methoxypolyethylene glycol-2000 (DMG-PEG 2000).

In some embodiments, the LNP comprises a phospholipid: a structural lipid: a pegylated lipid: and an ionizable lipid with the mole ratio within the ranges of phospholipid: 5%-20%, structural lipid: 18.5%-58.5%, pegylated lipid: 1%-4%, ionizable lipid: 30%-70%, and the total summation of the mole ratio of the lipids is 100%.

In some embodiments, the LNP comprises a phospholipid: a structural lipid: a pegylated lipid: and an ionizable lipid with the mole ratio within the ranges of phospholipid: 5%-20%, structural lipid: 18.5%-58.5%, pegylated lipid: 1%-4%, ionizable lipid: 40%-55%, and the total summation of the mole ratio of the lipids is 100%.

In some embodiments, the LNP comprises a phospholipid: a structural lipid: a pegylated lipid: and an ionizable lipid with the mole ratio within the ranges of phospholipid: 5%-20%, structural lipid: 18.5%-58.5%, pegylated lipid: 1%-4%, ionizable lipid: 40%-50%, and the total summation of the mole ratio of the lipids is 100%.

In some embodiments, the LNP comprises a phospholipid: a structural lipid: a pegylated lipid: and an ionizable lipid with the mole ratio within the ranges of phospholipid: 5%-20%, structural lipid: 18.5%-58.5%, pegylated lipid: 1%-4%, ionizable lipid: 45%-50%, and the total summation of the mole ratio of the lipids is 100%.

In some embodiments, the LNP comprises a phospholipid: a structural lipid: a pegylated lipid: and an ionizable lipid with the mole ratio within the ranges of phospholipid: 5%-20%, structural lipid: 18.5%-58.5%, pegylated lipid: 1%-4%, ionizable lipid: 46%-49%, and the total summation of the mole ratio of the lipids is 100%.

In some embodiments, an ionizable lipid may comprise about 41%, 42%, 43%, 44%, 45%, 46%, 47%, 48% or 49% of the LNP. In some embodiments, the LNP comprises a phospholipid: a structural lipid: a pegylated lipid: and an ionizable lipid with a mole ratio of 10:40.5:1.5:48.

In some embodiments, the phospholipid is DOPE or DSPC.

In some embodiments, the pegylated lipid comprises PEG-modified phosphatidylethanolamines, PEG-modified phosphatidic acids, PEG-modified ceramides, PEG-modified dialkylamines, PEG-modified diacylglycerols, PEG-modified dialkylglycerols, or combinations thereof. In some embodiments, a PEGylated lipid may be PEG-c-DOMG, PEG-DMG, PEG-DLPE, PEG-DMPE, PEG-DPPC, or a PEG-DSPE lipid, or any combination thereof.

In some embodiments, the pegylated lipid comprises 1,2-dimyristoyl-rac-glycero-3-methoxypolyethylene glycol-2000 (DMG-PEG 2000), DMG-PEG 3500, DMG-PEG 5000, DTDAM-PEG 2000 (ALC-0159), DTDAM-PEG 5000, DMG-C-PEG 2000, DMG-C-PEG 5000, DSG-PEG 2000, DSG-PEG 5000, DPG-PEG 2000, DPG-PEG 5000, or any combination thereof.

In some embodiments, the structural lipid comprises cholesterol, fecosterol, sitosterol, ergosterol, campesterol, stigmasterol, brassicasterol, tomatidine, tomatine, ursolic acid, alpha-tocopherol, or combinations thereof. In some embodiments, the structural lipid is cholesterol. In some embodiments, the structural lipid includes cholesterol and a corticosteroid (such as prednisolone, dexamethasone, prednisone, and hydrocortisone), or a combination thereof.

In some embodiments, the LNP comprises a DSPC:cholesterol:DMG-PEG 2000:and an ionizable lipid with the mole ratio within the ranges of DSPC: 5%-20%, cholesterol: 30%-55%, DMG-PEG 2000: 1%-4%, ionizable lipid: 30%-70%, the total summation of the mole ratio of the lipids is 100%.

In some embodiments, the LNP comprises a DSPC:cholesterol:DMG-PEG 2000:and an ionizable lipid with the mole ratio within the ranges of DSPC: 5%-20%, cholesterol: 30%-55%, DMG-PEG 2000: 1%-4%, ionizable lipid: 40%-55%, the total summation of the mole ratio of the lipids is 100%, optionally 10:40.5:1.5:48.

In some embodiments, the LNP comprises a DSPC: cholesterol: 1,2-dimyristoyl-rac-glycero-3-methoxypolyethylene glycol-2000 (DMG-PEG 2000): and an ionizable lipid with the mole ratio within the ranges of DSPC: 5%-20%, cholesterol: 30%-55%, DMG-PEG 2000: 1%-4%, ionizable lipid: 40%-50%, the total summation of the mole ratio of the lipids is 100%.

In some embodiments, the LNP comprises a DSPC: cholesterol: 1,2-dimyristoyl-rac-glycero-3-methoxypolyethylene glycol-2000 (DMG-PEG 2000): and an ionizable lipid with the mole ratio within the ranges of DSPC: 5%-20%, cholesterol: 30%-55%, DMG-PEG 2000: 1%-4%, ionizable lipid: 45%-50%, the total summation of the mole ratio of the lipids is 100%.

In some embodiments, the LNP comprises a DSPC: cholesterol: 1,2-dimyristoyl-rac-glycero-3-methoxypolyethylene glycol-2000 (DMG-PEG 2000): and an ionizable lipid with the mole ratio within the ranges of DSPC: 5%-20%, cholesterol: 30%-55%, DMG-PEG 2000: 1%-4%, ionizable lipid: 46%-49%, the total summation of the mole ratio of the lipids is 100%.

In some embodiments, ionizable lipid may comprise about 41%, 42%, 43%, 44%, 45%, 46%, 47%, 48% or 49% of the LNP. In some embodiments, the LNP comprises a DSPC:cholesterol:DMG-PEG 2000:and an ionizable lipid with the mole ratio of 10:40.5:1.5:48.

In some embodiments, the LNP has a pH of 5 to 6.

In some embodiments, the LNP has a particle size of about 40-300 nm, optionally the LNP has a particle size of no greater than 160 nm, and optionally the LNP has a particle size of about 140-160 nm.

In some embodiments, there is a liquid composition comprising a lyophilized RNA electrostatically adsorbed to at least one exterior portion of a lipid nanoparticle (LNP), at an ionizable lipid:RNA (N/P) molar ratio ranging from 5:1 to 12:1, optionally at a N/P molar ratio ranging from 5:1 to 9:1, and optionally at a N/P molar ratio of 7:1.

In some embodiments, the LNP comprises an ionizable lipid of Formula I.

In some embodiments, the LNP comprises an ionizable lipid of Formula II.

In some embodiments, the LNP comprises an ionizable lipid of selected from the group consisting of:

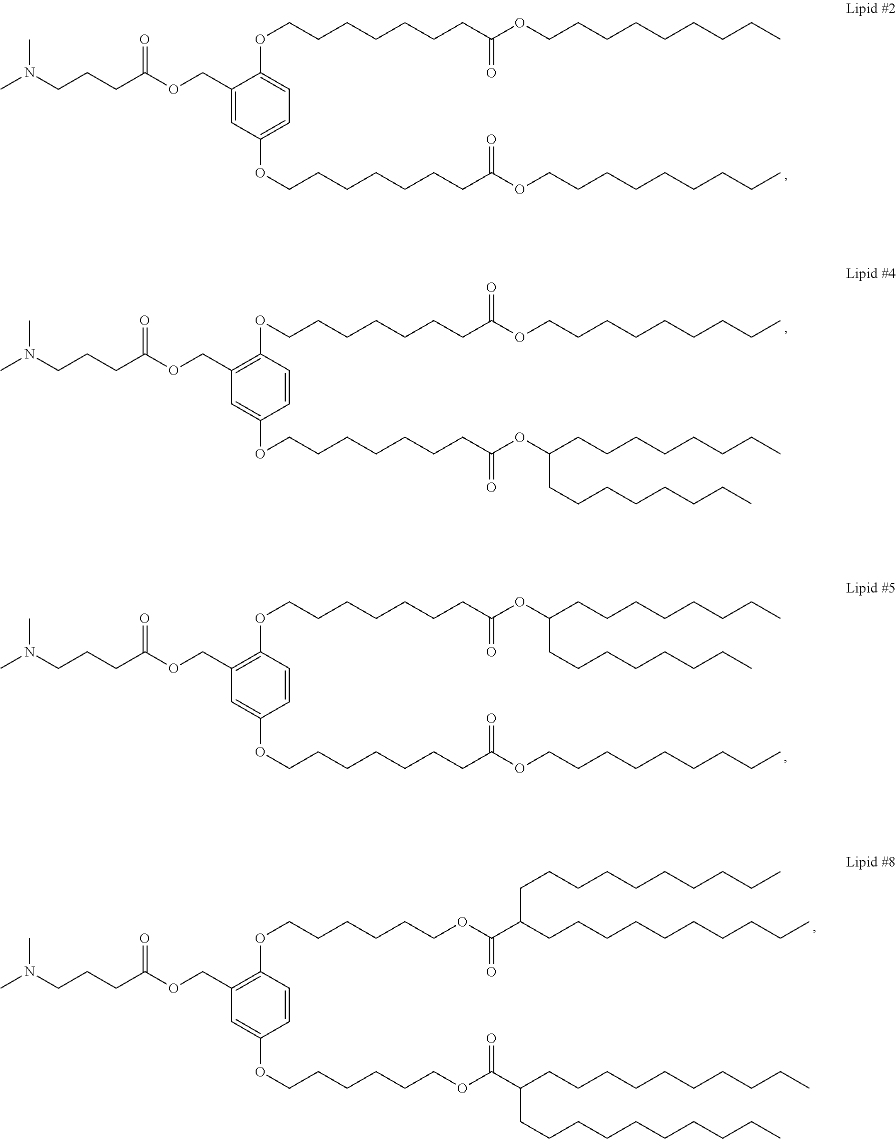

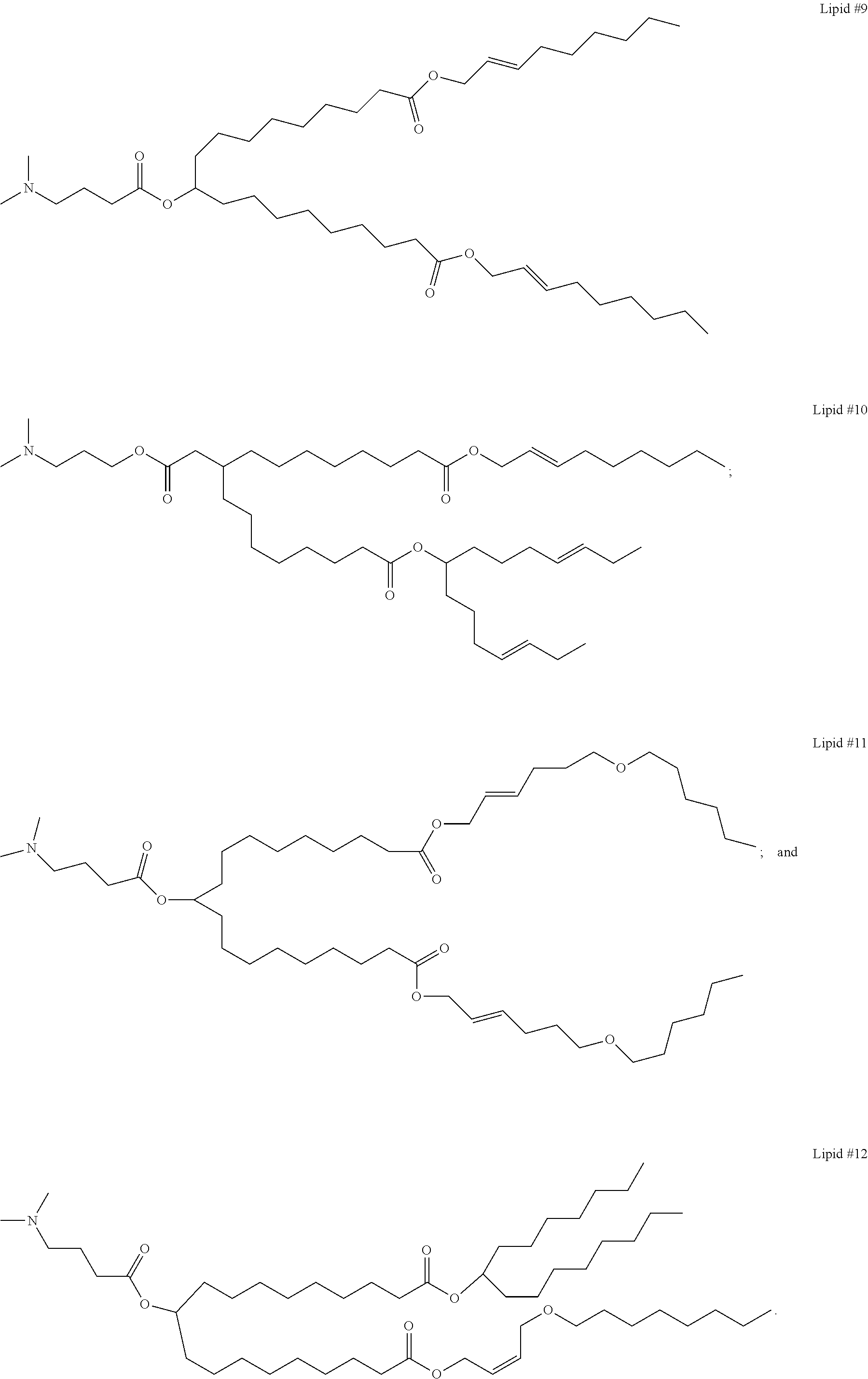

In some embodiments, the LNP comprises an ionizable lipid, phospholipid, cholesterol, and a pegylated lipid.

In some embodiments, the LNP comprises an ionizable lipid, 1,2-diastearoyl-sn-glycero-3-phosphocholine (DSPC), cholesterol, and a pegylated lipid, optionally 1,2-dimyristoyl-rac-glycero-3-methoxypolyethylene glycol-2000 (DMG-PEG 2000).

In some embodiments, the LNP comprises an ionizable lipid, 1,2-Dioleoyl-sn-glycero-3-phosphoethanolamine (DOPE), cholesterol, and a pegylated lipid, optionally 1,2-dimyristoyl-rac-glycero-3-methoxypolyethylene glycol-2000 (DMG-PEG 2000).

In some embodiments, the LNP comprises a phospholipid: cholesterol: a pegylated lipid: and an ionizable lipid with the mole ratio within the ranges of phospholipid: 5%-20%, cholesterol: 18.5%-58.5%, pegylated lipid: 1%-4%, ionizable lipid: 30%-70%, and the total summation of the mole ratio of the lipids is 100%.

In some embodiments, the LNP comprises a phospholipid: cholesterol: a pegylated lipid: and an ionizable lipid with the mole ratio within the ranges of phospholipid: 5%-20%, cholesterol: 18.5%-58.5%, pegylated lipid: 1%-4%, ionizable lipid: 40%-55%, and the total summation of the mole ratio of the lipids is 100%.

In some embodiments, the LNP comprises a phospholipid: cholesterol: a pegylated lipid: and an ionizable lipid with the mole ratio within the ranges of phospholipid: 5%-20%, cholesterol: 18.5%-58.5%, pegylated lipid: 1%-4%, ionizable lipid: 40%-50%, and the total summation of the mole ratio of the lipids is 100%.

In some embodiments, the LNP comprises a phospholipid: cholesterol: a pegylated lipid: ionizable lipid with the mole ratio within the ranges of phospholipid: 5%-20%, cholesterol: 18.5%-58.5%, pegylated lipid: 1%-4%, ionizable lipid: 45%-50%, and the total summation of the mole ratio of the lipids is 100%.

In some embodiments, the LNP comprises a phospholipid: cholesterol: a pegylated lipid: and an ionizable lipid with the mole ratio within the ranges of phospholipid: 5%-20%, cholesterol: 18.5%-58.5%, pegylated lipid: 1%-4%, ionizable lipid: 46%-49%, and the total summation of the mole ratio of the lipids is 100%.

In some embodiments, ionizable lipid may comprise about 41%, 42%, 43%, 44%, 45%, 46%, 47%, 48% or 49% of the LNP. In some embodiments, the LNP comprises a phospholipid:cholesterol:a pegylated lipid: and an ionizable lipid with a mole ratio of 10:40.5:1.5:48.

In some embodiments, the phospholipid is DOPE or DSPC.

In some embodiments, the pegylated lipid comprises 1,2-dimyristoyl-rac-glycero-3-methoxypolyethylene glycol-2000 (DMG-PEG 2000), DMG-PEG 3500, DMG-PEG 5000, DTDAM-PEG 2000 (ALC-0159), DTDAM-PEG 5000, DMG-C-PEG 2000, DMG-C-PEG 5000, DSG-PEG 2000, DSG-PEG 5000, DPG-PEG 2000, DPG-PEG 5000, or any combination thereof.

In some embodiments, the LNP comprises a DSPC:cholesterol:DMG-PEG 2000:and an ionizable lipid with the mole ratio within the ranges of DSPC: 5%-20%, cholesterol: 18.5%-58.5%, DMG-PEG 2000: 1%-4%, ionizable lipid: 30%-70%, the total summation of the mole ratio of the lipids is 100%.

In some embodiments, the LNP comprises a DSPC:cholesterol:DMG-PEG 2000:and an ionizable lipid with the mole ratio within ranges of DSPC: 5%-20%, cholesterol: 30%-55%, DMG-PEG 2000: 1%-4%, ionizable lipid: 40%-55%, the total summation of the mole ratio of the lipids is 100%.

In some embodiments, the LNP comprises a DSPC:cholesterol:DMG-PEG 2000:and an ionizable lipid with the mole ratio within the ranges of DSPC: 5%-20%, cholesterol: 30%-55%, DMG-PEG 2000: 1%-4%, ionizable lipid: 40%-50%, the total summation of the mole ratio of the lipids is 100%.

In some embodiments, the LNP comprises a DSPC:cholesterol:DMG-PEG 2000:and an ionizable lipid with the mole ratio within ranges of DSPC: 5%-20%, cholesterol: 30%-55%, DMG-PEG 2000: 1%-4%, ionizable lipid: 45%-50%, the total summation of the mole ratio of the lipids is 100.

In some embodiments, the LNP comprises a DSPC:cholesterol:DMG-PEG 2000:and an ionizable lipid with the mole ratio within the ranges of DSPC: 5%-20%, cholesterol: 30%-55%, PEG: 1%-4%, ionizable lipid: 46%-49%, the total summation of the mole ratio of the lipids is 100%.

In some embodiments, ionizable lipid may comprise about 41%, 42%, 43%, 44%, 45%, 46%, 47%, 48% or 49% of the LNP. In some embodiments, the LNP comprises a DSPC: cholesterol: DMG-PEG 2000:and an ionizable lipid with a mole ratio of 10:40.5:1.5:48.

In some embodiments, the LNP has a pH of 5 to 6.

In some embodiments, the LNP has a particle size of about 40-300 nm, optionally the LNP has a particle size of no greater than 160 nm, and optionally the LNP has a particle size of about 140-160 nm.

In some embodiments, the concentration of RNA is 10-120 μg/mL, optionally 30-60 μg/mL.

In some embodiments, there is a method of lyophilizing an RNA composition, comprising: a) providing a liquid comprising RNA and at least one suitable protective agent, b) adding the RNA liquid into a freeze drying chamber, c) freezing the liquid, wherein the freezing is performed at defined temperatures in four steps, d) reducing the pressure in the freeze drying chamber to a pressure below atmospheric pressure, e) drying the frozen liquid to obtain a lyophilized composition comprising the RNA and at least one suitable protective agent, optionally wherein there are primary and secondary drying steps, and f) equilibrating the pressure in the freeze drying chamber to atmospheric pressure and removing the lyophilized composition.

In some embodiments, the suitable protective agent comprises a polyol, a polymer, a sugar or any combination thereof.

In some embodiments, the concentration of the protective agent ranges from 0.5% to 30% by weight.

In some embodiments, the pH of the protective agent is in the range of 3-8, and a total ion strength less than 0.2 mol kg⁻¹.

In some embodiments, the suitable protective agent comprises 50 mM citrate buffer, pH 6.0, 7.5% (w/v) sucrose.

In some embodiments, 1-150 μg/ml of RNA liquid is added to the freeze drying chamber.

In some embodiments, the first freezing step freezes the RNA from 25° C. to 2° C. with full cooling capacity.

In some embodiments, the second freezing step freezes the RNA at 2° C. for 1 hour.

In some embodiments, the third freezing step freezes the RNA from 2° C. to −50° C., optionally with full cooling capacity.

In some embodiments, the fourth freezing step freezes the RNA at −50° C. for no less than 3 hours.

In some embodiments, the pressure in the freeze drying chamber is reduced to 0.01 mbar.

In some embodiments, the first primary drying step dries the RNA at a temperature of −50° C. to −47° C. for 1 hour.

In some embodiments, the second primary drying step dries the RNA at −47° C. for 20 hours.

In some embodiments, wherein the third primary drying step dries the RNA at −47° C. to −20° C. for 3 hours.

In some embodiments, the fourth primary drying step dries the RNA at −20° C. for 1 hour.

In some embodiments, the pressure in the freeze drying chamber is raised to 0.02 mbar.

In some embodiments, the first secondary drying step dries the RNA at −22° C. to 4° C. for 3 hours.

In some embodiments, the second secondary drying step dries the RNA at 4° C. for 2 hours.

In some embodiments, the pressure in the freeze drying chamber is raised to 0.08 mbar.

In some embodiments, the third secondary drying step dries the RNA from 4° C. to 30° C. for 4 hours.

In some embodiments, the fourth secondary drying step dries the RNA at 30° C. for 4 hours.

In some embodiments, the water content of the lyophilized RNA is less than 10%.

In some embodiments, the water content of the lyophilized RNA is less than 5%.

In some embodiments, the water content of the lyophilized RNA is less than 3%.

In some embodiments, the lyophilized RNA is at 2-8° C.

In some embodiments, the lyophilized RNA is mixed with LNP.

In some embodiments, the LNP is a liquid solution added to the lyophilized RNA.

In some embodiments, wherein the lyophilized RNA and LNP are mixed at room temperature.

In some embodiments, the lyophilized RNA and LNP are mixed prior to administration.

In some embodiments, there is a method of making the composition of any of the embodiments, comprising mixing the lyophilized RNA and the LNP.

In some embodiments, the LNP is a liquid solution and is added to the lyophilized RNA.

In some embodiments, the lyophilized RNA and LNP are mixed at room temperature.

In some embodiments, the lyophilized RNA and LNP are mixed prior to administration.

In some embodiments, the amount of LNP added to the lyophilized RNA provides a ionizable lipid: RNA (N:P) molar ratio ranging from 2:1 to 12:1, optionally ranging from 5:1 to 9:1, and optionally is 7:1.

In some embodiments, the RNA concentration of the mixed composition is 10-120 μg/mL, optionally 30-60 μg/mL.

In some embodiments, the RNA is electrostatically adsorbed to at least one exterior portion of the LNP.

In some embodiments, a method of administering the composition of any of the embodiments to the subject.

In some embodiments, the composition is administered to the subject intramuscularly, intravenously, intra-arterially, intradermally, subcutaneously, intraperitoneally, intraventricularly, or intracranially.

In some embodiments, the administration of the composition enhances an immune response in the subject.

In some embodiments, the composition is administered to the subject at least two times.

In some embodiments, the composition is administered to the subject about 1-8 weeks following the prime dose.

In some embodiments, the composition is administered to the subject at a dose of 1-100 mg, optionally 8, 10 mg or 30 mg RNA.

In some embodiments, a kit comprising the RNA composition of any of the embodiments, a liquid LNP solution composition of any of the embodiments, and instructions for use.

In some embodiments, the set of compositions are stored in separate glass vials.

In some embodiments, wherein the liquid LNP solution composition is at a temperature of 2-8° C.

In some embodiments, wherein the lyophilized RNA composition is stored at room temperature.

In some embodiments, the lyophilized RNA composition is stored at a temperature of 2-8° C.

Provided herein is a lipid of Formula I:

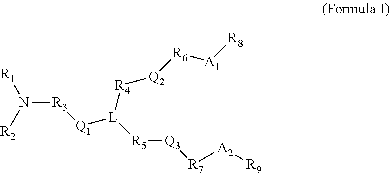

or a pharmaceutically acceptable salt thereof, wherein:
R₁ and R₂ are each, independently C₁-C₆ alkyl;
R₃ is C₁-C₅ alkyl;
Q₁, Q₂ and Q₃ are each independently —O—, —S—, —C(O)O—, —OC(O)—, —S—S—, —C(O)S—, —SC(O)—, —OC(S)—, or —C(S)O—;
L is C₁-C₃ alkyl;
R₄ and R₅ are each, independently C₁-C₁₀ alkyl;
R₆ and R₇ are each, independently C₁-C₁₀ alkyl or C₁-C₁₀ alkenyl;
A₁ and A₂ are each independently a bond, —O—, —S—, —C(O)O—, —OC(O)—, —S—S—, —C(O)S—, —SC(O)—, —OC(S)—, or —C(S)O—;
R₈ and R₉ are each, independently, C₁-C₃₀ alkyl.

In some embodiments, L is

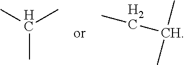

Provided herein, is a lipid of Formula II:

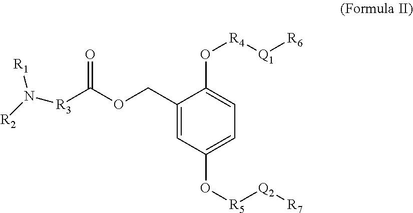

or a pharmaceutically acceptable salt thereof, wherein:
R₁ and R₂ are each, independently, C₁-C₆ alkyl;
R₃ is C₁-C₅ alkyl;
R₄ and R₅ are each independently C₁-C₁₈ alkyl;
Q₁ and Q₂ are each independently —O—C(O)—, —C(O)—O—, —O—C(S)—, —C(S)—O—; —S—S—,
R₆ and R₇ are each independently a C₁₈-C₃₂ alkyl.

In some embodiments, R₁ and R₂ are methyl. In some embodiments, Q₁ and Q₂ are each, independently, —C(O)—O— or —O—C(O)—. In some embodiments, R₃ is C₃ alkyl. Ins some embodiments, a lipid is selected form the group consisting of:

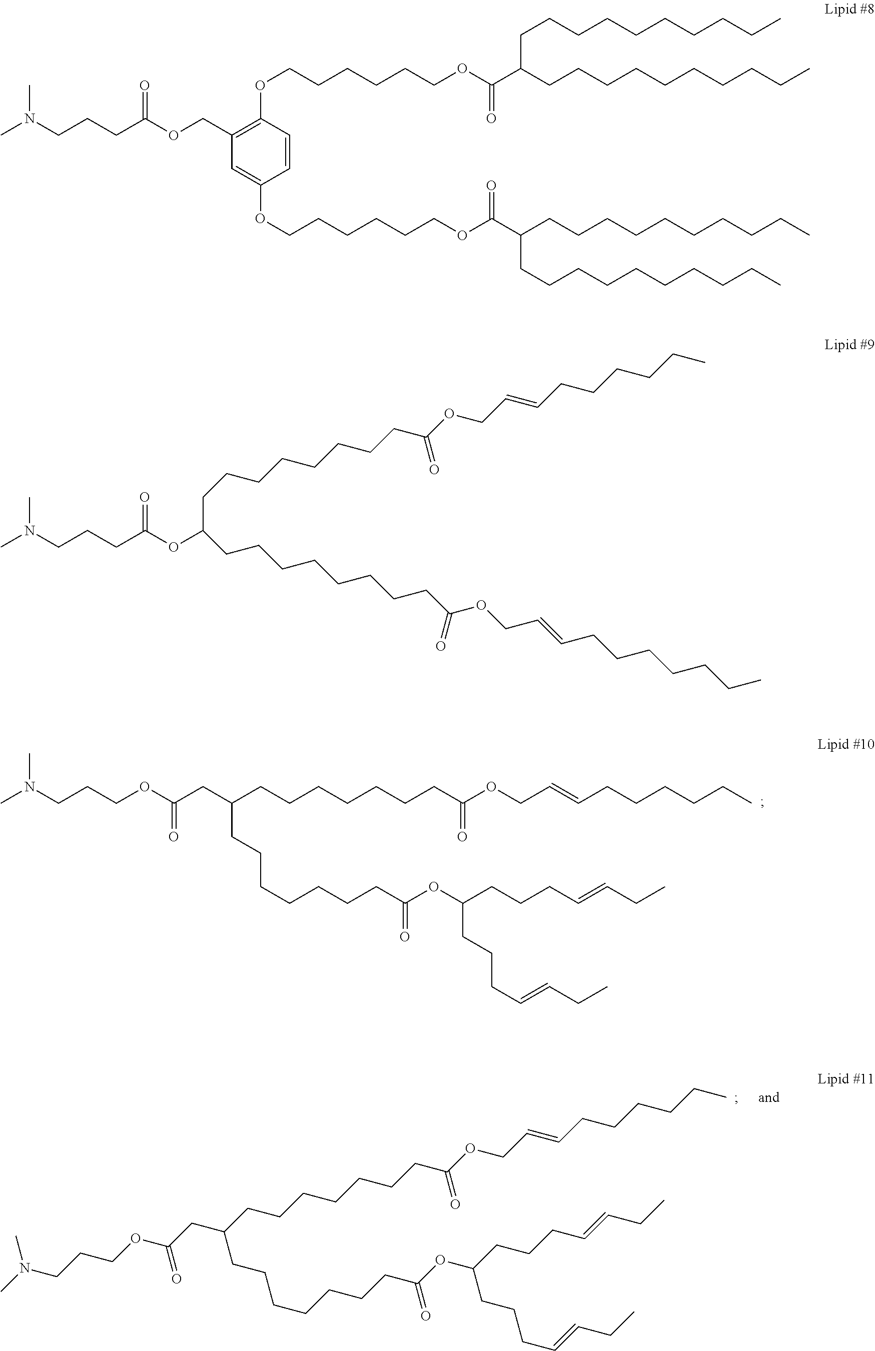

In some embodiments provided herein is a pharmaceutically acceptable composition comprising a lipid of any of the above embodiments of a lipid, and a pharmaceutically acceptable carrier.

In some embodiments, provided herein is a lipid nanoparticle (LNP) comprising an ionizable lipid of Formula I:

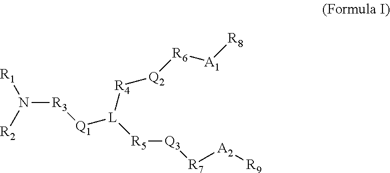

wherein:
R₁ and R₂ are each, independently C₁-C₆ alkyl;
R₃ is C₁-C₅ alkyl;
Q₁, Q₂ and Q₃ are each independently —O—, —S—, —C(O)O—, —OC(O)—, —S—S—, —C(O)S—, —SC(O)—, —OC(S)—, or —C(S)O—;
L is C₁-C₃ alkyl;
R₄ and R₅ are each, independently C₁-C₁₀ alkyl;
R₆ and R₇ are each, independently C₁-C₁₀ alkyl, C₁-C₁₀ alkenyl;
A₁ and A₂ are each independently a bond, —O—, —S—, —C(O)O—, —OC(O)—, —S—S—, —C(O)S—, —SC(O)—, —OC(S)—, or —C(S)O—; and
R₈ and R₉ are each, independently C₁-C₃₀ alkyl.

In some embodiments, the L is

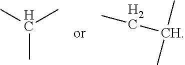

In some embodiments, the LNP comprises an ionizable lipid of Formula II:

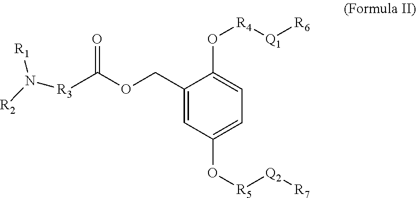

wherein:
R₁ and R₂ are each independently C₁ to C₆ alkyl;
R₃ is C₁ to C₅ alkyl;
R₄ and R₅ are each independently C₁ to C₁₈ alkyl group
Q₁ and Q₂ are each independently —O—C(O)—, —C(O)—O—, —O—C(S)—, —C(S)—O—; —S—S—, and
R₆ and R₇ are each independently C₁ to C₃₂ alkyl.

In some embodiments, R₆ and R₇ are each independently C₁-C₂₂ alkyl.

In some embodiments, R₄ and R₅ are each independently C₅-C₁₀ alkyl group.

In some embodiments, R₆ and R₇ are each independently C₇-C₁₇ alkyl.

In some embodiments, the LNP comprises a lipid selected from the group consisting of:

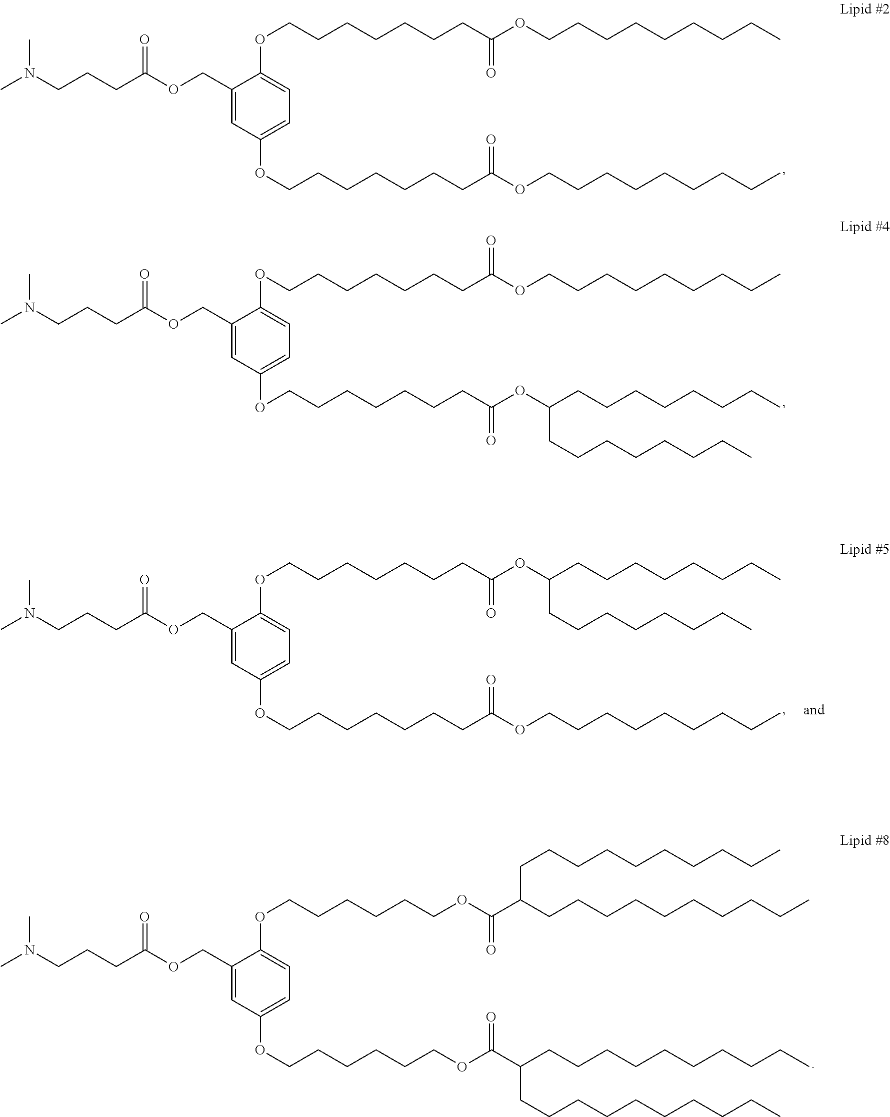

In some embodiments, R₁ and R₂ are methyl.

In some embodiments, Q₁ and Q₂ are each, independently, —C(O)—O— or —O—C(O)—.

In some embodiments, R₃ is a straight chained C₃ alkyl.

In some embodiments, the LNP comprises a lipid selected from the group consisting of:

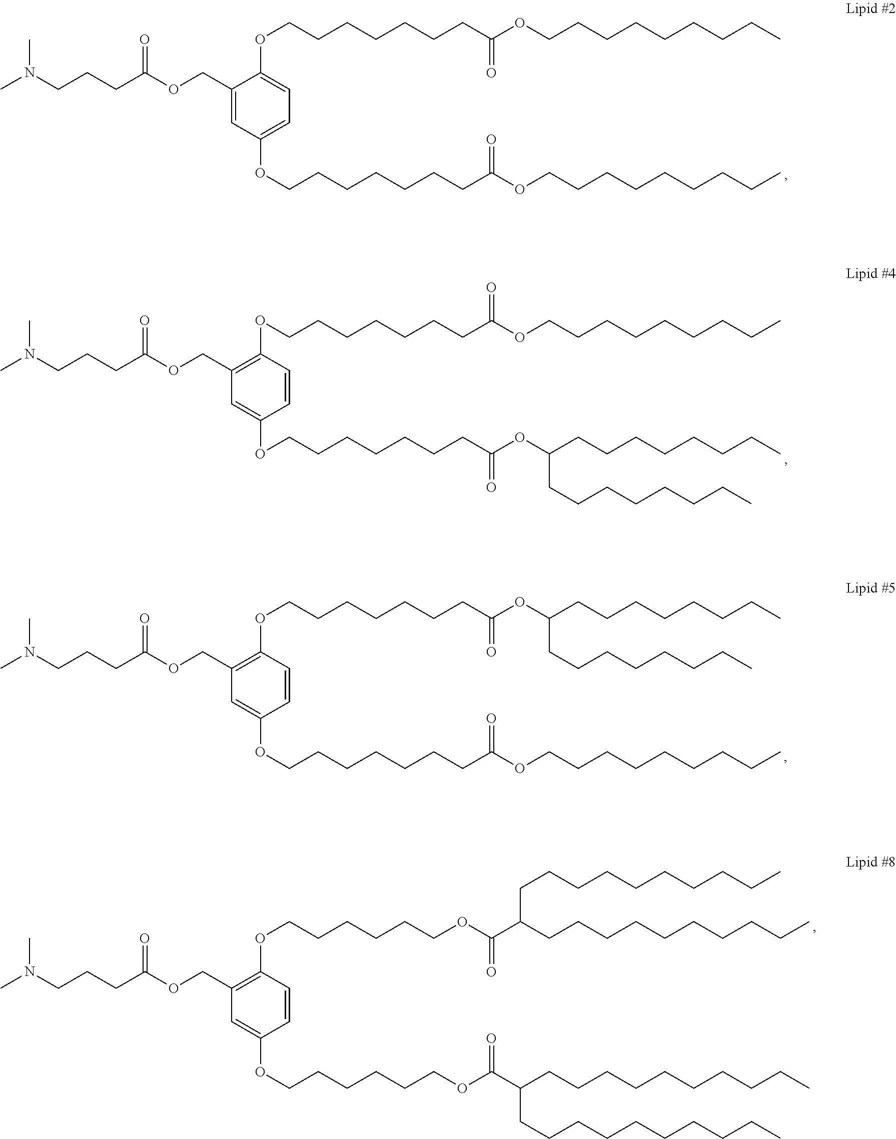

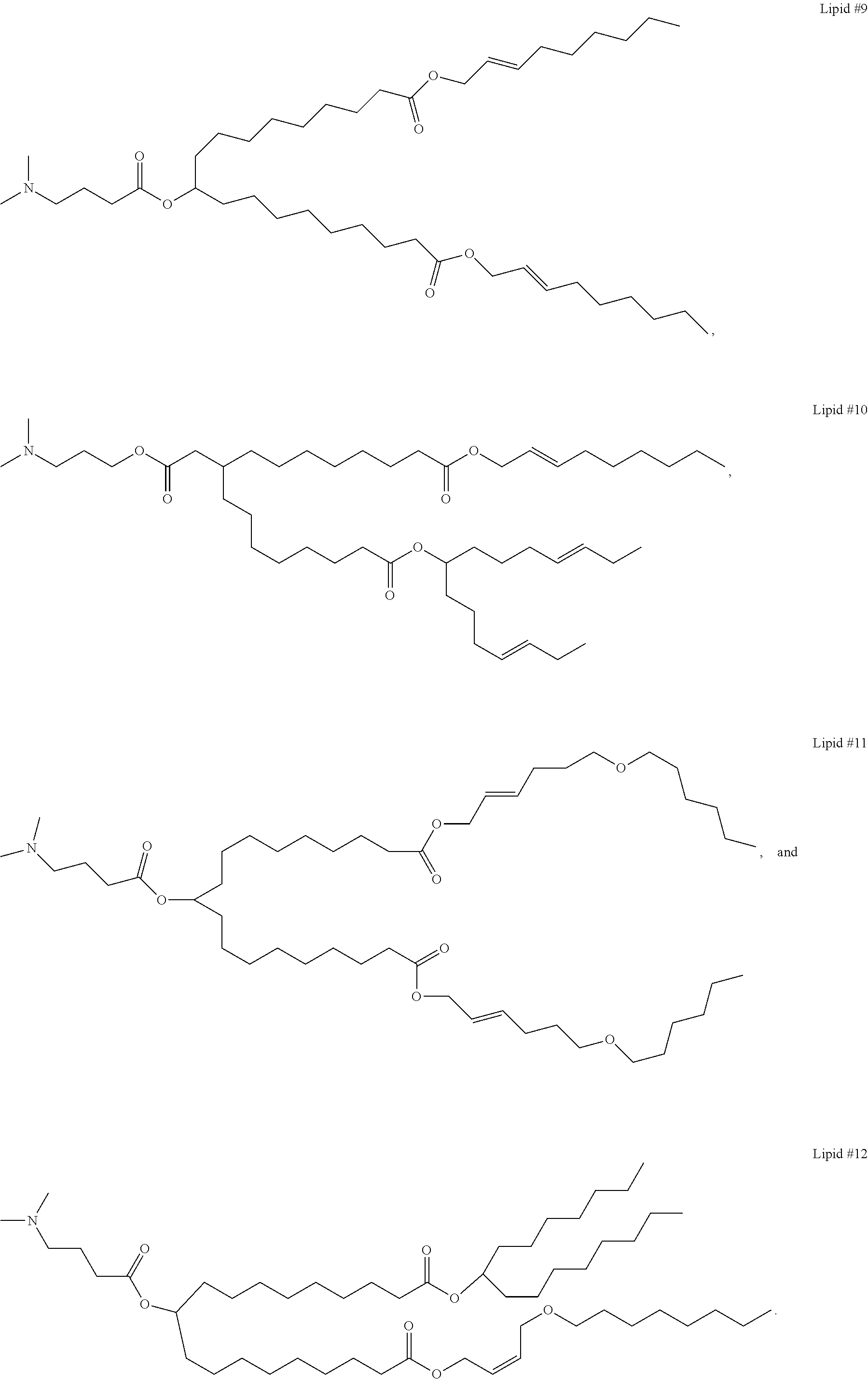

In some embodiments, the LNP further comprises a phospholipid, a structural lipid, and a pegylated lipid.

In some embodiments, the LNP comprises a phospholipid: a structural lipid: a pegylated lipid: and an ionizable lipid with the mole ratio within the ranges of phospholipid: 5%-20%, structural lipid: 18.5%-58.5%, pegylated lipid: 1%-4%, ionizable lipid: 30%-70%, and the total summation of the mole ratio of the lipids is 100%.

In some embodiments, the LNP comprises a DSPC: cholesterol: 1,2-dimyristoyl-rac-glycero-3-methoxypolyethylene glycol-2000 (DMG-PEG 2000): and an ionizable lipid with the mole ratio within the ranges of DSPC: 5%-20%, cholesterol: 30%-55%, DMG-PEG 2000: 1%-4%, ionizable lipid: 30%-70%, the total summation of the mole ratio of the lipids is 100%, optionally the mole ratio is 10:40.5:1.5:48.

In some embodiments, the LNP comprises an ionizable lipid, 1,2-dioleoyl-sn-glycero-3-phosphoethanolamine (DOPE), cholesterol, and a pegylated lipid, optionally 1,2-dimyristoyl-rac-glycero-3-methoxypolyethylene glycol-2000 (DMG-PEG 2000).

In some embodiments, the LNP comprises an ionizable lipid, 1,2-diastearoyl-sn-glycero-3-phosphocholine (DSPC), cholesterol, and a pegylated lipid, optionally 1,2-dimyristoyl-rac-glycero-3-methoxypolyethylene glycol-2000 (DMG-PEG 2000).

In some embodiments, the LNP has a pH of 5 to 6.

In some embodiments, the LNP has a particle size of about 40-300 nm, optionally the LNP has a particle size of no greater than 160 nm, optionally the LNP has a particle size of about 140-160 nm, and optionally the LNP has a particle size of about 90-120 nm.

In some embodiments, the LNP is stable when stored at 2-8° C.

In some embodiments, the average size of the LNP changes less than 40 nm when stored at 2-8° C. for more than 7 days, 14 days, or 30 days.

In some embodiments, the average size of the LNP changes less than 30 nm, 20 nm, or optionally 10 nm.

In some embodiments, provided here is a method of delivering polynucleotide into cells for expression of a polypeptide of interest using any of the LNP described above.

In some embodiments, the polynucleotide is electrostatically absorbed to at least one exterior portion of or encapsulated in the LNP.

In some embodiments, the polynucleotide is protected to reduce degradation during delivery into cells.

In some embodiments, the polynucleotide is in a lyophilized form before mixing with the LNP.

In some embodiments, the polynucleotide is in a liquid form before mixing with the LNP.

Described herein is a self-replicating RNA (srRNA) comprising a nucleotide sequence encoding a varicella-zoster virus (VZV) antigen.

In some embodiments, the VZV antigen comprises a VZV glycoprotein E (gE) antigen.

In some embodiments, the gE antigen comprises the VZV Oka strain gE protein.

In some embodiments, the gE antigen comprises the mature, extracellular domain sequence of the gE antigen or an immunogenic fragment thereof.

In some embodiments, the sequence of the extracellular domain of the gE antigen comprises the sequence shown in SEQ ID NO: 3.

In some embodiments, the nucleotide sequence encoding the VZV antigen is operably linked to a promoter.

In some embodiments, the srRNA comprises a 5′ cap untranslated region (UTR), one or more non-structural genes, a promoter, and a 3′ terminal polyadenylated (polyA) region.

In some embodiments, one or more non-structural genes comprises four non-structural genes (nsp1-4) and the promoter comprises a 26S subgenomic promoter.

In some embodiments, the nucleotide sequence encoding the VZV antigen is operably linked to the promoter.

In some embodiments, the srRNA comprises, from 5′ to 3′, the 5′ UTR, the one or more non-structural genes, the promoter, the nucleotide sequence encoding the VZV antigen, and the 3′ polyA region.

In some embodiments, the srRNA lacks one or more nucleotide sequences encoding one or more structural protein sequences, optionally the nucleotide sequence encoding VZV antigen is inserted in place of the one or more nucleotide sequences encoding the one or more structural protein sequences.

In some embodiments, the srRNA is a TC-83 VEEV srRNA

In some embodiments, srRNA sequence comprises the sequence shown in SEQ ID NO: 4.

In some embodiments, the srRNA comprises an mRNA cap.

In some embodiments, the mRNA cap comprises m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm-, where Nm denotes any nucleotide with a 2′ O methylation.

In some embodiments, the srRNA is lyophilized.

In some embodiments, the lyophilized srRNA is at a temperature at or below 22° C., optionally about 2-8° C.

In some embodiments, there is a composition comprising the srRNA of any of the above claims and a delivery vehicle.

In some embodiments, the delivery vehicle comprises a lipid nanoparticle (LNP).

In some embodiments, the LNP comprises an ionizable lipid, heptadecan-9-yl 8-(3-(((4-(dimethylamino)butanoyl)oxy)methyl)-4-((8-(nonyloxy)-8-oxooctyl)oxy)phenoxy)octanoate.

In some embodiments, the LNP comprises an ionizable lipid, 1,2-Diastearoyl-sn-glycero-3-phosphocholine (DSPC), Cholesterol, and 1,2-dimyristoyl-sn-glycero-3-phosphoethanolamine-N-[methoxy(polyethylene glycol)-2000] (DMG-PEG2000).

In some embodiments, the LNP comprises an ionizable lipid, 1,2-Dioleoyl-sn-glycero-3-phosphoethanolamine (DOPE), Cholesterol, and 1,2-dimyristoyl-sn-glycero-3-phosphoethanolamine-N-[methoxy(polyethylene glycol)-2000] (DMG-PEG2000).

In some embodiments, LNP comprises an ionizable lipid, 1,2-Dioleoyl-sn-glycero-3-phosphoethanolamine (DOPE), Cholesterol, and a pegylated lipid comprising a polyethylene glycol moiety.

In some embodiments, the srRNA is adsorbed to the surface of the LNP.

In some embodiments, the composition comprises a DSPC: cholesterol: PEG: ionizable lipid mole ratio ranging from 5:20:0:20 to 25:70:5:60, optionally 10:48:2:40, at a N:P (lipid:srRNA) ratio ranging from 2:1 to 12:1, optionally 8:1.

In some embodiments, the composition comprises a DOPE: cholesterol: PEG: ionizable lipid mole ratio ranging from 5:20:0:20 to 25:70:5:60, optionally 10:48:2:40, at a N:P (lipid:srRNA) ratio ranging from 2:1 to 12:1, optionally 8:1.

In some embodiments, the composition has a particle size of about 40-300 nm.

In some embodiments, the composition enhances an immune response in a subject following administration.

In some embodiments, the immune response comprises an antigen-specific adaptive immune response.

In some embodiments, the adaptive immune response comprises B cells, CD4+ T cells, and/or CD8+ T cells.

In some embodiments, the adaptive immune response comprises CD8+ T cells.

In some embodiments, the composition lacks a separate adjuvant component.

In some embodiments, there is a method of enhancing an immune response to VZV in a subject, comprising administering the composition of any embodiment to the subject.

In some embodiments, there is a method of preventing or treating shingles or a VZV-related disease in a subject, comprising administering the composition of an embodiment to the subject.

In some embodiments, the composition is administered to the subject intramuscularly.

In some embodiments, the composition is administered to the subject about 4 weeks following the prime dose.

In some embodiments, the composition is administered to the subject at least two times.

In some embodiments, the composition is administered to the subject at a dose of 1-100 μg, optionally 10 μg or 30 μg.

In some embodiments, the composition enhances an immune response in the subject following administration.

In some embodiments, the immune response comprises an antigen-specific adaptive immune response.

In some embodiments, the adaptive immune response comprises B cells, CD4+ T cells, and/or CD8+ T cells.

In some embodiments, the adaptive immune response comprises CD8+ T cells.

In some embodiments, there is a method of making the composition of any embodiment, comprising mixing the srRNA with the delivery vehicle.

In some embodiments, the srRNA is lyophilized, and optionally at a temperature of 2-8° C.

In some embodiments, the delivery vehicle is an LNP, and optionally in a liquid state, optionally at a temperature of 2-8° C.

In some embodiments, there is a kit comprising the srRNA of any embodiment, a delivery vehicle, and instructions for use.

In some embodiment, the srRNA of the kit is lyophilized, and optionally at a temperature of 2-8° C.

In some embodiments, the delivery vehicle of the kit is an LNP, and optionally in a liquid state, optionally at a temperature of 2-8° C.

Described herein, in some embodiments, is a composition or set of compositions separately comprising each of (1) a lyophilized polynucleotide composition and (2) a liquid lipid nanoparticle (LNP) solution. In some embodiments, the LNP comprises an ionizable lipid of Formula II:

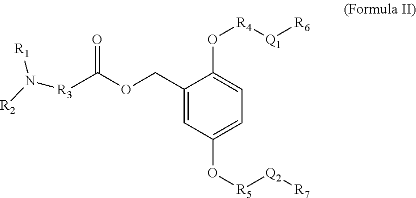

wherein: R₁ and R₂ are each independently C₁ to C₆ alkyl; R₃ is C₁ to C₅ alkyl; R₄ and R₅ are each independently C₁ to C₁₈ alkyl group Q₁ and Q₂ are each independently —O—C(O)—, —C(O)—O—, —O—C(S)—, —C(S)—O—; —S—S—, and R₆ and R₇ are each independently C₁ to C₃₂ alkyl. In some embodiments, R₄ and R₅ are each independently C₅-C₁₀ alkyl group; and R₆ and R₇ are each independently C₇-C₁₇ alkyl. In some embodiments, R₁ and R₂ are methyl, Q₁ and Q₂ are each, independently, —C(O)—O— or —O—C(O)—, and R₃ is a straight chained C₃ alkyl. In some embodiments, the ionizable lipid is:

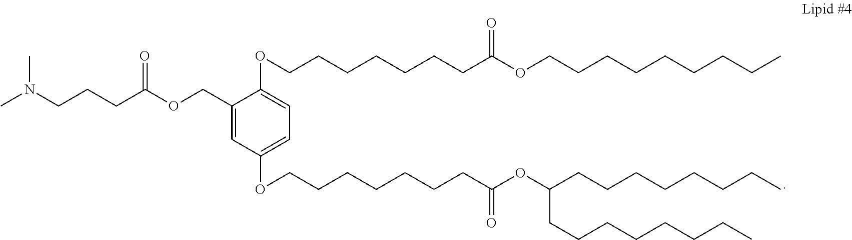 [SMILES: CCCCCCCCCOC(=O)CCCCCCCOc1ccc(OCCCCCCCC(=O)OC(CCCCCCCC)CCCCCCCC)cc1COC(=O)CCCN(C)C]

In some embodiments, the lyophilized polynucleotide composition comprises a self-replicating RNA. In some embodiments, the lyophilized polynucleotide encodes a protein, a polypeptide, or an antigen. In some embodiments, the lyophilized polynucleotide comprises at least one of a messenger RNA (mRNA), a ribosomal RNA (rRNA), transfer RNA (tRNA), viral RNA, retroviral RNA, self-replicating RNA (replicon RNA), small interfering RNA (siRNA), microRNA, small nuclear RNA (snRNA), small-hairpin (sh) RNA, a riboswitch, a ribozyme, an oligonucleotide, or an aptamer. In some embodiments, the lyophilized polynucleotide is a self-replicating RNA. The composition of claim 1, wherein the lyophilized polynucleotide is a not a self-replicating RNA. In some embodiments, the water content of the lyophilized polynucleotide composition is less than 5%. In some embodiments, the lyophilized polynucleotide composition is stored at about 2-8° C. In some embodiments, the lyophilized polynucleotide composition comprises at least one suitable protective agent. In some embodiments, the suitable protective agent comprises a polyol, a polymer, a sugar, or any combination thereof. In some embodiments, the suitable protective agent comprises sucrose. In some embodiments, the concentration of the protective agent ranges from 0.5% to 30% by weight of the lyophilized polynucleotide composition. In some embodiments, the LNP comprises an ionizable lipid of Formula I:

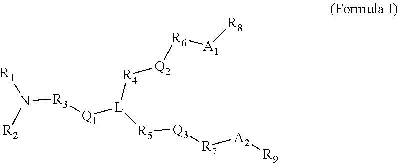

wherein: R₁ and R₂ are each, independently C₁-C₆ alkyl; R₃ is C₁-C₅ alkyl; Q₁, Q₂ and Q₃ are each independently —O—, —S—, —C(O)O—, —OC(O)—, —S—S—, —C(O)S—, —SC(O)—, —OC(S)—, or —C(S)O—; L is C₁-C₃ alkyl; R₄ and R₅ are each, independently C₁-C₁₀ alkyl; R₆ and R₇ are each, independently C₁-C₁₀ alkyl, C₁-C₁₀ alkenyl; A₁ and A₂ are each independently a bond, —O—, —S, C(O)O—, —OC(O)—, —S—S—, —C(O)S—, —SC(O)—, —OC(S), or —C(S)O—; and R₈ and R₉ are each, independently C₁-C₃₀ alkyl. In some embodiments, L is

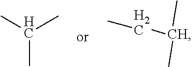

wherein the CH₂ moiety is attached to Q₁. In some embodiments, the ionizable lipid is selected form the group consisting of:

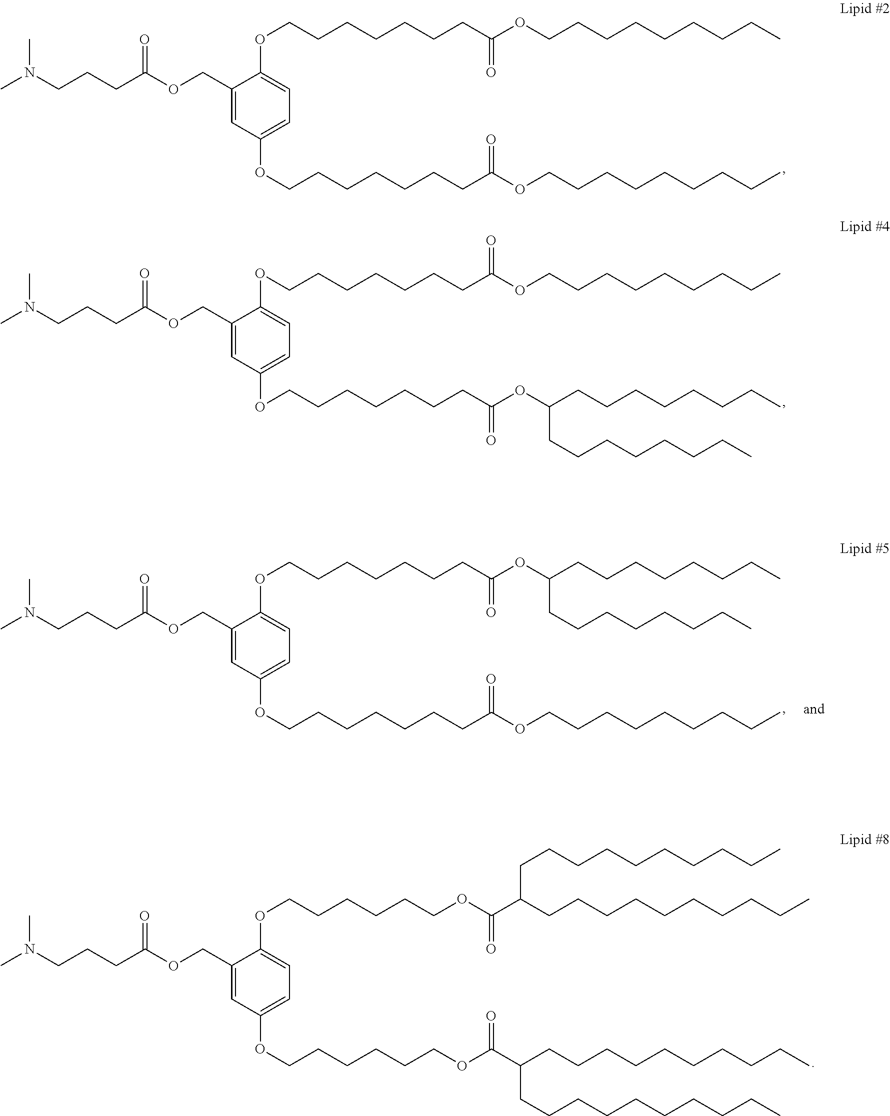

In some embodiments, the ionizable lipid is selected form the group consisting of:

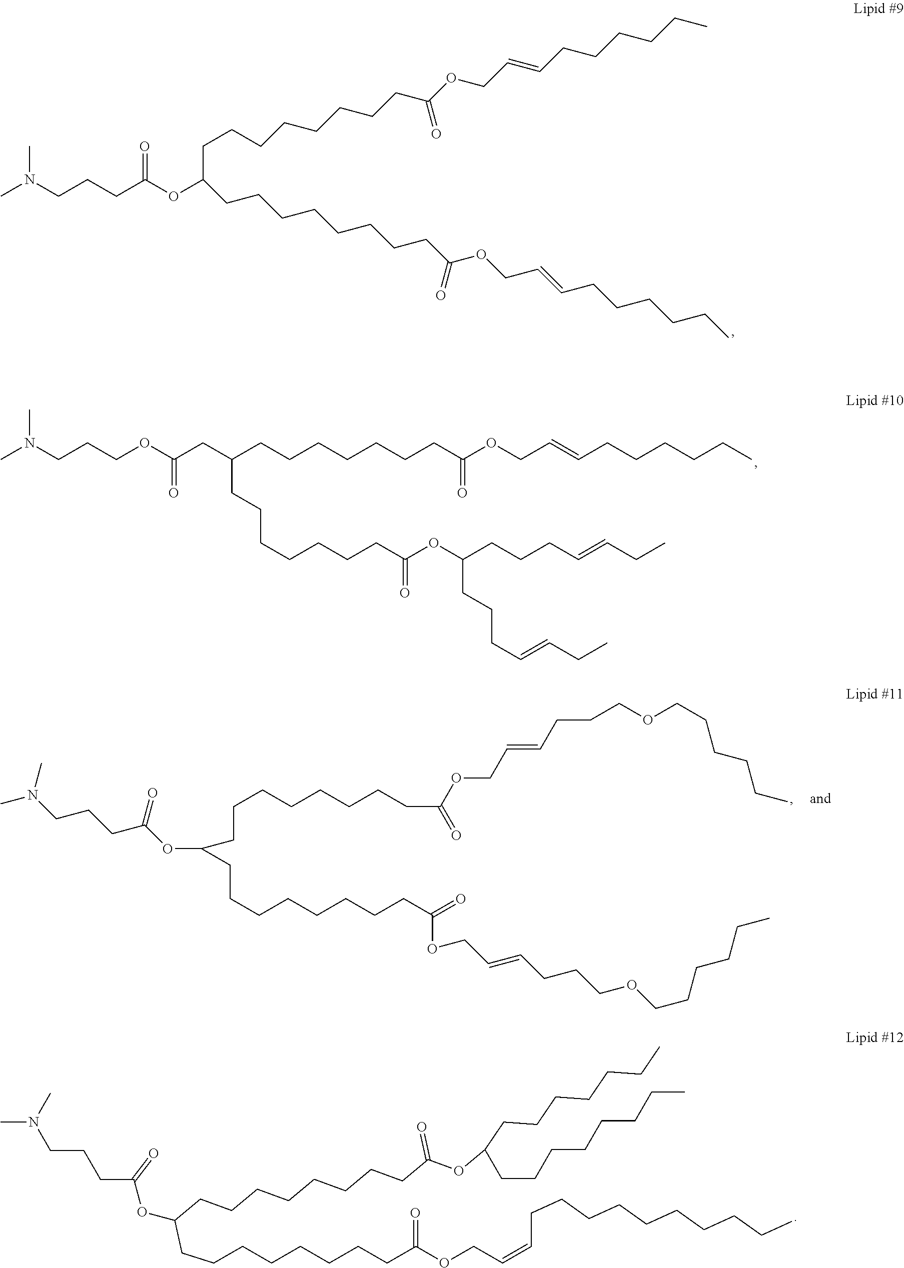

In some embodiments, the LNP comprises an ionizable lipid, a phospholipid, a structural lipid, and a pegylated lipid. In some embodiments, the LNP comprises the ionizable lipid, 1,2-dioleoyl-sn-glycero-3-phosphoethanolamine (DOPE), cholesterol, and the pegylated lipid. In some embodiments, the LNP comprises the ionizable lipid, 1,2-diastearoyl-sn-glycero-3-phosphocholine (DSPC), cholesterol, and the pegylated lipid. In some embodiments, the pegylated lipid is 1,2-dimyristoyl-rac-glycero-3-methoxypolyethylene glycol-2000 (DMG-PEG 2000). In some embodiments, the LNP comprises the phospholipid: the structural lipid: the pegylated lipid: and the ionizable lipid in a mole ratio within the ranges of phospholipid: 5%-20%, structural lipid: 18.5%-58.5%, pegylated lipid: 1%-4%, ionizable lipid: 30%-70%, wherein the total summation of the mole ratio of the lipids is 100%. In some embodiments, the LNP comprises DSPC: cholesterol: 1,2-dimyristoyl-rac-glycero-3-methoxypolyethylene glycol-2000 (DMG-PEG 2000): and the ionizable lipid in a mole ratio within the ranges of DSPC: 5%-20%, cholesterol: 30%-55%, DMG-PEG 2000: 1%-4%, ionizable lipid: 30%-70%, wherein the total summation of the mole ratio of the lipids is 100%. In some embodiments, the LNP solution has a pH of 5 to 6. In some embodiments, the LNP has a particle size of about 40-300 nm, optionally the LNP has a particle size of no greater than 160 nm, optionally the LNP has a particle size of about 140-160 nm, and optionally the LNP has a particle size of about 90-120 nm. In some embodiments, the lyophilized polynucleotide composition comprises SEQ ID NO: 4.

Described herein, in some embodiments, is a self-replicating RNA (srRNA) vector comprising in 5′ to 3′ order: a) a Cap1 cap; b) a 5′ UTR; c) one or more structural genes; d) a gene of interest (GOI); e) a 3′ UTR comprising about 180 to about 400 nucleotides; and f) a poly A tail comprising about 60 to about 100 nucleotides. In some embodiments, the 3′ UTR comprises a nucleic acid sequence having least about 80% identity to the nucleic acid sequence SEQ ID NO: 6. In some embodiments, the srRNA vector comprises at least 1.5×, 2.0×, 2.5×, 3.0×, or more GOI expression as compared to an unmodified vector. In some embodiments, the srRNA vector comprises at least 2×, 3×, 4×, 5×, 6×, or more improved immune response as compared to an unmodified vector. In some embodiments, the GOI is a varicella-zoster virus (VZV) antigen. In some embodiments, the VZV antigen comprises a VZV glycoprotein E (gE) antigen.

Described herein, in some embodiments, is a self-replicating RNA (srRNA) comprising a nucleotide sequence encoding a varicella-zoster virus (VZV) antigen. In some embodiments, the VZV antigen comprises a VZV glycoprotein E (gE) antigen. In some embodiments, the gE antigen comprises a VZV Oka strain gE protein. In some embodiments, the gE antigen comprises the mature, extracellular domain sequence of the gE antigen or an immunogenic fragment thereof. In some embodiments, the sequence of the extracellular domain of the gE antigen comprises SEQ ID NO:3. In some embodiments, the nucleotide sequence encoding the VZV antigen is operably linked to a promoter. In some embodiments, the srRNA comprises a 5′ cap untranslated region (UTR), one or more non-structural genes, a promoter, and a 3′ terminal polyadenylated (polyA) region. In some embodiments, the one or more non-structural genes comprises four non-structural genes (nsp1-4) and the promoter comprises a 26S subgenomic promoter. In some embodiments, the srRNA comprises, from 5′ to 3′, the 5′ UTR, the one or more non-structural genes, the promoter, the nucleotide sequence encoding the VZV antigen, and the 3′ polyA region. In some embodiments, the srRNA lacks one or more nucleotide sequences encoding one or more structural protein sequences, optionally wherein the nucleotide sequence encoding VZV antigen is inserted in place of the one or more nucleotide sequences encoding the one or more structural protein sequences. In some embodiments, the srRNA is a TC-83 VEEV srRNA. In some embodiments, the srRNA sequence comprises SEQ ID NO:2. In some embodiments, the srRNA sequence comprises SEQ ID NO:4. In some embodiments, the srRNA comprises a polyA tail comprising 30-100 nucleotides in length. In some embodiments, the srRNA comprises a 3′ UTR comprising 100-400 nucleotides in length. In some embodiments, the srRNA comprises an mRNA cap. In some embodiments, the mRNA cap comprises m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm-, where Nm denotes any nucleotide with a 2′ O methylation. In some embodiments, the srRNA comprises a Cap1 cap, a VZV glycoprotein E (gE) antigen, a 3′ UTR comprising about 270 nucleotides in length, and a polyA tail comprising about 65 nucleotides in length.

Described herein, in some embodiments, is a composition or set of compositions separately comprising each of (1) a lyophilized polynucleotide composition and (2) a liquid lipid nanoparticle (LNP) solution wherein the lyophilized polynucleotide is a self-replicating RNA, and the LNP comprises an ionizable lipid with the formula:

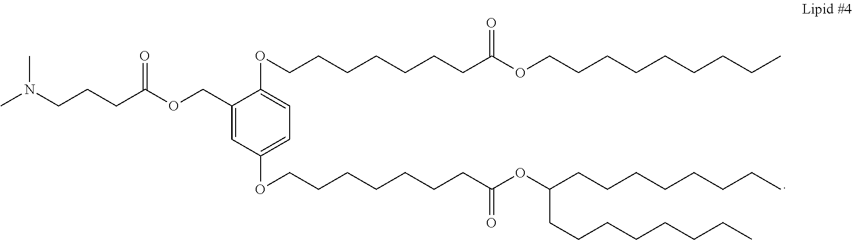 [SMILES: CCCCCCCCCOC(=O)CCCCCCCOc1ccc(OCCCCCCCC(=O)OC(CCCCCCCC)CCCCCCCC)cc1COC(=O)CCCN(C)C]

In some embodiments, the lyophilized polynucleotide comprises a polyA tail 30-100 nucleotides in length. In some embodiments, the lyophilized polynucleotide comprises a cap structure selected from the group consisting of m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm-, where Nm denotes any nucleotide with a 2′ O methylation. In some embodiments, the lyophilized polynucleotide comprises a nucleotide sequence encoding a varicella-zoster virus (VZV) antigen. In some embodiments, the lyophilized polynucleotide comprises a VZV glycoprotein E (gE) antigen. In some embodiments, the lyophilized polynucleotide comprises a 3′ UTR 180 to 400 nucleotides in length. In some embodiments, the lyophilized polynucleotide comprises a Cap1 cap, a VZV glycoprotein E (gE) antigen, a 3′ UTR comprising about 270 nucleotides in length, and a polyA tail comprising about 65 nucleotides in length. In some embodiments, the lyophilized polynucleotide comprises a Cap1 cap, SARS-CoV-2 spike protein (RBD), a 3′ UTR comprising about 270 nucleotides in length, and a polyA tail comprising about 65 nucleotides in length.

Described herein, in some embodiments, is a self-replicating RNA (srRNA) comprising SEQ ID NO:4 encoding a varicella-zoster virus (VZV) gE antigen.

### BRIEF DESCRIPTION OF THE DRAWINGS

FIGS. 1A-1B show the structures of ionizable lipids used to test the “ready-to-use” RNA formulations.

FIG. 2 is a Western Blot probed for the spike glycoprotein of SARS-CoV-2 of Vero E6 cell line samples transfected with either the “ready-to-use” or LNP-encapsulated S-2P RNA.

FIG. 3 shows a treatment protocol of BALB/c mice vaccinated with either “ready-to-use” or LNP-encapsulated S-2P RNA encoding the spike glycoprotein of SARS-CoV-2. Arrows point to days of blood collection.

FIG. 4 shows the geometric mean titer of BALB/c mice vaccinated with either “ready-to-use” or LNP-encapsulated S-2P RNA encoding the spike glycoprotein of SARS-CoV-2 at the indicated days.

FIG. 5 shows a treatment protocol with srRNA vaccine for cynomolgus monkey studies.

FIG. 6 shows VZV gE specific antibody titers in serum of cynomolgus monkeys on the indicated days; arrows indicate an administered dose of srRNA vaccine.

FIG. 7 shows antibody avidity in serum of immunized cynomolgus monkeys on the indicated days.

FIG. 8 shows individual cytokine expression levels on day 90 of peripheral blood mononuclear cells from cynomolgus monkeys immunized with 10 ug or 30 ug of srRNA vaccine.

FIG. 9 shows an exemplary construction of the srRNA encoding the VZV glycoprotein E antigen.

FIG. 10A and FIG. 10B show the relative potency of a lyophilized RNA, stored at the indicated temperature and selected at the indicated time points, tested using an in vitro potency assay, which compares the total intensity of fluorescence signal against a reference standard.

FIG. 11 shows a treatment protocol with a VZV srRNA vaccine for mice studies.

FIG. 12 shows the serum antibody levels in immunized mice determined by ELISA. The arrows below the graph indicate the time of the VZV srRNA vaccination. Each data point shown is the average EC50 of individual mouse sera in each group (n=12 for group G1-G6; n=8 for group G7). Bars represent geometric mean±geometric standard deviation (SD).

FIG. 13A and FIG. 13B show the frequencies of antigen-specific T-cell subsets measured from splenocytes of immunized mice following in vitro restimulation with a peptide pool spanning the VZV gE protein. Bars represent the average of individuals in each group with mean±SD. (**, p<0.01).

FIG. 14 shows the frequencies of cytokine-expressing VZV gE-specific T cells as measured by flow cytometry. The results of CD4+ T cell immune response. Bars represent the average of individuals in each group with mean±SD. (**, p<0.01 versus G5 or G7).

FIG. 15 shows an exemplary construction of a vector structure with an elongated 3′ UTR and/or polyA tail.

FIG. 16 shows exemplary srRNA structures.

FIG. 17A shows a Western blot probed for RBD expression.

FIG. 17B shows the relative RBD expression of the Western Blot across samples.

FIG. 18 shows in vivo BLI imaging of mice treated with Structure 1, Structure 3, Structure 4, or vehicle.

FIG. 19 is the quantification of the total flux of the mice treated shown in FIG. 18. Significant differences between the Structure 1 group and the others groups are indicated by an asterisk vertically atop other asterisk(s) (*p<0.05,**p<0.01).

FIG. 20A shows the immunization schedule of the mice in the experiment for FIG. 20B.

FIG. 20B shows the quantification of total IgG antibody titers in mice vaccinated with dual antigen VZV gE srRNA vaccines.

FIG. 21A and FIG. 21B show frequencies of cytokine-expressing VZV gE-specific T cells as measured by flow cytometry. FIG. 21A shows the results of CD4+ T cell immune response. FIG. 21B shows the results of CD8+ T cell immune response. The bars represent the average of individuals in each group with mean±SD. (*p<0.05, **p<0.01).

### DETAILED DESCRIPTION

### Lyophilization of RNA

The present disclosure provides a method for the lyophilization of RNA. The present disclosure further concerns a lyophilized composition obtainable by the method provided herein, as well as a pharmaceutical composition and a kit containing the lyophilized composition.

Nucleic acids, both DNA and RNA, have been used for vaccines, either in naked or in complexed form. The application of RNA in vaccines or other therapeutics represents a favored tool in modern molecular medicine as RNA exhibits some superior properties over DNA. Transfection of DNA molecules may lead to complications. For example, application of DNA molecules bears the risk that the DNA integrates into the host genome. Integration of foreign DNA into the host genome can have an influence on the expression of host genes and can trigger the expression of an oncogene or the inactivation of a tumor suppressor gene. Furthermore, an essential gene, and, as a consequence, the product of such an essential gene, may also be inactivated by the integration of the foreign DNA into the coding region of the gene. The result of such an event may be particularly dangerous if the DNA is integrated into a gene, which is involved in regulation of cell growth. Notwithstanding the risks associated with its application, DNA still represents an important tool. However, these risks do not occur if RNA is used instead of DNA.

An advantage of using RNA rather than DNA is that no virus-derived promoter element has to be administered in vivo and no integration into the genome may occur. Furthermore, the RNA, in order to exert its function, does not need to overcome the barrier to the nucleus.

However, a main disadvantage of the use of RNA is its instability. Even though it is understood that DNA, such as naked DNA, when introduced into a patient circulatory system, is typically not stable and therefore may have little chance of affecting most disease processes (see Poxon et al., Pharmaceutical development and Technology, 5(1), 115-122 (2000)), the problem of stability becomes even more prominent for RNA. It is generally known that the physico-chemical stability of RNA molecules in solution is low. RNA is susceptible to hydrolysis by ubiquitous ribonucleases or by divalent cations and is typically rapidly degraded, e.g., already after a few hours or days in solution. Rapid degradation occurs even in the absence of RNases, e.g., when RNA is stored in solution at room temperature for a few hours or days.

To avoid such rapid degradation, RNA (in solution) is typically stored at −20° C. or even −80° C. and under RNAse free conditions to prevent degradation of the RNA. Such storage conditions, however, may not sufficiently prevent a loss of function over time. Additionally, applying such conditions is very cost-intensive, especially for shipping and storage, e.g., whenever such low temperatures need to be guaranteed.

One method to increase the stabilization RNA comprises lyophilization or freeze-drying the RNA. Lyophilization is a method known and recognized worldwide, which is used to enhance storage stability of temperature sensitive biomolecules. During lyophilization, a solvent, such as water, is typically removed from a frozen sample via sublimation.

The process of lyophilization is usually characterized by a primary and a secondary drying step. During the primary drying step, free, i.e. unbound, water surrounding the biomolecule, and optionally further components, evaporates from the frozen solution. Subsequently, water, which is bound by the biomolecule on a molecular basis, may be removed in a secondary drying step by adding thermal energy. In both cases, the hydration sphere around the biomolecule is lost.

During lyophilization, a sample containing a biomolecule is initially cooled below the freezing point of the solution, consequently freezing the water contained therein. Depending, amongst other parameters, on temperature, cooling rate (freezing rate), and the time for freezing, crystals may be formed. This exerts physical stress on the biomolecule and other components of the solution, which may lead to a damage of the biomolecule such as—in the case of a nucleic acid—breakage of strands, loss of supercoiling, etc. Furthermore, due to the decrease of volume and loss of the hydration sphere, autocatalytic degradation processes are favored e.g., by traces of transition metals. In addition, the concentration of traces of acids and bases can result in significant changes of the pH value. Lyophilization involves two types of stress, namely freezing and drying. Both types of stress are known to damage nucleic acids, such as non-viral vectors or plasmid DNA. In the literature, a number of cryoprotectants and lyoprotectants are discussed for lyophilization purposes to prevent these damages. In this context, cryoprotectants are understood as excipients, which allow influencing the structure of the ice and/or the eutectical temperature or glass transition temperature of the mixture. Lyoprotectants are typically excipients, which partially or totally replace the hydration sphere around a molecule and may thus at least partially prevent catalytic and hydrolytic processes.

In the specific context of DNA, lyophilization causes the removal of the hydration sphere around the DNA, where it appears that there are approximately 20 water molecules per nucleotide pair bound most tightly to DNA. These water molecules do not form an ice-like structure upon low-temperature cooling. Upon DNA dehydration in the presence of hygroscopic salts at 0% relative humidity, only five or six water molecules remain (Tao et al., Biopolymers, 28, 1019-1030 (1989)).

Lyophilization may increase the stability of DNA under long-term storage, but may also cause some damage due to the initial lyophilization process, potentially through changes in the DNA secondary structure, breaks of the nucleic acid chain(s) or the concentration of reactive elements such as contaminating metals. Lyophilization can also cause damage upon the initial lyophilization process in other nucleic acid, e.g., RNA. Agents that can substitute for non-freezable water, such as some carbohydrates, can demonstrate cryoprotective properties with respect to DNA and other molecules during lyophilization of intact bacteria (Israeli et al, Cryobiology, 30, 519-523 (1993); or Rudolph et al, Arch. Biochem. Biophys., 245, 134-143 (1986)). During lyophilization, specific carbohydrates, such as several sugars, appear to play a central role in the stabilization of nucleic acid molecules. However, when using cryoprotectants and lyoprotectants, no general rule may be applied with respect to their impact on different groups of compounds. Therefore, herein a cryoprotectant is used.

Lyoprotective properties are particularly described for sucrose, glucose, and trehalose. They allow the restoration of at least, in part, the transfection efficiency, which is otherwise lost in many cases after lyophilisation (Maitani et al, 2008, supra; Yadava, P., M. Gibbs, et al. (2008). AAPS PharmSciTech 9(2): 335-41; Werth, S., B. Urban-Klein, et al. (2006), J Control Release 112(2): 257-70; Brus, C, E. Kleemann, et al. (2004), J Control Release 95(1): 119-31; Poxon, S. W. and J. A. Hughes (2000), Pharm Dev Technol 5(1): 115-22; Anchordoquy, T. J., J. F. Carpenter, et al. (1997), Arch Biochem Biophys 348(1): 199-206).

Sugars are able to prevent loss in activity due to the lyophilization process mainly by preventing particle fusion/aggregation especially in the case of liposome complexed nucleic acids (Yadava et al, 2008, supra; Katas, H., S. Chen, et al. (2008), J Microencapsul: 1-8; Molina et al, supra, 2001). Particularly, Poxon et al. (2000, supra) investigated the effect of lyophilization on plasmid DNA activity. Poxon et al. (2000, supra) hypothesized, that a change in the DNA conformation from supercoiled to open circular and linear form would be indicative of damage of the plasmid DNA. However, the percentage of supercoiled DNA did not change after lyophilization and subsequent DMED treatment, suggesting that other effects are responsible for the loss of transfection efficiency. Poxon et al. (2000, supra) found that a decrease in plasmid DNA activity as measured by an in vitro transfection assay can be ameliorated by the use of carbohydrates during lyophilization of the plasmid DNA. Glucose (monosaccharide), sucrose and lactose (disaccharides) were used as lyoprotectants. Poxon et al. (2000, supra), however, only carried out investigations using plasmid DNA.

Though a number of prior art documents suggest the stabilization of nucleic acids during lyophilization in the context of plasmid DNA, only a few publications focus on stabilization of other nucleic acids, such as RNAs, e.g., during lyophilization and long-term storage. Furthermore, lyophilization under controlled conditions is rarely described for RNA.

Therefore, it is an object of the present disclosure to provide a method for lyophilization of RNA, which is scalable, reproducible, and applicable for the production of pharmaceuticals. In particular, it is an object of the present disclosure to provide a method for lyophilization of RNA, by which the integrity and the biological activity of the RNA is preferably maintained. It is a further object of the disclosure to provide a composition comprising RNA, which is suitable for storage also at ambient temperature and over extended periods.

The present disclosure solves these problems as demonstrated by the claimed subject matter. The present disclosure provides a method for lyophilizing RNA. In particular, the present disclosure teaches a method for lyophilizing RNA, wherein the method comprises the following steps:
a. providing a liquid comprising RNA and at least one suitable protective agent,
b. adding the RNA liquid into a freeze drying chamber,
c. freezing the liquid, wherein the freezing is performed at defined temperatures in four steps,
d. reducing the pressure in the freeze drying chamber to a pressure below atmospheric pressure,
e. drying the frozen liquid to obtain a lyophilized composition comprising the RNA and at least one suitable protective agent, wherein there are primary and secondary drying steps, and
f. equilibrating the pressure in the freeze drying chamber to atmospheric pressure and removing the lyophilized composition.

In certain embodiments, the steps (a) to (f) are performed in the order above. In some embodiments, they may be carried out in an alternative order. In some embodiments, the single steps may be performed concomitantly or may overlap. The method can be suitable for use at an industrial scale.

The method according to the present disclosure can be used to produce a composition comprising RNA in a reproducible manner. The composition comprising RNA according to the present disclosure can advantageously be stored, shipped and applied, e.g., in the medical field (for example, as a vaccine or a therapeutic), without the use of freezers, while the integrity and the biological activity of the RNA in the composition remain high.

In some embodiments, the lyophilized composition obtainable by a disclosed method is characterized by a residual moisture content, which is preferably in the range from about 0.1% (w/w) to about 10% (w/w), optionally in the range from about 1% (w/w) to about 8% (w/w), optionally in the range from about 2% (w/w) to about 5% (w/w), optionally in the range from about 2% (w/w) to 4%, optionally about 3% (w/v), optionally less than 3% (w/v), based on the total weight of the lyophilized composition.

In some embodiments, the liquid provided in step a) of the present disclosure comprises at least one cryoprotectant, wherein the cryoprotectant is a carbohydrate. Such carbohydrates may comprise any carbohydrate suitable for the preparation of a pharmaceutical composition, preferably, without being limited thereto, monosaccharides, such as e.g., glucose, fructose, sucrose, galactose, sorbose, mannose, and mixtures thereof; disaccharides, such as e.g., lactose, maltose, sucrose, trehalose, cellobiose, etc., and mixtures thereof; polysaccharides, such as raffinose, melezitose, maltodextrins, dextrans, dextrins, cellulose, starches, etc., and mixtures thereof; and alditols, such as glycerol, mannitol, xylitol, maltitol, lactitol, xylitol sorbitol, pyranosyl sorbitol, myoinositol, etc., and mixtures thereof. Preferred examples of sugars that may be included in the liquid provided in step a) of the present disclosure include lactose, mannose, mannitol, sucrose or trehalose. Generally, a sugar that is preferred in this context, has a high water displacement activity and a high glass transition temperature. Furthermore, a sugar suitable for use in the liquid provided in step a) of the present disclosure is preferably hydrophilic but not hygroscopic. In addition, the sugar preferably has a low tendency to crystallize.

In some embodiments, the liquid provided in step a) of the present disclosure comprises at least 0.01% (w/w), preferably at least 0.1% (w/w), at least 0.5% (w/w), at least 1% (w/w), at least 2.5% (w/w), at least 5% (w/w), at least 10% (w/w), or at least 15% (w/w) of a cryoprotectant, wherein the cryoprotectant is preferably a carbohydrate component, such as a sugar. In some embodiments, the liquid provided in step a) of the present disclosure comprises a cryoprotectant, optionally a carbohydrate, optionally a sugar, at a concentration in a range from 0.1% to 40% (w/w), optionally at a concentration in a range from 1% to 20% (w/w), optionally at a concentration of 7.5% (w/w). In certain embodiments, the liquid provided in step a) of the present disclosure comprises RNA at a concentration of about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, or about 95 μg/mL.

### In Vitro Transcription of RNA

In some embodiments, the RNA of the present disclosure comprises an RNA polynucleotide, such as a messenger RNA (mRNA) or a self-replicating RNA. mRNA, for example, is transcribed in vitro from template DNA, referred to as an “in vitro transcription template.”

In vitro transcription of RNA is known in the art and is described, e.g., in International Publication WO2014/152027, which is incorporated by reference herein in its entirety. For example, in some embodiments, the RNA transcript is generated using a non-amplified, linearized DNA template in an in vitro transcription reaction to generate the RNA transcript. In some embodiments the RNA transcript is capped via enzymatic capping. In some embodiments, the RNA transcript is purified via chromatographic methods, e.g., use of an oligo dT substrate. Some embodiments exclude the use of DNase. In some embodiments the RNA transcript is synthesized from a non-amplified, linear DNA template coding for the gene of interest via an enzymatic in vitro transcription reaction utilizing a T7 phage RNA polymerase and nucleotide triphosphates of the desired chemistry. Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides.

As used herein, the terms “termini” or “terminus,” when referring to polypeptides or polynucleotides, refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N-termini and C-termini. Alternatively, the termini of the polypeptides may be modified such that they begin or end, as the case may be, with a non-polypeptide based moiety such as an organic conjugate.

In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

In some embodiments, the RNA described herein comprises an elongated polyA tail. In some embodiments, the RNA described herein comprises a polyA tail between 30-100 nucleotides in length. In some embodiments, the RNA described herein comprises a polyA tail between 35-100 nucleotides in length. In some embodiments, the RNA described herein comprises a polyA tail between 40-100 nucleotides in length. In some embodiments, the RNA described herein comprises a polyA tail between 45-100 nucleotides in length. In some embodiments, the RNA described herein comprises a polyA tail between 50-100 nucleotides in length. In some embodiments, the RNA described herein comprises a polyA tail between 55-100 nucleotides in length. In some embodiments, the RNA described herein comprises a polyA tail between 60-100 nucleotides in length. In some embodiments, the RNA described herein comprises a polyA tail between 65-100 nucleotides in length. In some embodiments, the RNA described herein comprises a polyA tail between 70-100 nucleotides in length. In some embodiments, the RNA described herein comprises a polyA tail between 80-100 nucleotides in length. In some embodiments, the RNA described herein comprises a polyA tail between 90-100 nucleotides in length. In some embodiments, the RNA described herein comprises a polyA tail between 95-100 nucleotides in length. In some embodiments, the RNA described herein comprises a polyA tail between 30-95 nucleotides in length. In some embodiments, the RNA described herein comprises a polyA tail between 30-90 nucleotides in length. In some embodiments, the RNA described herein comprises a polyA tail between 30-85 nucleotides in length. In some embodiments, the RNA described herein comprises a polyA tail between 30-80 nucleotides in length. In some embodiments, the RNA described herein comprises a polyA tail between 30-75 nucleotides in length. In some embodiments, the RNA described herein comprises a polyA tail between 30-70 nucleotides in length. In some embodiments, the RNA described herein comprises a polyA tail between 30-65 nucleotides in length. In some embodiments, the RNA described herein comprises a polyA tail between 30-60 nucleotides in length. In some embodiments, the RNA described herein comprises a polyA tail between 30-55 nucleotides in length. In some embodiments, the RNA described herein comprises a polyA tail between 30-50 nucleotides in length. In some embodiments, the RNA described herein comprises a polyA tail between 30-45 nucleotides in length. In some embodiments, the RNA described herein comprises a polyA tail of 10, 15, 20, 25, 30, 35, 40, 45, 50, 55, 60, 65, 70, 75, 80, 85, 90, 95, 100, 110, 120, 130, 140, or more than 140 nucleotides in length.

A “5′ untranslated region” (5′ UTR) refers to a region of an RNA (e.g. mRNA or other coding RNA) that is directly upstream (i.e., 5′) from the start codon (i.e., the first codon of an RNA, such as mRNA or other coding RNA, transcript translated by a ribosome) that does not encode a polypeptide.

A “3′ untranslated region” (3′ UTR) refers to a region of an RNA (e.g. mRNA or other coding RNA) that is directly downstream (i.e., 3′) from the stop codon (i.e., the codon of an RNA, such as mRNA or other coding RNA, transcript that signals a termination of translation) that does not encode a polypeptide.

In some embodiments, the RNA described herein has an elongated 3′ UTR. In some embodiments, the RNA described herein has a 3′ UTR between 100-500 nucleotides in length. In some embodiments, the RNA described herein has a 3′ UTR between 125-500 nucleotides in length. In some embodiments, the RNA described herein has a 3′ UTR between 150-500 nucleotides in length. In some embodiments, the RNA described herein has a 3′ UTR between 175-500 nucleotides in length. In some embodiments, the RNA described herein has a 3′ UTR between 200-500 nucleotides in length. In some embodiments, the RNA described herein has a 3′ UTR between 225-500 nucleotides in length. In some embodiments, the RNA described herein has a 3′ UTR between 250-500 nucleotides in length. In some embodiments, the RNA described herein has a 3′ UTR between 275-500 nucleotides in length. In some embodiments, the RNA described herein has a 3′ UTR between 300-500 nucleotides in length. In some embodiments, the RNA described herein has a 3′ UTR between 325-500 nucleotides in length. In some embodiments, the RNA described herein has a 3′ UTR between 100-476 nucleotides in length. In some embodiments, the RNA described herein has a 3′ UTR between 100-450 nucleotides in length. In some embodiments, the RNA described herein has a 3′ UTR between 100-425 nucleotides in length. In some embodiments, the RNA described herein has a 3′ UTR between 100-400 nucleotides in length. In some embodiments, the RNA described herein has a 3′ UTR between 100-375 nucleotides in length. In some embodiments, the RNA described herein has a 3′ UTR between 100-350 nucleotides in length. In some embodiments, the RNA described herein has a 3′ UTR between 300-350 nucleotides in length. In some embodiments, the RNA described herein has a 3′ UTR about 330 nucleotides in length. In some embodiments, the RNA described herein has a 3′UTR about 270 nucleotides in length. In some embodiments, the RNA described herein comprises a 3′ UTR of 50, 75, 100, 125, 150, 175, 200, 225, 250, 275, 300, 325, 350, 375, 400 or more than 440 nucleotides in length.

An “open reading frame” is a continuous stretch of DNA or RNA beginning with a start codon (e.g., methionine (ATG or AUG)), and ending with a stop codon (e.g., TAA, TAG or TGA, or UAA, UAG or UGA) and typically encodes a polypeptide (e.g., protein). It will be understood that the sequences may further comprise additional elements, e.g., 5′ and 3′ UTRs, but that those elements, unlike the ORF, need not necessarily be present in a vaccine or a therapeutic of the present disclosure.

A “polyA tail” is a region of RNA (e.g. mRNA or other coding RNA) that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect RNA from enzymatic degradation, e.g., in the cytoplasm, and aids in transcription termination, and/or export of the RNA from the nucleus and translation.

### Polynucleotides

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences will recite “T”s in a representative DNA sequence, but where the sequence represents RNA (e.g., mRNA), the “T”s would be substituted by “U”s.

Thus, any of the RNA polynucleotides encoded by a DNA identified by a particular sequence identification number may also comprise the corresponding RNA (e.g., mRNA or other coding RNA) sequence encoded by the DNA, where each “T” of the DNA sequence is substituted with “U.”

It should be understood that the RNA polynucleotides as provided herein are synthetic molecules, i.e., they are not naturally-occurring molecules. That is, the RNA polynucleotides of the present disclosure are isolated RNA polynucleotides. As is known in the art, “isolated polynucleotides” refer to polynucleotides that are substantially physically separated from other cellular material (e.g., separated from cells and/or systems that produce the polynucleotides) or from other material that hinders their use in the vaccines or therapeutics of the present disclosure. Isolated polynucleotides are substantially pure in that they have been substantially separated from the substances with which they may be associated in living or viral systems. Thus, RNA polynucleotides are not associated with living or viral systems, such as cells or viruses. The RNA polynucleotides do not include viral components (e.g., viral capsids, viral enzymes, or other viral proteins, for example, those needed for viral-based replication), and the RNA polynucleotides are not packaged within, encapsulated within, linked to, or otherwise associated with a virus or viral particle. In some embodiments, the RNA comprise a lipid nanoparticle that comprises, consists of, or consists essentially of, one RNA polynucleotides (e.g., RNA polynucleotides encoding one VZV antigen).

Any sequence may be codon optimized. Codon optimization methods are known in the art. Codon optimization, in some embodiments, may be used to match codon frequencies in target and host organisms to ensure proper folding; bias GC content to increase RNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and RNA degradation sites; adjust translational rates to allow the various domains of the protein to fold properly; or reduce or eliminate problem secondary structures within the polynucleotide. In some embodiments, the open reading frame (ORF) sequence is optimized using optimization algorithms. In some embodiments, a sequence encoding an antigen, e.g., a spike protein of SARS-CoV-2, is codon optimized. In some embodiments, a sequence encoding an antigen, e.g., a VZV antigen, is codon optimized.

In some embodiments, the RNA may include at least one RNA polynucleotide encoding at least one antigenic polypeptide having at least one of: a modification, at least one 5′ terminal cap, and formulation with a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

In some embodiments, the RNA may include a Cap1 cap.

When transfected into mammalian cells, the modified RNAs typically have a stability of between 12-18 hours, or greater than 18 hours, e.g., 24, 36, 48, 60, 72, or greater than 72 hours.

Varicella zoster virus (VZV) vaccines, as provided herein, comprise at least one self-replicating ribonucleic acid (srRNA) polynucleotide encoding at least one VZV antigenic polypeptide.

In some embodiments, at least one RNA polynucleotide of a VZV vaccine is encoded by SEQ ID NO: 4. In some embodiments, the srRNA sequence comprises the nucleic acid sequence of SEQ ID NO: 4. In some embodiments, the srRNA sequence comprises a nucleic acid sequence having at least 70% (e.g., 75%, 80%, 90%, 95%, 97%, 98%, or 99%) sequence identity to the nucleic acid sequence of SEQ ID NO: 4. In some embodiments, the srRNA sequence comprises a nucleic acid sequence having at least 75% sequence identity to the nucleic acid sequence of SEQ ID NO: 4. In some embodiments, the srRNA sequence comprises a nucleic acid sequence having at least 80% sequence identity to the nucleic acid sequence of SEQ ID NO: 4. In some embodiments, the srRNA sequence comprises a nucleic acid sequence having at least 90% sequence identity to the nucleic acid sequence of SEQ ID NO: 4. In some embodiments, the srRNA sequence comprises a nucleic acid sequence having at least 95% sequence identity to the nucleic acid sequence of SEQ ID NO: 4. In some embodiments, the srRNA sequence comprises a nucleic acid sequence having at least 97% sequence identity to the nucleic acid sequence of SEQ ID NO: 4. In some embodiments, the srRNA sequence comprises a nucleic acid sequence having at least 98% sequence identity to the nucleic acid sequence of SEQ ID NO: 4. In some embodiments, the srRNA sequence comprises a nucleic acid sequence having at least 99% sequence identity to the nucleic acid sequence of SEQ ID NO: 4.

In some embodiments, the RNA sequence comprises the nucleic acid sequence of SEQ ID NO: 5. In some embodiments, the RNA sequence comprises a nucleic acid sequence having at least 70% (e.g., 75%, 80%, 90%, 95%, 97%, 98%, or 99%) sequence identity to the nucleic acid sequence of SEQ ID NO: 5. In some embodiments, the RNA sequence comprises a nucleic acid sequence having at least 75% sequence identity to the nucleic acid sequence of SEQ ID NO: 5. In some embodiments, the RNA sequence comprises a nucleic acid sequence having at least 80% sequence identity to the nucleic acid sequence of SEQ ID NO: 5. In some embodiments, the RNA sequence comprises a nucleic acid sequence having at least 90% sequence identity to the nucleic acid sequence of SEQ ID NO: 5. In some embodiments, the RNA sequence comprises a nucleic acid sequence having at least 95% sequence identity to the nucleic acid sequence of SEQ ID NO: 5. In some embodiments, the RNA sequence comprises a nucleic acid sequence having at least 97% sequence identity to the nucleic acid sequence of SEQ ID NO: 5. In some embodiments, the RNA sequence comprises a nucleic acid sequence having at least 98% sequence identity to the nucleic acid sequence of SEQ ID NO: 5. In some embodiments, the RNA sequence comprises a nucleic acid sequence having at least 99% sequence identity to the nucleic acid sequence of SEQ ID NO: 5.

### Varicella Zoster Virus (VZV)

VZV is an alpha-herpesvirus that exists as a spherical multilayered structure approximately 200 nm in diameter. The viral genome is surrounded by a protein capsid structure that is covered by an amorphous layer of tegument proteins. These two structures are surrounded by a lipid envelope that is studded with viral glycoproteins, each about 8 nm in length, that are displayed on the exterior of the virion, and encloses the 100 nm nucleocapsid which is comprised of 162 hexameric and pentameric capsomeres arranged in an icosahedral form. The tegument, which is comprised of virally-encoded proteins and enzymes, is located in the space between the nucleocapsid and the viral envelope. The viral envelope is acquired from host cell membranes and contains viral-encoded glycoproteins.

VZV is closely related to the herpes simplex viruses (HSV), sharing much genome homology. The VZV genome is the smallest of the human herpesviruses and encodes at least 71 unique proteins (ORF0-ORF68) with three more opening reading frames (ORF69-ORF71) that duplicate earlier open reading frames (ORF64-62, respectively). Only a fraction of the encoded proteins form the structure of the virus particle. Among those proteins are nine glycoproteins: ORFS (gK), ORF9A (gN), ORF14 (gC), ORF31 (gB), ORF37 (gH), ORF50 (gM), ORF60 (gL), ORF67 (gI), and ORF68 (gE). The known envelope glycoproteins (gB, gC, gE, gH, gI, gK, gL, gN, and gM) correspond with those in HSV; however, there is no equivalent of HSV gD. VZV also fails to produce the LAT (latency-associated transcripts) that play an important role in establishing HSV latency (herpes simplex virus). The encoded glycoproteins gE, gI, gB, gH, gK, gL, gC, gN, and gM function in different steps of the viral replication cycle. The most abundant glycoprotein found in infected cells, as well as in the mature virion, is glycoprotein E (gE, ORF 68), which is a major component of the virion envelope and is essential for viral replication. Glycoprotein I (gI, ORG 67) forms a complex with gE in infected cells, which facilitates the endocytosis of both glycoproteins and directs them to the trans-Golgi network (TGN) where the final viral envelope is acquired. Glycoprotein I (gI) is required within the TGN for VZV envelopment and for efficient membrane fusion during VZV replication. VZV gE and gI are found complexed together on the infected host cell surface. Antibodies to gE, gB, and gH are prevalent after natural infection and following vaccination and have been shown to neutralize viral activity in vitro.

### Antigens

The lyophilized RNA herein is designed to contain or encode for a substance that produces an immune response in a subject. Aside from mRNA, the RNA described herein can be one of several non-coding types of RNA, such as a ribosomal RNA (rRNA) or a transfer RNA (tRNA). The terms “RNA” or “RNA molecule” further encompass other coding RNA molecules, such as viral RNA, retroviral RNA, self-replicating RNA (replicon RNA), small interfering RNA (siRNA), microRNA, small nuclear RNA (snRNA), small-hairpin (sh) RNA, riboswitches, ribozymes or aptamers.

In certain embodiments, the RNA is a long RNA or a long RNA molecule. The term “long RNA” as used herein typically refers to an RNA molecule, preferably as described herein, which preferably comprises at least 30 nucleotides. Alternatively, a long RNA may comprise at least 35, 40, 45, 50, 55, 60, 65, 70, 75, 80, 85, 90, 95, 100, 150, 200, 250, 300, 350, 400, 450 or at least 500 nucleotides. A long RNA may comprise at least 1000 nucleotides, or at least 2000 nucleotides. A long RNA, in the context of the present disclosure, may comprise from 30 to 50,000 nucleotides, from 30 to 20,000 nucleotides, from 100 to 20,000 nucleotides, from 200 to 20,000 nucleotides, from 200 to 15,000 nucleotides or from 500 to 20,000 nucleotides. The term “long RNA” as used herein is not limited to a certain type of RNA, but merely refers to the number of nucleotides contained in said RNA. In certain embodiments, the RNA as used herein is a long mRNA.

In the present disclosure, the RNA may be a coding RNA molecule encoding a protein or a peptide, which may be selected, without being restricted thereto, e.g., from therapeutically active proteins or peptides, selected from adjuvant proteins, from antigens, e.g., tumour antigens, pathogenic antigens (e.g., animal antigens, from viral antigens, from protozoan antigens, from bacterial antigens), allergenic antigens, autoimmune antigens, or further antigens, preferably as defined herein, from allergens, from antibodies, from immunostimulatory proteins or peptides, from antigen-specific T-cell receptors, or from any other protein or peptide suitable for a specific (therapeutic) application, wherein the coding RNA molecule may be transported into a cell, a tissue or an organism, and the protein may be expressed subsequently in this cell, tissue or organism. In certain embodiments, the RNA provided in the liquid in step a) of the present disclosure is an RNA molecule, where “step a) of the present disclosure” refers to step a) of the methods of lyophilizing an RNA composition in which a liquid comprising a RNA and at least one suitable protective agent is provided.

In certain embodiments, the RNA in the liquid provided in step a) of present disclosure may be an immunostimulatory RNA molecule, such as any RNA molecule known in the art, which is capable of inducing an immune response, preferably an innate immune response. Such an immunostimulatory RNA may be any (double-stranded or single-stranded) RNA, e.g., a coding RNA, as defined herein. In certain embodiments, the immunostimulatory RNA is a non-coding RNA. The immunostimulatory RNA may be a single-stranded, a double-stranded, or a partially double-stranded RNA, optionally a single-stranded RNA or a circular or linear RNA, preferably, a linear RNA. In various embodiments, the immunostimulatory RNA may be a linear single-stranded RNA. Even more preferably, the immunostimulatory RNA may be a long, linear single-stranded RNA.

An immunostimulatory RNA may also occur as a short RNA oligonucleotide. As used herein, an immunostimulatory RNA may be selected from any class of RNA molecules, found in nature or being prepared synthetically, and which can induce an innate immune response and may support an adaptive immune response induced by an antigen.

In some embodiments, an antigenic polypeptide is a VZV glycoprotein. For example, a VZV glycoprotein may be VZV gE, gI, gB, gH, gK, gL, gC, gN, or gM. The UniProtKB accession numbers for the glycoproteins are: gI: P09258; gB: P09257; gH: P09260; gK: P09261; gL: Q71S15 and Q775I7; gC: P09256; gN: Q0Q872; and gM: P09298. In some embodiments, the antigenic polypeptide is a VZV gE polypeptide. An antigenic polypeptide can be encoded by one or more nucleotide sequences. Such nucleotide sequence(s) encoding an antigenic polypeptide can be included in a vector such as srRNA.

The present disclosure includes variant VZV antigenic polypeptides. In some embodiments, the variant VZV antigenic polypeptide is a variant VZV gE polypeptide. The variant VZV gE polypeptides are designed to avoid ER/golgi retention of polypeptides, leading to increased surface expression of the antigen. In some embodiments, the variant gE polypeptides are truncated to remove the ER retention portion or the cytoplasmic tail portion of the polypeptide. In some embodiments, the variant VZV gE polypeptides are mutated to reduce VZV polypeptide localization to the ER/golgi/TGN. Such modifications inhibit ER trapping and, as such, expedite trafficking to the cell membrane.

Thus, in some embodiments, the VZV glycoprotein is a variant gE polypeptide. VZV gE has targeting sequences for the TGN in its C-terminus and is transported from the ER to the TGN in infected and gE-transfected cells. Most gE in the TGN appears to be retrieved by endocytosis from the plasma membrane and delivered to the TGN by endosomes, which is followed by recycling to the plasma membranes. gE is accumulated in TGN, along with other VZV proteins (e.g., tegument proteins) associated with the production of fully enveloped VZV virions. Thus, mutations to reduce TGN localization and endocytosis aids in the trafficking of gE to the cell membrane.

The variant VZV gE polypeptide can be any truncated VZV gE polypeptide lacking the anchor domain (ER retention domain). For example, the variant VZV gE polypeptide can be a truncated VZV gE polypeptide comprising at least amino acids 1-573 of the sequence shown in SEQ ID NO: 3, as well as polypeptide fragments having fragment sizes within the recited size ranges. In one embodiment, the truncated VZV gE polypeptide comprises amino acids 1-573 of the sequence shown in SEQ ID NO: 3. In some embodiments, the variant VZV gE polypeptide is a truncated polypeptide lacking the carboxy terminal tail domain. Thus in some embodiments, the truncated VZV gE polypeptide comprises amino acids 1-573 of the sequence shown in SEQ ID NO: 3.

In some embodiments, the VZV gE comprises the amino acid sequence of SEQ ID NO: 3. In some embodiments, the VZV gE comprises an amino acid sequence having at least 70% (e.g., 75%, 80%, 90%, 95%, 97%, 98%, or 99%) sequence identity to the amino acid sequence of SEQ ID NO: 3. In some embodiments, the VZV gE comprises an amino acid sequence having at least 75% sequence identity to the amino acid sequence of SEQ ID NO: 3. In some embodiments, the VZV gE comprises an amino acid sequence having at least 80% sequence identity to the amino acid sequence of SEQ ID NO: 3. In some embodiments, the VZV gE comprises an amino acid sequence having at least 90% sequence identity to the amino acid sequence of SEQ ID NO: 3. In some embodiments, the VZV gE comprises an amino acid sequence having at least 95% sequence identity to the amino acid sequence of SEQ ID NO: 3. In some embodiments, the VZV gE comprises an amino acid sequence having at least 97% sequence identity to the amino acid sequence of SEQ ID NO: 3. In some embodiments, the VZV gE comprises an amino acid sequence having at least 98% sequence identity to the amino acid sequence of SEQ ID NO: 3. In some embodiments, the VZV gE comprises an amino acid sequence having at least 99% sequence identity to the amino acid sequence of SEQ ID NO: 3.

In some embodiments, the VZV gE comprises the nucleic acid sequence of SEQ ID NO: 1. In some embodiments, the VZV gE comprises a nucleic acid sequence having at least 70% (e.g., 75%, 80%, 90%, 95%, 97%, 98%, or 99%) sequence identity to the nucleic acid sequence of SEQ ID NO: 1. In some embodiments, the VZV gE comprises a nucleic acid sequence having at least 75% sequence identity to the nucleic acid sequence of SEQ ID NO: 1. In some embodiments, the VZV gE comprises a nucleic acid sequence having at least 80% sequence identity to the nucleic acid sequence of SEQ ID NO: 1. In some embodiments, the VZV gE comprises a nucleic acid sequence having at least 90% sequence identity to the nucleic acid sequence of SEQ ID NO: 1. In some embodiments, the VZV gE comprises a nucleic acid sequence having at least 95% sequence identity to the nucleic acid sequence of SEQ ID NO: 1. In some embodiments, the VZV gE comprises a nucleic acid sequence having at least 97% sequence identity to the nucleic acid sequence of SEQ ID NO: 1. In some embodiments, the VZV gE comprises a nucleic acid sequence having at least 98% sequence identity to the nucleic acid sequence of SEQ ID NO: 1. In some embodiments, the VZV gE comprises a nucleic acid sequence having at least 99% sequence identity to the nucleic acid sequence of SEQ ID NO: 1.

In some embodiments, the variant VZV gE polypeptide has at least one mutation in one or more motif(s) associated with ER retention, wherein the mutation(s) in one or more motif(s) results in decreased retention of the VZV gE polypeptide in the ER and/or golgi. For example, the variant VZV gE polypeptide can be a full-length or truncated VZV gE polypeptide having a Y569A mutation.

In some embodiments, the RNA elicits an immune response against coronavirus selected from the group consisting of 229E (alpha coronavirus), NL63 (alpha coronavirus), OC43 (beta coronavirus), HKU1 (beta coronavirus), MERS-CoV (MERS), SARS-CoV (SARS), and SARS-CoV-2 (COVID-19. In some embodiments, the virus is SARS-CoV-2. In some embodiments, the RNA elicits an immune response against SARS-CoV-2.

In some embodiments, the RNA elicits an immune response against a SARS-CoV-2 variant selected from the group consisting of Wuhan-Hu-1, Alpha, Beta, Gamma, Delta, Epsilon Eta, Iota, Kappa, 1.617.3, Mu, Zeta, and Omicron. In some embodiments, the RNA elicits an immune response against a SARS-CoV-2 variant selected from the group consisting of Wuhan-Hu-1 and Delta.

In some embodiments, the antigenic polypeptide includes a viral envelope protein, viral spike protein, viral membrane protein, or viral capsid protein. In some embodiments, the antigenic polypeptide comprises a viral spike protein.

### Self-Replicating RNA

A polynucleotide can also include RNAs such as a self-replicating RNA. The RNA described herein can be a self-replicating RNA. A self-replicating RNA molecule (replicon) can, when delivered to a vertebrate cell even without any proteins, lead to the production of multiple daughter RNAs by transcription from itself (via an antisense copy which it generates from itself). A self-replicating RNA molecule can thus typically be a +−strand molecule which can be directly translated after delivery to a cell, and this translation provides an RNA-dependent RNA polymerase which then produces both antisense and sense transcripts from the delivered RNA. Thus the delivered RNA can lead to the production of multiple daughter RNAs. These daughter RNAs, as well as collinear subgenomic transcripts, may be translated themselves to provide in situ expression of an encoded immunogen, or may be transcribed to provide further transcripts with the same sense as the delivered RNA which are translated to provide in situ expression of the immunogen. The overall results of this sequence of transcriptions are a large amplification in the number of the introduced replicon RNAs and so the encoded immunogen becomes a major polypeptide product of the cells.

One suitable system for achieving self-replication in this manner is to use an alphavirus-based replicon. These replicons can be +−stranded RNAs which lead to translation of a replicase (or replicase-transcriptase) after delivery to a cell. The replicase can be translated as a polyprotein which auto-cleaves to provide a replication complex which creates genomic −−strand copies of the +−strand delivered RNA. These −−strand transcripts can themselves be transcribed to give further copies of the +−stranded parent RNA and also to give a subgenomic transcript which encodes the immunogen. Translation of the subgenomic transcript can thus lead to in situ expression of the immunogen by the infected cell. Suitable alphavirus replicons can use a replicase from a Sindbis virus, a SemLiki forest virus, an eastern equine encephalitis virus, a Venezuelan equine encephalitis virus, etc. Mutant or wild-type virus sequences can be used e.g., the attenuated TC83 mutant of VEEV has been used in replicons.

A self-replicating RNA molecule can encode (i) an RNA-dependent RNA polymerase which can transcribe RNA from the self-replicating RNA molecule and (ii) an immunogen. The polymerase can be an alphavirus replicase e.g., comprising one or more of alphavirus proteins nsp1, nsp2, nsp3 and nsp4.

Whereas natural alphavirus genomes encode structural virion proteins in addition to the non-structural replicase polyprotein, a self-replicating RNA molecule described herein can lack one or more or all alphavirus structural proteins. Thus a self-replicating RNA can lead to the production of genomic RNA copies of itself in a cell, but not to the production of RNA-containing virions. The inability to produce these virions means that, unlike a wild-type alphavirus, the self-replicating RNA molecule generally does not perpetuate itself in infectious form. The alphavirus structural proteins which are used for perpetuation in wild-type viruses are typically absent from self-replicating RNAs described herein and their place is taken by gene(s) encoding the immunogen of interest, such that the subgenomic transcript encodes the immunogen rather than the structural alphavirus virion proteins.

Thus a self-replicating RNA molecule may have two open reading frames. The first (5′) open reading frame encodes a replicase; the second (3′) open reading frame encodes an immunogen. In some embodiments the RNA may have additional (e.g., downstream) open reading frames, e.g., to encode further immunogens (see below) or to encode accessory polypeptides.

Self-replicating RNA molecules can have various lengths but they are typically 5,000-25,000 nucleotides long, e.g., 8,000-15,000 nucleotides, or 9,000-12,000 nucleotides. In some embodiments, the self-replicating RNA comprises the nucleic acid sequence of SEQ ID NO: 2. In some embodiments, the self-replicating RNA comprises the nucleic acid sequence of SEQ ID NO: 2. In some embodiments, the self-replicating RNA comprises a nucleic acid sequence having at least 70% (e.g., 75%, 80%, 90%, 95%, 97%, 98%, or 99%) sequence identity to the nucleic acid sequence of SEQ ID NO: 2. In some embodiments, the self-replicating RNA comprises a nucleic acid sequence having at least 75% sequence identity to the nucleic acid sequence of SEQ ID NO: 2. In some embodiments, the self-replicating RNA comprises a nucleic acid sequence having at least 80% sequence identity to the nucleic acid sequence of SEQ ID NO: 2. In some embodiments, the self-replicating RNA comprises a nucleic acid sequence having at least 90% sequence identity to the nucleic acid sequence of SEQ ID NO: 2. In some embodiments, the self-replicating RNA comprises a nucleic acid sequence having at least 95% sequence identity to the nucleic acid sequence of SEQ ID NO: 2. In some embodiments, the self-replicating RNA comprises a nucleic acid sequence having at least 97% sequence identity to the nucleic acid sequence of SEQ ID NO: 2. In some embodiments, the self-replicating RNA comprises a nucleic acid sequence having at least 98% sequence identity to the nucleic acid sequence of SEQ ID NO: 2. In some embodiments, the self-replicating RNA comprises a nucleic acid sequence having at least 99% sequence identity to the nucleic acid sequence of SEQ ID NO: 2.

Described herein, in some embodiments, are self-replicating RNA molecules comprising one more components (e.g., Cap1, a modified 3′ UTR, an extended polyA tail, or combinations thereof) that improve the gene of interest (GOI) expression, the immunogenicity, the scalability and manufacturability, or combinations thereof. In some embodiments, In some embodiments, the self-replicating RNA molecules comprise at least 1.5×, 2.0×, 2.5×, 3.0×, or more GOI expression as compared to an unmodified vector. In some embodiments, the srRNA vector comprises at least 2×, 5×, 10×, 20×, 25×, 30×, or more improved immune response as compared to an unmodified vector. In some embodiments, the unmodified srRNA vector comprises a polyA tail about 10, 15, 20, 25, 30, 35, 40, 45, 50, 55, 60, 70, 75, 80, 85, 90, 100, 110, 120, 130, or 140 nucleotides in length. In some embodiments, an unmodified srRNA vector comprises a Cap that is not Cap 1 (e.g. Cap 0). In some embodiments, an unmodified srRNA vector comprises no modifications in one or more components (cap, UTR, or polyA tail) as compared to a parental vector.

A self-replicating RNA molecule described herein may have a 5′ cap (e.g., a 7-methylguanosine). This cap can enhance in vivo translation of the RNA. The 5′ nucleotide of a RNA molecule useful with the present disclosure may have a 5′ triphosphate group. In a capped RNA this may be linked to a 7-methylguanosine via a 5′-to-5′ bridge. In some embodiments, the RNA cap may include m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm-—where Nm denotes any nucleotide with a 2′ O methylation.

A self-replicating RNA molecule may have a 3′ poly-A tail. It may also include a poly-A polymerase recognition sequence (e.g., AAUAAA) near its 3′ end.

In some embodiments, the self-replicating RNA described herein comprises an elongated polyA tail. In some embodiments, the self-replicating RNA described herein comprises a polyA tail between 30-100 nucleotides in length. In some embodiments, the self-replicating RNA described herein comprises a polyA tail between 35-100 nucleotides in length. In some embodiments, the self-replicating RNA described herein comprises a polyA tail between 40-100 nucleotides in length. In some embodiments, the self-replicating RNA described herein comprises a polyA tail between 45-100 nucleotides in length. In some embodiments, the self-replicating RNA described herein comprises a polyA tail between 50-100 nucleotides in length. In some embodiments, the self-replicating RNA described herein comprises a polyA tail between 55-100 nucleotides in length. In some embodiments, the self-replicating RNA described herein comprises a polyA tail between 60-100 nucleotides in length. In some embodiments, the self-replicating RNA described herein comprises a polyA tail between 65-100 nucleotides in length. In some embodiments, the self-replicating RNA described herein comprises a polyA tail between 70-100 nucleotides in length. In some embodiments, the self-replicating RNA described herein comprises a polyA tail between 80-100 nucleotides in length. In some embodiments, the self-replicating RNA described herein comprises a polyA tail between 90-100 nucleotides in length. In some embodiments, the self-replicating RNA described herein comprises a polyA tail between 95-100 nucleotides in length. In some embodiments, the self-replicating RNA described herein comprises a polyA tail between 30-95 nucleotides in length. In some embodiments, the self-replicating RNA described herein comprises a polyA tail between 30-90 nucleotides in length. In some embodiments, the self-replicating RNA described herein comprises a polyA tail between 30-85 nucleotides in length. In some embodiments, the self-replicating RNA described herein comprises a polyA tail between 30-80 nucleotides in length. In some embodiments, the self-replicating RNA described herein comprises a polyA tail between 30-75 nucleotides in length. In some embodiments, the self-replicating RNA described herein comprises a polyA tail between 30-70 nucleotides in length. In some embodiments, the self-replicating RNA described herein comprises a polyA tail between 30-65 nucleotides in length. In some embodiments, the self-replicating RNA described herein comprises a polyA tail between 30-60 nucleotides in length. In some embodiments, the self-replicating RNA described herein comprises a polyA tail between 30-55 nucleotides in length. In some embodiments, the self-replicating RNA described herein comprises a polyA tail between 30-50 nucleotides in length. In some embodiments, the self-replicating RNA described herein comprises a polyA tail between 30-45 nucleotides in length. In some embodiments, the self-replicating RNA described herein comprises a polyA tail of 10, 15, 20, 25, 30, 35, 40, 45, 50, 55, 60, 65, 70, 75, 80, 85, 90, 95, 100, 110, 120, 130, 140, or more than 140 nucleotides in length.

In some embodiments, the self-replicating RNA described herein has an elongated 3′ UTR. In some embodiments, the self-replicating RNA described herein has a 3′ UTR between 180-400 nucleotides in length. In some embodiments, the self-replicating RNA described herein has a 3′ UTR between 100-500 nucleotides in length. In some embodiments, the self-replicating RNA described herein has a 3′ UTR between 125-500 nucleotides in length. In some embodiments, the self-replicating RNA described herein has a 3′ UTR between 150-500 nucleotides in length. In some embodiments, the self-replicating RNA described herein has a 3′ UTR between 175-500 nucleotides in length. In some embodiments, the self-replicating RNA described herein has a 3′ UTR between 200-500 nucleotides in length. In some embodiments, the self-replicating RNA described herein has a 3′ UTR between 225-500 nucleotides in length. In some embodiments, the self-replicating RNA described herein has a 3′ UTR between 250-500 nucleotides in length. In some embodiments, the self-replicating RNA described herein has a 3′ UTR between 275-500 nucleotides in length. In some embodiments, the self-replicating RNA described herein has a 3′ UTR between 300-500 nucleotides in length. In some embodiments, the self-replicating RNA described herein has a 3′ UTR between 325-500 nucleotides in length. In some embodiments, the self-replicating RNA described herein has a 3′ UTR between 100-476 nucleotides in length. In some embodiments, the self-replicating RNA described herein has a 3′ UTR between 100-450 nucleotides in length. In some embodiments, the self-replicating RNA described herein has a 3′ UTR between 100-425 nucleotides in length. In some embodiments, the self-replicating RNA described herein has a 3′ UTR between 100-400 nucleotides in length. In some embodiments, the self-replicating RNA described herein has a 3′ UTR between 100-375 nucleotides in length. In some embodiments, the self-replicating RNA described herein has a 3′ UTR between 100-350 nucleotides in length. In some embodiments, the self-replicating RNA described herein has a 3′ UTR between 300-350 nucleotides in length. In some embodiments, the self-replicating RNA described herein has a 3′ UTR about 330 nucleotides in length. In some embodiments, the self-replicating RNA described herein has a 3′ UTR about 270 nucleotides in length. In some embodiments, the self-replicating RNA described herein comprises a 3′ UTR of 50, 75, 100, 125, 150, 175, 200, 225, 250, 275, 300, 325, 350, 375, 400 or more than 440 nucleotides in length.

In some embodiments, the 3′ UTR comprises the nucleic acid sequence of SEQ ID NO: 6. In some embodiments, the 3′ UTR comprises a nucleic acid sequence having at least 70% (e.g., 75%, 80%, 90%, 95%, 97%, 98%, or 99%) sequence identity to the nucleic acid sequence of SEQ ID NO: 6. In some embodiments, the 3′ UTR comprises a nucleic acid sequence having at least 75% sequence identity to the nucleic acid sequence of SEQ ID NO: 6. In some embodiments, the 3′ UTR comprises a nucleic acid sequence having at least 80% sequence identity to the nucleic acid sequence of SEQ ID NO: 6. In some embodiments, the 3′ UTR comprises a nucleic acid sequence having at least 90% sequence identity to the nucleic acid sequence of SEQ ID NO: 6. In some embodiments, the 3′ UTR comprises a nucleic acid sequence having at least 95% sequence identity to the nucleic acid sequence of SEQ ID NO: 6. In some embodiments, the 3′ UTR comprises a nucleic acid sequence having at least 97% sequence identity to the nucleic acid sequence of SEQ ID NO: 6. In some embodiments, the 3′ UTR comprises a nucleic acid sequence having at least 98% sequence identity to the nucleic acid sequence of SEQ ID NO: 6. In some embodiments, the 3′ UTR comprises a nucleic acid sequence having at least 99% sequence identity to the nucleic acid sequence of SEQ ID NO: 6.

In some embodiments, the self-replicating RNA molecule described herein comprises a Cap (e.g., m7G (Cap 0), m7GpppNm-, where Nm denotes any nucleotide with a 2′ O methylation (Cap 1), N6,2′-O-dimethyladenosine (m6AM), m7G(5′)ppp(5′)G (mCAP), or anti-reverse cap analogs (ARCA), optionally m7G or m7GpppNm-—where Nm denotes any nucleotide with a 2′ O methylation), a 5′ UTR, one or more alphavirus proteins (e.g., nsp1, nsp2, nsp3 and nsp4), a gene of interest (e.g., VZV), a 3′ UTR, and a poly A tail. In some embodiments, the 3′ UTR is an elongated UTR comprising about 50, 75, 100, 125, 150, 175, 200, 225, 250, 275, 300, 325, 350, 375, 400 or more than 440 nucleotides in length. In some embodiments, the 3′ UTR comprises about 330 nucleotides in length. In some embodiments, the 3′ UTR comprises about 270 nucleotides in length. In some embodiments, the polyA tail comprises about 10, 15, 20, 25, 30, 35, 40, 45, 50, 55, 60, 65, 70, 75, 80, 85, 90, 95, 100, 110, 120, 130, 140, or more than 140 nucleotides in length. In some embodiments, the polyA tail comprises about 65 nucleotides in length. In some embodiments, the polyA tail comprises about 95 nucleotides in length.

In some embodiments, the self-replicating RNA molecule or vector described herein comprises Cap 0, a 5′ UTR, one or more alphavirus proteins (e.g., nsp1, nsp2, nsp3 and nsp4), a gene of interest (e.g., VZV), a 3′ UTR comprising about 180-400 (e.g., about 270 or about 330 nucleotides) nucleotides in length, and a poly A tail comprising about 40 to about 100 nucleotides in length (e.g., about 40, about 65, or about 95 nucleotides in length). In some embodiments, the self-replicating RNA molecule described herein comprises Cap 1, a 5′ UTR, one or more alphavirus proteins (e.g., nsp1, nsp2, nsp3 and nsp4), a gene of interest (e.g., VZV), a 3′ UTR comprising about 100-400 (e.g., about 270 or about 330 nucleotides) nucleotides in length, and a poly A tail comprising about 40 to about 100 nucleotides in length (e.g., about 40, about 65, or about 95 nucleotides in length).

In some embodiments, the self-replicating RNA (srRNA) molecule or vector comprises, in 5′ to 3′ order, a) a Cap1 cap, b) a 5′ UTR, c) one or more structural proteins; d) a gene of interest (GOI); e) a 3′ UTR comprising about 180 to about 400 nucleotides; and f) a poly A tail comprising about 60 to about 100 nucleotides. In some embodiments, the srRNA molecule or vector comprises at least 1.5×, 2.0×, 2.5×, 3.0×, or more GOI expression as compared to an unmodified molecule or vector. In some embodiments, the srRNA molecule or vector comprises at least 2×, 5×, 10×, 20×, 25×, 30×, or more improved immune response as compared to an unmodified molecule or vector. In some embodiments, the GOI is a varicella-zoster virus (VZV) antigen. In some embodiments, the VZV antigen comprises a VZV glycoprotein E (gE) antigen.

A self-replicating RNA molecule will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

A self-replicating RNA molecule described herein can be prepared by in vitro transcription (IVT). IVT can use a cDNA template created and propagated in plasmid form in bacteria, or created synthetically (for example, by gene synthesis and/or polymerase chain-reaction (PCR) engineering methods). For instance, a DNA-dependent RNA polymerase (such as the bacteriophage T7, T3 or SP6 RNA polymerases) can be used to transcribe the RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

### The Immunogen

RNA molecules described herein may encode a polypeptide immunogen. Self-replicating RNA molecules described herein can encode a polypeptide immunogen. After administration of the RNA, the immunogen is translated in vivo and can elicit an immune response in the recipient. The immunogen may elicit an immune response against an antigen, virus, and/or viral antigen. The immune response may comprise an antibody response. The immune response may comprise B cells, CD4+ T cells, and/or CD8+ T cells. The immune response may comprise CD8+ T cells. The immunogen will typically elicit an immune response which recognizes the corresponding antigen such as a viral polypeptide. The immunogen will typically be a surface polypeptide, e.g., an adhesin, a hemagglutinin, an envelope glycoprotein, a spike glycoprotein, etc. In some embodiments, the immunogen elicits an immune response against the spike glycoprotein of SARS-CoV-2. In some embodiments, the immunogen elicits an immune response against the varicella-zoster virus. In some embodiments, the immunogen is VZV glycoprotein E (gE).

In some embodiments, the VZV srRNA vaccine described herein provides improved immune response. In some embodiments, the immune response is about 1×, 2×, 3×, 4×, 5×, 6×, or more improved as compared to a subject that does not receive the srRNA vaccine or receives a different VZV vaccine.

In some embodiments, the VZV srRNA vaccine described herein provides improved humoral response. In some embodiments, the humoral response is about 50×, 60×, 70×, 80×, 90×, 100×, 110×, 120×, 130×, 140×, 150×, 160×, 170×, 180×, 190×, 200×, or more improved as compared to a subject that does not receive the srRNA vaccine or receives a different VZV vaccine.

### Nanoparticle Formulations

In the present disclosure, a method is provided to mix lyophilized RNA with a delivery vehicle. A delivery vehicle can be non-virion particles, i.e., they are not a virion. Thus, in some embodiments, the delivery vehicle does not comprise a protein capsid. By avoiding the need to create a capsid, a delivery vehicle does not utilize a packaging cell line, thus permitting easier up-scaling for commercial production and minimizing the risk that dangerous infectious viruses will inadvertently be produced. Various materials are suitable delivery particles which can deliver RNA to a vertebrate cell in vivo. Two delivery materials are (i) amphiphilic lipids which can form liposomes and (ii) non-toxic and biodegradable polymers which can form microparticles. Other delivery methods may include, but are not limited to, exosomes and cationic nano-emulsions.

Where delivery is by liposome, the RNA can be encapsulated or adsorbed; where delivery is by polymeric microparticle, the RNA can be encapsulated or adsorbed. A third delivery material is the particulate reaction product of a polymer, a crosslinker, a RNA, and a charged monomer. In certain embodiments, the delivery particle described herein comprises a liposome adsorbing RNA molecules which encode an antigen.

The RNA can be encapsulated within liposomes. This means that RNA inside the particles is separated from any external medium by the delivery material, and encapsulation has been found to protect RNA from RNase digestion. Encapsulation can take various forms. For example, in some embodiments, the delivery material forms a outer layer around an aqueous RNA-containing core. In some embodiments, RNA can be adsorbed to the particles. This means, in some embodiments, that RNA is not separated from any external medium by the delivery material, unlike the RNA genome of a natural virus. In some embodiments, RNAs are formulated in a lipid nanoparticle.

In some embodiments, an RNA delivery vehicle is a nanoparticle (e.g., LNP) that comprises at least one lipid. In some embodiments, the lipid comprises an ionizable lipid of Formula I.

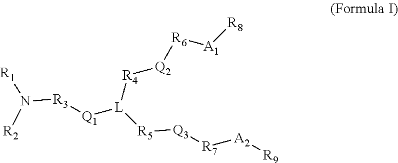

R₁ and R₂ are each, independently C₁-C₆ alkyl;
R₃ is C₁-C₅ alkyl;
Q₁, Q₂ and Q₃ are each independently —O—, —S—, —C(O)O—, —OC(O)—, —S—S—, —C(O)S—, —SC(O)—, —OC(S)—, or —C(S)O—;
L is C₁-C₃ alkyl
R₄ and R₅ are each, independently C₁-C₁₀ alkyl;
R₆ and R₇ are each, independently C₁-C₁₀ alkyl, C₁-C₁₀ alkenyl;
A₁ and A₂ are each independently a bond, —O—, —S, C(O)O—, —OC(O)—, —S—S—, —C(O)S—, —SC(O)—, —OC(S)—, or —C(S)O—;
R₈ and R₉ are each, independently C₁-C₃₀ alkyl.

In some embodiments, L is

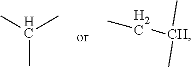

In some embodiments, the lipid comprises an ionizable lipid of Formula II

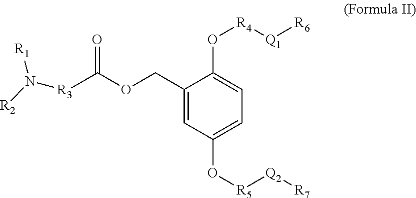

wherein:
R₁ and R₂ are each independently C₁ to C₆ alkyl;
R₃ is C₁ to C₅ alkyl;
R₄ and R₅ are each independently C₁ to C₁₈ alkyl group
Q₁ and Q₂ are each independently —O—C(O)—, —C(O)—O—, —O—C(S)—, —C(S)—O—; —S—S—, and
R₆ and R₇ are each independently C₁ to C₃₂ alkyl.

In some embodiments, R₁ and R₂ are methyl.

In some embodiments, Q₁ and Q₂ are each independently —C(O)—O— or —O—C(O)—.

In some embodiments, R₃ is a straight chained C₃ alkyl.

In some embodiments, R4 and R5 are each independently C5-C10 alkyl group.

In some embodiments, R6 and R7 are each independently C1-C22 alkyl. In some embodiments, R6 and R7 are each independently C7-C17 alkyl.

In some embodiments, the lipid may be Lipid #2,

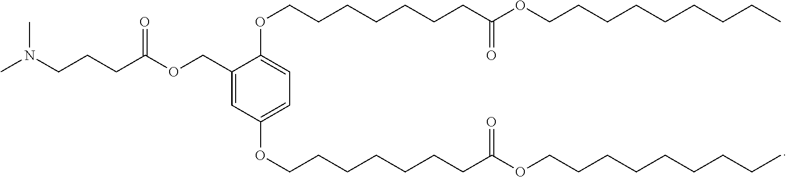 [SMILES: CCCCCCCCCOC(=O)CCCCCCCOc1ccc(OCCCCCCCC(=O)OCCCCCCCCC)c(COC(=O)CCCN(C)C)c1]

In some embodiments, the lipid may be Lipid #4,

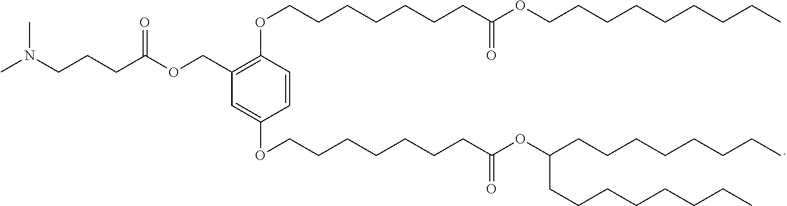 [SMILES: CCCCCCCCCOC(=O)CCCCCCCOc1ccc(OCCCCCCCC(=O)OC(CCCCCCCC)CCCCCCCC)cc1COC(=O)CCCN(C)C]

In some embodiments, the lipid may be Lipid #5,

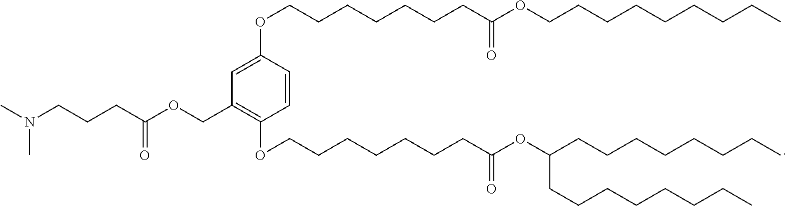 [SMILES: CCCCCCCCCOC(=O)CCCCCCCOc1ccc(OCCCCCCCC(=O)OC(CCCCCCCC)CCCCCCCC)c(COC(=O)CCCN(C)C)c1]

In some embodiments, the lipid may be Lipid #8,

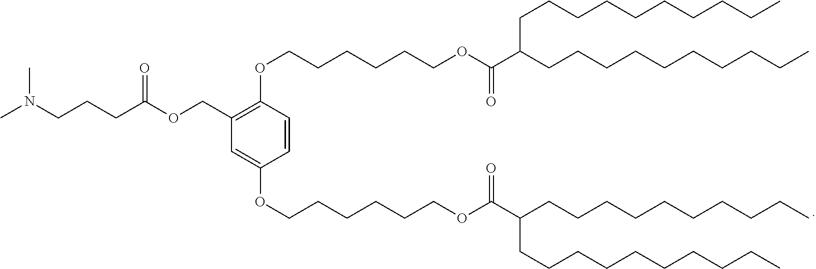 [SMILES: CCCCCCCCCCC(CCCCCCCCCC)C(=O)OCCCCCCOc1ccc(OCCCCCCOC(=O)C(CCCCCCCCCC)CCCCCCCCCC)c(COC(=O)CCCN(C)C)c1]

In some embodiments the lipid may be Lipid #9,

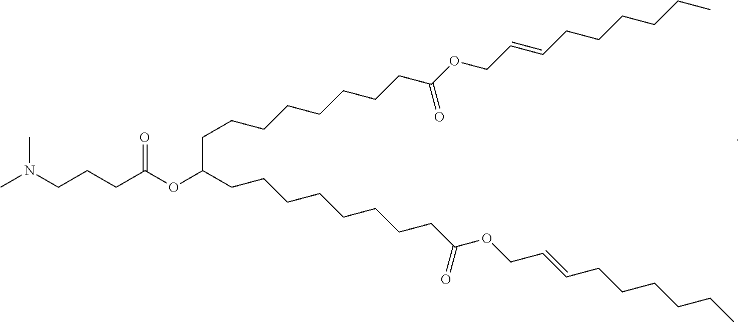 [SMILES: CCCCCC/C=C/COC(=O)CCCCCCCCC(CCCCCCCCC(=O)OC/C=C/CCCCCC)OC(=O)CCCN(C)C]

In some embodiments, the lipid may be Lipid #10,

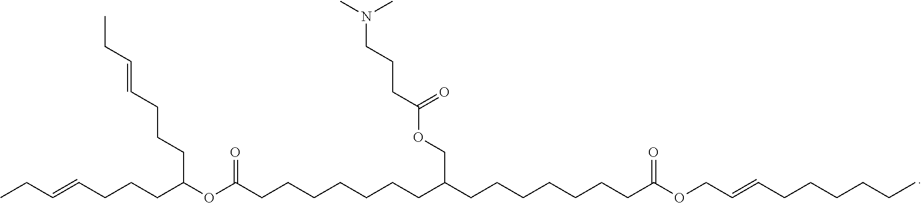 [SMILES: CC/C=C/CCCC(CCC/C=C/CC)OC(=O)CCCCCCCC(CCCCCCCC(=O)OC/C=C/CCCCCC)COC(=O)CCCN(C)C]

In some embodiments the lipid may be Lipid #11,

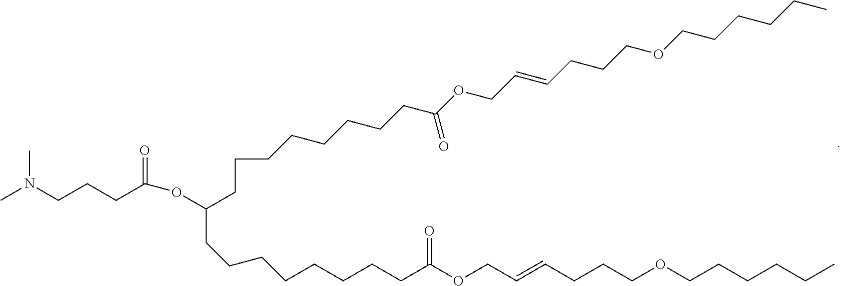 [SMILES: CCCCCCOCCC/C=C/COC(=O)CCCCCCCCC(CCCCCCCCC(=O)OC/C=C/CCCOCCCCCC)OC(=O)CCCN(C)C]

In some embodiments the lipid may be Lipid #12,

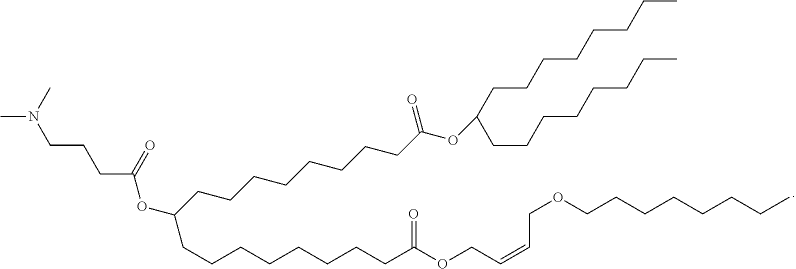 [SMILES: CCCCCCCCOC/C=C\COC(=O)CCCCCCCCC(CCCCCCCCC(=O)OC(CCCCCCCC)CCCCCCCC)OC(=O)CCCN(C)C]

In some embodiments, pharmaceutically acceptable salts of the lipids described herein include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate, trifluoroacetate, and undecanoate.

In some embodiments, the lipid may be a cationic lipid, also called ionizable lipid. In some embodiments, useful cationic lipids generally contain a nitrogen atom that is positively charged under physiological conditions e.g., as a tertiary or quaternary amine. This nitrogen can be in the hydrophilic head group of an amphiphilic surfactant. The lipid may be selected from, but is not limited to, 1,2-dioleoyloxy-3-(trimethylammonio)propane (DOTAP), 3′-[N—(N′,N′-Dimethylaminoethane)-carbamoyl] cholesterol (DC cholesterol), dimethyldioctadecyl-ammonium (DDA e.g., the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP). Other useful cationic lipids are: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC), cetyl trimethylammonium chloride (CTAC), N, N′, N′-polyoxyethylene (10)—N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), dialkyldimetylammonium salts, [1-(2,3-dioleyloxy)-propyl]-N,N,N,trimethylammonium chloride, 1,2-diacyl-3-(trimethylammonio)propane (acyl group=dimyristoyl, dipalmitoyl, distearoyl, dioleoyl), 1,2-diacyl-3-(dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio)butanoate), N-alkyl pyridinium salts (e.g., cetylpyridinium bromide and cetylpyridinium chloride), N-alkylpiperidinium salts, dicationic bolaform electrolytes (Cl2Me6; Cl2BU6), dialkylglycetylphosphorylcholine, lysolecithin, L-α dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (C₁₂GluPhCₙN⁺), ditetradecyl glutamate ester with pendant amino group (C12GluPhCₙN^{|}), cationic derivatives of cholesterol, including but not limited to cholesteryl-3 β-oxysuccinamidoethylenetrimethylammonium salt, cholesteryl-3 β-oxysuccinamidoethylenedimethylamine, cholesteryl-3 β-carboxyamidoethylenetrimethylammonium salt, and cholesteryl-3 β-carboxyamidoethylenedimethylamine.

In some embodiments, an RNA delivery vehicle is a nanoparticle that comprises at least one lipid. In some embodiments, the lipid may be a neutral lipid. In some embodiments, the lipid may be a phospholipid. The phospholipid may be selected from, but is not limited to, DDPC, 1,2-Didecanoyl-sn-Glycero-3-phosphatidylcholine, DEPA, 1,2-Dierucoyl-sn-Glycero-3-Phosphate, DEPC, 1,2-Erucoyl-sn-Glycero-3-phosphatidylcholine, DEPE, 1,2-Dierucoyl-sn-Glycero-3-phosphatidylethanolamine, DEPG, 1,2-Dipalmitoylphosphatidylglycerol, DLOPC

1,2-Linoleoyl-sn-Glycero-3-phosphatidylcholine, DLPA, 1,2-Dilauroyl-sn-Glycero-3-Phosphate, DLPC, 1,2-Dilauroyl-sn-Glycero-3-phosphatidylcholine, DLPE 1,2-Dilauroyl-sn-Glycero-3-phosphatidylethanolamine, DLPG 1,2-Dilauroyl-sn-Glycero-3(phosphorylglycerol), DLPS 1,2-Dilauroyl-sn-Glycero-3-phosphatidylserine, DMG, 1,2-Dimyristoyl-sn-glycero-3-phosphoethanolamine, DMPA, 1,2-Dimyristoyl-sn-Glycero-3-Phosphate, DMPC, 1,2-Dimyristoyl-sn-Glycero-3-phosphatidylcholine, DMPE, 1,2-Dimyristoyl-sn-Glycero-3-phosphatidylethanolamine, DMPG, 1,2-Dimyristoyl-sn-glycero-3-phosphoglycerol, DMPS, 1,2-Dimyristoyl-sn-Glycero-3-phosphatidylserine, DOPA, 1,2-Dioleoyl-sn-Glycero-3-Phosphate, DOPC, 1,2-Dioleoyl-sn-Glycero-3-phosphatidylcholine, DOPE, 1,2-Dioleoyl-sn-Glycero-3-phosphatidylethanolamine, DOPG, 1,2-Dipalmitoylphosphatidylglycerol, DOPS, 1,2-Dioleoyl-sn-Glycero-3-phosphatidylserine, DPPA, 1,2-Dipalmitoyl-sn-Glycero-3-Phosphate, DPPC, 1,2-Dipalmitoyl-sn-Glycero-3-phosphatidylcholine, DPPE, 1,2-Dipalmitoyl-sn-Glycero-3-phosphatidylethanolamine, DPPG, 1,2-Dipalmitoyl-sn-glycero-3-phosphoglycerol, DPPS, 1,2-Dipalmitoyl-sn-Glycero-3-phosphatidylserine, DPyPE, 1,2-diphytanoyl-sn-glycero-3-phosphoethanolamine, DSPA, 1,2-Distearoyl-sn-Glycero-3-Phosphate, DSPC, 1,2-Distearoyl-sn-Glycero-3-phosphatidylcholine, DSPE, 1,2-Diostearpyl-sn-Glycero-3-phosphatidylethanolamine, DSPG, 1,2-Distearoyl-sn-Glycero-3-phosphorylglycerol, DSPS, 1,2-diundecanoyl-sn-glycero-phosphocholine, DUPC, 1,2-Distearoyl-sn-Glycero-3-phosphatidylserine, EPC, Egg-PC, HEPC, Hydrogenated Egg PC, HSPC, High purity Hydrogenated Soy PC, HSPC, Hydrogenated Soy PC, Lysopc Myristic, 1-Myristoyl-sn-Glycero-3-phosphatidylcholine, LYSOPC PALMITIC, 1-Palmitoyl-sn-Glycero-3-phosphatidylcholine, LYSOPC STEARIC, 1-Stearoyl-sn-Glycero-3-phosphatidylcholine, Milk Sphingomyelin, MPPC, 1-Myristoyl,2-palmitoyl-sn-Glycero 3-phosphatidyl choline, MSPC, 1-Myristoyl,2-stearoyl-sn-Glycero-3-phosphatidylcholine, PMPC, 1-Palmitoyl,2-myristoyl-sn-Glycero-3-phosphatidylcholine, POPC, 1-Palmitoyl,2-oleoyl-sn-Glycero-3-phosphatidylcholine, POPE, 1-Palmitoyl-2-oleoyl-sn-Glycero-3-phosphatidylethanolamine, POPG, 1,2-Dioleoyl-sn-Glycero-3-Phospho-rac-(1-glycerol)], PSPC, 1-Palmitoyl,2-stearoyl-sn-Glycero-3-phosphatidylcholine, SMPC, 1-Stearoyl,2-myristoyl-sn-Glycero-3-phosphatidylcholine, SOPC, 1-Stearoyl,2-oleoyl-sn-Glycero-3-phosphatidylcholine, SPPC, 1-Stearoyl,2-palmitoyl-sn-Glycero-3-phosphatidylcholine.

In some embodiments, an RNA delivery vehicle is a nanoparticle that comprises at least one lipid. In some embodiments, the lipid may be a PEGylated lipid (PEG). In some embodiments, the PEGylated lipid comprises a polyethylene glycol moiety. In some embodiments, a PEG lipid may include but not limited to PEG-modified phosphatidylethanolamines, PEG-modified phosphatidic acids, PEG-modified ceramides, PEG-modified dialkylamines, PEG-modified diacylglycerols, PEG-modified dialkylglycerols, or combinations thereof. In some embodiments, a PEGylated lipid may be PEG-c-DOMG, PEG-DMG, PEG-DLPE, PEG-DMPE, PEG-DPPC, or a PEG-DSPE lipid. In some embodiments, the PEGylated lipid is DMG-PEG, i.e. PEG-conjugated 1,2-dimyristoyl-sn-glycero-3-phosphoethanolamine-N-[methoxy(polyethylene glycol). In some embodiments, the lipid is DMG-PEG 2000, i.e. 1,2-dimyristoyl-rac-glycero-3-methoxypolyethylene glycol-2000. In some embodiments, the pegylated lipid comprises 1,2-dimyristoyl-rac-glycero-3-methoxypolyethylene glycol-2000 (DMG-PEG 2000), DMG-PEG 3500, DMG-PEG 5000, DTDAM-PEG 2000 (ALC-0159), DTDAM-PEG 5000, DMG-C-PEG 2000, DMG-C-PEG 5000, DSG-PEG 2000, DSG-PEG 5000, DPG-PEG 2000, DPG-PEG 5000, or any combination thereof.

In some embodiments, an RNA delivery vehicle is a nanoparticle that comprises at least one lipid. In some embodiments, the lipid may be a structural 1 lipid. In some embodiments, structural lipids can include, but are not limited to, cholesterol, fecosterol, sitosterol, ergosterol, campesterol, stigmasterol, brassicasterol, tomatidine, tomatine, ursolic acid, alpha-tocopherol, or combinations thereof. In some embodiments, the structural lipid is cholesterol. In some embodiments, the structural lipid includes cholesterol and a corticosteroid (such as prednisolone, dexamethasone, prednisone, and hydrocortisone), or a combination thereof.

In some embodiments, a lipid nanoparticle (LNP) formulation comprises, consists essentially of, or consists of (i) a neutral phospholipid (ii) a sterol, e.g., cholesterol; (iii) a pegylated lipid optionally and (iv) a ionizable lipid with the molar ratio within ranges of neutral phospholipid: 5%-20%, sterol: 18.5%-58.5%, pegylated lipid: 1%-4%, ionizable lipid: 30%-70%, the total summation of the mole ratio of the lipids is 100%, optionally 10:40.5:1.5:48.

In some embodiments, the LNP comprises a DSPC:cholesterol:DMG-PEG 2000:and an ionizable lipid with the mole ratio within the ranges of DSPC: 5%-20%, cholesterol: 18.5%-58.5%, DMG-PEG 2000: 1%-4%, ionizable lipid: 30%-70%, and the total summation of the mole ratio of the lipids is 100%.

In some embodiments, the LNP comprises a DSPC:cholesterol:DMG-PEG 2000:and an ionizable lipid with the mole ratio within the ranges of DSPC: 5%-20%, cholesterol: 30%-55%, DMG-PEG 2000: 10.5%-43%, ionizable lipid: 40%-55%, the total summation of the mole ratio of the lipids is 100.

In some embodiments, the LNP comprises a DSPC:cholesterol:DMG-PEG 2000:ionizable lipid with the mole ratio within the ranges of DSPC: 5%-20%, cholesterol: 30%-55%, DMG-PEG 2000: 1%-4%, ionizable lipid: 40%-50%, the total summation of the mole ratio of the lipids is 100%.

In some embodiments, the LNP comprises a DSPC:cholesterol:DMG-PEG 2000:ionizable lipid with the mole ratio within the ranges of DSPC: 5%-20%, cholesterol: 30%-55%, DMG-PEG 2000: 1%-4%, ionizable lipid: 45%-50%, the total summation of the mole ratio of the lipids is 100.

In some embodiments, the LNP comprises a DSPC:cholesterol:DMG-PEG 2000:ionizable lipid with the mole ratio within the ranges of DSPC: 5%-20%, cholesterol: 30%-55%, DMG-PEG 2000: 1%-4%, ionizable lipid: 46%-49%, the total summation of the mole ratio of the lipids is 100%.

In some embodiments, ionizable lipid may comprise about 41%, 42%, 43%, 44%, 45%, 46%, 47%, 48% or 49% of the LNP. In some embodiments, the LNP comprises a DSPC:cholesterol:DMG-PEG 2000:and an ionizable lipid with a mole ratio of 10:40.5:1.5:48.

In some embodiments, the LNP has a pH of 5 to 6.

In some embodiments, the RNA of the present disclosure may be formulated in lipid nanoparticles having a diameter of about 40 nm −300 nm, optionally the LNP has a particle size of no greater than 160 nm, and optionally the LNP has a particle size of about 140-160 nm.

### Lipids

Provided herein is a lipid of Formula I:

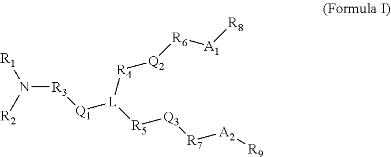

or a pharmaceutically acceptable salt thereof, wherein:
R₁ and R₂ are each, independently C₁-C₆ alkyl;
R₃ is C₁-C₅ alkyl;
Q₁, Q₂ and Q₃ are each independently —O—, —S—, —C(O)O—, —OC(O)—, —S—S—, —C(O)S—, —SC(O)—, —OC(S)—, or —C(S)O—;
L is C₁-C₃ alkyl;
R₄ and R₅ are each, independently C₁-C₁₀ alkyl;
R₆ and R₇ are each, independently C₁-C₁₀ alkyl or C₁-C₁₀ alkenyl;
A₁ and A₂ are each independently a bond, —O—, —S—, —C(O)O—, —OC(O)—, —S—S—, —C(O)S—, —SC(O)—, —OC(S)—, or —C(S)O—;
R₈ and R₉ are each, independently, C₁-C₃₀ alkyl.

In some embodiments, L is

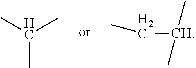

Provided herein is a lipid of Formula II:

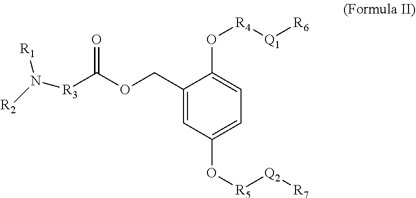

or a pharmaceutically acceptable salt thereof, wherein:
R₁ and R₂ are each, independently, C₁-C₆ alkyl;
R₃ is C₁-C₅ alkyl;
R₄ and R₅ are each independently C₁-C₁₈ alkyl;
Q₁ and Q₂ are each independently —O—C(O)—, —C(O)—O—, —O—C(S)—, —C(S)—O—; —S—S—,
R₆ and R₇ are each independently a C₁₈-C₃₂ alkyl.

In some embodiments, R₁ and R₂ are methyl.

In some embodiments, Q₁ and Q₂ are each, independently, —C(O)—O— or —O—C(O)—. In some embodiments, R₃ is C₃ alkyl.

In certain embodiments, a lipid selected form the group consisting of:

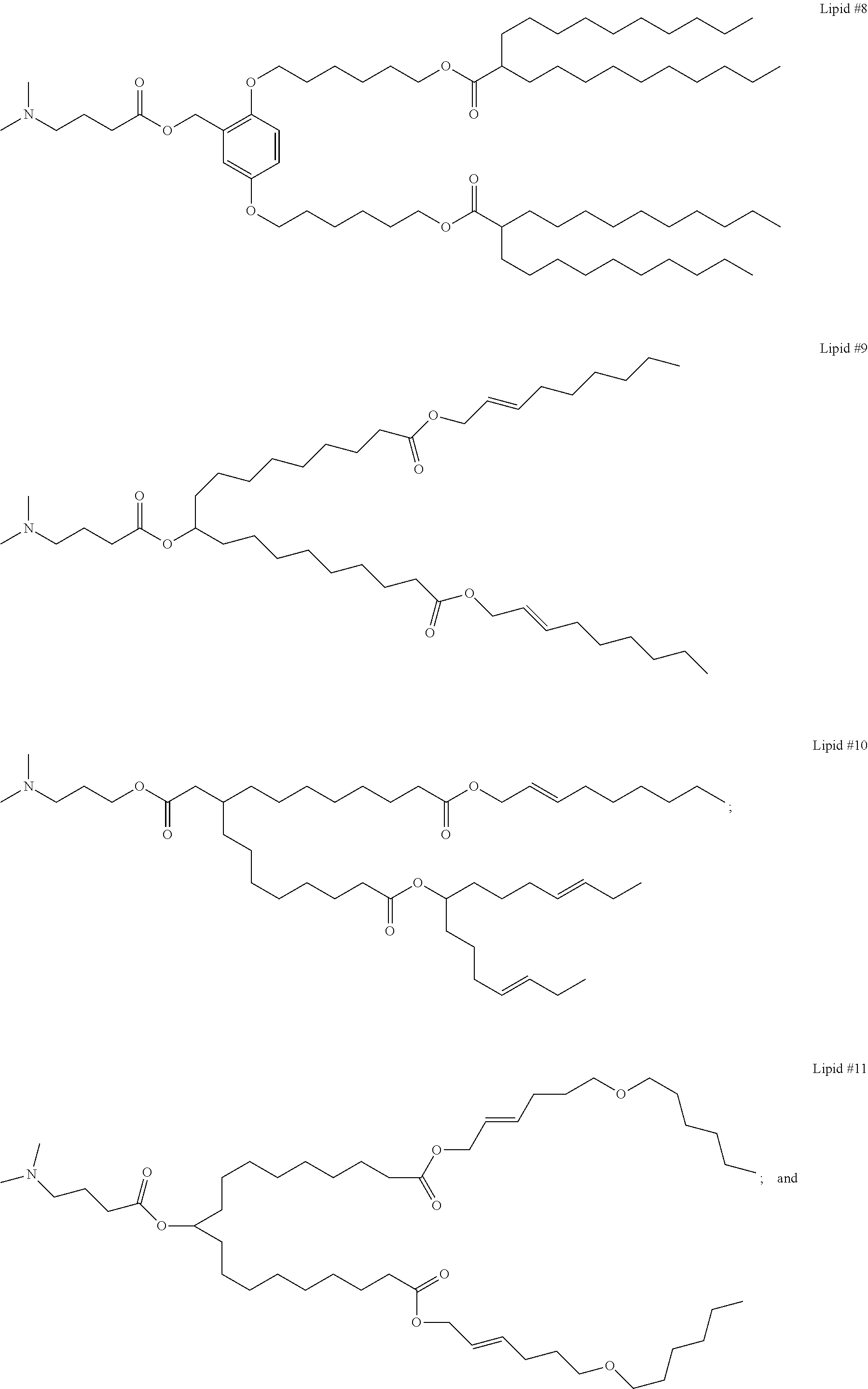

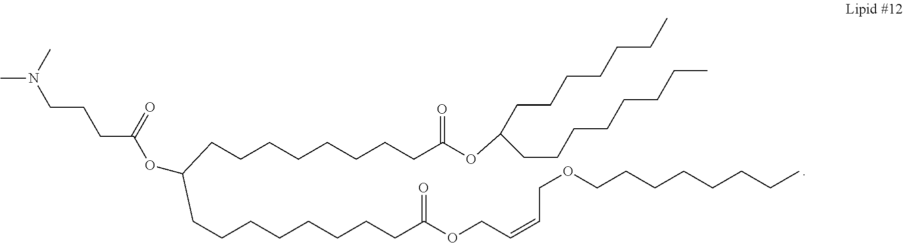 [SMILES: CCCCCCCCOC/C=C\COC(=O)CCCCCCCCC(CCCCCCCCC(=O)OC(CCCCCCC)CCCCCCCC)OC(=O)CCCN(C)C]

In certain embodiments, provided herein is a pharmaceutically acceptable composition comprising a lipid of any of the above embodiments, and a pharmaceutically acceptable carrier.

In some embodiments, pharmaceutically acceptable salts of the lipids described herein include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate, trifluoroacetate, and undecanoate.

### Methods of Treatment

Provided herein are compositions (e.g., pharmaceutical compositions), methods, kits and reagents for prevention and/or treatment of diseases or conditions in humans and other mammals.

The disclosure herein provides a method of mixing the lyophilized RNA with a liquid LNP solution to make a pharmaceutical composition. In certain embodiments, the liquid LNP solution is added to the lyophilized RNA. In certain embodiments, the lyophilized RNA and liquid LNP solution are mixed at room temperature. In certain embodiments, the lyophilized RNA and liquid LNP solution are mixed prior to clinical use.

Prophylactic protection from an antigen can be achieved following administration of a RNA vaccine or a therapeutic of the present disclosure. In certain embodiments, it is sufficient to administer the vaccine or therapeutic twice. It is possible, although less desirable, to administer the vaccine or therapeutic to an infected individual to achieve a therapeutic response.

A method of eliciting an immune response in a subject against an antigen is provided in aspects of the present disclosure. The method involves administering to the subject a RNA vaccine or therapeutic comprising a RNA polynucleotide having an open reading frame encoding at least one antigenic polypeptide, thereby inducing in the subject an immune response. An “anti-antigenic polypeptide antibody” is a serum antibody the binds specifically to the antigenic polypeptide.

A “prophylactically effective dose” as used herein is a therapeutically effective dose that prevents infection with the virus at a clinically acceptable level. In some embodiments, the therapeutically effective dose is a dose listed in a package insert for the vaccine or therapeutic. A traditional vaccine, as used herein, refers to a vaccine other than the RNA vaccine or therapeutic of the present disclosure. For instance, a traditional vaccine includes, but is not limited, to live microorganism vaccines, killed microorganism vaccines, subunit vaccines, protein antigen vaccines, DNA vaccines, VLP vaccines, etc. In exemplary embodiments, a traditional vaccine is a vaccine that has achieved regulatory approval and/or is registered by a national drug regulatory body, for example, the Food and Drug Administration (FDA) in the United States or the European Medicines Agency (EMA).

A method of eliciting an immune response in a subject is provided in aspects of the present disclosure. The method involves administering to the subject a RNA comprising an RNA polynucleotide. In certain embodiments, the RNA polynucleotide has an open reading frame encoding at least one antigenic polypeptide, thereby inducing in the subject an immune response specific to the antigenic polypeptide, wherein the anti-antigenic polypeptide antibody titer in the subject is increased following vaccination.

Provided herein are compositions (e.g., pharmaceutical compositions), methods, kits and reagents for prevention and/or treatment of VZV in humans and other mammals. VZV RNA vaccines can be used as therapeutic or prophylactic agents. They may be used to prevent and/or treat an infectious disease. In exemplary aspects, the VZV RNA vaccines of disclosed herein are used to provide prophylactic protection from varicella and herpes zoster. Varicella is an acute infectious disease caused by VZV. The primary varicella zoster virus infection that results in chickenpox (varicella) may result in complications, including viral or secondary bacterial pneumonia. Even when the clinical symptoms of chickenpox have resolved, VZV remains dormant in the nervous system of the infected person in the trigeminal and dorsal root ganglia and may reactivate later in life, travelling from the sensory ganglia back to the skin where it produces a disease (rash) known as shingles or herpes zoster, and can also cause a number of neurologic conditions ranging from aseptic meningitis to encephalitis. The VZV vaccines of the present disclosure can be used to prevent and/or treat both the primary infection (Chicken pox) and also the re-activated viral infection (shingles or herpes zoster) and may be particularly useful for prevention and/or treatment of immunocompromised and elderly patients to prevent or to reduce the severity and/or duration of herpes zoster.

A method of eliciting an immune response in a subject against an antigen (e.g., VZV) is provided in aspects of the present disclosure. The method involves administering to the subject a srRNA vaccine (e.g., VZV srRNA) comprising at least one srRNA polynucleotide having an open reading frame encoding at least one antigenic polypeptide (e.g., a VZV antigen), thereby inducing in the subject an immune response specific to an antigenic polypeptide (e.g., a VZV antigen). An “anti-antigenic polypeptide antibody” is a serum antibody the binds specifically to the antigenic polypeptide.

A method of eliciting an immune response in a subject against an antigen (e.g., a VZV antigen) is provided in aspects of the present disclosure. The method involves administering to the subject a RNA vaccine comprising at least one RNA polynucleotide having an open reading frame encoding at least one antigenic polypeptide (e.g., a VZV antigen), thereby inducing in the subject an immune response specific to an antigenic polypeptide (e.g., a VZV antigen), wherein anti-antigenic polypeptide antibody titer in the subject is increased following vaccination relative to anti-antigenic polypeptide antibody titer in a subject vaccinated with a prophylactically effective dose of a traditional vaccine against the antigen (e.g., a VZV antigen). An “anti-antigenic polypeptide antibody” is a serum antibody the binds specifically to the antigenic polypeptide.

A prophylactically effective dose is a therapeutically effective dose that prevents infection with the virus at a clinically acceptable level. In some embodiments, the therapeutically effective dose is a dose listed in a package insert for the vaccine. A traditional vaccine, as used herein, refers to a vaccine other than the srRNA vaccine of the present disclosure. For instance, a traditional vaccine includes, but is not limited, to live microorganism vaccines, killed microorganism vaccines, subunit vaccines, protein antigen vaccines, DNA vaccines, VLP vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

### Modes of Administration

In some embodiments, the RNA vaccine or therapeutic may be administered to a subject (e.g., parenteral and transmucosal (e.g., buccal, sublingual, palatal, gingival, nasal, vaginal, rectal, or transdermal)). Parenteral administration includes, e.g., intravenous, intramuscular, intra-arterial, intradermal, subcutaneous, intraperitoneal, intraventricular, and intracranial administration. In some embodiments, the RNA vaccine or therapeutic may be administered intramuscularly. In some embodiments, the RNA vaccine or therapeutic is administered systemically. Other modes of delivery include, but are not limited to, the use of liposomal formulations, intravenous infusion, transdermal patches, etc.

In some embodiments, the vaccine or therapeutic may be administered to the subject at a dose of 1 μg-100 μg, optionally 8, 10, or 30 μg. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

In some embodiments, the vaccine or therapeutic may be administered to the subject more than once. In some embodiments, the vaccine or therapeutic may be administered to the subject at least two times. In some embodiments, the second dose may be administered to the subject about 3 weeks following the prime dose. In some embodiments, the second dose may be administered to the subject about 8 weeks following the initial or prime dose. In some embodiments, the second dose may be administered to the subject about 7 weeks following the prime dose. In some embodiments, the second dose may be administered to the subject about 6 weeks following the prime dose. In some embodiments, the second dose may be administered to the subject about 5 weeks following the prime dose. In some embodiments, the second dose may be administered to the subject about 4 weeks following the prime dose. In some embodiments, the second dose may be administered to the subject about 3 weeks following the prime dose. In some embodiments, the second dose may be administered to the subject about 2 weeks following the prime dose. In some embodiments, the second dose may be administered to the subject about 1 week following the prime dose.

A VZV srRNA vaccine may be administered to a subject, e.g., intramuscularly. The vaccine may be administered to the subject at a dose of about 1 μg to about 100 μg, optionally 10 μg or 30 μg. In some embodiments, the vaccine is administered at a dose of at least or about 1, 5, 10, 15, 20, 25, 30, 35, 40, 45, 50, 55, 60, 65, 70, 75, 80, 85, 90, 95, 100, 105, 110 μg or more. In some embodiments, the vaccine is administered at a dose in a range of about 1 μg to about 5, 10, 15, 20, 25, 30, 35, 40, 45, 50, 55, 60, 65, 70, 75, 80, 85, 90, 95, 100, 105, or 110 mg. In some embodiments, the vaccine is administered at a dose in a range of about 10 μg to about 15, 20, 25, 30, 35, 40, 45, 50, 55, 60, 65, 70, 75, 80, 85, 90, 95, 100, 105, or 110 μg. In some embodiments, the vaccine is administered at a dose in a range of about 20 μg to about 25, 30, 35, 40, 45, 50, 55, 60, 65, 70, 75, 80, 85, 90, 95, 100, 105, or 110 μg. The vaccine may be administered to the subject at least one time. The vaccine may be administered to the subject at least two times. The vaccine may be administered to the subject at least three times. The second dose may be administered to the subject about 9 weeks following the prime dose The second dose may be administered to the subject about 8 weeks following the prime dose The second dose may be administered to the subject about 7 weeks following the prime dose The second dose may be administered to the subject about 6 weeks following the prime dose The second dose may be administered to the subject about 5 weeks following the prime dose The second dose may be administered to the subject about 4 weeks following the prime dose. The second dose may be administered to the subject about 3 weeks following the prime dose. The second dose may be administered to the subject about 2 weeks following the prime dose.

### Kits

In some embodiments, the present disclosure also provides kits comprising: i) a lyophilized RNA; ii) a delivery vehicle, such as a liquid LNP solution; iii) instructions for mixing the lyophilized RNA with the delivery vehicle to prepare an immunogenic composition; and iv) instructions for administration of the immunogenic composition to stimulate an immune response against the antigen in a mammalian subject, such as a human subject in need thereof.

The present disclosure also provides kits comprising: i) a lyophilized srRNA comprising a VZV gE antigen; ii) a delivery vehicle, such as an LNP; iii) instructions for mixing the first composition with the second composition to prepare an immunogenic composition; and iv) a set of instructions for administration of the immunogenic composition to stimulate an immune response against the gE antigen in a mammalian subject, such as a human subject in need thereof.

In some embodiments, the lyophilized RNA and liquid LNP solution are stored in separate glass vials. In some embodiments, the liquid LNP solution composition is stored at a temperature of 2-8° C. In some embodiments, the lyophilized RNA composition is stored at room temperature. In some embodiments, the lyophilized RNA composition is stored at a temperature of 2-8° C. In some embodiments, the liquid LNP solution is added to the lyophilized RNA. In some embodiments, the lyophilized RNA and liquid LNP solution are mixed at room temperature. In some embodiments, the lyophilized RNA and liquid LNP solution are mixed prior to clinical use.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical arts.

Throughout the description, where systems are described as having, including, or comprising specific components, or where processes and methods are described as having, including, or comprising specific steps, it is contemplated that, additionally, there are systems of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of an apparatus or a method described herein can be combined in a variety of ways without departing from the spirit and scope of the present invention, whether explicit or implicit herein. For example, where reference is made to a particular component of a system, that component can be used in various embodiments of systems of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10%, ±5%, ±3% or ±2% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, formulations specifying a percentage are by weight unless otherwise specified. Further, if a variable is not accompanied by a definition, then the previous definition of the variable controls.

The term “dried RNA” or “dried mRNA” as used herein has to be understood as RNA that has been lyophilized, or spray-dried, or spray-freeze dried as defined herein to obtain a temperature stable dried RNA (powder).

“Cryoprotectants” are known in the art and include without limitation, sucrose, trehalose, and glycerol. A cryoprotectant exhibiting low toxicity in biological systems is generally used.

The terms “lyophilization” include the related terms “cryodesiccation,” “lyophilizing,” or “freeze drying,” and typically relates to a process which allows reduction of a solvent (e.g., water) content of a frozen sample (preferably a solution containing an RNA molecule and a cryoprotectant as described herein) in one or more steps via sublimation. In the context of the present disclosure, lyophilization is typically carried out by freezing a sample and subsequently drying the sample via sublimation, optionally by reducing the surrounding pressure and/or by heating the sample so that the solvent sublimes directly from the solid phase to the gas phase.

The term “nucleic acid,” in its broadest sense, includes any compound and/or substance that comprises a polymer of nucleotides. These polymers can be referred to as polynucleotides.

The term “messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo.

The term “dose” as used herein in reference to an immunogenic composition refers to a measured portion of the immunogenic composition taken by (administered to or received by) a subject at any one time.

The term “immunization” refers to a process that increases a mammalian subject's reaction to an antigen and therefore improves its ability to resist or overcome infection and/or resist disease.

The term “vaccination” as used herein refers to the introduction of a vaccine into a body of a subject, preferably, a mammalian subject such as a human.

The term “prophylactically effective dose” includes the related terms “effective dose” and “therapeutically effective dose,” and as used herein refers to a dose that prevents infection with the virus at a clinically acceptable level.

The term “alkyl” refers to a radical of a straight-chain or branched saturated hydrocarbon group having from 1 to 32 carbon atoms (“C₁-C₃₂ alkyl”). In some embodiments, an alkyl group has 1 to 12 carbon atoms (“C₁-C₁₂ alkyl”). In some embodiments, an alkyl group has 1 to 10 carbon atoms (“C₁-C₁₀ alkyl”). In some embodiments, an alkyl group has 1 to 9 carbon atoms (“C₁-C₉ alkyl”). In some embodiments, an alkyl group has 1 to 7 carbon atoms (“C₁-C₇ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). In some embodiments, an alkyl group has 1 to 30 carbon atoms (“C₁-C₃₀ alkyl”). In some embodiments, an alkyl group has 1 to 22 carbon atoms (“C₁-C₂₂ alkyl”). In some embodiments, an alkyl group has 5 to 10 carbon atoms (“C₅-C₁₀ alkyl”). In some embodiments, an alkyl group has 7 to 17 carbon atoms (“C₇-C₁₇ alkyl”). In some embodiments, an alkyl group has 10 to 32 carbon atoms (“C₁₀-C₃₂ alkyl”).

The term “alkenyl” refers to a radical of a straight-chain or branched hydrocarbon group having from 2 to 20 carbon atoms, one or more carbon-carbon double bonds, and no triple bonds (“C₂-C₂₀alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₅ alkenyl”). In some embodiments, an alkenyl group has 2 to 6 carbon atoms (“C₂-C₆alkenyl”). In some embodiments, an alkenyl group has 2 to 5 carbon atoms (“C₂-C₅ alkenyl”).

The term “suitable protective agent” includes the related terms “cryoprotectant” and “protective agent”, and does not cause or enhance degradation of the RNA.

### EXAMPLES

### Example 1. Manufacture of S-2P RNA

The manufacturing of S-2P RNA included three steps. First, the plasmid DNA encoding the spike glycoprotein of SARS-CoV-2 was amplified, extracted and then purified by methods known in the art. Next, the plasmid was linearized by methods known in the art, and then purified by chromatographic purification and ethanol precipitation. Using the linearized plasmid DNA as a template, the RNA was enzymatically synthesized in vitro, followed by purification and storage at −80° C.

### Example 2. Lyophilization of S-2P RNA

With the use of well-suited protective agents and excipients, lyophilization of S-2P RNA was successfully conducted. In brief, about 0.5 mL of 100 μg/mL S-2P RNA in citrate buffer, pH 6 supplemented with about 3-12% (w/v) sucrose was filled into glass vials such that each vial contained about 50 μg RNA.

The lyophilization process includes freezing, primary drying and secondary drying (see detailed settings in Table 1), eventually yielding RNA in the lyophilized form with very low moisture content (<3%). Like many other lyophilized products, the lyophilized RNA can be stored at 2-8° C. or room temperature for a long period of time with non-compromised bioactivity.

TABLE 1
Parameters of the RNA lyophilization process

| Lyophilization | Time | Temperature | Pressure |
|---|---|---|---|
| step | [h:min] | (° C.) | (mbar) |
| Freezing | 00:01 | 25 to 2 | Atm |
| Freezing | 01:00 | 2 | Atm |
| Freezing | 00:01 | 2 to −50 | Atm |
| Freezing | 03:00 | −50 | Atm |
| Primary drying | 01:00 | −50 to −47 | 0.01 |
| Primary drying | 20:00 | −47 | 0.01 |
| Primary drying | 03:00 | −47 to −20 | 0.01 |
| Primary drying | 01:00 | −20 | 0.01 |
| Secondary drying | 03:00 | −22 to 4 | 0.02 |
| Secondary drying | 02:00 | 4 | 0.02 |
| Secondary drying | 04:00 | 4 to 30 | 0.08 |
| Secondary drying | 04:00 | 30 | 0.08 |

### Example 3. Preparation of Lipid Nanoparticles

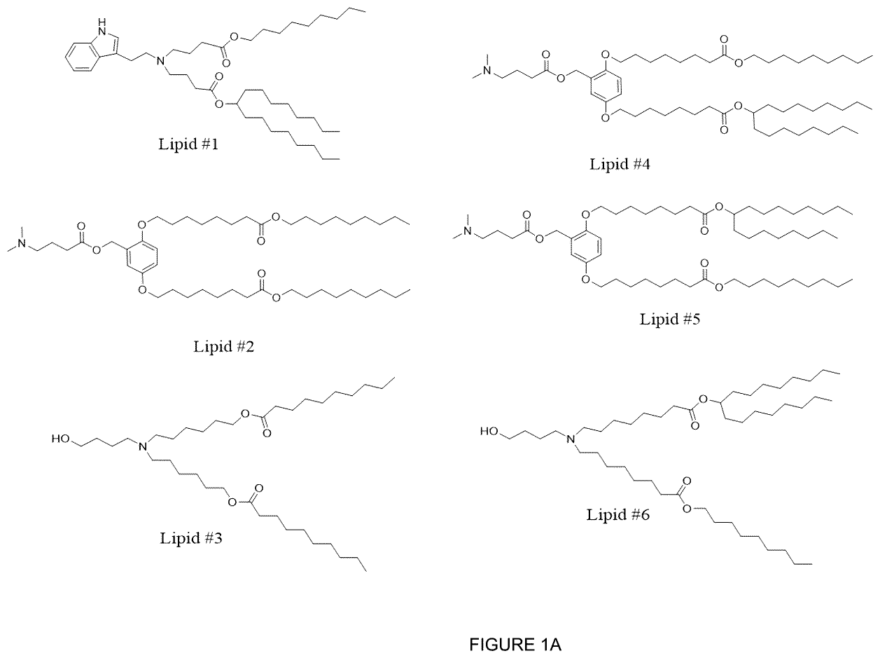

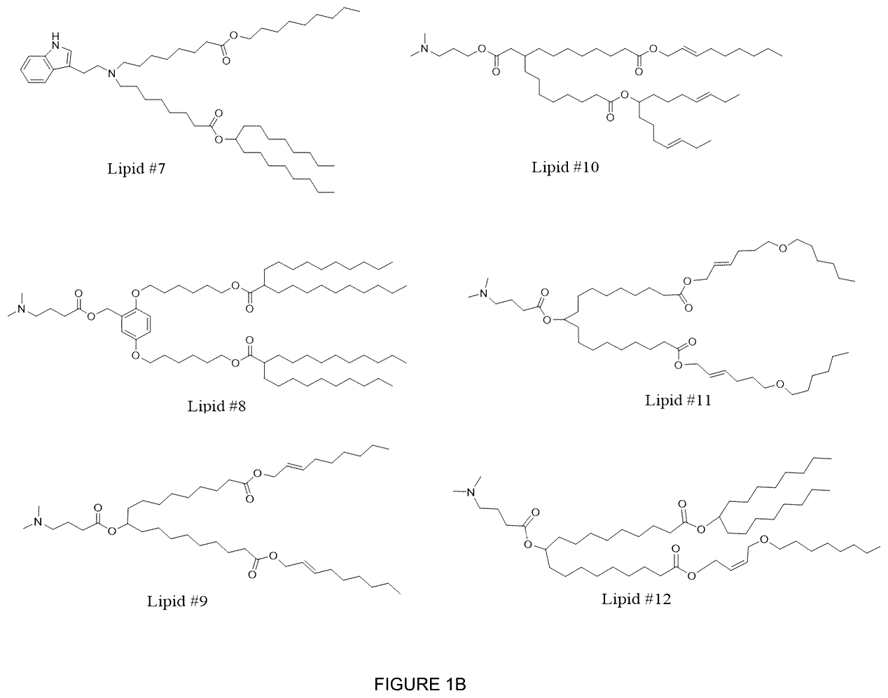

Lipid nanoparticles (LNPs) were formed or formulated by rapid mixing of an ethanol phase and an aqueous phase using a microfluidic device. The aqueous phase included a 50 mM citrate buffer (pH 5.5). The ethanol phase included an ionizable lipid (as described herein), cholesterol (Jiangsu Southeast Nanomaterials), 1,2-diastearoyl-sn-glycero-3-phosphocholine (DSPC) (Jiangsu Southeast Nanomaterials) and 1,2-dimyristoyl-rac-glycero-3-methoxypolyethylene glycol-2000 (DMG-PEG2000) (SINOPEG). A series of ionizable lipids were evaluated. The chemical structures of these lipids are shown in FIG. 1. These four lipid components were mixed at defined molar ratios in ethanol, for instance, at a molar ratio of 48:40.5:10:1.5 (ionizable lipid:cholesterol:DSPC:DMG-PEG2000). The generated LNP particles were purified in citrate buffer (containing sucrose) and characterized with respect to particle size, polydispersity index (PDI) and ionizable lipid concentration. The LNP solution was then diluted to the target concentration and stored at 2-8° C.

In the case of a filled LNP, for example, an LNP-encapsulated S-2P RNA liquid drug product, the preparation protocol is essentially the same, except that it was an aqueous solution of S-2P RNA in citrate buffer pH 5-6 that constitutes the aqueous phase for LNP formation. The LNP-encapsulated RNA formulation is also referred to as “RNA-LNP” in this disclosure. Regarding characterization, particle size and polydispersity index (PDI) of the RNA-LNP were determined by Dynamic Light Scattering (DLS); RNA encapsulation efficiency and concentration were determined by means of Ribogreen® assay and RNA purity was determined by agarose gel electrophoresis.

### Example 4. Preparation and Characterization of “Ready-to-Use” RNA Products

The “ready-to-use” RNA product was manufactured by transferring a predetermined amount of already formed LNP in solution to a sealed vial containing the lyophilized RNA composition, followed by mixing at room temperature. The amount of LNP added per vial (e.g., 30-100 μg RNA per vial) was determined based on the molar ionizable lipid:RNA (N/P) molar ratio of 7. In this disclosure, “ready-to-use” S-2P RNA formulations (also referred to as “LNP(S-2P RNA)”) containing 30-100 μg/mL S-2P RNA were prepared, and then characterized with respect to particle size, polydispersity index, as well as RNA concentration, complexation efficiency, and purity using the same methods as described above in Example 3.

### Example 5. Preparation and Characterization of “Ready-to-Use” S-2P RNA—Proof of Concept (PoC) Study

Various batches of LNP were manufactured as descried in Example 3 with ionizable lipid (Lipid #5 in FIG. 1A) and different molar ratios of ionizable lipid:cholesterol:DSPC:DMG-PEG2000 to compared key characteristics, all rendering the same particle size (80±15 nm) and polydispersity index (0.10±0.05). Upon adding the LNP to lyophilized S-2P RNA, the latter was completely reconstituted immediately while simultaneously mixed with the LNP, yielding a homogenous dispersion within a few seconds. The resulting “ready-to-use” RNA formulation manifested as a colorless and slightly opalescent dispersion, identical to the currently commercially available LNP-encapsulated mRNA counterpart. A RNA concentration of at least 100 μg/mL was successfully manufactured, and is sufficiently high for vaccine applications. Besides appearance, the key pharmaceutical characteristics of these two S-2P RNA formulations manufactured following these two different approaches were also compared. As shown in Table 2, these two tested formulations had essentially the same particle size distribution, RNA encapsulation, complexation efficiency, and RNA purity. These data indicate that the novel “ready-to-use” RNA formulations have favorable pharmaceutical characteristics comparable to those of the currently commercially available LNP-encapsulated counterparts.

TABLE 2
Representative pharmaceutical characteristics of “ready-
to-use” RNA and LNP-encapsulated RNA formulations

| | | | RNA | |
|---|---|---|---|---|
| Formulation | Size | | encapsulation/complexation | RNA |
| description | (nm) | PDI | efficiency | purity |
| S-2P RNA-LNP (LNP-encapsulated) | 110 | 0.13 | ≥90% | ≥90% |
| LNP(S-2P RNA) (“ready-to-use”)* | 120 | 0.10 | ≥80% | ≥90% |

### Example 6. In Vitro Transfection

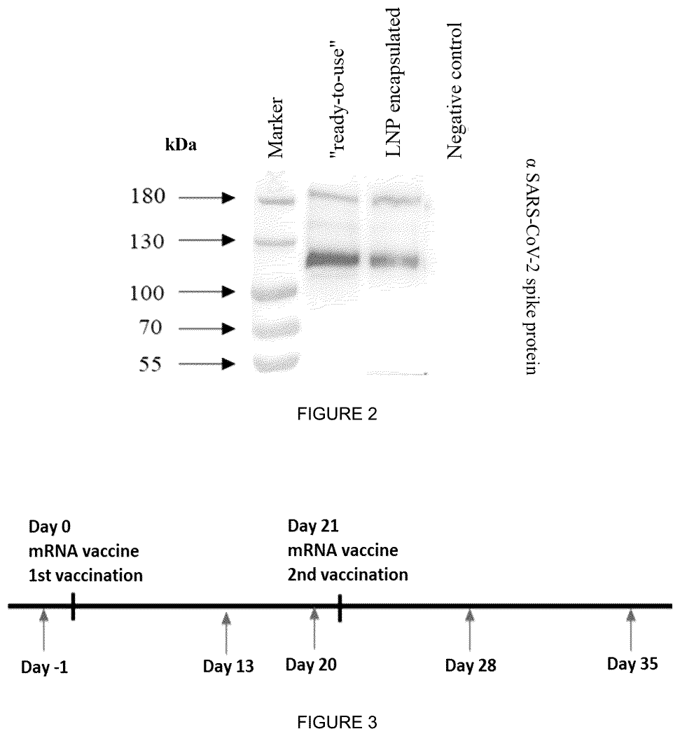

In the PoC studies, to assess the transfection efficiency of S-2P RNA formulations in vitro, 3×10⁵ Vero E6 cells per well were seeded in 6-well plates. 4 μg of the “ready-to-use” RNA formulation, formulated with Lipid #4, or the LNP-encapsulated S-2P RNA formulation, formulated with Lipid #9, was transfected into the Vero E6 cells and the spike glycoprotein of SARS-CoV-2 in cells was detected using western blot. Briefly, at 24 hour post transfection, cells transfected with RNA formulations were lysed by NP-40 Lysis buffer (50 mM Tris pH 7.4, 150 mM NaCl, 1% NP40, sodium pyrophosphate, β-glycerophosphate, sodium orthovanadate, sodium fluoride, EDTA, leupeptin). The mixtures were centrifuged at 13,000 rpm for 5 minutes at 4° C. The supernatants were collected and boiled with SDS for 12 minutes at 95° C., separated in a 6% SDS-PAGE gel and transferred to nitrocellulose filter membranes. After blocking with 5% BSA, the membranes were first blotted with primary antibodies (1:1000) (SARS-CoV-2 (2019-nCoV) Spike Rabbit PAb, 40592-T62, Sino Biological), and then incubated with horseradish peroxidase (HRP) conjugated secondary antibodies (1:10000) (IgG(H+L) (HRP-labeled Goat Anti-Rabbit IgG(H+L))), Beyotime). Finally, they were visualized with Chemiluminescent Reagent (Chemiluminescent HRP Substrate, WBKLS0500, Millipore). At 24 hours post transfection, the spike glycoprotein of SARS-CoV-2 in cells were detected using Western Blot. Based on the S-2P protein expression levels shown in FIG. 2, the “ready-to-use” RNA formulation described herein are at least as effective as the widely utilized LNP-encapsulated RNA formulation.

In the formulation screening studies (Example 3), the in vitro transfection efficiency of “ready-to-use” S-2P RNA test items were assessed in both BHK-21 and Vero E6 cell lines. Briefly, 3×10⁵ Vero E6 or BHK-21 cells per well were seeded in 6-well plates. 4 μg of each assessed “ready-to-use” S-2P RNA test item was transfected into the cells. At 48 hours post transfection, RBD expression in cell culture supernatants was quantified with a commercial SARS-CoV-2 (2019-nCoV) Spike RBD ELISA kit (KIT40592, Sino Biological) according to the manufacturer's instruction. The supernatants were diluted 200 fold. Final concentrations of RBD were calculated based on the linear standard curve of absorbance at 450 nm. Briefly, the detection wells were pre-coated with monoclonal antibody specific for Spike RBD protein. After incubation with samples or standards at room temperature (RT) for two hours, samples unbound to immobilized antibody were removed through washing steps. The detection antibodies were then added to wells for one-hour incubation at RT. After washing, substrate solution was added to each well while protected from light. Stop solutions were added to each well after 20 minutes and the absorbance at 450 nm was measured. The results are displayed in Table 4.

### Example 7. In Vivo Evaluation of “Ready-to-Use” S-2P RNA

Animal studies were carried out at Yangtze Delta Region Research Institute of Tsinghua University (Zhejiang). BALB/c mice (6-8 weeks of age) were placed into groups of n=4. On day 0 (prime injection) and day 21 (boost), three groups of mice were immunized intramuscularly with either “ready-to-use” S-2P RNA formulation (8 μg) or LNP-encapsulated S-2P RNA formulation (5 μg) and buffer vehicle, respectively. For the “ready-to use formulation”, the formulation used was: 48:40.5:10:1.5 (Lipid #4:cholesterol:DSPC:DMG-PEG2000). For the LNP encapsulated formulation, the formulation used was: 40:48.5:10:1.5 (Lipid #9:cholesterol:DSPC: DMG-PEG2000). Serum was collected prior to the first vaccination, as well as on day 13, 20, 28 and 35. All collected samples were cryopreserved following standard protocols. (FIG. 3).

### Example 8. Quantification of Antibody Binding Titers Against SARS-CoV-2 Spike Protein by ELISA Assay

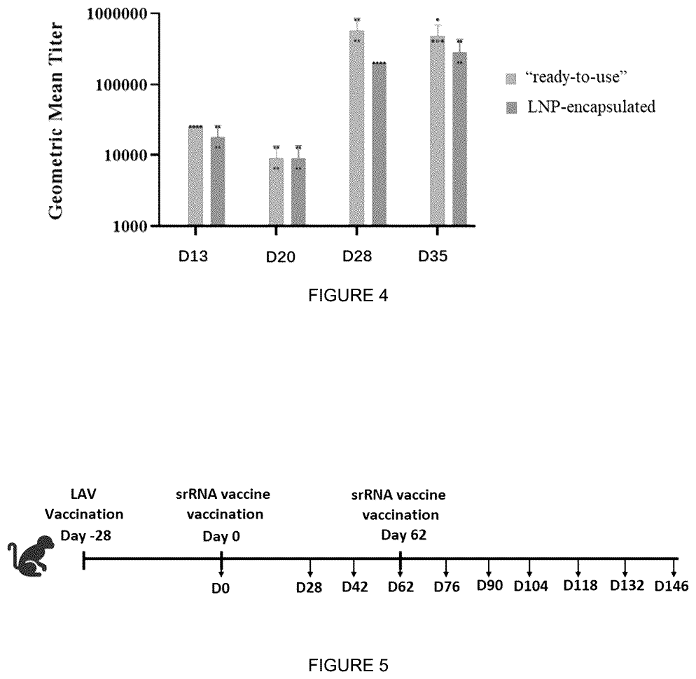

Antibody binding titers against SARS-CoV-2 spike protein (RBD, Histag) (GenScript Z03483) were quantified by enzyme-linked immunosorbent assay (ELISA). Briefly, 0.5 μg/mL SARS-CoV-2 spike protein (RBD, His tag) diluted in carbonate-bicarbonate buffer was pre-coated onto 96-well clear polystyrene microplate (Corning) overnight at 4° C. After washing with PBS-T (0.05% Tween-20 in PBS) three times, the coated plates were blocked with 300 μL blocking buffer (15% normal goat serum and 2% bovine serum albumin in PBS-T) for 1 h at 37° C. Serum samples were 2-fold serially diluted in blocking buffer, transferred to the plates, and incubated for 1 h at 37° C. After washing, plates were incubated with HRP-conjugated rabbit anti-mouse IgG H+L (Abcam, ab6728) for 1 h at 37° C. Plates were washed and incubated with TMB Single-Component Substrate solution (Solarbio, PR1200) for 7 min at 37° C., and the reaction was stopped with ELISA Stop Solution (Solarbio, C1058). Absorbance was read at 450 nm on a microplate reader ((VARIOSKAN LUX, ThermoFisher) and ELISA titers were determined using non-linear 4-parameter variable slope analysis in GraphPad Prism 8 software (FIG. 4).

### Example 9. Screening LNP Compositions Suitable for “Ready-to-Use” RNA Products

Two formulation variables, namely i) ionizable lipid structure; and ii) lipid component ratios were systemically assessed in a series of experiments to identify the scope of LNP compositions. Other relevant formulation parameters such as the pH and N/P utilized in the manufacturing process were also assessed.

All formulations assessed in the following screening studies were each prepared following the standard protocol as described in Example 3 for comparison, without further optimization of formulation or manufacturing process parameters.

### Example 10. Ionizable Lipid Structures

To assess the effect of ionizable lipid structure on the product pharmaceutical profile, a series of blank LNPs (i.e. without RNA) were prepared using twelve different ionizable lipids (see FIGS. 1A-1B for chemical structures), respectively, along with three other lipid components (DMG-PEG, DSPC and cholesterol) following the protocol described in Example 3. Despite being constituted of different ionizable lipids, these twelve blank-LNP formulations employed the same lipid molar ratios, i.e. 48:40.5:10:1.5 (ionizable lipid:cholesterol:DSPC:DMG-PEG2000), as utilized in Example 3.

Each generated blank-LNP formulation was added to and mixed with lyophilized S2P RNA, altogether yielding a series of “ready-to-use” S2P RNA test items containing the different ionizable lipids. The pharmaceutical characteristics (Table 3) and in vitro transfection efficiency (Table 4) of these “ready-to-use” RNA test items were compared head-to-head. In parallel, the stability profiles of these twelve blank-LNP formulations (i.e. without RNA complexation) were assessed at 2-8° C. (see Table 5).

TABLE 3
Pharmaceutical characteristics of blank-LNP and “ready-to-use”
S2P RNA test items manufactured using different ionizable lipids

| Ionizable | | | “Ready-to-use” S2P RNA formulation | | |
|---|---|---|---|---|---|
| lipid | Blank-LNP | | Size | | RNA complexation |
| ID | Size (nm) | PDI | (nm) | PDI | efficiency |
| #1 | 81 | 0.20 | 166 | 0.18 | 46% |
| #2 | 120 | 0.09 | 211 | 0.21 | 63% |
| #3 | 142 | 0.22 | 230 | 0.26 | 82% |
| #4 | 88 | 0.13 | 173 | 0.12 | 42% |
| #5 | 91 | 0.2 | 137 | 0.13 | 44% |
| #6 | 84 | 0.14 | 170 | 0.20 | 43% |
| #7 | 67 | 0.25 | 92 | 0.21 | 28% |
| #8 | 86 | 0.13 | 130 | 0.11 | 34% |
| #9 | 207 | 0.09 | 280 | 0.18 | 34% |
| #10 | 91 | 0.05 | 204 | 0.21 | 34% |
| #11 | 165 | 0.09 | 266 | 0.11 | 34% |
| #12 | 85 | 0.04 | 172 | 0.14 | 27% |

Test items were manufactured at fixed lipid molar ratios (48 mol % ionizable lipid/40.5 mol % cholesterol/10 mol % DSPC/1.5 mol % DMG-PEG). In all cases, 0.6 mL of blank-LNP was added to lyophilized S2P RNA (55 ug/vial) at a ionizable lipid:RNA (N/P) molar ratio of 7.

TABLE 4
In vitro transfection efficiency comparison between “ready-
to-use” S2P RNA test items manufactured with different
ionizable lipids, expressed as RBD expression (determined by ELISA)

| | |
|---|---|
| | “Ready-to-use” RNA transfection: |
| Ionizable | RBD expression (ng/mL) |

| lipid ID | in BHK-21 cells | in Vero E6 cells |
|---|---|---|
| #1 | 0.49 | 0.46 |
| #2 | 1.34 | 0.97 |
| #3 | <LOD | <LOD |
| #4 | 3.07 | 2.60 |
| #5 | 2.10 | 2.58 |
| #6 | 3.64 | 4.24 |
| #7 | <LOD | <LOD |
| #8 | <LOD | <LOD |
| #9 | 0.50 | 0.16 |
| #10 | 0.33 | 0.25 |
| #11 | 0.52 | 0.20 |
| #12 | 0.19 | <LOD |
| neg ctrl | <LOD | <LOD |

TABLE 5
Stability profile of blank-LNP test items containing different ionizable lipids
(2-8° C.) with respect to particle size and polydispersity index

| | | | T = 2 weeks | | | T = 1 month | | |
|---|---|---|---|---|---|---|---|---|
| | T = 0 | | | | Change in | | | Change in |
| Ionizable | Size | | Size | | size (nm) | Size | | size (nm) |
| lipid ID | (nm) | PDI | (nm) | PDI | vs. T0 | (nm) | PDI | vs. T0 |
| #1 | 81 | 0.20 | 83 | 0.17 | +2 | 106 | 0.19 | +25 |
| #2 | 120 | 0.09 | 122 | 0.09 | +2 | 121 | 0.07 | +1 |
| #3 | 142 | 0.22 | 137 | 0.18 | −5 | 143 | 0.19 | +1 |
| #4 | 88 | 0.13 | 79 | 0.06 | −9 | 88 | 0.09 | 0 |
| #5 | 91 | 0.20 | 90 | 0.13 | −1 | 93 | 0.14 | +2 |
| #6 | 84 | 0.14 | 107 | 0.17 | +23 | 124 | 0.24 | +40 |
| #7 | 67 | 0.25 | 65 | 0.22 | −2 | 79 | 0.26 | +12 |
| #8 | 86 | 0.13 | 83 | 0.07 | −3 | 90 | 0.17 | +4 |
| #9 | 207 | 0.09 | 214 | 0.05 | +7 | 229 | 0.10 | +22 |
| #10 | 91 | 0.05 | 107 | 0.11 | +16 | 120 | 0.21 | +29 |
| #11 | 165 | 0.09 | 207 | 0.04 | +42 | 224 | 0.06 | +59 |
| #12 | 85 | 0.04 | 93 | 0.06 | +8 | 97 | 0.11 | +12 |

Test items were manufactured at fixed lipid molar ratios (48 mol % ionizable lipid/40.5 mol % cholesterol/10 mol % DSPC/1.5 mol % DMG-PEG)

As shown in Table 3, particle size and PDI of the resulting “ready-to-use” RNA test items were influenced by the choice of ionizable lipids, but still fall within the acceptable range for the intended application following, e.g., intramuscular administration. However, the RNA complexation efficiency read-outs differed among the twelve test items, with the formulations containing Lipids #2 and #3 being the highest relative to the others tested. This data indicates that ionizable lipids structurally resembling Lipids #1-#6 can yield “ready-to-use” RNA products with sufficiently high RNA complexation efficiency (as evidenced by >40% RNA complexation efficiency in the present experiment, without formulation and process parameter optimization).

The in vitro transfection efficiency of these twelve “ready-to-use” S2P RNA test items were compared in BHK-21 as well as Vero E6 cell lines at equal doses (4 μg). As evidenced by the expression levels (Table 4), the test items containing ionizable Lipids #4/#5/#6 yielded the highest transfection efficiency, followed by the test item containing Lipid #2.

Besides the pharmaceutical characteristics and transfection efficiency, the ionizable lipid structure can also impact the stability profile of the blank-LNP. As shown in Table 5, these twelve LNP formulations demonstrated divergent stability profiles at 2-8° C. due to the incorporation of different ionizable lipids. Among all test items assessed, LNP test items including Lipid #2, #3, #4, #5 or #8 demonstrated quantifiable stability with unaltered size upon storage at 2-8° C. for 1 month.

Taking all evaluations tested herein into account, Lipids #4 and #5 are the leading ionizable lipids among all structures tested, followed by Lipid #2. It appears that ionizable lipids with these structures yields blank-LNP with quantifiable stability at 2-8° C., which upon mixing with lyophilized RNA generates “ready-to-use” RNA formulation with favorable pharmaceutical characteristics and in vitro transfection efficiency. Ionizable Lipids #4, #5 and #2 have in common a head group of 4-N,N dimethylamino-butanoate, a linker of 2-methyl-1,4 phenylene-dioxyl, alkyl tails independently of octanoate, and each alkyl tail with C18-C26, these lipids are examples of Formula (II).

### Example 11. Molar Fractions of Lipid Components Constituting the LNP

An LNP described herein generally comprises four lipid-based components, namely: 1) ionizable lipid, 2) phospholipid (e.g., DSPC), 3) cholesterol and 4) a PEGylated lipid (e.g., DMG-PEG2000). Each of these components, as well as the relative ratios thereof, can play a role in the composition of the LNP. For the development of “ready-to-use” RNA formulations, LNP composed of lipids at different ratios were screened to identify lipid compositions well suited for “ready-to-use” RNA products.

To assess the ratio boundaries of each lipid component, first, fourteen blank-LNP formulations containing the same lipid components but at different feed ratios were synthesized. As shown in Table 6, in formulation 1-6 and 8, the amount of DMG-PEG was altered between 0.25 and 4 mol % while fixing the other lipid components (except for minor ratio changes for cholesterol, as needed). In formulation 7-9, the amount of DSPC was adjusted between 5-20 mol % while the cholesterol amount varied concomitantly. The amount of ionizable lipid increased from 20 to 70 mol % in formulation 10-12, 8, 13 and 14 while the cholesterol amount reduced from 68.5% to 18.5 mol % accordingly. With this experimental design, the molar fraction of lipid components independently or jointly effects on the formation and pharmaceutical characteristics of the LNP were assessed.

TABLE 6
Pharmaceutical characteristics of blank-LNP composed of ionizable Lipid #4,
cholesterol, DSPC, and DMG-PEG2000 at various molar ratios

| | | | | | Blank-LNP | | | | |
|---|---|---|---|---|---|---|---|---|---|
| | Molar ratio (%) | | | | T = 0 | | T = 8 weeks | | |
| | Ionizable | | | DMG- | Size | | Size | | Change in size |
| # | Lipid #4 | Chol | DSPC | PEG | (nm) | PDI | (nm) | PDI | vs. T0 (nm) |
| f1 | 50 | 39.8 | 10 | 0.25 | 174 | 0.07 | 175 | 0.07 | +1 |
| f2 | 50 | 39.5 | 10 | 0.5 | 146 | 0.02 | 167 | 0.08 | +20 |
| f3 | 50 | 39 | 10 | 1 | 114 | 0.14 | 112 | 0.07 | −2 |
| f4 | 50 | 38 | 10 | 2 | 73 | 0.22 | 82 | 0.18 | +9 |
| f5 | 50 | 37 | 10 | 3 | 98 | 0.14 | 118 | 0.29 | +20 |
| f6 | 50 | 36 | 10 | 4 | 116 | 0.24 | 133 | 0.24 | +17 |
| f7 | 50 | 28.5 | 20 | 1.5 | 79 | 0.14 | 90 | 0.13 | +11 |
| f8 | 50 | 38.5 | 10 | 1.5 | 81 | 0.19 | 79 | 0.18 | −2 |
| f9 | 50 | 43.5 | 5 | 1.5 | 78 | 0.19 | 76 | 0.18 | −2 |
| f10 | 20 | 68.5 | 10 | 1.5 | 90 | 0.23 | 1339 | 0.34 | +1249 |
| f11 | 30 | 58.5 | 10 | 1.5 | 86 | 0.23 | 84 | 0.19 | −2 |
| f12 | 40 | 48.5 | 10 | 1.5 | 81 | 0.17 | 78 | 0.15 | −3 |
| f13 | 60 | 28.5 | 10 | 1.5 | 73 | 0.22 | 78 | 0.17 | +5 |
| f14 | 70 | 18.5 | 10 | 1.5 | 75 | 0.22 | 78 | 0.17 | +3 |

The generated fourteen blank-LNP formulations were characterized in terms of particle size and PDI, both at the end of manufacturing (T=0) and 8-week storage at 2-8° C. As shown in Table 6, this pilot screening work points to a general ratio range for each lipid component that enables the attainment of LNP with generally favorable particle size (≤150 nm), PDI (≤0.25) and stability profile at 2-8° C. (defined as size increase ≤20 nm in 8 weeks), namely DMG-PEG2000 between at least 1-4 mol %, DSPC between at least 5-20 mol %, ionizable lipid between at least 30-70 mol % and cholesterol between at least 18.5-58.5 mol %.

A series of LNP formulations using two structurally divergent ionizable lipids were prepared, i.e. Lipid #4 and Lipid #6. In this experiment, the fraction of each ionizable lipid was set between 20-70 mol % out of total lipids, while the fraction of other lipid components was assigned out of the acceptable range as described in the paragraph above. The pharmaceutical characteristics of the blank-LNP and resulting “ready-to-use” RNA formulations, as well as the stability profile of the blank-LNP test items (2-8° C.) were evaluated.

As shown in Table 7, with ionizable Lipid #4, a molar fraction of 30-60 mol % results in not only acceptable particle size distribution, but also relatively high RNA complexation efficiency (>40%). As for ionizable Lipid #6, the highest RNA complexation efficiency was achieved when its molar fraction was kept between 30-50 mol %.

TABLE 7
Pharmaceutical characteristics of blank-LNP and resulting “ready-to-use”
RNA test items containing lipids at different molar ratios

| | | | | | | | “Ready-to-use” S2P RNA | | |
|---|---|---|---|---|---|---|---|---|---|
| | Ionizable | | | DMG- | Blank-LNP | | | | RNA |
| Ionizable | lipid | Chol | DSPC | PEG | Size | | Size | | complex. |
| lipid ID | mol % | mol % | mol % | mol % | (nm) | PDI | (nm) | PDI | efficiency |
| #4 | 20 | 57.0 | 20 | 3.0 | 82 | 0.18 | 157 | 0.21 | 54% |
| | 30 | 55.0 | 12 | 3.0 | 75 | 0.2 | 125 | 0.21 | 61% |
| | 40 | 48.5 | 10 | 1.5 | 90 | 0.12 | 169 | 0.18 | 51% |
| | 45 | 43.5 | 10 | 1.5 | 87 | 0.04 | 151 | 0.12 | 50% |
| | 50 | 38.5 | 10 | 1.5 | 83 | 0.11 | 147 | 0.13 | 48% |
| | 60 | 30.0 | 8.5 | 1.5 | 76 | 0.15 | 143 | 0.13 | 44% |
| | 70 | 22.5 | 6 | 1.5 | 77 | 0.21 | 146 | 0.16 | 40% |
| #6 | 20 | 57.0 | 20 | 3.0 | 100 | 0.08 | 199 | 0.22 | 19% |
| | 30 | 55.0 | 12 | 3.0 | 85 | 0.15 | 167 | 0.23 | 38% |
| | 40 | 48.5 | 10 | 1.5 | 109 | 0.18 | 192 | 0.18 | 41% |
| | 45 | 43.5 | 10 | 1.5 | 118 | 0.23 | 162 | 0.22 | 64% |
| | 50 | 38.5 | 10 | 1.5 | 174 | 0.29 | 202 | 0.25 | 29% |
| | 60 | 30.0 | 8.5 | 1.5 | 116 | 0.27 | 176 | 0.23 | 19% |
| | 70 | 22.5 | 6 | 1.5 | 93 | 0.21 | 155 | 0.22 | 11% |

Besides pharmaceutical characterization, head-to-head comparison of these “ready-to-use” S2P RNA test items was also performed regarding transfection efficiency in BHK-21 and Vero E6 cell lines at equal dose (4 μg). Based on the determined RBD expression levels (Table 8, determined by ELISA), an ionizable lipid fraction between 30-60 mol % was found in this study to work optimally for the transfection of “ready-to-use’ RNA formulation in cells.

TABLE 8
In vitro transfection of “ready-to-use” RNA test items manufactured with different
ionizable lipids and lipid ratios in BHK-21 and Vero E6 cell lines, respectively;
presented as RBD expression levels (determined by ELISA)

| | Lipid component ratio (mol %) | | | | RBD expression (ng/mL) | |
|---|---|---|---|---|---|---|
| Ionizable | Ionizable | | | DMG- | in BHK-21 | in Vero E6 |
| lipid ID | lipid | Chol | DSPC | PEG | cells | cells |
| #4 | 20 | 57.0 | 20.0 | 3.0 | 0.082 | <LOD |
| | 30 | 55.0 | 12.0 | 3.0 | 1.655 | 0.129 |
| | 40 | 48.5 | 10.0 | 1.5 | 1.517 | 1.204 |
| | 45 | 43.5 | 10.0 | 1.5 | 2.090 | 1.842 |
| | 50 | 38.5 | 10.0 | 1.5 | 1.887 | 1.633 |
| | 60 | 30.0 | 8.5 | 1.5 | 0.812 | 1.239 |
| | 70 | 22.5 | 6.0 | 1.5 | <LOD | 0.0939 |
| #6 | 20 | 57.0 | 20 | 3.0 | 0.260 | <LOD |
| | 30 | 55.0 | 12 | 3.0 | 2.015 | 0.892 |
| | 40 | 48.5 | 10 | 1.5 | 2.461 | 2.813 |
| | 45 | 43.5 | 10 | 1.5 | 2.382 | 2.539 |
| | 50 | 38.5 | 10 | 1.5 | 1.428 | 2.056 |
| | 60 | 30.0 | 8.5 | 1.5 | 1.015 | 0.811 |
| | 70 | 22.5 | 6.0 | 1.5 | 0.258 | 0.243 |

Regarding the stability profile at 2-8 BC, blank-LNP test items comprising ionizable Lipid #4 were overall more stable than those comprising Lipid #6. As shown in Table 9, Lipid #4-containing LNP demonstrated good stability at 2-8° C. as long as the ionizable lipid fraction remained no greater than 60 mol %.

TABLE 9
Stability profile of blank-LNP formulations comprising lipids at different ratios at 2-8° C.

| | | | | | Blank-LNP | | | | |
|---|---|---|---|---|---|---|---|---|---|
| Ionizable | | | | | | | T = 7 days | | |
| lipid | Molar ratios (mol %) | | | | T = 0 | | | | Change in |
| utilized in | Ionizable | | | DMG- | Size | | Size | | size (nm) |
| test item | lipid | Chol | DSPC | PEG | (nm) | PDI | (nm) | PDI | vs. T0 |
| Lipid #4 | 20 | 57.0 | 20.0 | 3.0 | 82 | 0.18 | 81 | 0.19 | −1 |
| | 30 | 55.0 | 12.0 | 3.0 | 75 | 0.20 | 76 | 0.12 | +1 |
| | 40 | 48.5 | 10.0 | 1.5 | 90 | 0.12 | 92 | 0.12 | +2 |
| | 45 | 43.5 | 10.0 | 1.5 | 87 | 0.04 | 91 | 0.13 | +4 |
| | 50 | 38.5 | 10.0 | 1.5 | 83 | 0.11 | 92 | 0.14 | +9 |
| | 60 | 30.0 | 8.5 | 1.5 | 76 | 0.15 | 97 | 0.13 | +21 |
| | 70 | 22.5 | 6.0 | 1.5 | 77 | 0.21 | 107 | 0.23 | +29 |
| Lipid #6 | 20 | 57.0 | 20 | 3.0 | 100 | 0.08 | 101 | 0.09 | +1 |
| | 30 | 55.0 | 12 | 3.0 | 85 | 0.15 | 95 | 0.19 | +10 |
| | 40 | 48.5 | 10 | 1.5 | 109 | 0.18 | 124 | 0.17 | +15 |
| | 45 | 43.5 | 10 | 1.5 | 118 | 0.23 | 142 | 0.27 | +24 |
| | 50 | 38.5 | 10 | 1.5 | 174 | 0.29 | 207 | 0.26 | +33 |
| | 60 | 30.0 | 8.5 | 1.5 | 116 | 0.27 | 147 | 0.24 | +31 |
| | 70 | 22.5 | 6.0 | 1.5 | 93 | 0.21 | 109 | 0.23 | +16 |

Based on all experimental evaluations, the ionizable lipid fraction is best at 30-60 mol % to allow for not only favorable pharmaceutical and biological performance of the “ready-to-use” RNA products but also long shelf-life of blank-LNP liquid product at 2-8° C. As for the other lipid components, it was demonstrated that DSPC between 5-20 mol %/DMG-PEG between 1-4 mol %/cholesterol between 18.5-58.5 mol % appeared to be well suited for the generation of “ready-to-use” RNA products.

### Example 12. pH of LNP Dispersion Formulation Added to Lyophilized RNA

In LNP formulations, the ionization state of the ionizable lipid can be dictated by the pH of the aqueous dispersion. In these embodiments, the pH of blank-LNP dispersion (and thereby of the “ready-to-use” RNA formulation) can affect the complexation efficiency of anionic RNA. RNA complexation favors to take place under acidic environment, at pHs below the pKa value (i.e. the negative base −10 logarithm of the acid dissociation constant) of the LNP liquid formulations.

The pKa of blank-LNP composed of ionizable lipids #1-12 is generally between 6.0-6.5. To gain insight into the pH range for these embodiments, three blank-LNP formulations were synthesized containing the same lipid compositions using Lipid #4, but dispersed in buffers at different pHs (pH 5.0, pH 5.5 and pH 6.0), with which three “ready-to-use” S2P RNA test items were prepared following the protocol: The LNP solution for S2P RNA formulation was gently shaken for about 5 to 10 seconds before use. Then, 1.0 mL of solution was aspirated using a syringe with a needle and slowly added to the lyophilized composition along the vial wall. Following complete injection, the solution was mixed by shaking upside down for about 30 seconds. After mixing, the solution appeared as a milky to white suspension. After mixing and being placed at room temperature for about 1 h, about 0.8 mL of S2P RNA formulation solution was aspirated using a syringe. As shown in Table 10, LNP dispersions at pH 5.0-6.0 were found to be suitable for RNA complexation in these embodiments and under the conditions tested.

TABLE 10
Pharmaceutical characteristics of blank-LNP dispersions at different pHs and the
resulting “Ready-to-use” RNA test items

| | | | | “Ready-to-use” S2P RNA formulation | | | |
|---|---|---|---|---|---|---|---|
| | Blank-LNP | | | | | RNA | RNA conc. |
| Test | Dispersion | Size | | | | complexation | determined |
| item | pH | (nm) | PDI | Size (nm) | PDI | efficiency | (μg/mL) |
| 1 | pH 5.0 | 98 | 0.15 | 177 | 0.13 | 42% | 85 |
| 2 | pH 5.5 | 95 | 0.11 | 159 | 0.14 | 40% | 87 |
| 3 | pH 6.0 | 90 | 0.09 | 164 | 0.15 | 36% | 83 |

Test items were manufactured with fixed lipid compositions (48 mol % ionizable Lipid #4/40.5 mol % cholesterol/10 % mol DSPC/1.5 mol % DMG-PEG).

### Example 13. Ratio Between RNA and Ionizable Lipid, Expressed as Ionizable Lipid:RNA (N/P) Molar Ratio

For LNP-encapsulated RNA formulation, a ionizable lipid:RNA (N/P) molar ratio of approx. 6 is often utilized (Int J Pharm. 2021 May 15; 601: 120586). To gain insight into the N/P ratio range suited for these embodiments, “ready-to-use” RNA formulations were generated by adding an intended amount of blank-LNP formulation (pH 5.5) to lyophilized S2P RNA based on a predetermined ionizable lipid:RNA molar ratio of 5, 7, or 9.

The effect of N/P molar ratio on the particle size, PDI and RNA complexation efficiency of the resulting “ready-to-use” RNA test items were characterized. As shown in Table 11, a higher N/P facilitated the formation of “ready-to-use” RNA products with higher RNA complexation efficiency and smaller hydrodynamic size. An N/P molar ratio above 5 but less than 9 appeared suitable for these embodiments under the conditions tested.

Table 12 summarizes the compositions and parameters for the “ready-to-use” RNA products.

TABLE 11
Pharmaceutical characteristics of different N/P molar ratio
and the generated “ready-to-use” RNA test items

| | | | | | RNA conc. |
|---|---|---|---|---|---|
| Test | N/P molar | Size | | RNA complexation | determined |
| item | ratio | (nm) | PDI | efficiency | (μg/mL) |
| 1 | 5 | 161 | 0.14 | 35% | 91 |
| 2 | 7 | 159 | 0.14 | 40% | 87 |
| 3 | 9 | 148 | 0.14 | 49% | 94 |

Test items were manufactured with fixed lipid compositions (48 mol % ionizable Lipid #4/40.5 mol % cholesterol/10 mol % DSPC/1.5 mol % DMG-PEG).

TABLE 12
Formulation compositions and parameters
suited for “ready-to- use” RNA products

| Variables | Range |
|---|---|
| Ionizable lipid chemical structure | 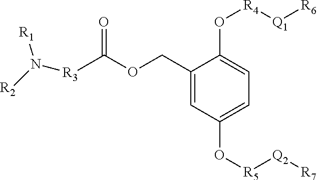 |
| Molar fraction | |
| of lipid | |
| components | |
| Ionizable lipid | 30-60 mol% |
| DSPC | At least 5-20 mol % |
| cholesterol | At least 18.5-58.5 mol % |
| DMG- | At least 1-4 mol % |
| PEG2000 | |
| Blank-LNP | Ideally 5-6 |
| dispersion pH | |
| N/P | Between 5-9 |

### Example 14. VZV Glycoprotein E (gE) Antigen

VZV glycoprotein E (gE) was selected as a candidate vaccine antigen. VZV gE is a type I membrane protein of 623 amino acids that comprises signal peptide, the main part of the protein, a hydrophobic anchor domain, and a C-terminal tail. The C-terminal domain of VZV gE includes a Y569A mutation that could modulate subcellular trafficking to the trans-Golgi network (TGN) and expression of VZV gE protein [1]. The srRNA vaccine construct include an RNA encoding a 573 amino acid carboxyl-terminal truncated VZV gE protein encoded by SEQ ID NO: 3 (Oka strain).

### Example 15. RNA In Vitro Transcription and Capping

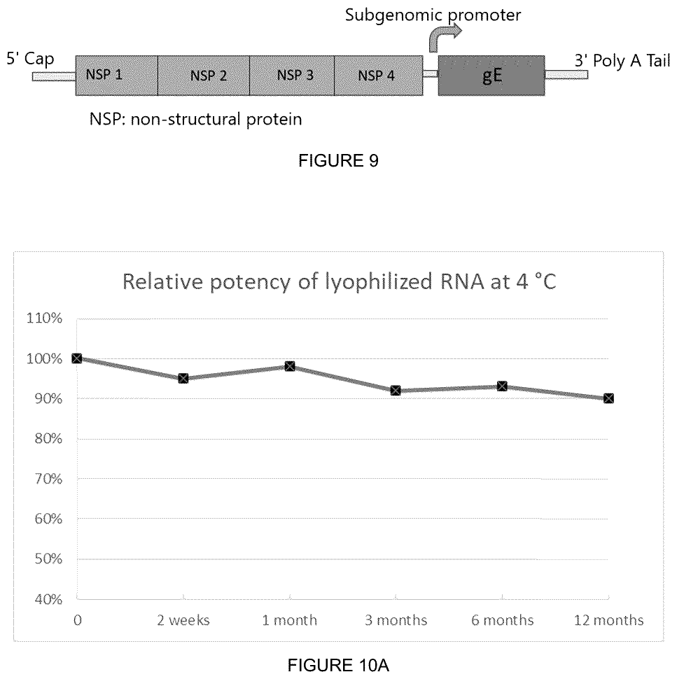

The self-replicating RNA (srRNA) backbone was based on an engineered alphavirus genome containing the genes encoding the non-structural proteins which allow RNA replication. The structural protein sequences were replaced with a gene sequence of VZV glycoprotein E. The srRNA vaccine construct comprises a 5′ cap untranslated region (UTR), four non-structural genes (nsp1-4), a 26S subgenomic promoter, VZV glycoprotein E gene, and a 3′ terminal polyadenylated tail encoded by SEQ ID NO: 4 (FIG. 9). To generate linear templates for RNA transcription, plasmid DNA was cut by restriction digest using BspQI enzyme (New England Biolabs, R0712L), and purified using PureLink® PCR Purification Kit (Invitrogen, K310002). RNA was transcribed using 5000 U/ml T7 polymerase (New England Biolabs, M0251L), 1000 U/ml RNase inhibitor (New England Biolabs, M0314L), 2 U/ml pyrophosphatase (New England Biolabs, M2403L), 2 mM Spermidine (Sigma, 5292), 10 mM DL-Dithiothreitol (Sigma, 43816), 6 mM rNTP (New England Biolabs, N0466S), 24 mM MgCl2 (Invitrogen, AM9530G) and 40 mM Tris-HCl (Sigma, T2694). The mixture was incubated and shaken at 37° C. for 3 hours. RNA transcripts were capped with vaccinia capping enzyme (New England Biolabs, M2080S) and mRNA cap 2′-O-Methyltransferase (New England Biolabs, M0366) using GTP (Invitrogen, R0461) and S-adenosyl-methionine (New England Biolabs, B9003S) as substrates to create a cap-1 structure. RNA was purified using LiCl precipitation.

### Example 16. Formulation Production

Lipid nanoparticles were formulated by rapid mixing of ethanol phase and aqueous phase using the microfluidic device (INano™ L system, Micro&Nano). The aqueous phase was a 50 mM citrate buffer (pH 6) containing the purified srRNA. The ethanol phase comprises an ionizable lipid, heptadecan-9-yl 8-(3-(((4-(dimethylamino)butanoyl)oxy)methyl)-4-((8-(nonyloxy)-8-oxooctyl)oxy)phenoxy)octanoate 1,2-Diastearoyl-sn-glycero-3-phosphocholine (DSPC) (Avanti, 850365P), Cholesterol (Sigma-Aldrich, C8667) and 1,2-dimyristoyl-sn-glycero-3-phosphoethanolamine-N-[methoxy(polyethylene glycol)-2000] (NOF, GM020). The RNA-LNPs were assembled with mole ratios 10:48:2:40 (DSPC:cholesterol: PEG 2000:LKY-XH15) at a NP lipid:RNA ratio of 8. Formulations were characterized for particle size, RNA concentration, encapsulation efficiency, and ability to protect from RNase digestion.

### Example 17. Modes of Vaccine Administration

All animal studies were carried out at Fangcheng Gang Lab of Shanghai HkeyBio Technology Co., Ltd. (Fangcheng Gang Spring Biological Technology Development Corporation Ltd, China), and all experiments involving laboratory animals were approved by the Institutional Animal Care and Use Committee (IACUC) of Fangcheng Gang Spring Biological Technology Development Corporation Ltd. To mimic natural VZV infection, two groups of three adult male cynomolgus monkeys were immunized once with live attenuated VZV (LAV) vaccine subcutaneously (SC) in the upper arm 28 days prior to study start (Day −28). On Day 0 and day 62, these monkeys were immunized intramuscularly with one dose of 10-30 μg VZV gE RNA/LNP respectively (FIG. 5). Serum and peripheral blood mononuclear cells (PBMC) were collected 14 days post-vaccination with a final collection occurring five months post-second vaccination. All samples were cryopreserved following standard protocols.

### Example 18. ELISA and Avidity Assay

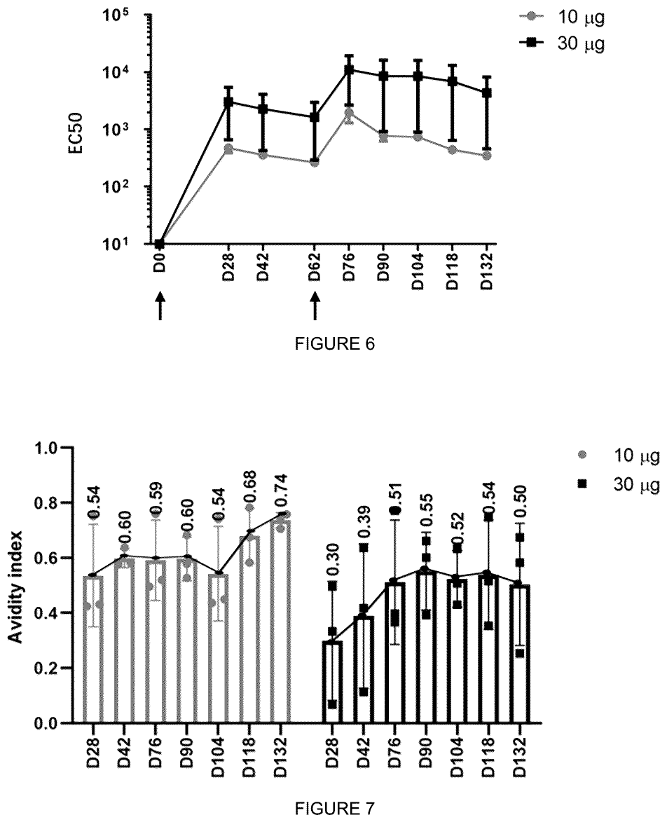

Antibody binding titers against VZV gE from the monkeys were quantified by enzyme-linked immunosorbent assay (ELISA). Briefly, 0.5 μg/ml recombinant VZV gE protein (Abeam, ab43050) diluted in carbonate-bicarbonate buffer was pre-coated onto 96-well clear polystyrene microplate (Corning) overnight at 4° C. After washing with PBS-T (0.05% Tween-20 in PBS) three times, the coated plates were blocked with 300 μl blocking buffer (15% goat serum and 2% bovine serum albumin in PBS-T) for 1 h at 37° C. Serum samples were 2-fold serially diluted in blocking buffer, transferred to the plates, and incubated for 1 h at 37° C. After washing, plates were incubated with HRP-conjugated goat anti-monkey IgG H+L (Abcam, ab112767) for 1 h at 37° C. Plates were washed and incubated with TMB Single-Component Substrate solution (Solarbio, PR1200) for 7 min at 37° C., and the reaction was stopped with ELISA Stop Solution (Solarbio, C1058). Absorbance was read at 450 nm on a microplate reader (SpectraMax Abs Plus, Molecular Devices). ELISA titers were determined using non-linear 4-parameter variable slope analysis in GraphPad Prism 8 software. The data not reaching EC50 was set to a baseline value of 10 as extrapolation beyond the data curve is imprecise (FIG. 6).

To evaluate antibody avidity, ELISAs were carried out as described above with minor modifications as needed. Following serum incubation, plates were washed three times with PBS-T and incubated with 8M urea (TCI, U0073) diluted in PBS for 5 min at room temperature. Plates were washed three times with PBS-T and the remainder of the ELISA was carried out as described above. Avidity index was calculated as the EC50 of wells treated with 8M urea divided by the EC50 of control wells without urea treatment (FIG. 7).

### Example 19. PBMC Cell Stimulation and Intracellular Cytokine Staining

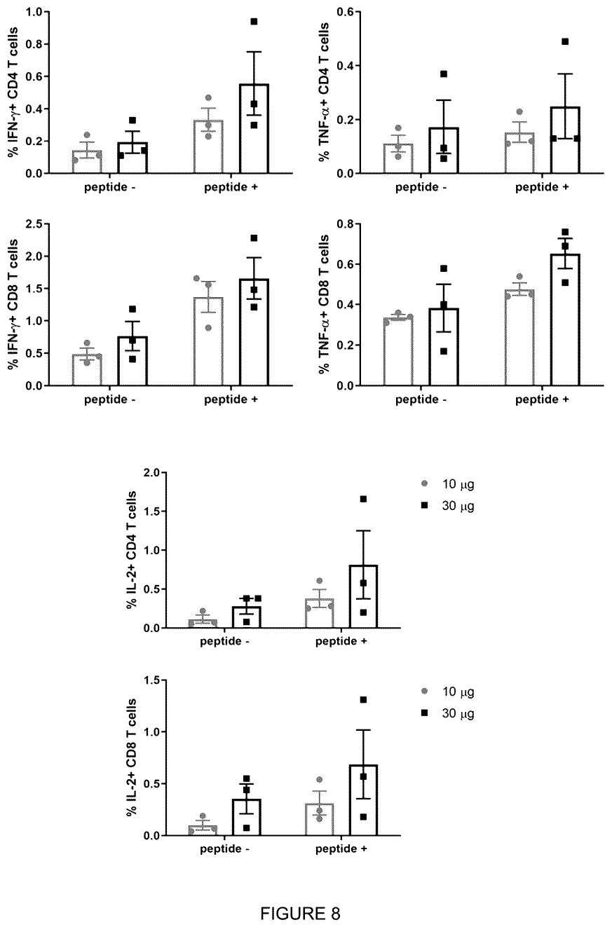

Cryopreserved cynomolgus PBMCs were quick-thawed in a 37° C. water bath and washed with medium (RPMI-1640 supplemented with 10% fetal bovine serum (FBS), 1× penicillin-streptomycin). Cells were distributed into a 96-well round-bottomed plate and were restimulated in vitro using a pool of peptides spanning the VZV gE protein (15 mers, overlapping by 11aa) at 2 μg/ml and CD28 monoclonal antibody (Invitrogen, 16-0289-81) and CD49d monoclonal antibody (Invitrogen, 16-0499-81) at 1.25 μg/ml. The plates were incubated at 37° C., 5% CO2 for 2 h, followed by treatment with a protein transport inhibitor cocktail (Invitrogen, 00-4980-93) overnight. Cells were washed with Dulbecco's phosphate-buffered saline (D-PBS) and stained with LIVE/DEAD™ fixable aqua dead cell stain (Invitrogen, L34965) for 30 min. Cells were washed with FACS wash buffer (D-PBS with 2% FBS) and incubated with fluorescently labeled antibodies for 30 min to stain cell surface proteins. Antibodies included mouse anti-human CD3 APC-Cy™7 (clone SP34-2), CD4 Brilliant Violet 421 (clone L200), and CD8 FITC (clone RPA-T8) (all from BD Biosciences). Cells were washed in FACS wash buffer and incubated with fixation/permeabilization working solution (Invitrogen, 00-51232-43) for 30 min in accordance with manufacturer instructions. Cells were washed twice with permeabilization buffer (Invitrogen, 00-8333-56) and incubated with fluorescently labeled antibodies for 30 min to detect intracellular cytokine expression. Antibodies included mouse anti-human IFN-γ PE (clone 4S.B3 from BD Biosciences) and TNF-α PerCP (clone MAb11) as well as rat anti-human IL-2 (clone MQ1-17H12) (all from Biolegend). Cells were washed with permeabilization buffer and resuspended in MACSQuant running buffer (Miltenyi Biotec). Fluorescent signals were acquired using MACSQuant16 (Miltenyi Biotec). Data were analyzed with FlowJo software (version X) (FIG. 8).

### Example 20. Efficacy of srRNA Vaccine Construct in Cynomolgus Monkeys

As outlined above, Cynomolgus monkeys (3 monkeys per group) were inoculated with LAV followed by 10 μg or 30 μg of srRNA vaccine encoded VZV gE on both Day 0 and Day 62 via intramuscularly delivery (FIG. 5).

The VZV gE-specific antibody binding titers were quantified by VZV gE ELISA. As shown in FIG. 6, VZV gE specific antibody titers were relatively low after LAV inoculation, however, the srRNA vaccine induced a high and persistent VZV gE specific antibody response after 2 immunizations. Moreover, a dose-titration effect was observed across srRNA vaccine doses. Antibody avidity in serum of immunized monkeys was also determined and is shown in FIG. 7. The avidity index demonstrates that both 10 μg and 30 μg of srRNA vaccine immunization resulted in rapid antibody affinity maturation (FIG. 7).

To measure the frequency and magnitude of antigen-specific T-cell subsets, PBMC from immunized Cynomolgus monkeys were stimulated with peptides spanning the VZV gE protein and interrogated for the ability to respond by producing IFN-γ and TNF-α as well as IL-2 in CD4+ T cells and CD8+ T cells. Individual cytokine expression data are shown in FIG. 8. At 4 weeks post-immunization 3 (day 90), srRNA vaccine elicits markedly increasing and high frequencies of VZV gE-specific CD4+ T cells, and much stronger CD8+ T cells.

Compared to Shingrix and conventional (non-replicating) mRNA, the srRNA vaccine elicits a comparable level of antibody response, stronger CD4+ T cell responses, and much stronger CD8+ T cell responses since Shingrix and conventional mRNA do not elicit detectable CD8+ T cell response. This striking difference in CD8+ T cell response is unexpected and offers advantages over the current standard of care.

### Example 21. Relative Potency of Lyophilized RNA

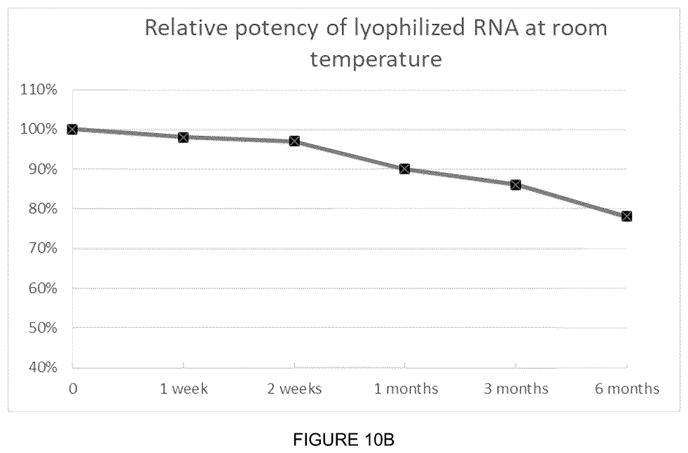

Lyophilized self-replicating RNA encoding a model protein (green fluorescence protein, GFP) was stored at about 4° C. or room temperature (about 20-24° C.). Data are shown in FIG. 10A and FIG. 10B. Samples were pulled at selected time points and tested using an in vitro potency assay, which compares the total intensity of fluorescence signal against a reference standard emitted by Baby Hamster Kidney cells when transfected by equal amount of RNA and lipofectamine 2000 mixture and cultured for 48 hours. Relative potency at Time 0 was set at 100%.

### Example 22. VZV srRNA Vaccine—Animal Experiment

Before administration, the srRNA component in a vial and LNP dispersion in a separate vial were equilibrated to room temperature for about 15 minutes. The LNP dispersion was gently shaken for 5-10 seconds. 0.6 mL LNP dispersion was drawn from the vial by a needle-syringe combination and added into the lyophilized srRNA in the first vial. Then, the vial was inverted up and down for approximately 30 seconds for thorough mixing to obtain the reconstituted vaccine, which appeared as a white to off-white suspension.

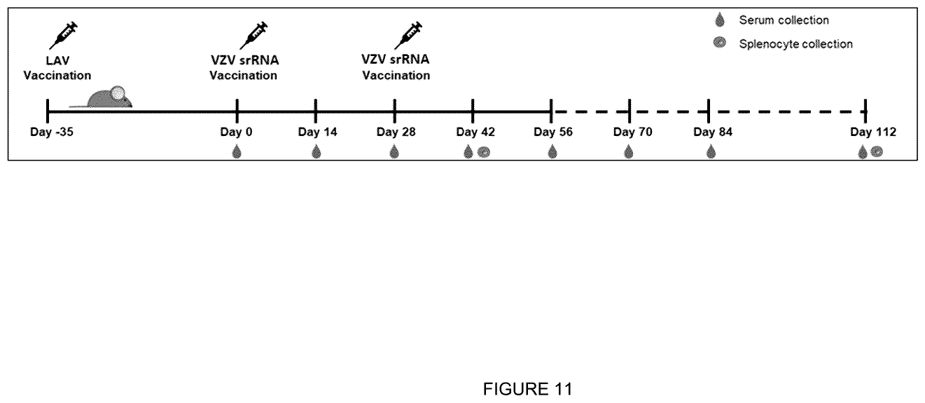

The animal experiment was conducted using 80 female SPF C57BL/6 mice, which were randomly assigned into 7 groups (Table 13) and immunized with test articles according to the predetermined immunization schedule as shown in the schematic illustration of FIG. 11.

TABLE 13
Mouse grouping and dosing information.

| | | Administration | |
|---|---|---|---|
| | Pre-immunization | of the VZV | Number |
| Group | of LAV vaccine | srRNA vaccine | of mice |
| 1 | No | Two doses, 15 μg | 12 |
| 2 | No | Single dose, 15 μg | 12 |
| 3 | Yes | Two doses, 15 μg | 12 |
| 4 | Yes | Single dose, 15 μg | 12 |
| 5 | Yes | Sterile saline | 12 |
| 6 | Yes | Two doses, 3 μg | 12 |
| 7 | No | N.A | 8 |

To mimic the natural VZV infection and subsequent latent status, mice in groups (G) 3-6 were pre-immunized with a live attenuated influence (LAV) vaccine (3.3 lg PFU) subcutaneously (s.c) in the scruff of the neck 35 days prior to study start (Day −35). Mice in G1, G3 and G6 were immunized twice by intramuscular (i.m.) administration of the VZV srRNA vaccine at 15 μg per dose (G1, G3) or 3 μg per dose (G6) in the quadriceps muscle of hindlimbs on Day 0 and Day 28, respectively. In groups G2 and G4, mice were intramuscularly immunized once with the VZV srRNA vaccine at 15 μg per dose on Day 28. In saline control group (G5), mice were intramuscularly administrated sterile saline on Day 0 and Day 28. The intramuscular (i.m.) injection in the quadriceps muscle of hindlimbs was performed at multiple sites with each site not exceeding 0.1 mL. In the naive control group (G7), mice were maintained without receiving any administration. The detailed information of mouse grouping and dosing was shown in Table 13.

Blood samples (50-100 μL per mouse) were collected at the indicated timepoints shown in the vaccination schedule (FIG. 7) and serum preparation was performed as stated in the protocol. Also, spleens were harvested from 50% mice of each group on Day 42 for isolating splenocytes in accordance with the protocol. The other 50% mice in the study were kept for a follow-up of the immune responses.

### Serum Preparation

Blood samples were collected in Eppendorf tubes and maintained on ice. After centrifugation at 1,500 g for 10 min at 4° C., the supernatant was immediately transferred to new tubes and stored at below −70° C.

### Splenocyte Isolation

To isolate splenocytes, fresh spleens were isolated from immunized female SPF C57BL/6 mice or control mice, and gently homogenized in PBS using a piston syringe. Then, the cell suspension was passed through a 70 μm cell strainer (BD Falcon). Red blood cells were lysed using red blood cell lysate buffer in accordance with manufacturer's instructions (Invitrogen). After washing twice with PBS, the cells were suspended in 0.5 mL medium (RPMI-1640 supplemented with 10% fetal bovine serum (FBS), 1× penicillin-streptomycin).

### Enzyme-Linked Immunosorbent Assay (ELISA)

Antibody titers against VZV gE were quantified by enzyme-linked immunosorbent assay (ELISA). Briefly, 0.5 μg/mL recombinant VZV gE protein diluted in ELISA coating buffer was pre-coated in 96-well clear polystyrene microplate overnight at 4° C. After washing with 0.5% PBS-T for three times, the coated plates were blocked with 300 μL ELISA blocking buffer per well for 1 h at 37° C. Serum samples were 2-fold serially diluted in blocking buffer, transferred to the coated plates, and incubated for 1 h at 37° C. After washing, plates were incubated with HRP-conjugated rabbit anti-mouse IgG (H+L) antibody for 1 h at 37° C. Plates were washed three times with 0.5% PBS-T and incubated with TMB single-component substrate solution for 7 min at 37° C. Then, the reaction was stopped with an ELISA stop solution. Absorbance was read at 450 nm on a microplate reader. To determine the antibody level, the half maximal effective concentration (EC50) was calculated for each sample using a non-linear 4-parameter variable slope analysis in GraphPad Prism 8 software. The value of EC50 was used to represent the antibody level for each sample.

### Cell Stimulation

Fresh splenocytes (2×106 in 200 μL medium) were seeded into a 96-well round-bottom microplate and were restimulated using a pool of peptides spanning the VZV gE protein (a pool of 138 15-mer peptides with 11-amino acids overlap, as listed in Appendix 7.6) at 1.25 μg/mL and CD28 monoclonal antibody plus CD49d monoclonal antibody at 1.25 μg/mL. The plate was incubated at 37° C. in a humid incubator with 5% CO2 for 2 h, followed by treatment with a protein transport inhibitor cocktail overnight.

### Cell Surface and Intracellular Staining

Cells were washed with Dulbecco's Phosphate Buffered Saline (DPBS) and stained with LIVE/DEAD™ fixable aqua dead cell stain for 30 min. Cells were washed with 200 μL fluorescence activated cell sorting (FACS) wash buffer, and incubated with fluorochrome-labeled primary antibodies for 30 min to stain cell surface proteins. Antibodies included anti-mouse CD3 APC-Vio 770 (clone REA641), anti-mouse CD4 VioBlue (clone REA604), and anti-mouse CD8 PerCP (clone REA601) (1 μL per well). Afterwards, cells were washed by FACS wash buffer and incubated with fixation/permeabilization working solution for 30 min in accordance with manufacturer's instructions. Then, cells were washed twice with permeabilization buffer and incubated with fluorochrome-labeled primary antibodies for 30 min to detect intracellular cytokine expression. Antibodies included anti-mouse IFN-γ FITC (clone REA638), anti-mouse TNF-α PE (clone REA636), and anti-mouse IL-2 APC (clone REA665) (1 μL per well). At last, cells were washed with 200 μL permeabilization buffer and resuspended in 200 uL PBS for flow cytometry analysis.

### Sample Analysis

Stained cells were analyzed by CytoFlex flow cytometer (Beckman). Data were analyzed with CytExpert software.

VZV gE-Specific Humoral Immune Response

The humoral response was determined by the binding titers of VZV gE-specific IgG antibody, which was quantified by ELISA in serum samples collected at the indicated timepoints post-immunization. The antibody level was represented by EC50 value that was determined as described.

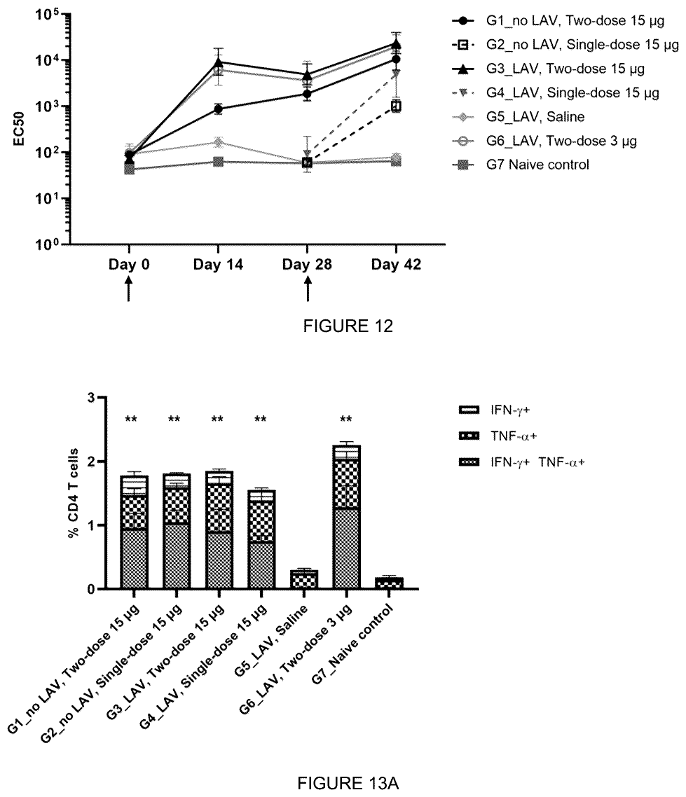

In this study, a LAV-primed mouse model was used to evaluate the immunogenicity of VZV srRNA vaccine. The data are shown in FIG. 12. LAV-primed mice injected with sterile saline (G5) showed that the level of VZV gE specific IgG antibodies increased slightly by 2-fold on Day 0 (p<0.0001) and by 3-fold at Day 14 (p<0.0001), but was similar on Day 28 and Day 42 in comparison to the naive control group (G7). Following LAV prime immunization, in the single-dose the VZV srRNA vaccine group (G4), the antibody level on Day 42 was 105-fold higher (p<0.05) than that in the group without vaccine immunization (G5). Meanwhile, in the two-dose immunization groups, the antibody levels on Day 14 post first immunization at 15 μg per dose (G3) and 3 μg per dose (G6) were also higher (70-fold; p<0.0001 and 46-fold; p<0.0001, respectively) as compared to G5. The humoral response was further enhanced after the second immunization, as evidenced by the remarkably higher antibody level on Day 42 in G3 and G6 (332-fold; p<0.0001 and 271-fold; p<0.0001, respectively) than that in G5. It was worth noting that the antibody levels elicited by the high dose (15 μg) were similar to that by the low dose (3 μg) on Day 14 (2.5-fold; p=0.241), Day 28 (2.3-fold; p=0.829) and Day 42 (1.6-fold; p=0.530). Taken together, in the presence of LAV pre-immunization, either a single-dose or a two-dose vaccination induced a strong VZV gE-specific humoral immune response. Furthermore, VZV srRNA vaccination at a low dose (3 μg per dose) induced a similar antibody level to that as a high dose (15 μg per dose) in mice.

To further evaluate the immunogenicity of VZV srRNA vaccine in the absence of LAV priming, the effect of either a single-dose or a two-dose vaccine immunization at 15 μg per dose on the humoral response was compared. Comparing to the naive control group (G7), one VZV srRNA vaccine immunization induced moderate up-regulation of VZV gE-specific antibody level by 15-fold (p<0.0001) on Day 14 in G1 and 17-fold (p<0.0001) at Day 42 in G2, respectively. Following the second immunization, the humoral response was significantly enhanced, and the antibody level on Day 42 in G1 was 197-fold (p<0.0001) higher than that in G7. Besides, the influence of LAV priming on the VZV gE-specific IgG antibody level induced by VZV srRNA vaccine immunization was assessed. In the groups of two-dose VZV srRNA vaccine immunization (G1 and G3), the antibody level in G3 was higher than that in G1 on Day 14 (14-fold; p<0.0001), Day 28 (3.4-fold; p<0.0001) and Day 42 (2.9-fold; p<0.01), respectively. In the groups of single dose immunization (G2 and G4), the VZV gE-antigen specific IgG antibody response in G4 was higher than G2 on Day 42 (9.6-fold; p<0.05). These results suggested that the priming of LAV affected the level of gE-specific IgG antibodies induced by VZV srRNA vaccine immediately after one immunization, whereas the influence of LAV pre-prime became less apparent after the second VZV srRNA immunization at the high dose. Therefore, administration of VZV srRNA vaccine in the absence of LAV priming also could elicit a remarkable VZV gE-specific humoral immune response.

VZV gE-Specific Cellular Immune Response

VZV antigen-specific T cell responses are vital for the recovery from primary VZV infection and in preventing reactivation of latent VZV. Therefore, the frequencies of antigen-specific T-cell subsets were measured in splenocytes of immunized mice following in vitro restimulation with a peptide pool spanning the VZV gE protein.

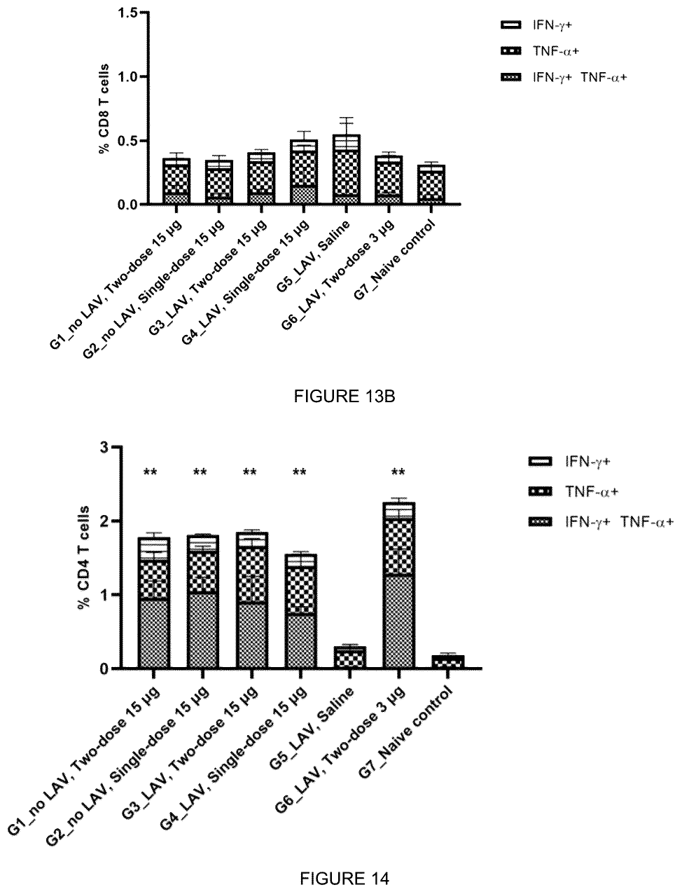

First, the effect of LAV priming on the VZV gE-specific cellular immune response was evaluated. Compared to the naive control (G7), the frequencies of total cytokine (IFN-γ and/or TNF-α)-expressing CD4+T or CD8+ T cells induced in the LAV priming control group (G5) did not increase significantly on Day 42 (p=0.034, p=0.181, respectively). Therefore, administration of LAV only was not capable of activating the VZV gE-specific T cell immune response in mice (FIG. 13A and FIG. 13B)

Following the LAV priming, immunization with two-doses of the VZV srRNA vaccine markedly elevated the frequencies of VZV gE-specific CD4+ T cells expressing IFN-γ and/or TNF-α (increased by 6.1-fold in G3, p<0.0001 and by 7.5-fold in G6, p<0.0001) as compared to the LAV-priming control group without VZV srRNA injection (G5) (FIG. 14). Moreover, the CD4+ T cell response in G6 was similar to that in G3 (p=0.122), indicating the low dose VZV srRNA vaccine was sufficient to stimulate a strong cellular immune response. Besides, similar to the two-dose VZV srRNA vaccine administration, the frequencies of total cytokine (IFN-γ and/or TNF-α)-expressing CD4+T in G4 were 5.2-fold higher than the control group (G5), suggesting the single-dose immunization of VZV srRNA vaccine also could significantly activate CD4+ T cell responses. These results demonstrated that either single-dose or two-dose of VZV srRNA vaccine following LAV pre-immunization activated a remarkable VZV gE-specific CD4+ T cellular immune response.

Lastly, the effect of LAV priming on the cellular immune response activated by VZV srRNA vaccine immunization was assessed. In the groups of two-dose VZV srRNA vaccine immunization with and without LAV-priming, the frequencies of total cytokine (IFN-γ and/or TNF-α)-expressing CD4+ T cells in G1 was similar to that in G3 (p=0.576). In the groups of single dose immunization (G2 and G4), the frequencies of total cytokine (IFN-γ and/or TNF-α)-expressing CD4+ T cells response without LAV-priming in G2 showed slightly higher than that in G4 with LAV-priming (p=0.044). Thus, administration of the VZV srRNA vaccine in the absence of LAV-priming also could elicit a VZV gE-specific CD4+ T cellular immune response. (FIG. 14).

### Example 23. srRNA Structure with an Elongated 3′ UTR and/or PolyA Tail

This Example tests modifications of the 5′ cap structure, controlling the length of the poly(A) tail, including modified nucleotides, codon or sequence optimization, as well as altering the 5′ and 3′ UTRs for improving the srRNA structure.

Natural eukaryotic mRNA has a 7-methylguanosine (m7G) cap coupled to the mRNA. 5′UTR, Capping of IVT mRNA includes at least three forms, i.e., cap-0 [m7G (5′) pppN1pN2p], cap-1 [m7G (5′) pppN1mpNp] and cap-2 [m7G (5′) pppN1mpN2mp]. Alphavirus 7-methylguanosine (m7G) cap structure (Cap 0) capping is facilitated by nsP1 in a manner, and it lacks this additional 2-O-methylation modification. Length of 5′ and 3′ UTRs vary among the alphavirus, the 5′ UTRs of SIN range in length from 58 to 59 and the 3′ UTRs range from 268 to 323 nucleotides. A minimum of 11-12 residues in the poly(A) tail is required for efficient production of negative strand RNA, the poly(A) tail functions in conjunction with the CSE to support negative strand synthesis as well as efficient translation.

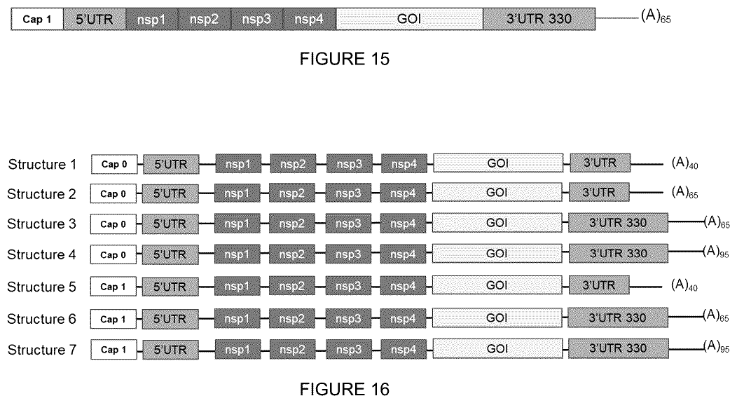

An exemplary srRNA vector structure is shown in FIG. 15. The vector comprises six parts: a 5′ cap, 5′ Untranslated Regions, viral nonstructural proteins (nsP1-4), subgenomic promoter, 3′ Untranslated Regions and poly(A) Tail. The 5′ UTR controls translation, and the 5′-terminal open reading frame (ORF) is translated into viral nonstructural proteins (nsP1-4). These proteins form the enzyme complex required for the replication of viral genome and transcription of the subgenomic RNA. The srRNA vector structure optimizes the Cap type, 3′ untranslated regions, and poly(A) tail to enhance RNA stability (extend the half-life) in vivo. The structure use Cap1 and optimized 3′ UTRs based on naturally occurring alphaviruses to make the vector mimic a real virus replicating in the body. Moreover, it has been observed that increasing the poly(A) tail length improves the efficiency of polysome generation, and consequently influences the protein expression levels. The disclosed elongated the poly(A) tail is extended to 65 residues or 95 residues to promote srRNA stability and translation of viral transcripts.

### Gene Synthesis and Recombinant Plasmid Cloning

DNA encoding a gene of interest (GOI) was chemically synthesized and cloned into restriction digestion linearized pUC57 plasmid vector. DNA synthesis and gene cloning were customized and ordered from Nanjing Genscript Co., Ltd. (Nanjing, China). srRNA replicon backbone was chemically synthesized by Suzhou Genwitz Co., Ltd. (Suzhou, China). GOI and replicon backbone were digested by ApaI and NotI, through T4 DNA ligase, the GOI sequence was inserted into the backbone, resulting in a recombinant plasmid. Through sequencing, the GOI was proved to be inserted into the expression backbone accurately.

### RNA Synthesis

The RNA was produced in vitro using T7 RNA polymerase-mediated transcription from a linearized DNA template from recombinant plasmid, which encodes codon-optimized RBD region of SARS-CoV-2 and incorporates the 5′ and 3′ untranslated regions and a poly-A tail. The VZV gE protein-encoding RNA was prepared in the same procedure.

### Cell Lines

HEK-293T and BHK-21 cells were purchased from the National Collection of Authenticated Cell Cultures (https://www.cellbank.org.cn/).

### Cell Culture and Transfection

HEK-293T and BHK-21 cultured in Dulbecco's modified Eagle's medium (DMEM, Gibco) supplemented with 10% fetal calf serum (Gibco) and penicillin/streptomycin antibiotics (100 U/ml penicillin, 100 μg/ml streptomycin; Gibco) and maintained at 37° C., 5% CO₂, and 90% relative humidity. 3×10⁵ cells per well were seeded in 6 well plates. 2 μg srRNAs per well for 6-well plates were transfected with lipofectamine 3000 transfection kit (Invitrogen, 2320817) on the next day when the cell culture must have >90% viability and be 70% confluent. Samples were collected after 24 hour and 48 hour post-transfection for the following detections.

### In Vitro Potency Assay

The RBD protein of SARS-CoV-2 in cell supernatant was detected through Western Blot. Cells transfected with RNA were lysed by NP-40 Lysis buffer (50 mM Tris pH 7.4, 150 mM NaCl, 1% NP40, sodium pyrophosphate, β-glycerophosphate, sodium orthovanadate, sodium fluoride, EDTA, leupeptin). The mixtures were centrifuged at 13,000 rpm for 5 minutes at 4° C. The supernatants were collected and boiled with SDS for 12 minutes at 95° C., separated in a 6% SDS-PAGE gel and transferred to nitrocellulose filter membranes. After blocking with 5% BSA, the membranes were first blotted with primary antibodies (1:1000) (SARS-CoV-2 (2019-nCoV) Spike Rabbit PAb, 40592-T62, Sino Biological), then incubated with horseradish peroxidase (HRP) conjugated secondary antibodies (1:10000) (IgG(H+L) (HRP-labeled Goat Anti-Rabbit IgG(H+L))), Beyotime), and visualized with Chemiluminescent Reagent (Chemiluminescent HRP Substrate, WBKLS0500, Millipore). The VZV gE protein was detected by the same method using Anti-VZV gE protein antibody (abeam, ab272686).

### Preparation of RNA-LNP

Lipid nanoparticles (LNP) were formulated by rapid mixing of the ethanol phase and the aqueous phase using a microfluidic device. The aqueous phase is a 50 mM citrate buffer (pH 6.0) and comprises a certain amount of RNA. The ethanol phase comprises an ionizable lipid (as described herein), cholesterol (Jiangsu Southeast Nanomaterials), 1,2-Diastearoyl-sn-glycero-3-phosphocholine (DSPC) (Jiangsu Southeast Nanomaterials), and 1,2-dimyristoyl-rac-glycero-3-methoxypolyethylene glycol-2000 (DMG-PEG2000) (SINOPEG). These four lipid components were mixed at a molar ratio of 40:48:10:2.0 (ionizable lipid:cholesterol:DSPC:DMG-PEG2000). The generated LNP were purified in citrate buffer (pH 6.0) and characterized with respect to particle size, polydispersity index (PDI), and RNA concentration. Finally, adjust the pH to 7.2. the RNA-LNP were then diluted to the target concentration to obtain the RNA-LNP product and stored at −80° C.

Regarding characterization, particle size and polydispersity index (PDI) of the RNA-LNP were determined by Dynamic Light Scattering (DLS).

### Bioluminescence Imaging (BLI)

Female BALB/C mice aged 6-8 weeks were purchased from Shanghai SLAC Laboratory Animal Co., Ltd. 2 μg of LNP-srRNAs were administrated into mice intramuscularly. Up to test point, mice were anesthetized in a chamber with 2.5% isoflurane and given intra-peritoneal injections of D-Luciferin K+ salt XenoLight (Perkin Elmer, P/N 122799, 150 mg/kg). Luminescence was detected with an IVIS Spectrum imaging system (PerkinElmer Lumina LT, Waltham, MA) while maintaining 2% isoflurane in the imaging chamber via a nose cone. Images were captured 10 min after luciferin administration with sequence set-up for autoexposure. The photon total flux values (photons/second), corresponding to the region of interest (ROI) marked around the bioluminescence signal, were analyzed using the Caliper Life Sciences software (Living IMAGE Software, Caliper).

### Antigen-Specific Antibody Responses

Blood was collected from the retro-orbital sinus of immunized mice, and serum prepared. Antigen-specific IgG responses were detected by enzyme linked immunosorbent assay (ELISA) using commercial antigens: VZV gE protein (abeam ab43050) and SARS-CoV-2 spike protein (RBD, Histag) (GenScript Z03483). Briefly, 0.5 μg/ml antigen diluted in carbonate-bicarbonate buffer was pre-coated onto a 96-well clear polystyrene microplate (Corning) overnight at 4° C. After washing with PBS-T (0.05% Tween-20 in PBS) three times, the coated plates were blocked with 300 μL blocking buffer (15% normal goat serum and 2% bovine serum albumin in PBS-T) for 1 h at 37° C. Serum samples were 2-fold serially diluted in blocking buffer, transferred to the plates, and incubated for 1 h at 37° C. After washing, plates were incubated with HRP-conjugated rabbit anti-mouse IgG H+L (Abcam, ab6728) for 1 h at 37° C. Plates were washed and incubated with TMB Single-Component Substrate solution (Solarbio, PR1200) for 7 min at 37° C., and the reaction was stopped with ELISA Stop Solution (Solarbio, C1058). Absorbance was read at 450 nm on a microplate reader ((VARIOSKAN LUX, ThermoFisher) and ELISA titers were determined using non-linear 4-parameter variable slope analysis in GraphPad Prism 8 software.

### Intracellular Cytokine Staining and Flow Cytometry-Cell Stimulation

Fresh splenocytes (2×10⁶ in 200 μL medium) were seeded into a 96-well round-bottom microplate and were restimulated using a pool of peptides spanning the VZV gE protein (a pool of 138 15-mer peptides with 11-amino acids overlap) at 1.25 μg/mL and CD28 monoclonal antibody plus CD49d monoclonal antibody at 1.25 μg/mL. The plate was incubated at 37° C. in a humid incubator with 5% CO₂ for 2 h, followed by treatment with a protein transport inhibitor cocktail overnight.

### Intracellular Cytokine Staining and Flow Cytometry-Cell Surface and Intracellular Staining

Cells were washed with Dulbecco's Phosphate Buffered Saline (DPBS) and stained with LIVE/DEAD™ fixable aqua dead cell stain for 30 min. Cells were washed with 200 μL fluorescence activated cell sorting (FACS) wash buffer and incubated with fluorochrome-labeled primary antibodies for 30 min to stain cell surface proteins. Antibodies included anti-mouse CD3 APC-Vio 770 (clone REA641), anti-mouse CD4 VioBlue (clone REA604), and anti-mouse CD8 PerCP (clone REA601) (1 μL per well). Afterwards, cells were washed by FACS wash buffer and incubated with fixation/permeabilization working solution for 30 min in accordance with manufacturer's instructions. Then, cells were washed twice with permeabilization buffer and incubated with fluorochrome-labeled primary antibodies for 30 min to detect intracellular cytokine expression. Antibodies included anti-mouse IFN-γ FITC (clone REA638), anti-mouse TNF-α PE (clone REA636), and anti-mouse IL-2 APC (clone REA665) (1 μL per well). At last, cells were washed with 200 μL permeabilization buffer and resuspended in 200 μL PBS for flow cytometry analysis.

Stained cells were analyzed by CytoFlex flow cytometer (Beckman). Data were analyzed with CytExpert software.

Construction and Quality Control of srRNA Vaccines

The srRNA structures used are shown in the FIG. 16. Three model GOI were used to evaluate the in vitro and in vivo efficacy. For in vitro efficacy evaluation, the SARS-CoV RBD srRNA constructs were produced via in vitro transcription (IVT) then transfection of cells by using lipofectamine 3000 followed by RBD protein expression analysis by WB. For in vivo efficacy evaluation, the VZV gE, and luciferase srRNA constructs were produced via in vitro transcription (IVT) and were formulated in LNPs containing ionizable lipid (Lipid #9), Cholesterol, DSPC, and DMG-PEG2000. For all in vivo experiments, LNPs were characterized for size, Polydispersity index (PDI), encapsulation efficiency and concentration (Table 14). As shown in Table 14, all characteristics of RNA-LNP for structures 1, 3-6 srRNA are suitable for delivering RNA.

TABLE 14
Representative pharmaceutical characteristics of RNA-LNP
Vaccine Test items

| | | | | RNA | |
|---|---|---|---|---|---|
| | | Size | | encapsulation | RNA conc. |
| SrRNA | | (nm) | PDI | ratio | (μg/mL) |
| Luciferase | Structure 1 | 104.93 | 0.099 | 97.6 | 35.4 |
| | Structure 3 | 95.53 | 0.097 | 95 | 35 |
| | Structure 4 | 95.68 | 0.077 | 96.8 | 34.6 |
| VZV gE | Structure 5 | 85 | 0.207 | 97 | 77.4 |
| | Structure 6 | 103 | 0.131 | 94.9 | 29.9 |
| | Structure 7 | 106 | 0.094 | 95.4 | 75.2 |

### RNA In Vitro Transfection

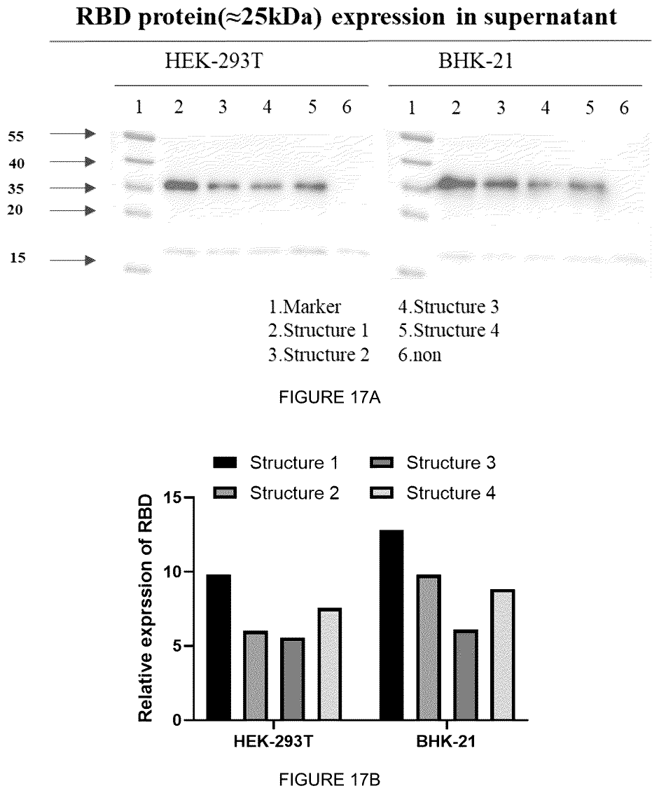

The transfection efficiency of RBD RNA in four structures were compared in HEK-293T and BHK-21 cell lines at the same dose (1 μg). In this study, 24 hours post transfection, Western Blot was used to detect the RBD protein in cell supernatant as shown in FIGS. 17A and 17B. Based on the results shown in FIGS. 17A and 17B, it can be concluded that the RNA of the four structures disclosed herein can express the RBD protein. Comparing the result of Structure 1 and Structure 2 in HEK-293T and BHK-21, the expression of RBD in Structure 2 is lower than in Structure 1. Comparing the result of Structure 2 and Structure 3 in HEK-293T and BHK-21 cells, the expression of RBD in Structure 3 is less than in Structure 2. Thus, this data shows that increasing the length of poly A tail can enhance the stability of RNA while slowing down the initial translation efficiency. Optimizing the 3′UTR to 3′UTR 330 also can slow down the initial translation efficiency and extend the time of in Vivo expression.

### Efficacy of RNA-LNP Vaccine in Mice

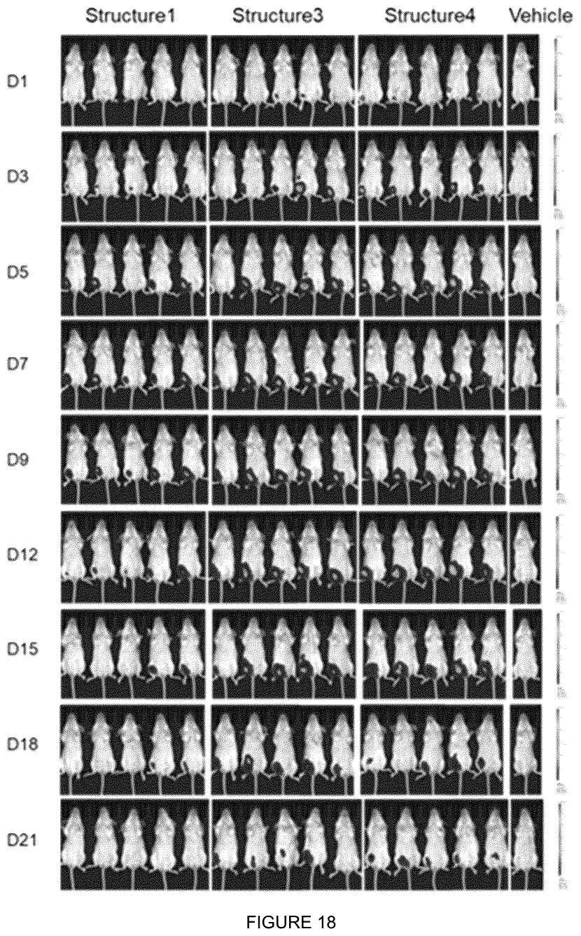

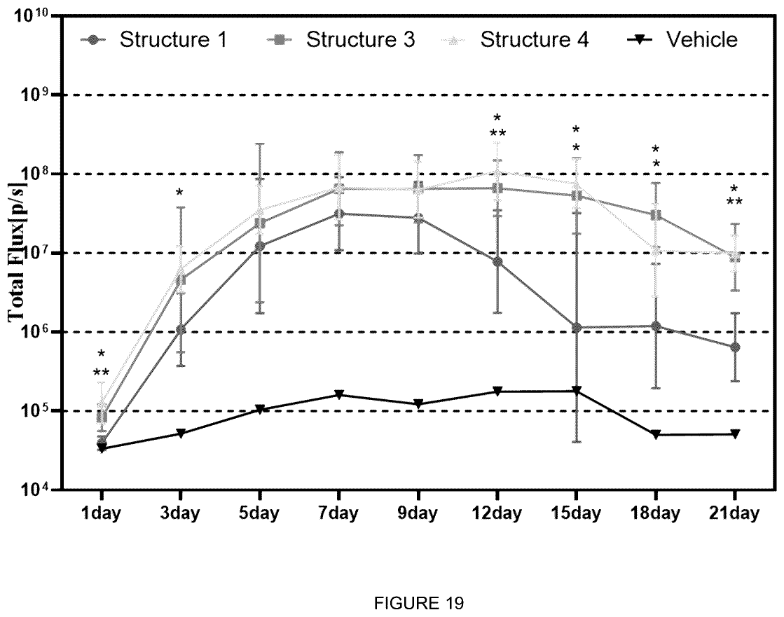

The in vitro expression results show that the optimized structure can reduce the initial expression amount. The next step is to verify whether the optimized structure can prolong the RNA expression cycle in animals. In vivo studies were carried out to compare the protein expression cycle in BALB/c mice upon vaccination with luciferase RNA-LNP encoding the luciferase protein. The in vivo expression of GOI in different structures in mice were evaluated. To visualize the expression of the RNA-LNP vaccines, luciferase encoding the RNA-LNP was prepared, and subjected to bioluminescence imaging (BLI) analysis using intramuscular injection (i.m.). Following i.m., robust expression of luciferase was seen in the injection site in BALB/c mice 5 days after injection, and the length of time of expression of Structure 3 and Structure 4 was longer than that of Structure 1 as shown in FIG. 18 and FIG. 19. Specifically, luciferase expression is detectable during days 5-9 for Structure 1, and days 5-21 for Structures 3 and 4. The optimized Structure 3 and Structure 4 with 3′UTR 330 and extended poly A tail can increase the in vivo half-life of the RNA.

In-Vivo Immunogenicity Evaluation of srRNA-VZV Vaccine in C57BL/6

It can be seen from the luciferase protein expression results in vivo that the disclosed srRNA structures with 3′UTR 330 and longer poly A tail can express proteins for a longer time than those srRNA structures without these features. The next step was to compare the antibody level produced by an expressed antigen protein. Here, the VZV gE protein in Structure 5, Structure 6 and Structure 7 were expressed, and compared the persistence of specific antibody response in Black6/C57 mice upon vaccination with VZVgE RNA-LNP. The srRNAs encoding VZVgE protein in Structure 5, Structure 6 and Structure 7 use Cap1 as the Cap 1. These three srRNA-VZV vaccines which used Structure 5, Structure 6, and Structure 7, as shown in FIG. 16.

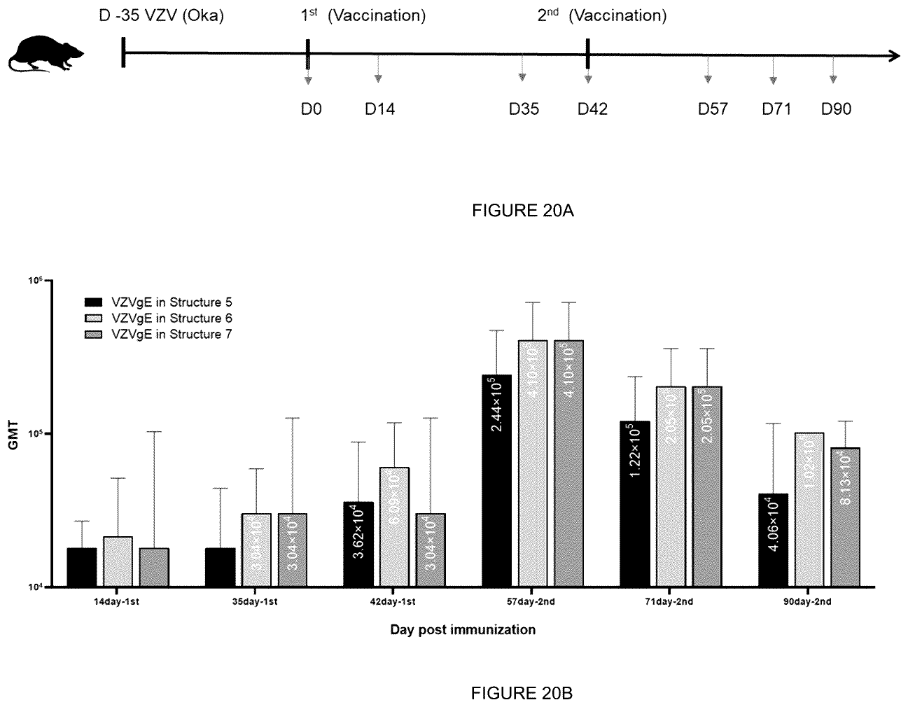

To mimic the natural VZV infection and subsequent latent status, mice were pre-immunized with a VARICELLA VACCINE, LIVE (LAV) vaccine (3.3 lg plaque forming unit (PFU)) subcutaneously (s.c) in the scruff of the neck 35 days prior to study start (Day −35). Mice were immunized twice by intramuscular (i.m.) administration of VZV vaccine at 2 μg per dose, with an interval of 42 days as shown in FIG. 20A, the schematic diagram of the VZVgE RNA-LNP vaccination process in mice.

Antibody titer is highly correlated with the protective effect and durability brought by the vaccine and therefore is used as the efficacy read-out in this study and presented in the form of Total IgG end point titer. As shown in FIG. 20B, post first injection, the Structure 6 vaccine induced the highest antibody response at day 14, day 35, and day 42 post the first vaccination. For all three RNA vaccines, antibody titers increased substantially after two immunizations. However, after two immunizations, the Structure 6 and Structure 7 vaccines seemed to elicit an even higher antibody response or equivalent antibody response for a long time, e.g. more than 90 days after the 1^{st} dose. These data prove that the Structure 6 and Structure 7 RNA-LNP vaccine can produce durable, specific antibodies in the body. Considering the stability of plasmid passage and the difficulty of gene synthesis for longer RNA, Structure 6 is more accessible for large-scale production. Both Structures 6 and 7 are suitable for prolonged srRNA expression of GOI and enhanced immunogenicity.

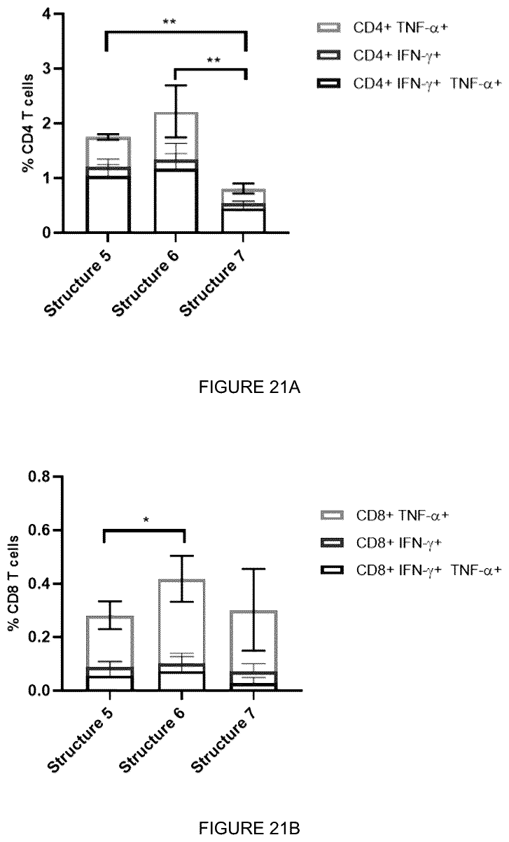

To measure the frequency and magnitude of antigen-specific T-cell subsets, PBMC from immunized mice were stimulated with peptides spanning the VZV gE protein and tested for the ability to respond by producing T cells expressing IFN-γ and/or TNF-α. On the 57^{th} day, some mice in each group were harvested, spleen cells were taken for flow cytometry, and the cellular immune response was measured. As shown in FIG. 21A, the frequencies of total cytokine (IFN-γ and/or TNF-α)-expressing CD4+ T cells in Structure 6 was higher than that for Structure 5 and Structure 7. Thus, Structure 6 could elicit a remarkable VZV gE-specific CD4+ T cellular immune response. As shown in FIG. 21B, the frequencies of total cytokine (IFN-γ and/or TNF-α)-expressing CD8+ T cells in Structure 6 was higher than that of Structure 5 and Structure 7. Thus, Structure 6 could also elicit a significant VZV gE-specific CD8+ T cellular immune response. The results showed that the vaccine prepared with the new self-replicating vector Structure 6 could induce the strongest immune response.

### Example 24. Synthesis of Lipid #4

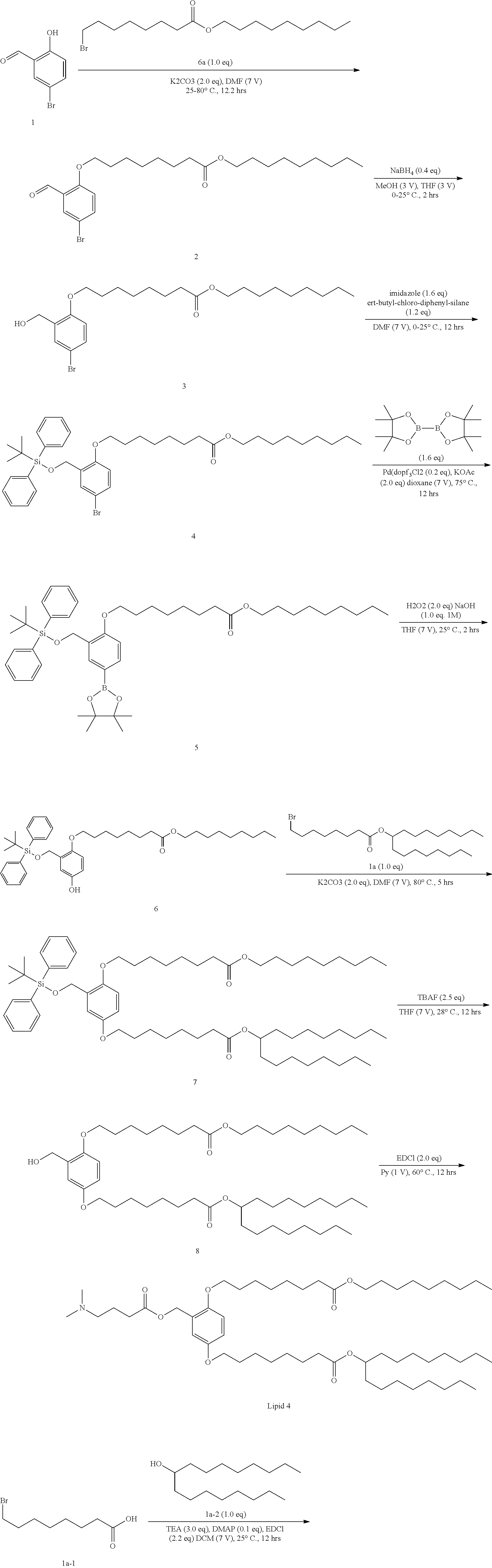

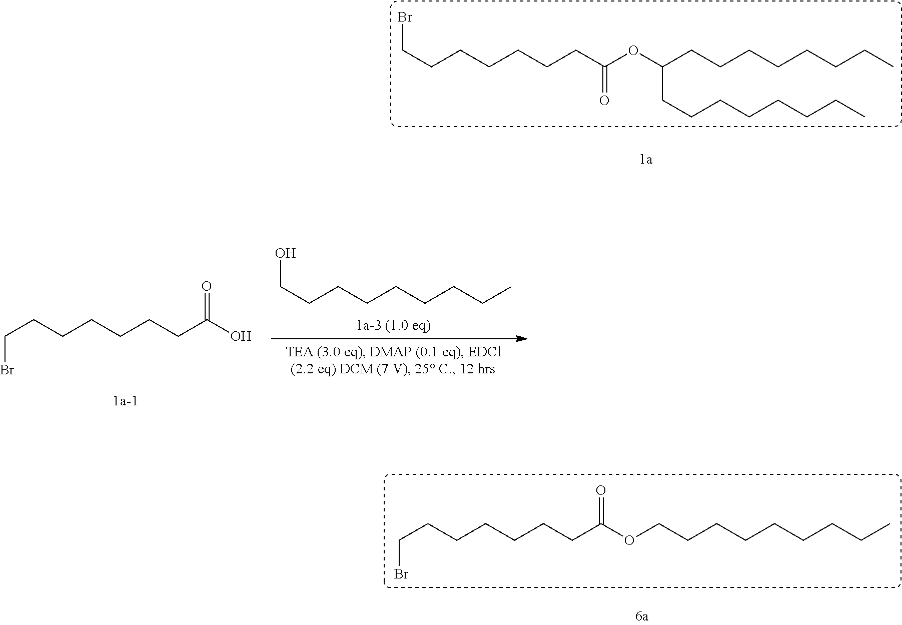

### Step 1. Synthesis of Compound 2

To a solution of compound 1 (60.0 g, 298 mmol) in DMF (420 mL), K₂CO₃ (82.5 g, 596 mmol) was added, the mixture was stirred at 25° C. for 10 mins, then compound 6a (104 g, 298 mmol) was added, the mixture was stirred at 80° C. for 12 hrs. TLC (Petroleum ether/Ethyl acetate=10/1, Product, Rf=0.6) showed compound 1 consumed completely and desired spot was detected. The reaction mixture was filtered and extracted with EtOAc (200 mL×2), and washed with brine 100 mL, dried over Na₂SO₄, concentrated under reduced pressure to give a residue. Compound 2 (95.0 g, crude) was obtained as brown oil.

### Step 2. Synthesis of Compound 3

To a solution of compound 2 (95.0 g, 202 mmol) in MeOH (475 mL) and THF (285 mL), NaBH₄ (3.06 g, 80.9 mmol) was added at 0° C., the mixture was stirred at 25° C. for 2 hrs. TLC (Petroleum ether/Ethyl acetate=10/1, Product, Rf=0.5) showed compound 2 consumed completely and desired spot was detected. The reaction mixture was quenched with MeOH 200 mL, and extract with EtOAc (200 mL×2), and washed with brine 100 mL, dried over Na₂SO₄, concentrated under reduced pressure to give a residue. Compound 3 (67.0 g, crude) was obtained as brown oil.

### Step 3. Synthesis of Compound 4

To a solution of Compound 3 (67.0 g, 142 mmol) in DMF (420 mL), imidazole (14.1 g, 213 mmol), tert-butyl-chloro-diphenyl-silane (46.7 g, 170 mmol) was added at 0° C., the mixture was stirred at 25° C. for 12 hrs. TLC (Petroleum ether/Ethyl acetate=10/1, Product, Rf=0.7) showed compound 3 was consumed completely and desired spot was detected. H₂O (300 mL) was added and the reaction mixture was extracted with DCM (400 mL×2), and washed with brine 100 mL, dried over Na₂SO₄, concentrated under reduced pressure. The residue was purified by column chromatography (SiO₂, Petroleum ether/Dichloromethane=1/0 to 0/1). Compound 4 (98.0 g, 117 mmol, 82.5% yield, 85.0% TLC purity) was obtained as yellow oil. The residue was purified by column chromatography (SiO₂, Petroleum ether/Dichloromethane=1/0 to 0/1). Compound 4 (98.0 g, 117 mmol, 82.5% yield, 85.0% purity) was obtained as yellow oil.

### Step 4. Synthesis of Compound 5

To a solution of Compound 4 (98.0 g, 138 mmol) in dioxane (630 mL), 4,4,5,5-tetramethyl-2-(4,4,5,5-tetramethyl-1,3,2-dioxaborolan-2-yl)-1,3,2-dioxaborolane (52.5 g, 207 mmol), Pd(dppf)Cl2 (20.2 g, 27.6 mmol), KOAc (27.1 g, 276 mmol) was added, the mixture was stirred at 75° C. for 12 hrs. TLC (Petroleum ether/Ethyl acetate=10/1, Product, Rf=0.5) showed compound 4 was consumed completely and desired spot was detected. The reaction mixture was filtered and extracted with DCM (300 mL×2), and washed with brine 100 mL, dried over Na₂SO₄, concentrated under reduced pressure to give a residue. The residue was purified by column chromatography (SiO₂, Petroleum ether/Dichloromethane=1/0 to 10/1). Compound 5 (62.5 g, 82.5 mmol, 59.8% yield) was obtained as brown oil.

### Step 5. Synthesis of Compound 6

A solution of compound 5 (62.5 g, 82.5 mmol) in THF (420 mL), H₂O₂ (18.7 g, 165 mmol, 30.0% purity), and NaOH (1 M, 82.5 mL) was stirred at 25° C. for 2 hrs. TLC (Petroleum ether/Ethyl acetate=10/1, Product, Rf=0.4) showed compound 5 consumed completely and desired spot was detected. The reaction mixture was quenched by addition Na₂SO₃ (250 mL aq.) at 0° C., and then extracted with EtOAc (200 mL×2). The combined organic layers were washed with brine 100 mL, dried over Na₂SO₄, filtered and concentrated under reduced pressure to give a residue. The residue was purified by column chromatography (SiO₂, Petroleum ether/Dichloromethane=1/0 to 5/1). Compound 6 (37.0 g, 57.1 mmol, 69.2% yield) was obtained as yellow oil.

### Step 6. Synthesis of Compound 7

To a solution of compound 6 (37.0 g, 57.1 mmol) in DMF (200 mL), K2CO3 (15.8 g, 114 mmol), compound 1a (26.4 g, 57.1 mmol) was added, the mixture was stirred at 80° C. for 5 hrs. TLC (Petroleum ether/Ethyl acetate=10/1, Product, Rf=0.6) showed compound 6 consumed completely and desired spot was detected. H₂O 500 mL was added and the reaction mixture was extracted with DCM (200 mL×2). The combined organic layers were washed with LiCl aqueous (100 mL×2) and washed with brine 100 mL, dried over Na₂SO₄, filtered and concentrated under reduced pressure to give a residue. The residue was purified by column chromatography (SiO₂, Petroleum ether/Dichloromethane=1/0 to 0/1). Compound 7 (41.0 g, 39.9 mmol, 69.7% yield) was obtained as yellow oil.

### Step 7. Synthesis of Compound 8

To a solution of compound 7 (41.0 g, 39.9 mmol) in THF (280 mL), TBAF (1 M, 79.8 mL) was added, the mixture was stirred 25° C. for 12 hrs. TLC (Petroleum ether/Ethyl acetate=10/1, Rf=0.50) indicated compound 7 was consumed, and desired spot was detected. The reaction mixture was filtered and concentrated under reduced pressure to give a residue. The residue was purified by column chromatography (SiO₂, Petroleum ether/Dichloromethane=1/0 to 3/1). Compound 8 (20.5 g, 25.9 mmol, 65.1% yield) was obtained as a yellow oil.

### Step 8. Synthesis of Compound dinonyl 8,8′-((2-(((4-(dimethylamino)butanoyl)oxy)methyl)-1,4-phenylene)bis(oxy))dioctanoate

To a solution of 4-(dimethylamino)butanoic acid (5.20 g, 39.6 mmol) in Py (20 mL), EDCI (10.1 g, 52.8 mmol) and compound 8 (20.5 g, 26.5 mmol) was added, the mixture was stirred at 50° C. for 12 hrs. TLC (Petroleum ether/Ethyl acetate=10/1, Product, Rf=0.01) showed compound 8 consumed completely and desired spot was detected. The reaction mixture concentrated under reduced pressure to give a residue. The residue was purified by column chromatography (SiO₂, Petroleum ether/Dichloromethane=10/1 to 0/1). Lipid #4 (11.9 g, 13.3 mmol, 50.3% yield, 99.4% purity) was obtained as a light yellow oil.

### Example 25. Synthesis of Lipid #5

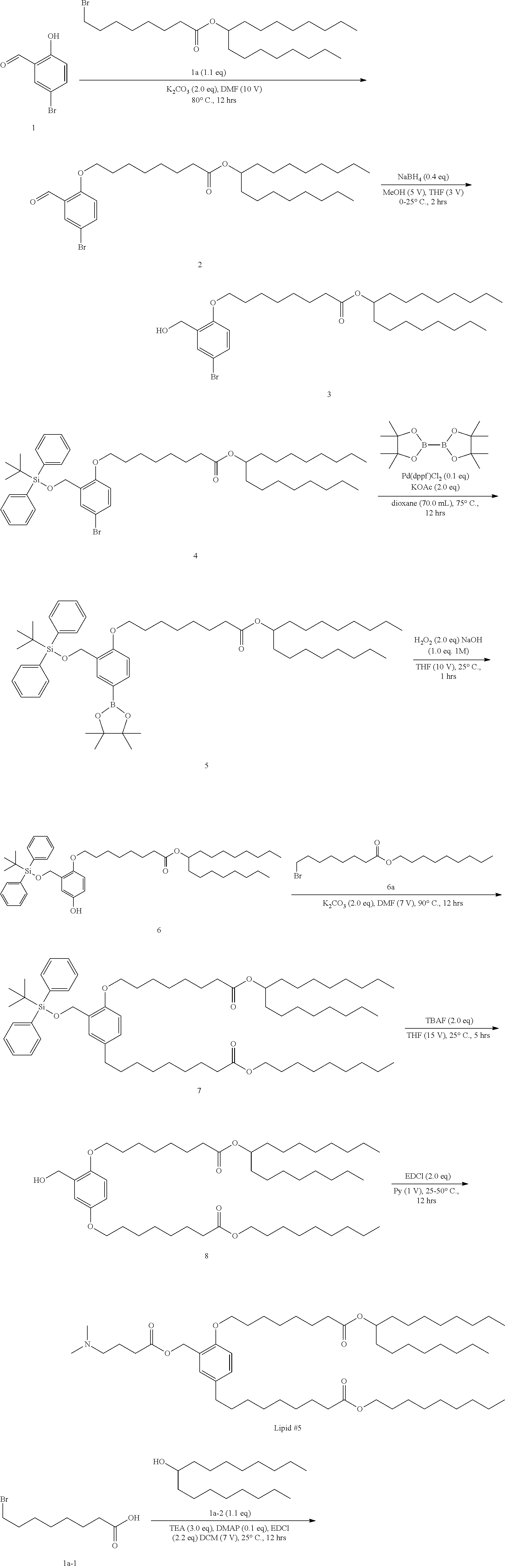

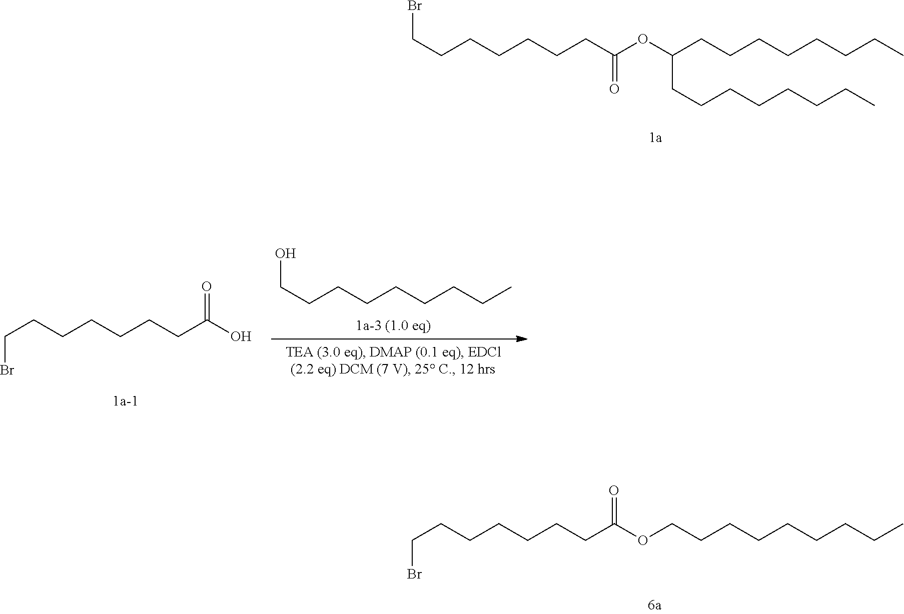

### Step 1. Synthesis of Compound 2

To a solution of compound 1 (25.0 g, 124 mmol) in DMF (175 mL), was added compound 1a (47.7 g, 136 mmol) and K₂CO₃ (34.3 g, 248 mmol). The mixture was stirred at 80° C. for 12 hrs. TLC (Petroleum ether/Ethyl acetate=10/1, Product, Rf=0.5) showed compound 1 consumed completely and desired spot was detected. The reaction mixture was diluted with H₂O 300 mL and THF 20.0 mL, then extracted with EtOAc (200 mL×3). The combined organic layers were washed with brine 50.0 mL, dried over Na₂SO₄, filtered and concentrated under reduced pressure to give a residue. Compound 2 (57.0 g, crude) was obtained as a brown oil.

### Step 2. Synthesis of Compound 3

A solution of compound 2 (20.0 g, 34.38 mmol) in THF (40.0 mL) and MeOH (100 mL), then the mixture was cooled to 0° C. and NaBH₄ (520 mg, 13.7 mmol) was added. The reaction was stirred at 25° C. for 3 hrs. TLC (Petroleum ether/Ethyl acetate=10/1, Product, Rf=0.3) showed compound 2 consumed completely and desired spot was detected. The reaction mixture was quenched with NH₄Cl aqueous solution 20.0 mL and extracted with EtOAc (50.0 mL×3). The combined organic layers were washed with brine 10.0 mL, dried over Na₂SO₄, filtered and concentrated under reduced pressure to give a residue. Compound 3 (20.0 g, crude) was obtained as a brown oil.

### Step 3. Synthesis of Compound 4

A solution of compound 2 (20.0 g, 34.38 mmol) in THF (40.0 mL) and MeOH (100 mL), then the mixture was cooled to 0° C. and NaBH₄ (520 mg, 13.7 mmol) was added. The reaction was stirred at 25° C. for 3 hrs. TLC (Petroleum ether/Ethyl acetate=10/1, Product, Rf=0.3) showed compound 2 consumed completely and desired spot was detected. The reaction mixture was quenched with NH₄Cl aqueous solution 20.0 mL and extracted with EtOAc (50.0 mL×3). The combined organic layers were washed with brine 10.0 mL, dried over Na₂SO₄, filtered and concentrated under reduced pressure to give a residue. Compound 3 (20.0 g, crude) was obtained as a brown oil.

### Step 4. Synthesis of Compound 5

A mixture of compound 4 (10.0 g, 12.1 mmol), KOAc (2.39 g, 24.3 mmol), 4,4,5,5-tetramethyl-2-(4,4,5,5-tetramethyl-1,3,2-dioxaborolan-2-yl)-1,3,2-dioxaborolane (3.71 g, 14.6 mmol), Pd(dppf)Cl₂ (890 mg, 1.22 mmol) in 1,4-dioxane (70.0 mL) was degassed and purged with N₂ for 3 times, and then the mixture was stirred at 75° C. for 12 hrs under N₂ atmosphere. TLC (Petroleum ether: Ethyl acetate=10/1, Rf=0.7) indicated the reaction was complete. The reaction was filtered and concentrated in reduced pressure. The residue was purified by column chromatography (SiO₂, Petroleum ether/Ethyl acetate=10/1 to 5/1). Compound 5 (13.0 g, crude) was obtained as a yellow oil.

### Step 5. Synthesis of Compound 6

To a solution of compound 5 (13.0 g, 14.9 mmol) in THF (130 mL) were added H₂O₂ (3.39 g, 29.9 mmol, 30% purity) and NaOH (1 M, 14.9 mL) and stirred at 25° C. for 1 hr. TLC (Petroleum ether/Ethyl acetate=10/1, Rf=0.43) indicated the reaction was complete. The reaction mixture was quenched by addition Na₂SO₃ 100 mL at 0° C., and then extracted with EtOAc (100 mL×2). The combined organic layers were washed with brine 100 mL, dried over Na₂SO₄, filtered and concentrated under reduced pressure to give a residue. The residue was purified by column chromatography (SiO₂, Petroleum ether/Ethyl acetate=10/1 to 1/1). Compound 6 (10.0 g, crude) was obtained as a yellow oil.

### Step 6. Synthesis of Compound 7

To a solution of compound 6 (9.50 g, 12.5 mmol) and nonyl 8-bromooctanoate (5.03 g, 14.4 mmol) in DMF (63.0 mL) were added K₂CO₃ (3.46 g, 25.0 mmol) and stirred at 90° C. for 12 hrs. TLC (Petroleum ether/Ethyl acetate=10/1, Rf=0.7) indicated the reaction was complete. The reaction mixture was extracted with solvent (100 mL×3). The combined organic layers were washed with brine 100 mL, dried over Na₂SO₄, filtered and concentrated under reduced pressure to give a residue. The residue was purified by column chromatography (SiO₂, Petroleum ether/Ethyl acetate=50/1 to 1/1). Compound 7 (6.30 g, 6.13 mmol, 48.9% yield) was obtained as a yellow oil.

### Step 7. Synthesis of Compound 8

To a solution of compound 7 (6.30 g, 6.13 mmol) in THF (30.0 mL) were added TBAF (1 M, 12.2 mL) and stirred at 25° C. for 5 hrs. TLC (Petroleum ether/Ethyl acetate=10/1, R_{f}=0.3) indicated the reaction was complete. The reaction mixture was extracted with EtOAc (50.0 mL×2). The combined organic layers were washed with brine 50.0 mL, dried over Na₂SO₄, filtered and concentrated under reduced pressure to give a residue. The residue was purified by column chromatography (SiO₂, Petroleum ether/Ethyl acetate=50/1 to 1/1). Compound 8 (6.00 g, crude) was obtained as a yellow oil.

### Step 8. Synthesis of Lipid #5

To a solution of compound 8 (6.00 g, 7.60 mmol), 4-(dimethylamino)butanoic acid (1.91 g, 11.4 mmol, HCl salt) in pyridine (6.00 mL) were added EDCI (2.91 g, 15.2 mmol) at 25° C. and stirred at 50° C. for 12 hrs. TLC (Petroleum ether/Ethyl acetate=1/1, Rf=0.1) indicated the reaction was complete. The reaction was concentrated in reduced pressure. The residue was purified by column chromatography (SiO₂, Petroleum ether/Ethyl acetate=10/1 to 0/1). Lipid #5 (3.00 g, 3.32 mmol, 43.7% yield, 100% purity) was obtained as a yellow oil.

### Example 26. Synthesis of Lipid #8

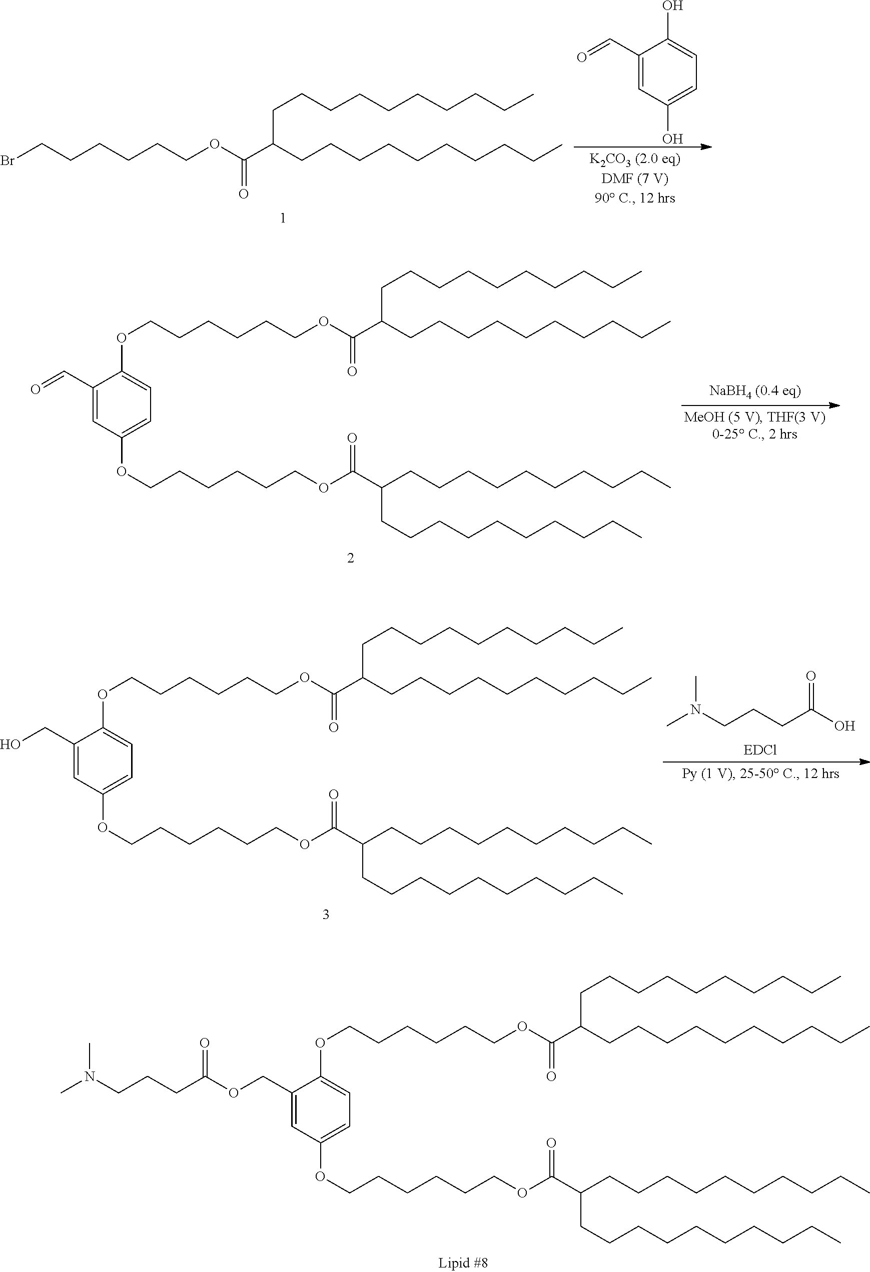

### Step 1. Synthesis of Compound 2

To a solution of compound 1 (4.92 g, 14.0 mmol) in DMF (17.0 mL) was added K₂CO₃ (4.01 g, 27.8 mmol) and 2,5-dihydroxybenzaldehyde (1.01 g, 7.0 mmol). The mixture was stirred at 90° C. for 12 hrs. TLC (Petroleum ether/Ethyl acetate=10/1) indicated compound 1 was consumed completely. The crude reaction mixture was poured into water (10.0 mL) and extracted with ethyl acetate (10.0 mL×2). The combined organic layers were washed with brine 10.0 mL, dried over Na₂SO₄, filtered and concentrated under reduced pressure to give a residue. The residue was purified by column chromatography (SiO₂, Petroleum ether/Ethyl acetate=200/1 to 50/1). Compound 2 (3.99 g, 4.06 mmol, 58.0% yield) was obtained as a brown oil.

### Step 2. Synthesis of Compound 3

A solution of compound 2 (3.99 g, 4.06 mmol) in THF (20.0 mL) and MeOH (100 mL), then the mixture was cooled to 0° C. and NaBH₄ (520 mg, 13.7 mmol) was added. The reaction was stirred at 25° C. for 3 hrs. TLC (Petroleum ether/Ethyl acetate=10/1) showed compound 2 consumed completely and desired spot was detected. The reaction mixture was quenched with NH₄Cl aqueous solution 20.0 mL and extracted with EtOAc (50.0 mL×3). The combined organic layers were washed with brine 10.0 mL, dried over Na₂SO₄, filtered and concentrated under reduced pressure to give a residue. The residue was purified by column chromatography (SiO₂, Petroleum ether/Ethyl acetate=50/1 to 10/1). Compound 3 (3.56 g, 3.61 mmol, 89.0% yield) was obtained as a brown oil.

### Step 3. Synthesis of Lipid #8

To a solution of compound 3 (3.56 g, 3.61 mmol), 4-(dimethylamino)butanoic acid (0.95 g, 5.41 mmol, HCl salt) in pyridine (6.00 mL) were added EDCI (1.46 g, 7.2 mmol) at 25° C. and stirred at 50° C. for 12 hrs. TLC (Petroleum ether/Ethyl acetate=1/1) indicated the reaction has completed. The reaction was concentrated in reduced pressure. The residue was purified by column chromatography (SiO₂, Petroleum ether/Ethyl acetate=10/1 to 0/1). Lipid #8 (2.42 g, 2.20 mmol, 61% yield) was obtained as a light yellow oil.

### Example 27. Synthesis of Lipid #9

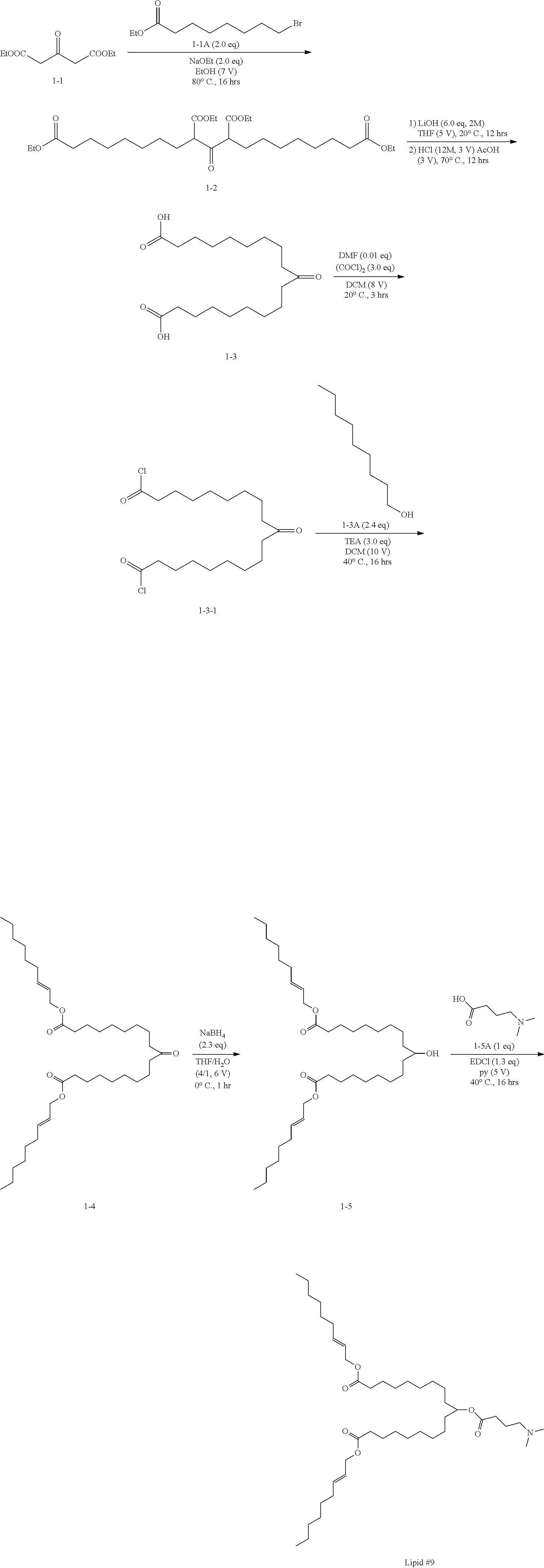

### Step 1 Synthesis of Compound 1-2

To a solution of NaOEt (33.7 g, 495 mmol) in EtOH (250 mL) was added compound 1-1 (100 g, 495 mmol, 90.1 mL). The solution was heated to 80° C. with stirring compound 1-1A (124 g, 495 mmol) was dropwise into the solution. The solution was stirred at 80° C. for 2 hrs. After another solution of NaOEt (33.7 g, 495 mmol) in EtOH (250 mL) added into the reaction solution, compound 1-1A (124 g, 495 mmol) was added dropwise. The solution was stirred at 80° C. for 12 hrs. LCMS showed the reaction was finished. The reaction suspension was concentrated under vacuum to give a residue. The residue was added to H₂O (100 mL), the pH of the solution was adjusted to 7 with the aqueous HCl solution (0.5 M). The solution was extracted with MTBE (150 mL×2), and the organic layer was washed with brine (100 mL×2). The combined organic layer was concentrated under vacuum to give a residue. The residue was purified by column chromatography (SiO₂, Petroleum ether/Ethyl acetate=250/1-1/10) to give compound 1-2 (166 g, 306 mmol, 61.9% yield) as a light brown oil.

### Step 2 Synthesis of Compound 1-3

To a solution of compound 1-2 (165 g, 304 mmol) in THF (450 mL) and MeOH (250 mL) was add a aqueous solution of LiOH·H₂O (4.0 M, 456 mL). The solution was stirred at 20° C. for 5 hrs. LCMS showed the reaction was completed. The reaction was concentrated under vacuum to give an intermediate product (150 g, crude) as a yellow solid, which was used to next step directly. A solution of the intermediate product (15.0 g, 34.8 mmol) in HCl (12.0 M, 53.5 mL) and AcOH (80.0 mL) was stirred at 70° C. for 12 hrs. LCMS showed the reaction was finished. The suspension was concentrated under reduced pressure to give a suspension. The pH of suspension was adjusted to 2 with HCl (12.0 M). Then the suspension was filtered, and filter cake was concentrated under reduced pressure to give compound 1-3 (9.50 g, 27.7 mmol, 79.6% yield) as a brown solid, which was used to next step directly.

### Step 3 Synthesis of Compound 1-3-1

To a solution of compound 1-3 (40.0 g, 116 mmol) in DCM (250 mL) was added DMF (85.3 mg, 1.17 mmol, 89.8 μL) and (COCl)₂ (30.4 g, 239 mmol, 20.9 mL). The solution was stirred at 20° C. for 3 hrs. A sample was taken to be quenched by MeOH. TLC (Petroleum ether Ethyl acetate=5:1, R_{f} of product=0.68) showed the starting material was consumed completely. The reaction solution was concentrated under vacuum to give a residue. The residue was dissolved in toluene (30.0 mL×2) and concentrated under vacuum twice to give compound 1-3-1 (44.0 g, crude) as black brown oil, which was used to next step directly.

### Step 4 Synthesis of Compound 1-4

To a solution of compound 1-3A (34.6 g, 243 mmol) in DCM (160 mL) was added TEA (35.2 g, 347 mmol, 48.4 mL). A solution of compound 1-3-1 (44.0 g, 115 mmol) in DCM (60.0 mL) was added dropwise the solution. The solution was stirred at 20° C. for 12 hrs. TLC (Petroleum ether: Ethyl acetate=5:1, R_{f} of product=0.79) showed the starting material was consumed completely. The reaction solution poured into the H₂O (250 mL), and the suspension was extracted with ethyl acetate (150 mL×2). The organic layer was dried over sodium sulfate and concentrated under reduced pressure to give a residue. The residue was purified by column chromatography (SiO₂, Petroleum ether/Ethyl acetate=500/1 to 0/1) to give compound 1-4 (21.0 g, 35.5 mmol, 30.6% yield) as a brown solid.

### Step 5 Synthesis of Compound 1-5

To a solution of compound 1-4 (21.0 g, 35.5 mmol) in THF (80.0 mL) and H₂O (20.0 mL) was added NaBH₄ (26.9 g, 710 mmol). The solution was stirred at 0° C. for 1 hr. TLC (Petroleum ether: Ethyl acetate=3:1, R_{f} of product=0.74) showed the reaction was finished. The reaction mixture was poured into the aqueous of NH₄Cl (50.0 mL), and the solution was extracted with ethyl acetate (10.0 mL×2). The organic layer was extracted with H₂O (10.0 mL×2). The organic layer was dried over sodium sulfate and concentrated under reduced pressure to give a residue. The residue was purified by column chromatography (SiO₂, Petroleum ether/Ethyl acetate=200/1 to 0/1) to give compound 1-5 (9.00 g, 15.2 mmol, 42.7% yield) as off-white oil.

### Step 6 Synthesis of Lipid #9

To a solution of compound 1-5 (14.0 g, 23.6 mmol) in Py (60.0 mL) was added compound 1-5A (7.92 g, 47.2 mmol). EDCI (9.05 g, 47.2 mmol) was added into the solution. The solution was stirred at 45° C. for 2 hrs. LCMS showed the reaction was finished. The reaction mixture was poured into the aqueous of NH₄Cl (80.0 mL), and the solution was extracted with ethyl acetate (30.0 mL×2). The organic layer was washed with H₂O (30.0 mL×2). The organic layer was dried over sodium sulfate and concentrated under reduced pressure to give a residue. The residue was purified by column chromatography (SiO₂, Petroleum ether/Ethyl acetate=50/1 to 0/1) to give Lipid #9 (15.0 g, 21.2 mmol, 90.0% yield) as a light yellow oil.

### Example 28. Synthesis of Lipid #11

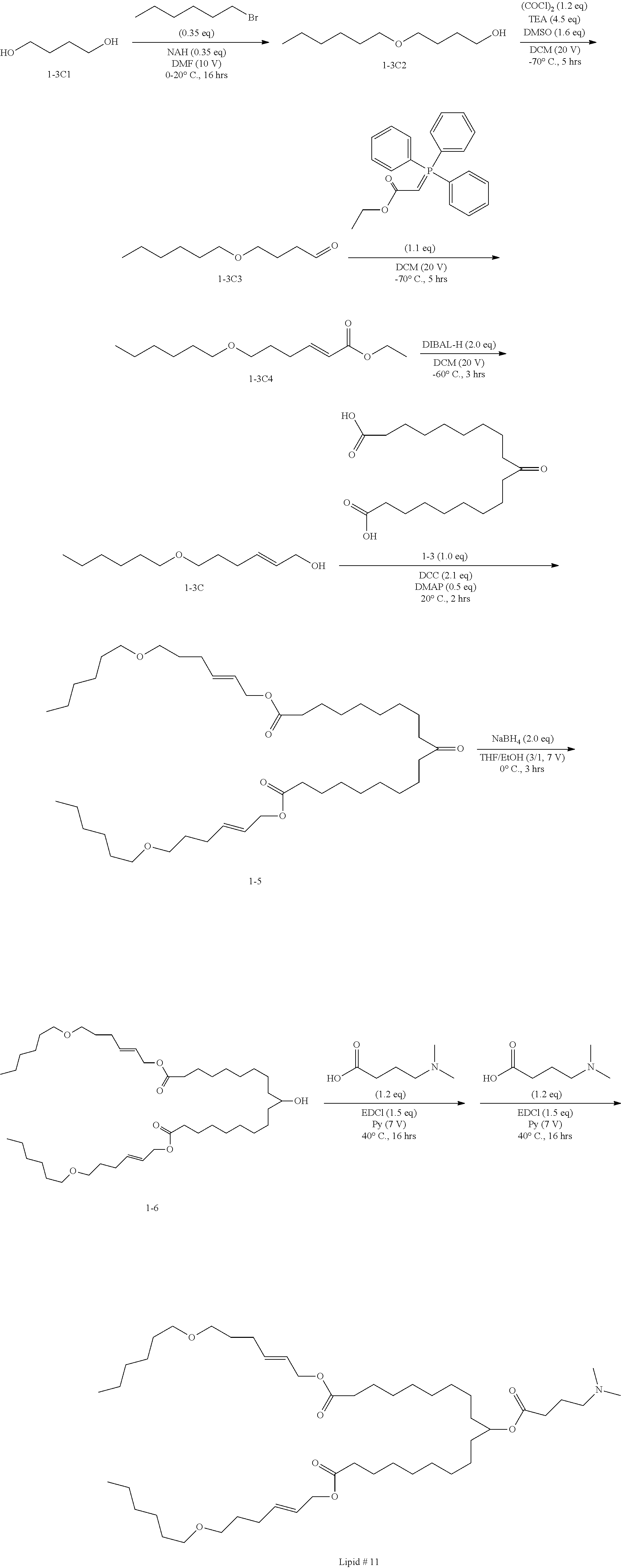

### Step 1 Synthesis of Compound 1-3C2

NaH (24.0 g, 599 mmol, 60% purity, 0.36 eq) was added dropwise to a solution of 1-3C1 (150 g, 1.66 mol, 147 mL, 1 eq) in DMF (1500 mL) at −10° C. After 30 mins, 1-bromohexane (98.9 g, 599 mmol, 83.8 mL, 0.36 eq) was added into the solution. The solution was stirred at 15° C. for 3 hrs. TLC (Petroleum ether: Ethyl acetate=1:1, R_{f} of product=0.40) showed the reaction was finished. The solution was poured into H₂O (2 L). The aqueous phase was extracted with EtOAc (1 L×3). The combined organic layer was washed with brine (2 L) and the result organic layer was dried over Na₂SO₄ and dried in vacuum to give a residue. The residue was purified by column chromatography (SiO₂, petroleum ether/Ethyl acetate=10:1 to 1:10) to give compound 1-3C2 (60.0 g, 344 mmol, 20.7% yield) as a yellow oil.

### Step 2 Synthesis of Compound 1-3C3

(COCl)₂ (43.9 g, 346 mmol, 30.3 mL, 1.30 eq) in DCM (1500 mL) was added to DMSO (33.3 g, 426 mmol, 33.3 mL, 1.60 eq) at −65° C. After 30 minutes, a solution of compound 1-3C2 (58 g, 266 mmol, 80% purity, 1 eq) was added to the mixture and after 30 minutes, TEA (121 g, 1.20 mol, 167 mL, 4.50 eq) was added. The solution was stirred at −65° C. for 30 minutes and for an additional 30 minutes at 15° C. TLC (Petroleum ether: Ethyl acetate=2:1, R_{f} of product=0.50) showed the reaction was finished. The solution was poured into H₂O (500 mL). The above solution was extracted with DCM (500 mL×2). The combined organic layer was dried in vacuum to give a residue. The residue was purified by column chromatography (SiO₂, Petroleum ether/Ethyl acetate=30:1 to 1:10) to give 1-3C3 (80 g, 245 mmol, 54.0% yield) as a colorless oil.

### Step 3 Synthesis of Compound 1-3C4

A solution of compound 1-3C₃ (38.0 g, 221 mmol, 1 eq) and ethyl 2-(triphenylphosphoranylidene) acetate (84.5 g, 243 mmol, 1.1 eq) in DCM (200 mL) was stirred at 15° C. for 6 hrs. TLC (Petroleum ether: Ethyl acetate=5:1, R_{f} of product=0.60) and LCMS showed the reaction was finished. The reaction solution was dried in vacuum to give a residue. The residue was triturated with petroleum ether (300 mL) at 15° C. for 30 mins. The above suspension was filtered, and the filtrate was dried in vacuum to give the crude. The crude was purified by column chromatography (SiO2, Petroleum ether/Ethyl acetate=100:1 to 30:1) to give 1-3C4 (40 g, 165 mmol, 74.8% yield) as a yellow oil.

### Step 4 Synthesis of Compound 1-3C

A solution of compound 1-3C4 (17.6 g, 72.6 mmol, 1 eq) in DCM (50 mL) was cooled to −65° C. DIBAL-H (1 M, 152.5 mL, 2.1 eq) was added dropwise into the above solution. The solution was stirred at −65° C. for 2 hrs. TLC (Petroleum ether: Ethyl acetate=5:1, R_{f} of product=0.20) showed the reaction was finished. The reaction solution was added dropwise to ice HCl (4M, 100 mL), and the pH of the solution was adjust to 2. Then the solution was extracted with DCM (300×3) and the combined organic layer was dried over Na₂SO₄ and dried in vacuum to give the crude. The crude was added dropwise to ice HCl aqueous solution (4M, 100 mL) until the pH value reached 2. Then the solution was extracted with DCM (300×3) and the combined organic layer was dried over Na₂SO₄ and dried in vacuum to give 1-3C (12 g, 59.91 mmol, 82.49% yield) as a yellow oil.

### Step 5 Synthesis of Compound 1-5

To a solution of DCC (7.95 g, 38.5 mmol, 7.79 mL, 2.1 eq) and 1-3 (6.28 g, 18.3 mmol, 1 eq) in DCM (50 mL) were added 1-3C (7.71 g, 38.5 mmol, 2.1 eq) and DMAP (1.12 g, 9.17 mmol, 0.5 eq). The solution was stirred at 15° C. for 2 hrs. TLC (Petroleum ether: Ethyl acetate=5:1, R_{f} of product=0.60) showed the reaction was finished. The reaction suspension was filtered and the filter cake was washed with DCM (50 mL×2). The filtrate was washed with HCl (2 M, 30 mL), and the combined organic layer was dried in vacuum to give a residue. The residue was purified by column chromatography (SiO₂, Petroleum ether/Ethyl acetate=80:1 to 20:1) to give compound 1-5 (8 g, 8.49 mmol, 46.3% yield) as a white solid, which was used to next step directly.

### Step 6 Synthesis of Compound 1-6

A solution of compound 1-5 (5 g, 7.1 mmol, 1.0 eq) in THF (30 mL) and EtOH (10 mL) was cooled to 0° C. NaBH₄ (0.96 g, 25.4 mmol, 3.59 eq) was added into the above solution. The solution was stirred at 0° C. for 5 hrs. TLC (Petroleum ether: Ethyl acetate=5:1, R_{f} of product=0.30) showed the reaction was finished. he solution was poured into ice-H₂O (100 mL), and the pH of the solution was adjust to 2 with ice-HCl (1 M). The above solution was extracted with EtOAc (100 mL×3). The combined organic layer was dried over Na₂SO₄ and concentrated in vacuum to give a residue. The residue was purified by column chromatography (SiO₂, Petroleum ether/Ethyl acetate=100:1 to 5:1) to give compound 1-6 (4 g, 5.64 mmol, 79.8% yield) as a colorless oil.

### Step 7 Synthesis of Lipid #11

To a solution of compound 1-6 (4 g, 5.64 mmol, 1 eq) and 4-(dimethylamino)butanoic acid (1.23 g, 7.33 mmol, 1.30 eq, HCl) in pyridine (30 mL) was added EDCI (2.70 g, 14.1 mmol, 2.5 eq). The solution was stirred at 40° C. for 6 hrs. TLC (Petroleum ether: Ethyl acetate=5:1, R_{f} of product=0.40) showed the reaction was finished. The solution was poured into H₂O (100 mL). The result solution was extracted with EtOAc (100 mL×3). The combined organic layer was washed with brine (200 mL) and concentrated in vacuum to give a residue. The residue was purified by column chromatography (SiO₂, Petroleum ether/Ethyl acetate=10:1 to 10:1) to give Lipid #11 (1.22 mmol, 22% yield) as a yellow oil.

### Example 29. Synthesis of Lipid #12

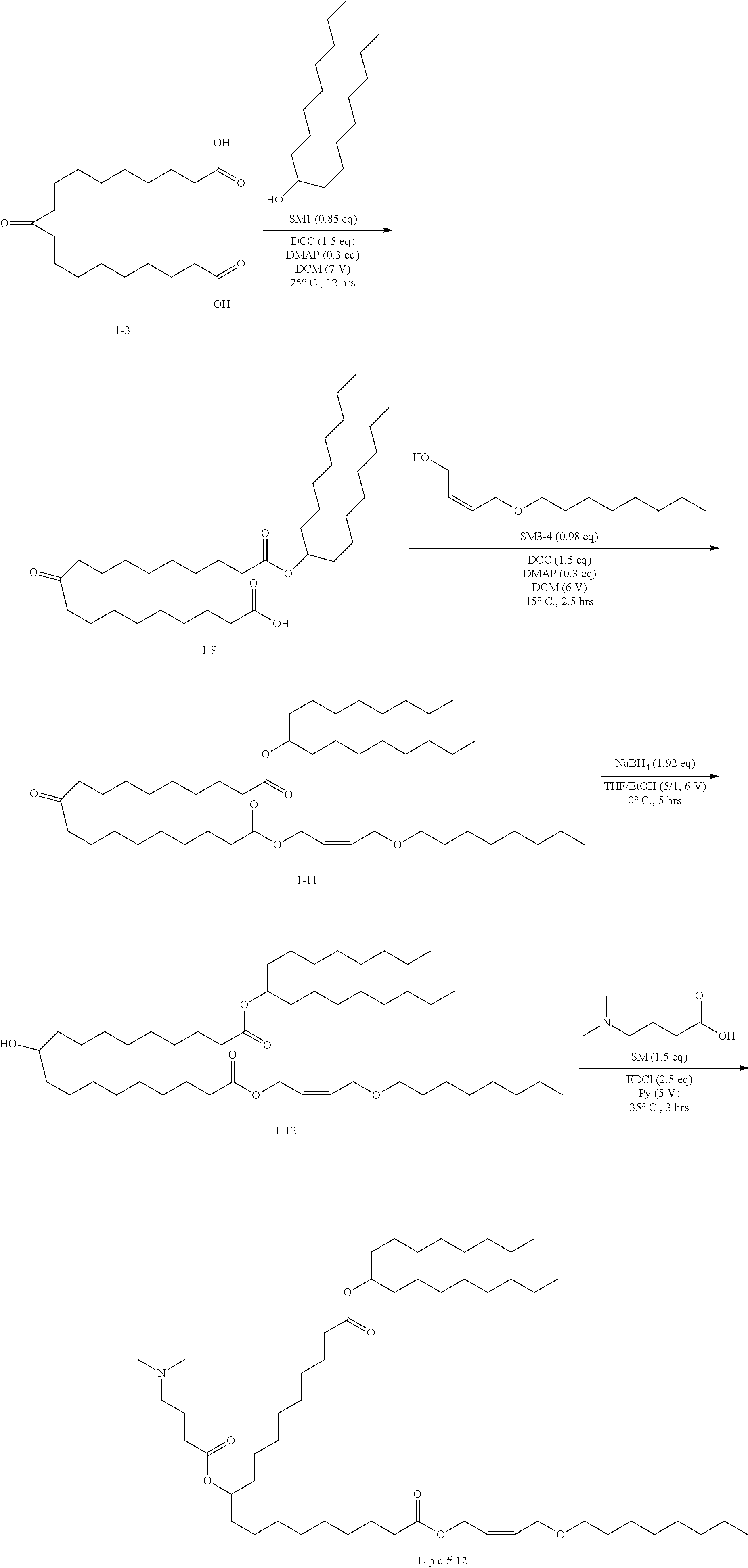

### Step 1—Synthesis of Compound 1-9

To a solution of compound 1-3 (26.0 g, 75.9 mmol) in DCM (200 mL) was added compound SM1 (16.6 g, 64.5 mmol), DCC (23.5 g, 114 mmol, 23.0 mL) and DMAP (2.78 g, 22.8 mmol). The solution was stirred at 25° C. for 12 hrs. TLC (Petroleum ether: Ethyl acetate=3:1, R_{f} of product=0.50) showed the reaction was finished. The solution was poured into H₂O (300 mL). Then the solution was extracted with DCM (300 mL×3). The combined organic layer was washed with H₂O (100 mL). The organic layer was dried in vacuum to give a residue. The residue was purified by column chromatography (SiO₂, Petroleum ether/Ethyl acetate=20/1 to 5/1) to give compound 1-9 (15.0 g, 25.8 mmol, 34.0% yield) as a yellow oil.

### Step 2—Synthesis of Compound 1-11

To a solution of compound 1-9 (11.5 g, 19.8 mmol) and DCC (5.72 g, 27.7 mmol, 5.61 mL) in DCM (60 mL) was added DMAP (725 mg, 5.94 mmol). The solution was stirred at 15° C. for 30 mins. Then compound SM3-4 (3.89 g, 19.40 mmol) was added into the above solution. The solution was stirred at 15° C. for 2 hrs. TLC (Petroleum ether: Ethyl acetate=5:1, R_{f} of product=0.60) showed the starting material was consumed completely. The solution was poured into H₂O (100 mL). Then the solution was extracted with MTBE (50 mL×3). The combined organic layer was washed with brine (30 mL), and the result organic layer was dried in vacuum to give a residue. The residue was purified by column chromatography (SiO₂, Petroleum ether/Ethyl acetate=80/1 to 30/1) to give a compound 1-11 (12.0 g, 15.7 mmol, 79.4% yield) as a colorless oil.

### Step 3—Synthesis of Compound 1-12

A solution of compound 1-11 (10.5 g, 13.8 mmol) in THF (50 mL) and EtOH (10 mL) was cooled to 0° C. NaBH₄ (1.00 g, 26.4 mmol) was added into the above solution. The solution was stirred at 0° C. for 5 hrs. TLC (Petroleum ether: Ethyl acetate=5:1, R_{f} of product=0.30) showed the reaction was finished. The solution was poured into ice-H₂O (100 mL), and the pH of the solution was adjust to 2 with ice-HCl (1 M). The above solution was extracted with EtOAc (100 mL×3). The combined organic layer was dried over Na₂SO₄ and concentrated in vacuum to give a residue. The residue was purified by column chromatography (SiO₂, Petroleum ether/Ethyl acetate=100/1 to 5/1) to give compound 1-12 (9.00 g, 11.8 mmol, 85.5% yield) as a colorless oil.

### Step 4—Synthesis of Lipid #12

To a solution of compound 1-12 (6.00 g, 7.84 mmol) and compound SM (1.97 g, 11.8 mmol) in Py (30 mL) was added EDCI (3.76 g, 19.6 mmol). The solution was stirred at 35° C. for 3 hrs. TLC (Petroleum ether: Ethyl acetate=1:1, R_{f} of product=0.30) showed the reaction was finished. The solution was poured into H₂O (50 mL). The above solution was extracted with EtOAc (50 mL×3). The combined organic layer was dried in vacuum to give a residue. The residue was purified by column chromatography (SiO₂, Petroleum ether/Ethyl acetate=50/1 to 1/50) to give Lipid #12 (1.10 g, 1.25 mmol, 16.0% yield) as a yellow oil.

### INCORPORATION BY REFERENCE

The entire disclosure of each of the patent documents and scientific articles cited herein are incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

SEQUENCE LISTING
Table of sequences

| | |
|---|---|
| SEQ ID NO: 1 | atgggcaccgtgaacaagcccgtggtgggcgtgctgatgggcttcggcatcatcaccggcaccctgagaatcaccaaccccg |
| (VZV glyco- | tgagagctagcgtgctgagatacgacgacttccacatcgacgaggacaagctggacaccaacagcgtgtacgagccctacta |
| protein E | ccacagcgaccacgccgagagcagctgggtgaacagaggcgagagcagcagaaaggcctacgaccacaacagcccctacatc |
| antigen | tggcctagaaacgactacgacggcttcctggagaacgcccacgagcaccacggcgtgtacaaccaaggcagaggcatcgaca |
| sequence) | gcggcgagagactgatgcagcccacacagatgagcgcccaagaggacctgggcgacgacaccggcatccacgtgatccccac |
| | cctgaacggcgacgacagacacaagatcgtgaacgtggatcagagacagtacggcgacgtgttcaagggcgacctgaacccc |
| | aagccccaagggcagagactgatcgaggtgagcgtggaggagaaccaccccttcaccctgagagcccccattcagagaatct |
| | acggcgtgagatacaccgagacctggagcttcctgcctagcctgacctgcaccggcgacgccgcccccgccattcagcacat |
| | ctgcctgaagcacaccacctgcttccaagacgtggtggtggacgtggactgcgccgagaacaccaaggaggatcagctggcc |
| | gagatcagctacagattccaaggcaagaaggaggccgatcagccctggatcgtggtgaacacaagcaccctgttcgacgagc |
| | tggagctggacccccccgagatcgagcccggcgtgctgaaggtgctgagaaccgagaagcagtacctgggcgtgtacatctg |
| | gaacatgagaggcagcgacggcacaagcacctacgccaccttcctggtgacctggaagggcgacgagaagacaagaaacccc |
| | acccccgcggtgacccctcagcctagaggcgccgagttccacatgtggaactaccacagccacgtgttcagcgtgggcgaca |
| | ccttcagcctggccatgcacctgcagtacaagatccacgaggcccccttcgacctgctgctggagtggctgtacgtgcccat |
| | cgaccccacctgtcagcccatgagactgtacagcacctgcctgtaccaccccaacgctcctcagtgcctgagccacatgaac |
| | agcggctgcaccttcacaagcccccacctggctcagagagtggctagcaccgtgtatcagaactgcgagcacgccgacaact |
| | acaccgcctactgcctgggcatcagccacatggagcctagcttcggcctgatcctgcacgacggcggcaccaccctgaagtt |
| | cgtggacacccccgagagcctgagcggcctgtacgtgttcgtggtgtacttcaacggccacgtggaggccgtggcctacacc |
| | gtggtgagcaccgtggaccacttcgtgaatgccatcgaggagagaggcttcccccccaccgccggccaacccccggccacca |
| | ccaagcccaaggagatcacccccgtgaatcccggcacaagccctcttatcagatacgcagcctggaccggaggcctggccgc |
| | cgtggtgctgctgtgcctggtgatcttcctgatctgcaccgccaagagaatgagagtgaaggccgctagagtggacaagtga |
| SEQ ID NO: 2 | ataggcggcgcatgagagaagcccagaccaattacctacccaaaatggagaaagttcacgttgacatcgaggaagacagccc |
| (VZV srRNA | attcctcagagctttgcagcggagcttcccgcagtttgaggtagaagccaagcaggtcactgataatgaccatgctaatgcc |
| backbone) | agagcgttttcgcatctggcttcaaaactgatcgaaacggaggtggacccatccgacacgatccttgacattggaagtgcgc |
| | ccgcccgcagaatgtattctaagcacaagtatcattgtatctgtccgatgagatgtgcggaagatccggacagattgtataa |
| | gtatgcaactaagctgaagaaaaactgtaaggaaataactgataaggaattggacaagaaaatgaaggagctcgccgccgtc |
| | atgagcgaccctgacctggaaactgagactatgtgcctccacgacgacgagtcgtgtcgctacgaagggcaagtcgctgttt |
| | accaggatgtatacgcggttgacggaccgacaagtctctatcaccaagccaataagggagttagagtcgcctactggatagg |
| | ctttgacaccaccccttttatgtttaagaacttggctggagcatatccatcatactctaccaactgggccgacgaaaccgtg |
| | ttaacggctcgtaacataggcctatgcagctctgacgttatggagcggtcacgtagagggatgtccattcttagaaagaagt |
| | atttgaaaccatccaacaatgttctattctctgttggctcgaccatctaccacgagaagagggacttactgaggagctggca |
| | cctgccgtctgtatttcacttacgtggcaagcaaaattacacatgtcggtgtgagactatagttagttgcgacgggtacgtc |
| | gttaaaagaatagctatcagtccaggcctgtatgggaagccttcaggctatgctgctacgatgcaccgcgagggattcttgt |
| | gctgcaaagtgacagacacattgaacggggagagggtctcttttcccgtgtgcacgtatgtgccagctacattgtgtgacca |
| | aatgactggcatactggcaacagatgtcagtgcggacgacgcgcaaaaactgctggttgggctcaaccagcgtatagtcgtc |
| | aacggtcgcacccagagaaacaccaataccatgaaaaattaccttttgcccgtagtggcccaggcatttgctaggtgggcaa |
| | aggaatataaggaagatcaagaagatgaaaggccactaggactacgagatagacagttagtcatggggtgttgttgggcttt |
| | tagaaggcacaagataacatctatttataagcgcccggatacccaaaccatcatcaaagtgaacagcgatttccactcattc |
| | gtgctgcccaggataggcagtaacacattggagatcgggctgagaacaagaatcaggaaaatgttagaggagcacaaggagc |
| | cgtcacctctcattaccgccgaggacgtacaagaagctaagtgcgcagccgatgaggctaaggaggtgcgtgaagccgagga |
| | gttgcgcgcagctctaccacctttggcagctgatgttgaggagcccactctggaagccgatgtcgacttgatgttacaagag |
| | gctggggccggctcagtggagacacctcgtggcttgataaaggttaccagctacgatggcgaggacaagatcggctcttacg |
| | ctgtgctttctccgcaggctgtactcaagagtgaaaaattatcttgcatccaccctctcgctgaacaagtcatagtgataac |
| | acactctggccgaaaagggcgttatgccgtggaaccataccatggtaaagtagtggtgccagagggacatgcaatacccgtc |
| | caggactttcaagctctgagtgaaagtgccaccattgtgtacaacgaacgtgagttcgtaaacaggtacctgcaccatattg |
| | ccacacatggaggagcgctgaacactgatgaagaatattacaaaactgtcaagcccagcgagcacgacggcgaatacctgta |
| | cgacatcgacaggaaacagtgcgtcaagaaagaactagtcactgggctagggctcacaggcgagctggtggatcctcccttc |
| | catgaattcgcctacgagagtctgagaacacgaccagccgctccttaccaagtaccaaccataggggtgtatggcgtgccag |
| | gatcaggcaagtctggcatcattaaaagcgcagtcaccaaaaaagatctagtggtgagcgccaagaaagaaaactgtgcaga |
| | aattataagggacgtcaagaaaatgaaagggctggacgtcaatgccagaactgtggactcagtgctcttgaatggatgcaaa |
| | caccccgtagagaccctgtatattgacgaagcttttgcttgtcatgcaggtactctcagagcgctcatagccattataagac |
| | ctaaaaaggcagtgctctgcggggatcccaaacagtgcggtttttttaacatgatgtgcctgaaagtgcattttaaccacga |
| | gatttgcacacaagtcttccacaaaagcatctctcgccgttgcactaaatctgtgacttcggtcgtctcaaccttgttttac |
| | gacaaaaaaatgagaacgacgaatccgaaagagactaagattgtgattgacactaccggcagtaccaaacctaagcaggacg |
| | atctcattctcacttgtttcagaggggggtgaagcagttgcaaatagattacaaaggcaacgaaataatgacggcagctgcc |
| | tctcaagggctgacccgtaaaggtgtgtatgccgttcggtacaaggtgaatgaaaatcctctgtacgcacccacctcagaac |
| | atgtgaacgtcctactgacccgcacggaggaccgcatcgtgtggaaaacactagccggcgacccatggataaaaacactgac |
| | tgccaagtaccctgggaatttcactgccacgatagaggagtggcaagcagagcatgatgccatcatgaggcacatcttggag |
| | agaccggaccctaccgacgtcttccagaataaggcaaacgtgtgttgggccaaggctttagtgccggtgctgaagaccgctg |
| | gcatagacatgaccactgaacaatggaacactgtggattattttgaaacggacaaagctcactcagcagagatagtattgaa |
| | ccaactatgcgtgaggttctttggactcgatctggactccggtctattttctgcacccactgttccgttatccattaggaat |
| | aatcactgggataactccccgtcgcctaacatgtacgggctgaataaagaagtggtccgtcagctctctcgcaggtacccac |
| | aactgcctcgggcagttgccactggaagagtctatgacatgaacactggtacactgcgcaattatgatccgcgcataaacct |
| | agtacctgtaaacagaagactgcctcatgctttagtcctccaccataatgaacacccacagagtgacttttcttcattcgtc |
| | agcaaattgaagggcagaactgtcctggtggtcggggaaaagttgtccgtcccaggcaaaatggttgactggttgtcagacc |
| | ggcctgaggctaccttcagagctcggctggatttaggcatcccaggtgatgtgcccaaatatgacataatatttgttaatgt |
| | gaggaccccatataaataccatcactatcagcagtgtgaagaccatgccattaagcttagcatgttgaccaagaaagcttgt |
| | ctgcatctgaatcccggcggaacctgtgtcagcataggttatggttacgctgacagggccagcgaaagcatcattggtgcta |
| | tagcgcggcagttcaagttttcccgggtatgcaaaccgaaatcctcacttgaagagacggaagttctgtttgtattcattgg |
| | gtacgatcgcaaggcccgtacgcacaatccttacaagctttcatcaaccttgaccaacatttatacaggttccagactccac |
| | gaagccggatgtgcaccctcatatcatgtggtgcgaggggatattgccacggccaccgaaggagtgattataaatgctgcta |
| | acagcaaaggacaacctggcggaggggtgtgcggagcgctgtataagaaattcccggaaagcttcgatttacagccgatcga |
| | agtaggaaaagcgcgactggtcaaaggtgcagctaaacatatcattcatgccgtaggaccaaacttcaacaaagtttcggag |
| | gttgaaggtgacaaacagttggcagaggcttatgagtccatcgctaagattgtcaacgataacaattacaagtcagtagcga |
| | ttccactgttgtccaccggcatcttttccgggaacaaagatcgactaacccaatcattgaaccatttgctgacagctttaga |
| | caccactgatgcagatgtagccatatactgcagggacaagaaatgggaaatgactctcaaggaagcagtggctaggagagaa |
| | gcagtggaggagatatgcatatccgacgactcttcagtgacagaacctgatgcagagctggtgagggtgcatccgaagagtt |
| | ctttggctggaaggaagggctacagcacaagcgatggcaaaactttctcatatttggaagggaccaagtttcaccaggcggc |
| | caaggatatagcagaaattaatgccatgtggcccgttgcaacggaggccaatgagcaggtatgcatgtatatcctcggagaa |
| | agcatgagcagtattaggtcgaaatgccccgtcgaagagtcggaagcctccacaccacctagcacgctgccttgcttgtgca |
| | tccatgccatgactccagaaagagtacagcgcctaaaagcctcacgtccagaacaaattactgtgtgctcatcctttccatt |
| | gccgaagtatagaatcactggtgtgcagaagatccaatgctcccagcctatattgttctcaccgaaagtgcctgcgtatatt |
| | catccaaggaagtatctcgtggaaacaccaccggtagacgagactccggagccatcggcagagaaccaatccacagagggga |
| | cacctgaacaaccaccacttataaccgaggatgagaccaggactagaacgcctgagccgatcatcatcgaagaggaagaaga |
| | ggatagcataagtttgctgtcagatggcccgacccaccaggtgctgcaagtcgaggcagacattcacgggccgccctctgta |
| | tctagctcatcctggtccattcctcatgcatccgactttgatgtggacagtttatccatacttgacaccctggagggagcta |
| | gcgtgaccagcggggcaacgtcagccgagactaactcttacttcgcaaagagtatggagtttctggcgcgaccggtgcctgc |
| | gcctcgaacagtattcaggaaccctccacatcccgctccgcgcacaagaacaccgtcacttgcacccagcagggcctgctcg |
| | agaaccagcctagtttccaccccgccaggcgtgaatagggtgatcactagagaggagctcgaggcgcttaccccgtcacgca |
| | ctcctagcaggtcggtctcgagaaccagcctggtctccaacccgccaggcgtaaatagggtgattacaagagaggagtttga |
| | ggcgttcgtagcacaacaacaatgacggtttgatgcgggtgcatacatcttttcctccgacaccggtcaagggcatttacaa |
| | caaaaatcagtaaggcaaacggtgctatccgaagtggtgttggagaggaccgaattggagatttcgtatgccccgcgcctcg |
| | accaagaaaaagaagaattactacgcaagaaattacagttaaatcccacacctgctaacagaagcagataccagtccaggaa |
| | ggtggagaacatgaaagccataacagctagacgtattctgcaaggcctagggcattatttgaaggcagaaggaaaagtggag |
| | tgctaccgaaccctgcatcctgttcctttgtattcatctagtgtgaaccgtgccttttcaagccccaaggtcgcagtggaag |
| | cctgtaacgccatgttgaaagagaactttccgactgtggcttcttactgtattattccagagtacgatgcctatttggacat |
| | ggttgacggagcttcatgctgcttagacactgccagtttttgccctgcaaagctgcgcagctttccaaagaaacactcctat |
| | ttggaacccacaatacgatcggcagtgccttcagcgatccagaacacgctccagaacgtcctggcagctgccacaaaaagaa |
| | attgcaatgtcacgcaaatgagagaattgcccgtattggattcggcggcctttaatgtggaatgcttcaagaaatatgcgtg |
| | taataatgaatattgggaaacgtttaaagaaaaccccatcaggcttactgaagaaaacgtggtaaattacattaccaaatta |
| | aaaggaccaaaagctgctgctctttttgcgaagacacataatttgaatatgttgcaggacataccaatggacaggtttgtaa |
| | tggacttaaagagagacgtgaaagtgactccaggaacaaaacatactgaagaacggcccaaggtacaggtgatccaggctgc |
| | cgatccgctagcaacagcgtatctgtgcggaatccaccgagagctggttaggagattaaatgcggtcctgcttccgaacatt |
| | catacactgtttgatatgtcggctgaagactttgacgctattatagccgagcacttccagcctggggattgtgttctggaaa |
| | ctgacatcgcgtcgtttgataaaagtgaggacgacgccatggctctgaccgcgttaatgattctggaagacttaggtgtgga |
| | cgcagagctgttgacgctgattgaggcggctttcggcgaaatttcatcaatacatttgcccactaaaactaaatttaaattc |
| | ggagccatgatgaaatctggaatgttcctcacactgtttgtgaacacagtcattaacattgtaatcgcaagcagagtgttga |
| | gagaacggctaaccggatcaccatgtgcagcattcattggagatgacaatatcgtgaaaggagtcaaatcggacaaattaat |
| | ggcagacaggtgcgccacctggttgaatatggaagtcaagattatagatgctgtggtgggcgagaaagcgccttatttctgt |
| | ggagggtttattttgtgtgactccgtgaccggcacagcgtgccgtgtggcagaccccctaaaaaggctgtttaagcttggca |
| | aacctctggcagcagacgatgaacatgatgatgacaggagaagggcattgcatgaagagtcaacacgctggaaccgagtggg |
| | tattctttcagagctgtgcaaggcagtagaatcaaggtatgaaaccgtaggaacttccatcatagttatggccatgactact |
| | ctagctagcagtgttaaatcattcagctacctgagaggggcccctataactctctacggctaacctgaatggactacgacat |
| | agtctagtccgccaagtaaggcgcgcccacccagcggccgcatacagcagcaattggcaagctgcttacatagaactcgcgg |
| | cgattggcatgccgccttaaaatttttattttatttttcttttcttttccgaatcggattttgtttttaatatttcaaaaaa |
| | aaaaaaaaaaaaaaaaaaaaaaaaaaaaaaaaaaagaagagctagggataacagggtaattgagcaaaaggccagcaaaagg |
| | ccaggaaccgtaaaaaggccgcgttgctggcgtttttccataggctccgcccccctgacgagcatcacaaaaatcgacgctc |
| | aagtcagaggtggcgaaacccgacaggactataaagataccaggcgtttccccctggaagctccctcgtgcgctctcctgtt |
| | ccgaccctgccgcttaccggatacctgtccgcctttctcccttcgggaagcgtggcgctttctcatagctcacgctgtaggt |
| | atctcagttcggtgtaggtcgttcgctccaagctgggctgtgtgcacgaaccccccgttcagcccgaccgctgcgccttatc |
| | cggtaactatcgtcttgagtccaacccggtaagacacgacttatcgccactggcagcagccactggtaacaggattagcaga |
| | gcgaggtatgtaggcggtgctacagagttcttgaagtggtggcctaactacggctacactagaagaacagtatttggtatct |
| | gcgctctgctgaagccagttaccttcggaaaaagagttggtagctcttgatccggcaaacaaaccaccgctggtagcggtgg |
| | tttttttgtttgcaagcagcagattacgcgcagaaaaaaaggatctcaagaagatcctttgatcttttctacggggtctgac |
| | gctcagtggaacgaaaactcacgttaagggattttggtcatgagattatcaaaaaggatcttcacctagatccttttaaatt |
| | aaaaatgaagttttaaatcaatctaaagtatatatgagtaaacttggtctgacagttagaaaaactcatcgagcatcaaatg |
| | aaactgcaatttattcatatcaggattatcaataccatatttttgaaaaagccgtttctgtaatgaaggagaaaactcaccg |
| | aggcagttccataggatggcaagatcctggtatcggtctgcgattccgactcgtccaacatcaatacaacctattaatttcc |
| | cctcgtcaaaaataaggttatcaagtgagaaatcaccatgagtgacgactgaatccggtgagaatggcaaaagtttatgcat |
| | ttctttccagacttgttcaacaggccagccattacgctcgtcatcaaaatcactcgcatcaaccaaaccgttattcattcgt |
| | gattgcgcctgagcgagacgaaatacgcgatcgctgttaaaaggacaattacaaacaggaatcgaatgcaaccggcgcagga |
| | acactgccagcgcatcaacaatattttcacctgaatcaggatattcttctaatacctggaatgctgttttcccagggatcgc |
| | agtggtgagtaaccatgcatcatcaggagtacggataaaatgcttgatggtcggaagaggcataaattccgtcagccagttt |
| | agtctgaccatctcatctgtaacatcattggcaacgctacctttgccatgtttcagaaacaactctggcgcatcgggcttcc |
| | catacaatcgatagattgtcgcacctgattgcccgacattatcgcgagcccatttatacccatataaatcagcatccatgtt |
| | ggaatttaatcgcggcctagagcaagacgtttcccgttgaatatggctcatactcttcctttttcaatattattgaagcatt |
| | tatcagggttattgtctcatgagcggatacatatttgaatgtatttagaaaaataaacaaataggggttccgcgcacatttc |
| | cccgaaaagtgccacctgacgtctaagaaaccattattatcatgacattaacctataaaaataggcgtatcacgaggccctt |
| | tcgtctagggataacagggtaattaatacgactcactatag |
| SEQ ID NO: 3 | MGTVNKPVVGVLMGFGIITGTLRITNPVRASVLRYDDFHIDEDKLDTNSVYEPYYHSDHAESSWVNRGESSRKAYDHNSPYI |
| (VZV glyco- | WPRNDYDGFLENAHEHHGVYNQGRGIDSGERLMQPTQMSAQEDLGDDTGIHVIPTLNGDDRHKIVNVDQRQYGDVFKGDLNP |
| protein E | KPQGQRLIEVSVEENHPFTLRAPIQRIYGVRYTETWSFLPSLTCTGDAAPAIQHICLKHTTCFQDVVVDVDCAENTKEDQLA |
| antigen | EISYRFQGKKEADQPWIVVNTSTLFDELELDPPEIEPGVLKVLRTEKQYLGVYIWNMRGSDGTSTYATFLVTWKGDEKTRNP |
| sequence) | TPAVTPQPRGAEFHMWNYHSHVFSVGDTFSLAMHLQYKIHEAPFDLLLEWLYVPIDPTCQPMRLYSTCLYHPNAPQCLSHMN |
| | SGCTFTSPHLAQRVASTVYQNCEHADNYTAYCLGISHMEPSFGLILHDGGTTLKFVDTPESLSGLYVFVVYFNGHVEAVAYT |
| | VVSTVDHFVNAIEERGFPPTAGQPPATTKPKEITPVNPGTSPLIRYAAWTGGLAAVVLLCLVIFLICTAKRMRVKAARVDK* |
| SEQ ID NO: 4 | ataggcggcgcatgagagaagcccagaccaattacctacccaaaatggagaaagttcacgttgacatcgaggaagacagccc |
| (VZV srRNA | attcctcagagctttgcagcggagcttcccgcagtttgaggtagaagccaagcaggtcactgataatgaccatgctaatgcc |
| sequence) | agagcgttttcgcatctggcttcaaaactgatcgaaacggaggtggacccatccgacacgatccttgacattggaagtgcgc |
| | ccgcccgcagaatgtattctaagcacaagtatcattgtatctgtccgatgagatgtgcggaagatccggacagattgtataa |
| | gtatgcaactaagctgaagaaaaactgtaaggaaataactgataaggaattggacaagaaaatgaaggagctcgccgccgtc |
| | atgagcgaccctgacctggaaactgagactatgtgcctccacgacgacgagtcgtgtcgctacgaagggcaagtcgctgttt |
| | accaggatgtatacgcggttgacggaccgacaagtctctatcaccaagccaataagggagttagagtcgcctactggatagg |
| | ctttgacaccaccccttttatgtttaagaacttggctggagcatatccatcatactctaccaactgggccgacgaaaccgtg |
| | ttaacggctcgtaacataggcctatgcagctctgacgttatggagcggtcacgtagagggatgtccattcttagaaagaagt |
| | atttgaaaccatccaacaatgttctattctctgttggctcgaccatctaccacgagaagagggacttactgaggagctggca |
| | cctgccgtctgtatttcacttacgtggcaagcaaaattacacatgtcggtgtgagactatagttagttgcgacgggtacgtc |
| | gttaaaagaatagctatcagtccaggcctgtatgggaagccttcaggctatgctgctacgatgcaccgcgagggattcttgt |
| | gctgcaaagtgacagacacattgaacggggagagggtctcttttcccgtgtgcacgtatgtgccagctacattgtgtgacca |
| | aatgactggcatactggcaacagatgtcagtgcggacgacgcgcaaaaactgctggttgggctcaaccagcgtatagtcgtc |
| | aacggtcgcacccagagaaacaccaataccatgaaaaattaccttttgcccgtagtggcccaggcatttgctaggtgggcaa |
| | aggaatataaggaagatcaagaagatgaaaggccactaggactacgagatagacagttagtcatggggtgttgttgggcttt |
| | tagaaggcacaagataacatctatttataagcgcccggatacccaaaccatcatcaaagtgaacagcgatttccactcattc |
| | gtgctgcccaggataggcagtaacacattggagatcgggctgagaacaagaatcaggaaaatgttagaggagcacaaggagc |
| | cgtcacctctcattaccgccgaggacgtacaagaagctaagtgcgcagccgatgaggctaaggaggtgcgtgaagccgagga |
| | gttgcgcgcagctctaccacctttggcagctgatgttgaggagcccactctggaagccgatgtcgacttgatgttacaagag |
| | gctggggccggctcagtggagacacctcgtggcttgataaaggttaccagctacgatggcgaggacaagatcggctcttacg |
| | ctgtgctttctccgcaggctgtactcaagagtgaaaaattatcttgcatccaccctctcgctgaacaagtcatagtgataac |
| | acactctggccgaaaagggcgttatgccgtggaaccataccatggtaaagtagtggtgccagagggacatgcaatacccgtc |
| | caggactttcaagctctgagtgaaagtgccaccattgtgtacaacgaacgtgagttcgtaaacaggtacctgcaccatattg |
| | ccacacatggaggagcgctgaacactgatgaagaatattacaaaactgtcaagcccagcgagcacgacggcgaatacctgta |
| | cgacatcgacaggaaacagtgcgtcaagaaagaactagtcactgggctagggctcacaggcgagctggtggatcctcccttc |
| | catgaattcgcctacgagagtctgagaacacgaccagccgctccttaccaagtaccaaccataggggtgtatggcgtgccag |
| | gatcaggcaagtctggcatcattaaaagcgcagtcaccaaaaaagatctagtggtgagcgccaagaaagaaaactgtgcaga |
| | aattataagggacgtcaagaaaatgaaagggctggacgtcaatgccagaactgtggactcagtgctcttgaatggatgcaaa |
| | caccccgtagagaccctgtatattgacgaagcttttgcttgtcatgcaggtactctcagagcgctcatagccattataagac |
| | ctaaaaaggcagtgctctgcggggatcccaaacagtgcggtttttttaacatgatgtgcctgaaagtgcattttaaccacga |
| | gatttgcacacaagtcttccacaaaagcatctctcgccgttgcactaaatctgtgacttcggtcgtctcaaccttgttttac |
| | gacaaaaaaatgagaacgacgaatccgaaagagactaagattgtgattgacactaccggcagtaccaaacctaagcaggacg |
| | atctcattctcacttgtttcagagggtgggtgaagcagttgcaaatagattacaaaggcaacgaaataatgacggcagctgc |
| | ctctcaagggctgacccgtaaaggtgtgtatgccgttcggtacaaggtgaatgaaaatcctctgtacgcacccacctcagaa |
| | catgtgaacgtcctactgacccgcacggaggaccgcatcgtgtggaaaacactagccggcgacccatggataaaaacactga |
| | ctgccaagtaccctgggaatttcactgccacgatagaggagtggcaagcagagcatgatgccatcatgaggcacatcttgga |
| | gagaccggaccctaccgacgtcttccagaataaggcaaacgtgtgttgggccaaggctttagtgccggtgctgaagaccgct |
| | ggcatagacatgaccactgaacaatggaacactgtggattattttgaaacggacaaagctcactcagcagagatagtattga |
| | accaactatgcgtgaggttctttggactcgatctggactccggtctattttctgcacccactgttccgttatccattaggaa |
| | taatcactgggataactccccgtcgcctaacatgtacgggctgaataaagaagtggtccgtcagctctctcgcaggtaccca |
| | caactgcctcgggcagttgccactggaagagtctatgacatgaacactggtacactgcgcaattatgatccgcgcataaacc |
| | tagtacctgtaaacagaagactgcctcatgctttagtcctccaccataatgaacacccacagagtgacttttcttcattcgt |
| | cagcaaattgaagggcagaactgtcctggtggtcggggaaaagttgtccgtcccaggcaaaatggttgactggttgtcagac |
| | cggcctgaggctaccttcagagctcggctggatttaggcatcccaggtgatgtgcccaaatatgacataatatttgttaatg |
| | tgaggaccccatataaataccatcactatcagcagtgtgaagaccatgccattaagcttagcatgttgaccaagaaagcttg |
| | tctgcatctgaatcccggcggaacctgtgtcagcataggttatggttacgctgacagggccagcgaaagcatcattggtgct |
| | atagcgcggcagttcaagttttcccgggtatgcaaaccgaaatcctcacttgaagagacggaagttctgtttgtattcattg |
| | ggtacgatcgcaaggcccgtacgcacaatccttacaagctttcatcaaccttgaccaacatttatacaggttccagactcca |
| | cgaagccggatgtgcaccctcatatcatgtggtgcgaggggatattgccacggccaccgaaggagtgattataaatgctgct |
| | aacagcaaaggacaacctggcggaggggtgtgcggagcgctgtataagaaattcccggaaagcttcgatttacagccgatcg |
| | aagtaggaaaagcgcgactggtcaaaggtgcagctaaacatatcattcatgccgtaggaccaaacttcaacaaagtttcgga |
| | ggttgaaggtgacaaacagttggcagaggcttatgagtccatcgctaagattgtcaacgataacaattacaagtcagtagcg |
| | attccactgttgtccaccggcatcttttccgggaacaaagatcgactaacccaatcattgaaccatttgctgacagctttag |
| | acaccactgatgcagatgtagccatatactgcagggacaagaaatgggaaatgactctcaaggaagcagtggctaggagaga |
| | agcagtggaggagatatgcatatccgacgactcttcagtgacagaacctgatgcagagctggtgagggtgcatccgaagagt |
| | tctttggctggaaggaagggctacagcacaagcgatggcaaaactttctcatatttggaagggaccaagtttcaccaggcgg |
| | ccaaggatatagcagaaattaatgccatgtggcccgttgcaacggaggccaatgagcaggtatgcatgtatatcctcggaga |
| | aagcatgagcagtattaggtcgaaatgccccgtcgaagagtcggaagcctccacaccacctagcacgctgccttgcttgtgc |
| | atccatgccatgactccagaaagagtacagcgcctaaaagcctcacgtccagaacaaattactgtgtgctcatcctttccat |
| | tgccgaagtatagaatcactggtgtgcagaagatccaatgctcccagcctatattgttctcaccgaaagtgcctgcgtatat |
| | tcatccaaggaagtatctcgtggaaacaccaccggtagacgagactccggagccatcggcagagaaccaatccacagagggg |
| | acacctgaacaaccaccacttataaccgaggatgagaccaggactagaacgcctgagccgatcatcatcgaagaggaagaag |
| | aggatagcataagtttgctgtcagatggcccgacccaccaggtgctgcaagtcgaggcagacattcacgggccgccctctgt |
| | atctagctcatcctggtccattcctcatgcatccgactttgatgtggacagtttatccatacttgacaccctggagggagct |
| | agcgtgaccagcggggcaacgtcagccgagactaactcttacttcgcaaagagtatggagtttctggcgcgaccggtgcctg |
| | cgcctcgaacagtattcaggaaccctccacatcccgctccgcgcacaagaacaccgtcacttgcacccagcagggcctgctc |
| | gagaaccagcctagtttccaccccgccaggcgtgaatagggtgatcactagagaggagctcgaggcgcttaccccgtcacgc |
| | actcctagcaggtcggtctcgagaaccagcctggtctccaacccgccaggcgtaaatagggtgattacaagagaggagtttg |
| | aggcgttcgtagcacaacaacaatgacggtttgatgcgggtgcatacatcttttcctccgacaccggtcaagggcatttaca |
| | acaaaaatcagtaaggcaaacggtgctatccgaagtggtgttggagaggaccgaattggagatttcgtatgccccgcgcctc |
| | gaccaagaaaaagaagaattactacgcaagaaattacagttaaatcccacacctgctaacagaagcagataccagtccagga |
| | aggtggagaacatgaaagccataacagctagacgtattctgcaaggcctagggcattatttgaaggcagaaggaaaagtgga |
| | gtgctaccgaaccctgcatcctgttcctttgtattcatctagtgtgaaccgtgccttttcaagccccaaggtcgcagtggaa |
| | gcctgtaacgccatgttgaaagagaactttccgactgtggcttcttactgtattattccagagtacgatgcctatttggaca |
| | tggttgacggagcttcatgctgcttagacactgccagtttttgccctgcaaagctgcgcagctttccaaagaaacactccta |
| | tttggaacccacaatacgatcggcagtgccttcagcgatccagaacacgctccagaacgtcctggcagctgccacaaaaaga |
| | aattgcaatgtcacgcaaatgagagaattgcccgtattggattcggcggcctttaatgtggaatgcttcaagaaatatgcgt |
| | gtaataatgaatattgggaaacgtttaaagaaaaccccatcaggcttactgaagaaaacgtggtaaattacattaccaaatt |
| | aaaaggaccaaaagctgctgctctttttgcgaagacacataatttgaatatgttgcaggacataccaatggacaggtttgta |
| | atggacttaaagagagacgtgaaagtgactccaggaacaaaacatactgaagaacggcccaaggtacaggtgatccaggctg |
| | ccgatccgctagcaacagcgtatctgtgcggaatccaccgagagctggttaggagattaaatgcggtcctgcttccgaacat |
| | tcatacactgtttgatatgtcggctgaagactttgacgctattatagccgagcacttccagcctggggattgtgttctggaa |
| | actgacatcgcgtcgtttgataaaagtgaggacgacgccatggctctgaccgcgttaatgattctggaagacttaggtgtgg |
| | acgcagagctgttgacgctgattgaggcggctttcggcgaaatttcatcaatacatttgcccactaaaactaaatttaaatt |
| | cggagccatgatgaaatctggaatgttcctcacactgtttgtgaacacagtcattaacattgtaatcgcaagcagagtgttg |
| | agagaacggctaaccggatcaccatgtgcagcattcattggagatgacaatatcgtgaaaggagtcaaatcggacaaattaa |
| | tggcagacaggtgcgccacctggttgaatatggaagtcaagattatagatgctgtggtgggcgagaaagcgccttatttctg |
| | tggagggtttattttgtgtgactccgtgaccggcacagcgtgccgtgtggcagaccccctaaaaaggctgtttaagcttggc |
| | aaacctctggcagcagacgatgaacatgatgatgacaggagaagggcattgcatgaagagtcaacacgctggaaccgagtgg |
| | gtattctttcagagctgtgcaaggcagtagaatcaaggtatgaaaccgtaggaacttccatcatagttatggccatgactac |
| | tctagctagcagtgttaaatcattcagctacctgagaggggcccctataactctctacggctaacctgaatggactacgaca |
| | tagtctagtccgccaagatgggcaccgtgaacaagcccgtggtgggcgtgctgatgggcttcggcatcatcaccggcaccct |
| | gagaatcaccaaccccgtgagagctagcgtgctgagatacgacgacttccacatcgacgaggacaagctggacaccaacagc |
| | gtgtacgagccctactaccacagcgaccacgccgagagcagctgggtgaacagaggcgagagcagcagaaaggcctacgacc |
| | acaacagcccctacatctggcctagaaacgactacgacggcttcctggagaacgcccacgagcaccacggcgtgtacaacca |
| | aggcagaggcatcgacagcggcgagagactgatgcagcccacacagatgagcgcccaagaggacctgggcgacgacaccggc |
| | atccacgtgatccccaccctgaacggcgacgacagacacaagatcgtgaacgtggatcagagacagtacggcgacgtgttca |
| | agggcgacctgaaccccaagccccaagggcagagactgatcgaggtgagcgtggaggagaaccaccccttcaccctgagagc |
| | ccccattcagagaatctacggcgtgagatacaccgagacctggagcttcctgcctagcctgacctgcaccggcgacgccgcc |
| | cccgccattcagcacatctgcctgaagcacaccacctgcttccaagacgtggtggtggacgtggactgcgccgagaacacca |
| | aggaggatcagctggccgagatcagctacagattccaaggcaagaaggaggccgatcagccctggatcgtggtgaacacaag |
| | caccctgttcgacgagctggagctggacccccccgagatcgagcccggcgtgctgaaggtgctgagaaccgagaagcagtac |
| | ctgggcgtgtacatctggaacatgagaggcagcgacggcacaagcacctacgccaccttcctggtgacctggaagggcgacg |
| | agaagacaagaaaccccacccccgcggtgacccctcagcctagaggcgccgagttccacatgtggaactaccacagccacgt |
| | gttcagcgtgggcgacaccttcagcctggccatgcacctgcagtacaagatccacgaggcccccttcgacctgctgctggag |
| | tggctgtacgtgcccatcgaccccacctgtcagcccatgagactgtacagcacctgcctgtaccaccccaacgctcctcagt |
| | gcctgagccacatgaacagcggctgcaccttcacaagcccccacctggctcagagagtggctagcaccgtgtatcagaactg |
| | cgagcacgccgacaactacaccgcctactgcctgggcatcagccacatggagcctagcttcggcctgatcctgcacgacggc |
| | ggcaccaccctgaagttcgtggacacccccgagagcctgagcggcctgtacgtgttcgtggtgtacttcaacggccacgtgg |
| | aggccgtggcctacaccgtggtgagcaccgtggaccacttcgtgaatgccatcgaggagagaggcttcccccccaccgccgg |
| | ccaacccccggccaccaccaagcccaaggagatcacccccgtgaatcccggcacaagccctcttatcagatacgcagcctgg |
| | accggaggcctggccgccgtggtgctgctgtgcctggtgatcttcctgatctgcaccgccaagagaatgagagtgaaggccg |
| | ctagagtggacaagtgataaggcgcgcccacccagcggccgcatacagcagcaattggcaagctgcttacatagaactcgcg |
| | gcgattggcatgccgccttaaaatttttattttatttttcttttcttttccgaatcggattttgtttttaatatttcaaaaa |
| | aaaaaaaaaaaaaaaaaaa |
| SEQ ID NO: 5 | GGGGAAUAAACUAGUAUUCUUCUGGUCCCCACAGACUCAGAGAGAACCCGCCACCAUGGCCUUCGUGUUCCUGGUGCUGCUG |
| S-2p RNA | CCCCUGGUGAGCAGCCAGUGCGUGAACCUGAGGACCAGAACCCAGCUGCCCCCCGCCUACACCAACAGCUUCACCAGAGGCG |
| Sequence | UGUACUACCCCGACAAGGUGUUCAGAAGCAGCGUGCUGCACAGCACCCAGGACCUGUUCCUGCCCUUCUUCAGCAACGUGAC |
| | CUGGUUCCACGCCAUCCACGUGAGCGGCACCAACGGCACCAAGAGAUUCGACAACCCCGUGCUGCCCUUCAACGACGGCGUG |
| | UACUUCGCCAGCACCGAGAAGUCCAACAUCAUCAGAGGCUGGAUCUUCGGCACCACCCUGGACAGCAAGACCCAGAGCCUGC |
| | UGAUCGUGAACAACGCCACCAACGUGGUGAUCAAGGUGUGCGAGUUCCAGUUCUGCAACGACCCCUUCCUGGACGUGUACUA |
| | CCACAAGAACAACAAGAGCUGGAUGGAGAGCGGCGUGUACAGCAGCGCCAACAACUGCACCUUCGAGUACGUGAGCCAGCCC |
| | UUCCUGAUGGACCUGGAGGGCAAGCAGGGCAACUUCAAGAACCUGAGAGAGUUCGUGUUCAAGAACAUCGACGGCUACUUCA |
| | AGAUCUACAGCAAGCACACCCCCAUCAACCUGGUGAGAGACCUGCCCCAGGGCUUCAGCGCCCUGGAGCCCCUGGUGGACCU |
| | GCCCAUCGGCAUCAACAUCACCAGAUUCCAGACCCUGCUGGCCCUGCACAGAAGCUACCUGACCCCCGGCGACAGCAGCAGC |
| | GGCUGGACCGCCGGCGCCGCCGCCUACUACGUGGGCUACCUGCAGCCCAGAACCUUCCUGCUGAAGUACAACGAGAACGGCA |
| | CCAUCACCGACGCCGUGGACUGCGCCCUGGACCCCCUGAGCGAGACCAAGUGCACCCUGAAAUCCUUCACCGUGGAGAAGGG |
| | CAUCUACCAGACCAGCAACUUCAGAGUGCAGCCCACCGAGAGCAUCGUGAGAUUCCCCAACAUCACCAACCUGUGCCCCUUC |
| | GGCGAGGUGUUCAACGCCACCAGAUUCGCCAGCGUGUACGCCUGGAACAGAAAGAGAAUCAGCAACUGCGUGGCCGACUACA |
| | GCGUCCUGUACAACAGCGCCAGCUUCAGCACCUUCAAGUGCUACGGCGUGAGCCCCACCAAGCUGAACGACCUGUGCUUCAC |
| | CAACGUGUACGCCGACAGCUUCGUGAUCAGAGGCGACGAGGUGAGACAGAUCGCCCCCGGCCAGACCGGCAAGAUCGCCGAC |
| | UACAACUACAAGCUGCCCGACGACUUCACCGGCUGCGUGAUCGCCUGGAACAGCAACAACCUGGACAGCAAGGUGGGCGGCA |
| | ACUACAACUACAGAUACAGACUGUUCAGAAAGAGCAACCUGAAGCCCUUCGAGAGAGACAUCAGCACCGAGAUCUACCAGGC |
| | CGGCAGCAAGCCCUGCAACGGCGUGGAGGGCUUCAACUGCUACUUCCCCCUGCAGAGCUACGGCUUCCAGCCCACCAACGGC |
| | GUGGGCUACCAGCCCUACAGAGUGGUGGUGCUGAGCUUCGAGCUGCUGCACGCCCCCGCCACCGUGUGCGGCCCCAAAAAGA |
| | GCACCAACCUGGUGAAGAACAAGUGCGUGAACUUCAACUUCAACGGCCUGACCGGCACCGGCGUGCUGACCGAGAGCAACAA |
| | GAAGUUCCUGCCCUUCCAGCAGUUCGGCAGAGACAUCGCCGACACCACAGACGCUGUGAGAGACCCCCAGACCCUGGAGAUC |
| | CUGGACAUCACCCCCUGCAGCUUCGGCGGCGUGAGCGUGAUCACCCCCGGCACCAACACCAGCAACCAGGUGGCCGUGCUGU |
| | ACCAGGGAGUGAACUGCACCGAGGUGCCCGUGGCCAUCCACGCCGACCAGCUGACCCCCACCUGGAGAGUGUACAGCACCGG |
| | CAGCAACGUGUUCCAGACCAGAGCCGGCUGCCUGAUCGGCGCCGAGCACGUGAACAACAGCUACGAGUGCGACAUCCCCA |
| SEQ ID NO: 6 | TATGTTACGTGCAAAGGTGATTGTCACCCCCCGAAAGACCATATTGTGACACACCCTCAGTATCACGCCCAAACATTTACAG |
| 3′ UTR | CCGCGGTGTCAAAAACCGCGTGGACGTGGTTAACATCCCTGCTGGGAGGATCAGCCGTAATTATTATAATTGGCTTGGTGCT |
| | GGCTACTATTGTGGCCATGTACGTGCTGACCAACCAGAAACATAATTGAATACAGCAGCAATTGGCAAGCTGCTTACATAGA |
| | ACTCGCGGCGATTGGCATGCCGCCTTAAAATTTTTATTTTATTTTTCTTTTCTTTTCCGAATCGGATTTTGTTTTTAATATT |
| | TC |

### CITATIONS

1. Moffat J, Mo C, Cheng J J, Sommer M, Zerboni L, Stamatis S, Arvin A M. Functions of the C-terminal domain of varicella-zoster virus glycoprotein E in viral replication in vitro and skin and T-cell tropism in vivo. J Virol. 2004 November; 78(22):12406-15. doi: 10.1128/sJVL.78.22.12406-12415.2004. PMID: 15507627; PMCID: PMC525039.

1. A composition or set of compositions separately comprising each of (1) a lyophilized polynucleotide composition and (2) a liquid lipid nanoparticle (LNP) solution,
wherein the LNP comprises an ionizable lipid of
(i) Formula II: 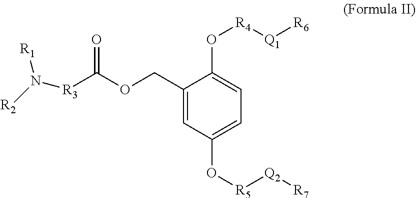
wherein:
R₁ and R₂ are each independently C₁ to C₆ alkyl;
R₃ is C₁ to C₅ alkyl;
R₄ and R₅ are each independently C₁ to C₁₈ alkyl group
Q₁ and Q₂ are each independently —O—C(O)—, —C(O)—O—, —O—C(S)—, —C(S)—O—; —S—S—, and
R₆ and R₇ are each independently C₁ to C₃₂ alkyl; or
(ii) Formula I 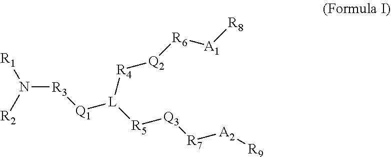
wherein:
R₁ and R₂ are each, independently C₁-C₆ alkyl;
R₃ is C₁-C₅ alkyl;
Q₁, Q₂ and Q₃ are each independently —O—, —S—, —C(O)O—, —OC(O)—, —S—S—, —C(O)S—, —SC(O)—, —OC(S)—, or —C(S)O—;
L is C₁-C₃ alkyl;
R₄ and R₅ are each, independently C₁-C₁₀ alkyl;
R₆ and R₇ are each, independently C₁-C₁₀ alkyl, C₁-C₁₀ alkenyl;
A₁ and A₂ are each independently a bond, —O—, —S—, —C(O)O—, —OC(O)—, —S—S—, —C(O)S—, —SC(O)—, —OC(S)—, or —C(S)O—; and
R₈ and R₉ are each, independently C₁-C₃₀ alkyl.

2.-4. (canceled)

5. The composition of claim 1, wherein the ionizable lipid is:

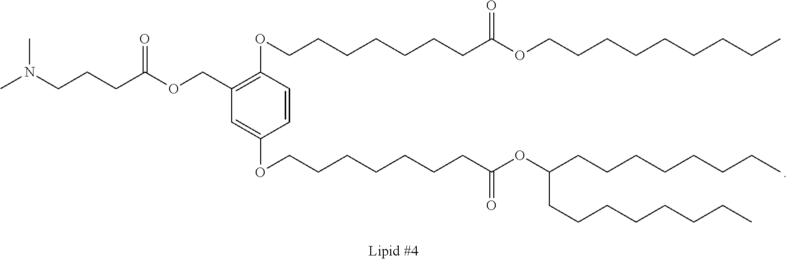 [SMILES: CCCCCCCCCOC(=O)CCCCCCCOc1ccc(OCCCCCCCC(=O)OC(CCCCCCCC)CCCCCCCC)cc1COC(=O)CCCN(C)C]

6. The composition of claim 1, wherein the lyophilized polynucleotide composition comprises a self-replicating RNA or encodes a protein, a polypeptide, or an antigen.

7.-18. (canceled)

19. The composition of claim 1, wherein the ionizable lipid is selected form the group consisting of:

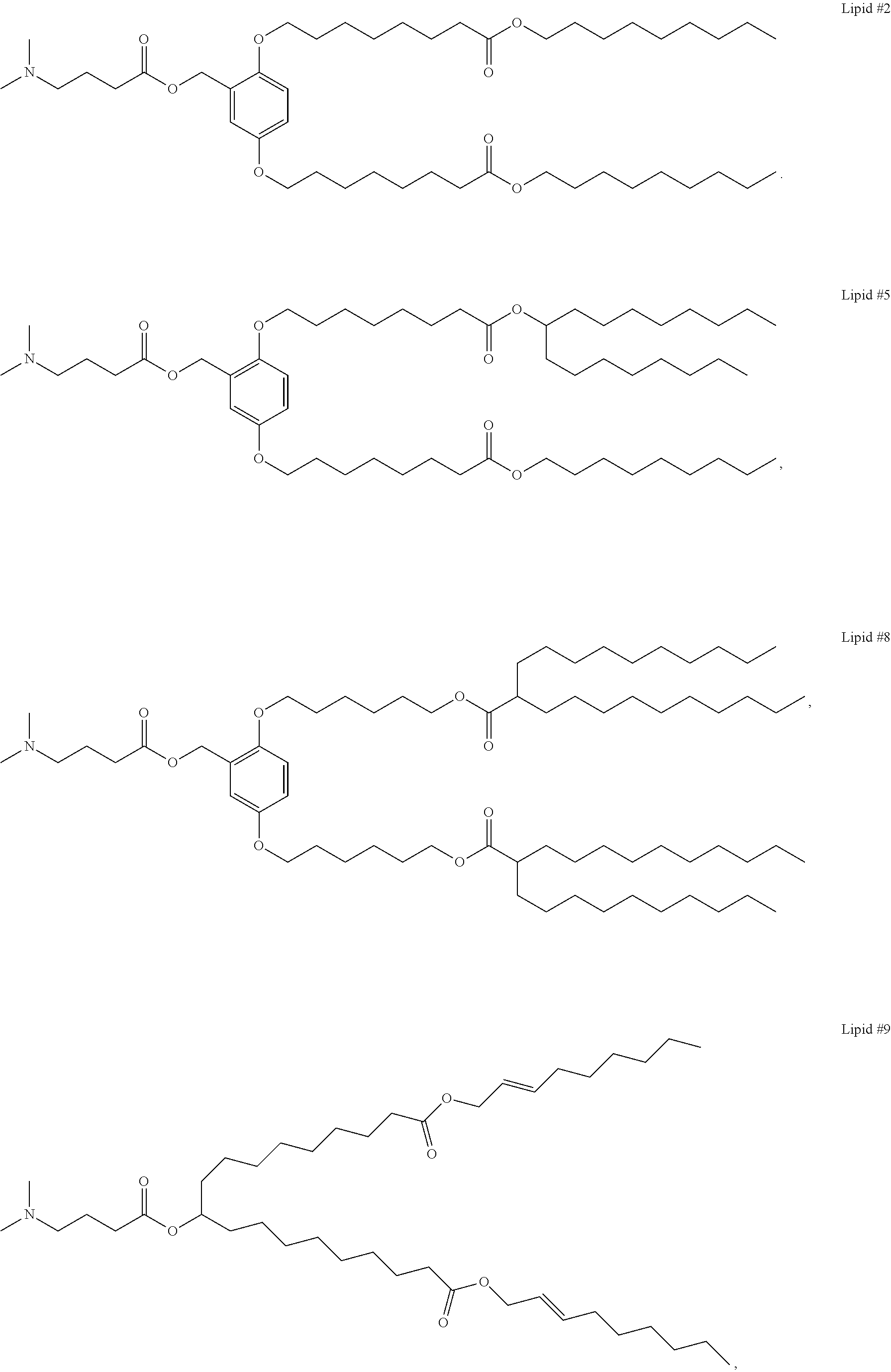

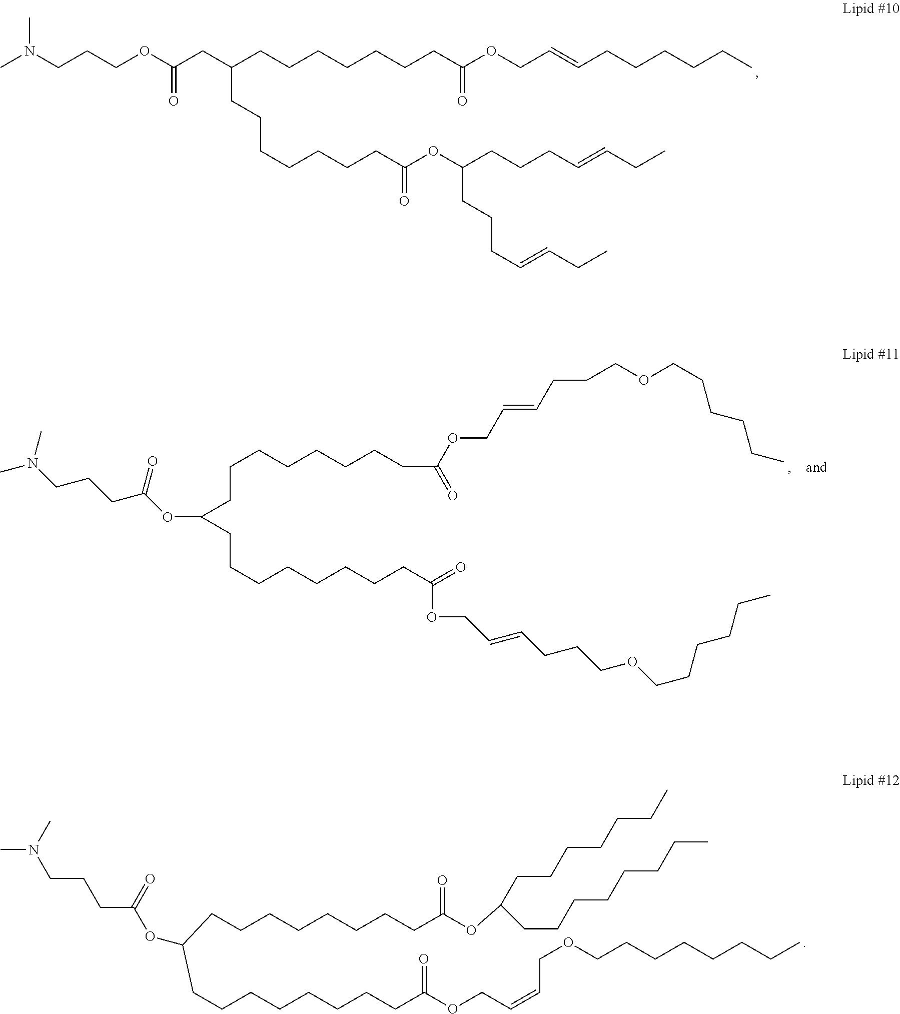

20.-29. (canceled)

30. A self-replicating RNA (srRNA) vector comprising in 5′ to 3′ order:
a) a Cap1 cap;
b) a 5′ UTR;
c) one or more structural genes;
d) a gene of interest (GOI);
e) a 3′ UTR comprising about 100 to about 400 nucleotides; and
f) a poly A tail comprising about 30 to about 100 nucleotides.

31. The srRNA vector of claim 30, wherein the 3′ UTR comprises a nucleic acid sequence having least about 80% identity to the nucleic acid sequence SEQ ID NO: 6.

32.-33. (canceled)

34. The srRNA vector of claim 30, wherein the GOI is a varicella-zoster virus (VZV) antigen, and wherein the VZV antigen comprises a VZV glycoprotein E (gE) antigen.

35.-46. (canceled)

47. The srRNA vector of claim 34, wherein the sequence of the VZV antigen comprises SEQ ID NO: 2 or SEQ ID NO: 4.

48.-52. (canceled)

53. The srRNA vector of claim 34, wherein the srRNA comprises a Cap1 cap, a VZV glycoprotein E (gE) antigen, a 3′ UTR comprising about 270 nucleotides in length, and a polyA tail comprising about 65 nucleotides in length.

54.-62. (canceled)

63. A method of preventing or treating shingles or a VZV-related disease in a subject, comprising administering the composition of claim 1 or the srRNA vector of claim 30 to a subject.

64.-71. (canceled)

72. A liquid composition comprising a lyophilized RNA electrostatically adsorbed to at least one exterior portion of or encapsulated in a lipid nanoparticle (LNP) comprising an ionizable lipid, with an ionizable lipid:RNA (N/P) molar ratio ranging from 5:1 to 12:1,
wherein the LNP comprises an ionizable lipid of
(i) Formula I: 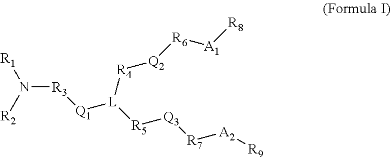
wherein:
R₁ and R₂ are each, independently C₁-C₆ alkyl;
R₃ is C₁-C₅ alkyl;
Q₁, Q₂ and Q₃ are each independently —O—, —S—, —C(O)O—, —OC(O)—, —S—S—, —C(O)S—, —SC(O)—, —OC(S)—, or —C(S)O—;
L is C₁-C₃ alkyl;
R₄ and R₅ are each, independently C₁-C₁₀ alkyl;
R₆ and R₇ are each, independently C₁-C₁₀ alkyl, C₁-C₁₀ alkenyl;
A₁ and A₂ are each independently a bond, —O—, —S—, —C(O)O—, —OC(O)—, —S—S—, —C(O)S—, —SC(O)—, —OC(S)—, or —C(S)O—; and
R₈ and R₉ are each, independently C₁-C₃₀ alkyl; or
(ii) Formula II 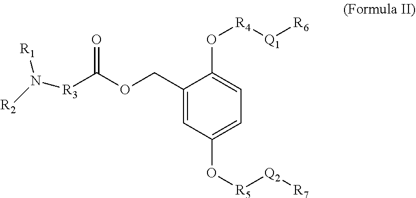
wherein:
R₁ and R₂ are each independently C₁ to C₆ alkyl;
R₃ is C₁ to C₅ alkyl;
R₄ and R₅ are each independently C₁ to C₁₈ alkyl group
Q₁ and Q₂ are each independently —O—C(O)—, —C(O)—O—, —O—C(S)—, —C(S)—O—; —S—S—, and
R₆ and R₇ are each independently C₁ to C₃₂ alkyl.

73.-76. (canceled)

76. The liquid composition of claim 72, wherein the ionizable lipid is selected from the group consisting of

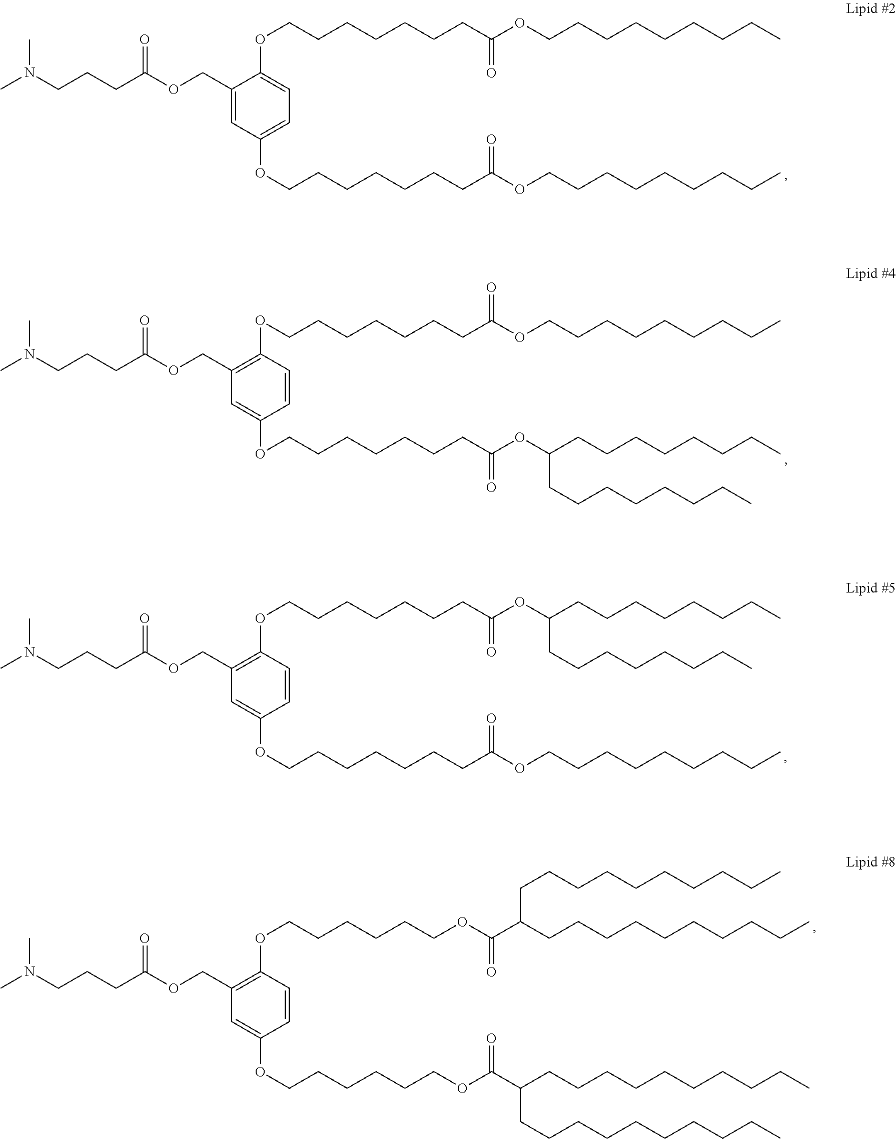

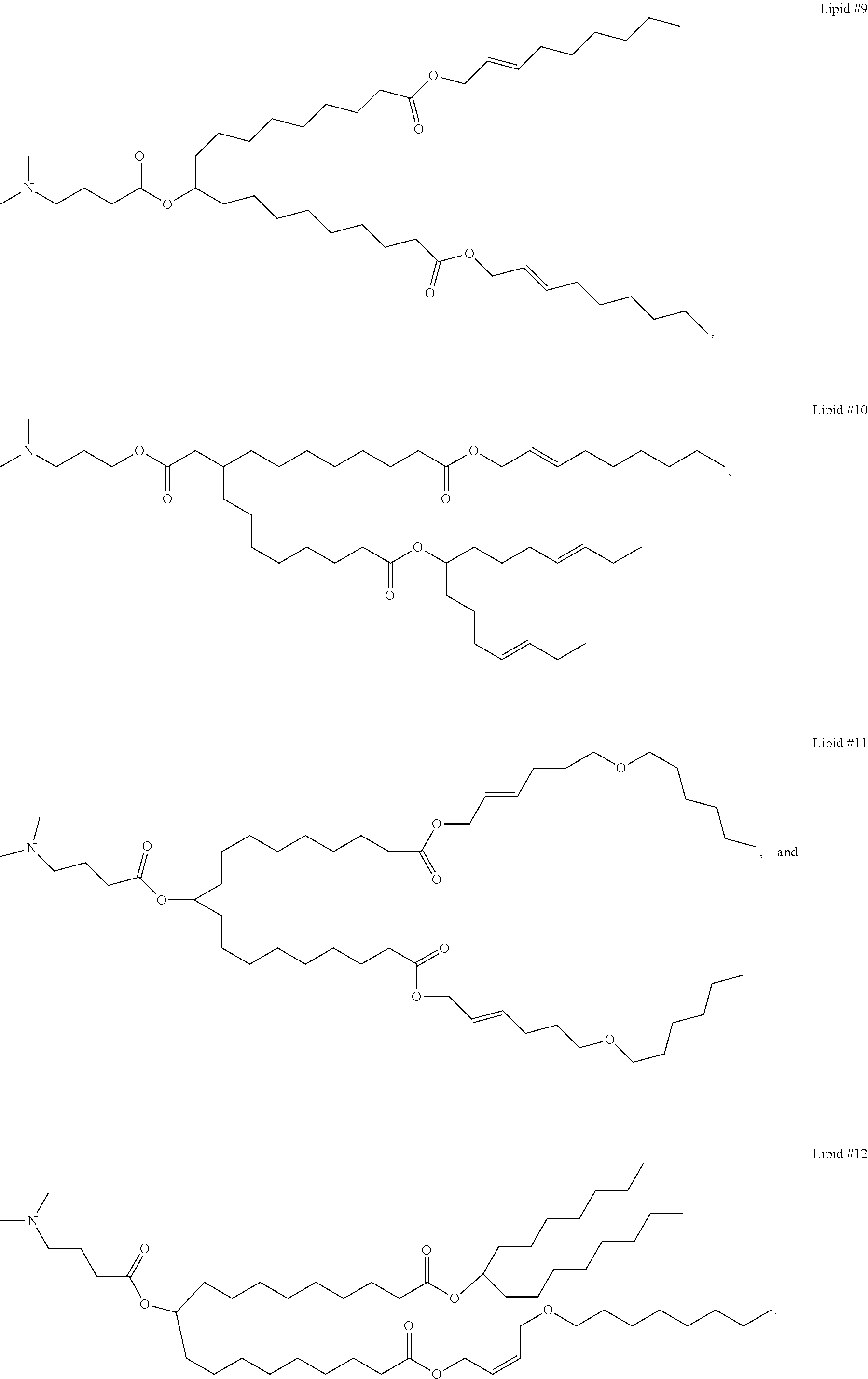

77.-85. (canceled)

86. A method of making the liquid composition of claim 72, comprising mixing the lyophilized RNA and the LNP, wherein the LNP is in a liquid solution and is added to the lyophilized RNA.

87.-92. (canceled)

93. A method of treating a disease or disorder comprising administering the composition of claim 72 to a subject.

94.-111. (canceled)

112. A lipid or a lipid nanoparticle (LNP) comprising an ionizable lipid of
(i) Formula I: 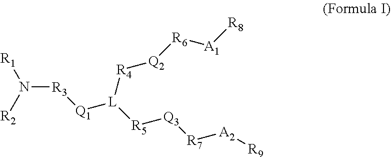
wherein:
R₁ and R₂ are each, independently C₁-C₆ alkyl;
R₃ is C₁-C₅ alkyl;
Q₁, Q₂ and Q₃ are each independently —O—, —S—, —C(O)O—, —OC(O)—, —S—S—, —C(O)S—, —SC(O)—, —OC(S)—, or —C(S)O—;
L is C₁-C₃ alkyl;
R₄ and R₅ are each, independently C₁-C₁₀ alkyl;
R₆ and R₇ are each, independently C₁-C₁₀ alkyl, C₁-C₁₀ alkenyl;
A₁ and A₂ are each independently a bond, —O—, —S—, —C(O)O—, —OC(O)—, —S—S—, —C(O)S—, —SC(O)—, —OC(S)—, or —C(S)O—; and
R₈ and R₉ are each, independently C₁-C₃₀ alkyl;
(ii) Formula II: 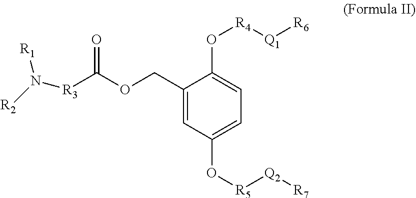
or a pharmaceutically acceptable salt thereof, wherein:
R₁ and R₂ are each, independently, C₁-C₆ alkyl;
R₃ is C₁-C₅ alkyl;
R₄ and R₅ are each independently C₁-C₁₈ alkyl;
Q₁ and Q₂ are each independently —O—C(O)—, —C(O)—O—, —O—C(S)—, —C(S)—O—; —S—S—,
R₆ and R₇ are each independently a C₁₈-C₃₂ alkyl; or
(iii) selected from the group consisting of 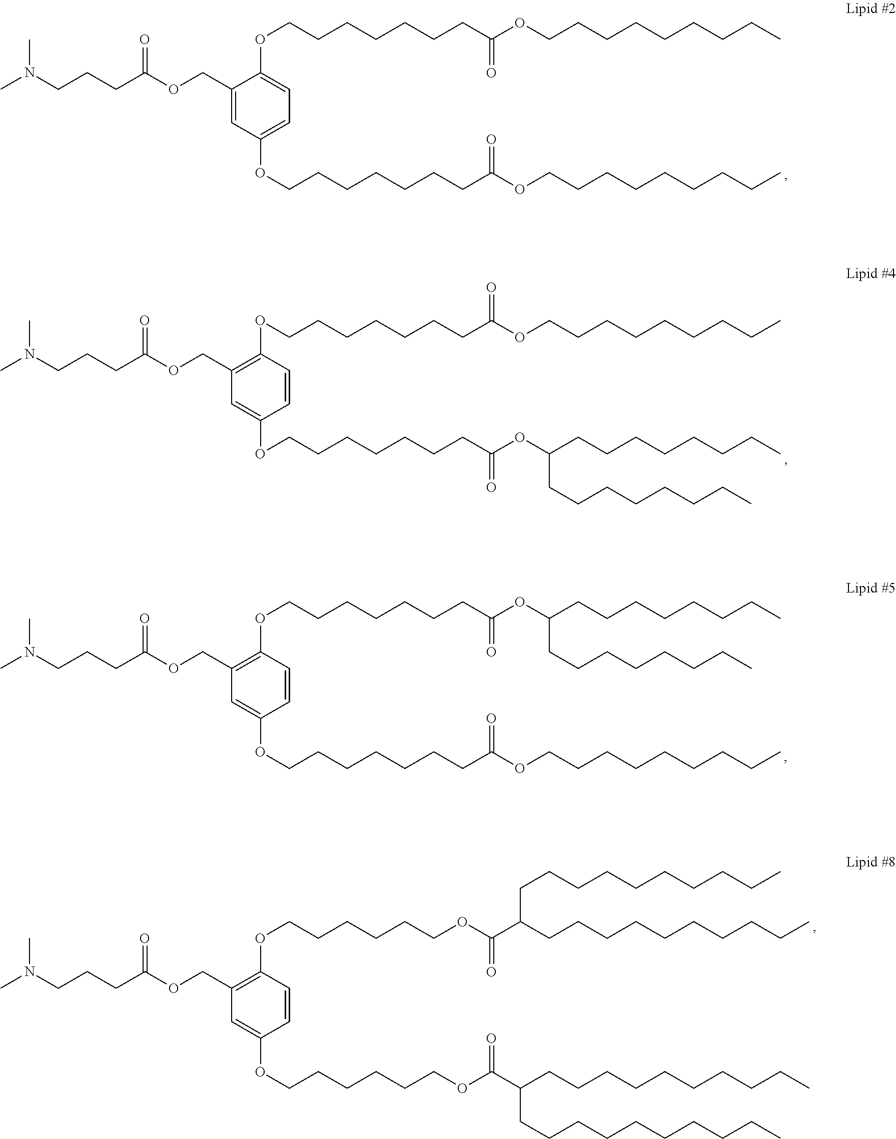 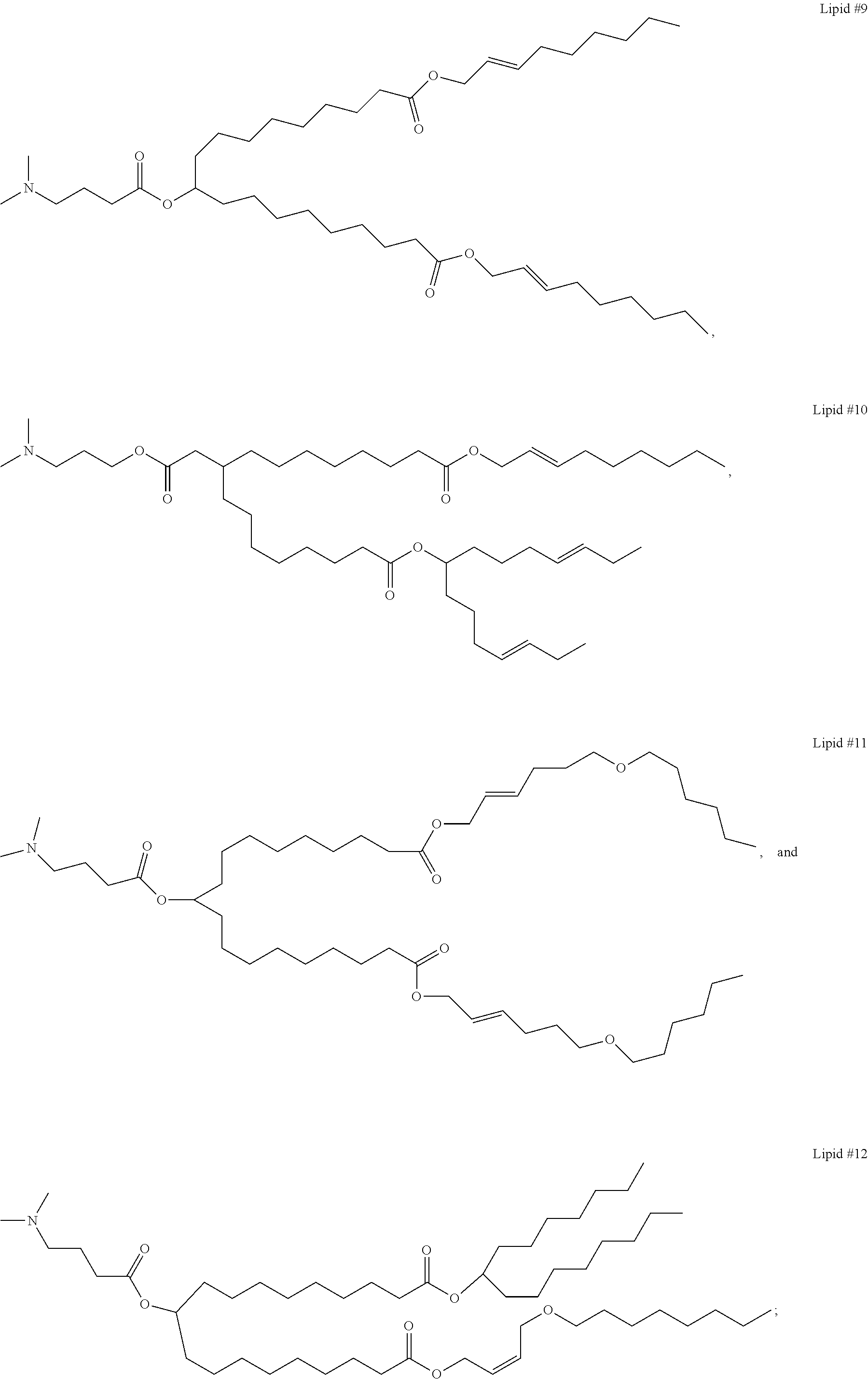
wherein the LNP further comprises a phospholipid, a structural lipid, and a pegylated lipid.

113.-116. (canceled)

117. The lipid or LNP of claim 112, wherein the LNP comprises the phospholipid:thestructurallipid:thepegylatedlipid:and the ionizable lipid in a mole ratio within the ranges of phospholipid: 5%-20%, structural lipid: 18.5%-58.5%, pegylated lipid: 1%-4%, ionizable lipid: 30%-70%, wherein the total summation of the mole ratio of the lipids is 100%.

118-122. (canceled)

123. The composition of claim 6, wherein the lyophilized polynucleotide comprises a polyA tail 30-100 nucleotides in length.

124. The composition of claim 6, wherein the lyophilized polynucleotide comprises a cap structure selected from the group consisting of m7G (Cap 0), m7GpppNm-(Cap 1), N6,2′-O-dimethyladenosine (m6AM), m7G(5′)ppp(5′)G (mCAP), and anti-reverse cap analogs (ARCA), wherein Nm denotes any nucleotide with a 2′ O methylation.

125. The composition of claim 6, wherein the lyophilized polynucleotide comprises a nucleotide sequence encoding a varicella-zoster virus (VZV) antigen.

126. The composition of claim 6, wherein the lyophilized polynucleotide comprises:
(i) comprises a Cap1 cap, a VZV glycoprotein E (gE) antigen, a 3′ UTR comprising about 270 nucleotides in length, and a polyA tail comprising about 65 nucleotides in length; or
(ii) a Cap1 cap, SARS-CoV-2 spike protein (RBD), a 3′ UTR comprising about 270 nucleotides in length, and a polyA tail comprising about 65 nucleotides in length.


SEQUENCE LISTING (6 sequences)

SEQ ID NO: 1 - DNA, 1722 bp, synthetic construct
  source 1..1722: mol_type = other DNA
atgggcaccgtgaacaagcccgtggtgggcgtgctgatgggcttcggcatcatcaccggcaccctgagaatcaccaaccccgtgagagctagcgtgctgagatacgacgacttccacatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgagacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcggtgacccctcagcctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgctcctcagtgcctgagccacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcacgacggcggcaccaccctgaagttcgtggacacccccgagagcctgagcggcctgtacgtgttcgtggtgtacttcaacggccacgtggaggccgtggcctacaccgtggtgagcaccgtggaccacttcgtgaatgccatcgaggagagaggcttcccccccaccgccggccaacccccggccaccaccaagcccaaggagatcacccccgtgaatcccggcacaagccctcttatcagatacgcagcctggaccggaggcctggccgccgtggtgctgctgtgcctggtgatcttcctgatctgcaccgccaagagaatgagagtgaaggccgctagagtggacaagtga

SEQ ID NO: 2 - DNA, 9636 bp, synthetic construct
  source 1..9636: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtaaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaagaagagctagggataacagggtaattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctagggataacagggtaattaatacgactcactatag

SEQ ID NO: 3 - protein, 573 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAARVDK

SEQ ID NO: 4 - DNA, 9466 bp, synthetic construct
  source 1..9466: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggcaccgtgaacaagcccgtggtgggcgtgctgatgggcttcggcatcatcaccggcaccctgagaatcaccaaccccgtgagagctagcgtgctgagatacgacgacttccacatcgacgaggacaagctggacaccaacagcgtgtacgagccctactaccacagcgaccacgccgagagcagctgggtgaacagaggcgagagcagcagaaaggcctacgaccacaacagcccctacatctggcctagaaacgactacgacggcttcctggagaacgcccacgagcaccacggcgtgtacaaccaaggcagaggcatcgacagcggcgagagactgatgcagcccacacagatgagcgcccaagaggacctgggcgacgacaccggcatccacgtgatccccaccctgaacggcgacgacagacacaagatcgtgaacgtggatcagagacagtacggcgacgtgttcaagggcgacctgaaccccaagccccaagggcagagactgatcgaggtgagcgtggaggagaaccaccccttcaccctgagagcccccattcagagaatctacggcgtgagatacaccgagacctggagcttcctgcctagcctgacctgcaccggcgacgccgcccccgccattcagcacatctgcctgaagcacaccacctgcttccaagacgtggtggtggacgtggactgcgccgagaacaccaaggaggatcagctggccgagatcagctacagattccaaggcaagaaggaggccgatcagccctggatcgtggtgaacacaagcaccctgttcgacgagctggagctggacccccccgagatcgagcccggcgtgctgaaggtgctgagaaccgagaagcagtacctgggcgtgtacatctggaacatgagaggcagcgacggcacaagcacctacgccaccttcctggtgacctggaagggcgacgagaagacaagaaaccccacccccgcggtgacccctcagcctagaggcgccgagttccacatgtggaactaccacagccacgtgttcagcgtgggcgacaccttcagcctggccatgcacctgcagtacaagatccacgaggcccccttcgacctgctgctggagtggctgtacgtgcccatcgaccccacctgtcagcccatgagactgtacagcacctgcctgtaccaccccaacgctcctcagtgcctgagccacatgaacagcggctgcaccttcacaagcccccacctggctcagagagtggctagcaccgtgtatcagaactgcgagcacgccgacaactacaccgcctactgcctgggcatcagccacatggagcctagcttcggcctgatcctgcacgacggcggcaccaccctgaagttcgtggacacccccgagagcctgagcggcctgtacgtgttcgtggtgtacttcaacggccacgtggaggccgtggcctacaccgtggtgagcaccgtggaccacttcgtgaatgccatcgaggagagaggcttcccccccaccgccggccaacccccggccaccaccaagcccaaggagatcacccccgtgaatcccggcacaagccctcttatcagatacgcagcctggaccggaggcctggccgccgtggtgctgctgtgcctggtgatcttcctgatctgcaccgccaagagaatgagagtgaaggccgctagagtggacaagtgataaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaa

SEQ ID NO: 5 - RNA, 2048 nt, synthetic construct
  source 1..2048: mol_type = other RNA
ggggaataaactagtattcttctggtccccacagactcagagagaacccgccaccatggccttcgtgttcctggtgctgctgcccctggtgagcagccagtgcgtgaacctgaggaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagtccaacatcatcagaggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaaatccttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtcctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacagatacagactgttcagaaagagcaacctgaagcccttcgagagagacatcagcaccgagatctaccaggccggcagcaagccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaaaaagagcaccaacctggtgaagaacaagtgcgtgaacttcaacttcaacggcctgaccggcaccggcgtgctgaccgagagcaacaagaagttcctgcccttccagcagttcggcagagacatcgccgacaccacagacgctgtgagagacccccagaccctggagatcctggacatcaccccctgcagcttcggcggcgtgagcgtgatcacccccggcaccaacaccagcaaccaggtggccgtgctgtaccagggagtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatcccca

SEQ ID NO: 6 - DNA, 330 bp, synthetic construct
  source 1..330: mol_type = other DNA
tatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttc